(19) United States
(12) Patent Application Publication
(10) Pub. No.: US 2025/0041399 A1
(43) Pub. Date: Feb. 6, 2025

(54) CMV-BASED HUMAN PAPILLOMAVIRUS VACCINES

(71) Applicant: VIR BIOTECHNOLOGY, INC., San Francisco, CA (US)

(72) Inventors: Ann M. ARVIN, San Francisco, CA (US); Eric BRUENING, Damascus, OR (US); Emily MARSHALL, Portland, OR (US); Leah B. SORIAGA, San Diego, CA (US)

(21) Appl. No.: 18/792,060

(22) Filed: Aug. 1, 2024

Related U.S. Application Data
(60) Provisional application No. 63/574,128, filed on Apr. 3, 2024.
(60) Provisional application No. 63/517,310, filed on Aug. 2, 2023.

(51) Int. Cl. A61K 39/12; A61K 39/00; A61P 31/20; C07K 14/005; C12N 7/00; C12N 15/86

(52) U.S. Cl. CPC A61K 39/12; A61P 31/20; C07K 14/005; C12N 7/00; C12N 15/86; A61K 2039/54; A61K 2039/572; C12N 2710/16143; C12N 2710/20022; C12N 2710/20034

(57) ABSTRACT

The disclosure relates to human papillomavirus (HPV) antigens and vectors for delivering the antigens. The disclosure also relates to immunogenic compositions comprising the same, and their uses.

### STATEMENT OF GOVERNMENT INTEREST

This invention was made with government support under R44 CA180177 awarded by the National Institutes of Health. The government has certain rights in the invention.

### REFERENCE TO AN ELECTRONIC SEQUENCE LISTING

This application contains a Sequence Listing which has been submitted electronically in xml format and is hereby incorporated by reference in its entirety. Said xml copy, created on Jul. 29, 2024, is named SeqList-368561-44501.xml and is 274,237 bytes in size.

### BACKGROUND

Viral infections are a significant cause of disease and death worldwide. Cervical cancer is caused by infection with certain types of human papillomavirus (HPV). Two HPV types (16 and 18) cause 70% of cervical cancers and precancerous cervical lesions. HPV is also linked to cancers of the head and neck (oropharynx), anus, vulva, vagina, skin, rectum, and penis. According to the World Health Organization, approximately 342,000 women died from cervical cancer in 2020. Worldwide, cervical cancer is the fourth most frequent cancer in women, with an estimated 604,000 new cases in 2020.

HPV is a member of the Papillomaviridae, a family of DNA viruses collectively known as papillomaviruses. Papillomaviruses replicate in the basal layer of the body surface tissues. Papillomaviruses are non-enveloped, meaning that the outer shell or capsid of the virus is not covered by a lipid membrane. A single viral protein, known as L1, forms a 55-60 nanometer capsid. Like most non-enveloped viruses, the capsid is geometrically regular and presents icosahedral symmetry.

HPV infects anogenital and oral mucosa and persists in local basal epithelium. HPV is a non-enveloped deoxyribonucleic acid (DNA) virus, with a circular genome of double-stranded DNA about 8,000 base pairs in length encoding six early proteins (E1, E2, E4, E5, E6, and E7) and two late proteins (L1 and L2). It is packaged within the L1 shell along with cellular histone proteins, which package the genomic viral DNA. HPV viral DNA exists in both episomal and integrated forms. The HPV genes E6 and E7 are involved in malignant conversion and inhibit the tumor suppressors p53 and RB. The HPV gene E1 is a helicase that unwinds the viral origin and recruits host cellular factors to replicate the viral genome. The HPV gene E2 is a transcriptional regulator that helps recruit the E1 helicase to the origin and also plays a role in genome partitioning.

HPV vaccines based on the L1 coat protein must be administered prior to exposure to the virus, and do not treat HPV infection or HPV-associated disease such as cancer. Additionally, there are no approved inhibitors of E6 or E7.

There is no known cure for an HPV infection, and current vaccines do not treat existing infections. There remains a need for therapeutic approaches to preventing and treating HPV infection.

### BRIEF SUMMARY

Disclosed herein are fusion proteins comprising HPV antigens and nucleic acids encoding the fusion proteins. In some embodiments, the present disclosure provides a fusion protein comprising one or more of E7, E6, E1, and E2, or portions or fragments thereof. In some embodiments, the present disclosure provides vectors encoding a fusion protein as described herein.

In some embodiments, the present disclosure provides a fusion protein comprising or consisting of: (a) an amino acid sequence having 90%, 91%, 92%, 93%, 94%, 95%, 96%, 97%, 98%, 99%, or 100% identity to the amino acid sequence according to SEQ ID NO:4; (b) the amino acid sequence according to SEQ ID NO:4; (c) an amino acid sequence having 90%, 91%, 92%, 93%, 94%, 95%, 96%, 97%, 98%, 99%, or 100% identity to the amino acid sequence according to SEQ ID NO: 1; (d) the amino acid sequence according to SEQ ID NO: 1; (e) an amino acid sequence having 90%, 91%, 92%, 93%, 94%, 95%, 96%, 97%, 98%, 99%, or 100% identity to the amino acid sequence according to SEQ ID NO:2; (f) the amino acid sequence according to SEQ ID NO:2; (g) an amino acid sequence having 90%, 91%, 92%, 93%, 94%, 95%, 96%, 97%, 98%, 99%, or 100% identity to the amino acid sequence according to SEQ ID NO:3; (h) the amino acid sequence according to SEQ ID NO:3; (i) (i) an amino acid sequence having 90%, 91%, 92%, 93%, 94%, 95%, 96%, 97%, 98%, 99%, or 100% identity to the amino acid sequence according to SEQ ID NO: 1; (ii) an amino acid sequence having 90%, 91%, 92%, 93%, 94%, 95%, 96%, 97%, 98%, 99%, or 100% identity to the amino acid sequence according to SEQ ID NO: 2; and (iii) an amino acid sequence having 90%, 91%, 92%, 93%, 94%, 95%, 96%, 97%, 98%, 99%, or 100% identity to the amino acid sequence according to SEQ ID NO: 3; (j) (i) the amino acid sequence according to SEQ ID NO:1; (ii) the amino acid sequence according to SEQ ID NO:2; and (iii) the amino acid sequence according to SEQ ID NO:3; (k) (i) an amino acid sequence having 90%, 91%, 92%, 93%, 94%, 95%, 96%, 97%, 98%, 99%, or 100% identity to the amino acid sequence according to SEQ ID NO: 1; and (ii) an amino acid sequence having 90%, 91%, 92%, 93%, 94%, 95%, 96%, 97%, 98%, 99%, or 100% identity to the amino acid sequence according to SEQ ID NO: 2; (1) (i) the amino acid sequence according to SEQ ID NO:1; and (ii) the amino acid sequence according to SEQ ID NO:2; (m) (i) an amino acid sequence having 90%, 91%, 92%, 93%, 94%, 95%, 96%, 97%, 98%, 99%, or 100% identity to the amino acid sequence according to SEQ ID NO: 1; and (ii) an amino acid sequence having 90%, 91%, 92%, 93%, 94%, 95%, 96%, 97%, 98%, 99%, or 100% identity to the amino acid sequence according to SEQ ID NO: 3; or (n) (i) the amino acid sequence according to SEQ ID NO: 1; and (ii) the amino acid sequence according to SEQ ID NO:3.

In some embodiments, the present disclosure provides a fusion protein encoded by a nucleic acid comprising a sequence having 90%, 91%, 92%, 93%, 94%, 95%, 96%, 97%, 98%, 99%, or 100% identity to the nucleic acid sequence according to any one of SEQ ID NOs:16, 13, 14, and 15.

In some embodiments, the present disclosure provides a nucleic acid molecule encoding a fusion protein described herein.

In some embodiments, the present disclosure provides a vector comprising a nucleic acid molecule encoding a fusion protein described herein. In some embodiments, the vector is a viral vector, e.g., a cytomegalovirus (CMV) vector, a RhCMV vector, a HCMV vector, or a recombinant HCMV vector.

In some embodiments, the present disclosure provides a vector comprising a nucleic acid sequence having at least 90%, 91%, 92%, 93%, 94%, 95%, 96%, 97%, 98%, 99%, or 100% identity to the nucleic acid sequence according to SEQ ID NO:17.

In some embodiments, the present disclosure provides a recombinant HCMV vector comprising a TR3 backbone and a nucleic acid sequence encoding a heterologous antigen according to SEQ ID NO: 4, wherein: (a) the vector does not express UL128 or UL130, or orthologs thereof; (b) the vector comprises a nucleic acid sequence encoding UL146 and UL147, or orthologs thereof; and (c) the nucleic acid encoding a heterologous antigen replaces all or part of UL78 and is operably linked to the UL78 promoter.

Also provided herein, in certain aspects, are pharmaceutical compositions and methods of using fusion proteins, nucleic acid molecules, vectors, and compositions as described herein.

### BRIEF DESCRIPTION OF THE DRAWINGS

FIG. 1 shows CD8+ T cell response in rhesus macaques administered RhCMV (rhesus cytomegalovirus) vectors expressing a human papillomavirus (HPV) HPV16/18 E6/E7 antigen. Rhesus macaques were administered either 68-1 RhCMV (n=4) or 68-1.2 RhCMV (n=4) expressing the antigen, each of which elicits either an MHC-II/E or MHC-I response, respectively. The frequency of HPV18 E6-specific CD8+ T cells in the CD8+ T cell memory population was evaluated weekly.

FIG. 2 shows CD8+ T cell responses in rhesus macaques administered HCMV (human cytomegalovirus) vectors expressing an HPV16/18 E6/E7 antigen. Rhesus macaques were administered either a HCMV vector expressing active (TR3-Δpp71-HPV Δ128-130 Δ146-147 ΔUS11) or inactive (VMID5 dUL146-147 dUS11 dUL82-HPV E6/E7) UL128 and UL130 genes, each of which elicits either an MHC-II/E or MHC-I response, respectively. The frequency of HPV18 E6-specific CD8+ T cells in the CD8+ T cell memory population was evaluated every two weeks.

FIG. 3 shows sequence components of a HPV16/18 E7/E6 fusion protein. The wild-type E7 and E6 sequences of each of HPV16 and HPV18 were modified to disrupt sequences required for oncogenic cell transformation. Lowercase “g” indicates glycines replacing conserved residues in the Rb-binding motif (shown in bold, italicized font) and replacing cysteines in order to disrupt zinc-finger domains (shown in bold font). The N-terminal p53, E6/AP binding domains, and C-terminal PDZ motifs (shown in dashed underlined font) were deleted.

FIG. 4 shows amino acid conservation of HPV E1 helicase. Data are shown for 29 HPV types and 136 representative species.

FIG. 5 shows amino acid conservation of HPV E1 helicase in HPV types associated with cervical cancer. Data are shown for the alpha(α) 9 species (types HPV16 and HPV31) and the alpha(α) 7 species (types HPV18 and HPV45). The rectangle indicates a contiguous segment from amino acid positions 310-610.

FIG. 6 shows amino acid conservation of HPV E1 helicase from amino acid positions 310-410 in four HPV types associated with cervical cancer (HPV16, HPV31, HPV18, and HPV45).

FIG. 7 shows amino acid conservation of HPV E1 helicase from amino acid positions 310-410 in two HPV types associated with cervical cancer (HPV16 and HPV31, alpha(α) 9 species).

FIG. 8 shows amino acid conservation of HPV E1 helicase from amino acid positions 317-417 in two HPV types associated with cervical cancer (HPV18 and HPV45, alpha(α) 7 species).

FIG. 9 shows amino acid conservation of HPV E2 helicase in HPV types associated with cervical cancer. Data are shown for the alpha(α) 9 species (types HPV16 and HPV31) and the alpha(α) 7 species (types HPV18 and HPV45). Rectangles indicate conservation within the N- and C-terminal domains.

FIG. 10 shows amino acid conservation from amino acid positions 1-101 of the HPV E2 helicase in two HPV types associated with cervical cancer (HPV16 and HPV31, alpha(α) 9 species).

FIG. 11 shows amino acid conservation from amino acid positions 1-101 of the HPV E2 helicase in two HPV types associated with cervical cancer (HPV18 and HPV45, alpha(α) 7 species).

FIGS. 12A-12B show selection of conserved sequence regions of HPV E1 helicase (FIG. 12A, amino acids 310-610) and HPV E2 helicase (FIG. 12B, amino acids 1-200) used for the construction of a HPV16/18 E7/E6 E1/E2 fusion protein.

FIG. 13 shows the structure and sequence of a HPV18 E7/E6-E16 E7/E6-HPV16 E1-HPV18 E1-HPV16 E2-HPV18 E2 fusion protein.

FIG. 14 shows the mean count of predicted MHC-I-restricted epitopes for 9mer peptides across the HPV16/18 E7/E6 E1/E2 fusion protein sequence as predicted by MetMHCpan 4.1 (the Immune Epitope Database (IEDB), <https.//services.healthtech.dtu.dk/services/NetMHCpan-4.1/>).

FIG. 15 shows immunogenicity of the HPV16/18 E7/E6 E1/E2 fusion protein when expressed in a CMV vector. Rhesus macaques (n=2) were administered a CMV vector expressing HPV16/18 E7/E6 E1/E2 fusion protein (CMV-HPV). PBMCs were isolated on day 35-42 post-inoculation and CD4+ and CD8+ T cell responses were detected by intracellular cytokine staining (ICS) of IFNγ and TNFα after stimulation with peptide pools corresponding to each component of the HPV fusion protein (HPV 16-E1, HPV16-E2, HPV16-E6, HPV16-E7, HPV 18-E1, HPV18-E2, HPV18-E6, and HPV18-E7).

### DETAILED DESCRIPTION

### I. Glossary

The following sections provide a detailed description of HPV antigens and related pharmaceutical compositions and methods of inducing an immune response, such as an anti-HPV response, and methods of treating or preventing HPV infections and HPV-associated cancers. Prior to setting forth this disclosure in more detail, it may be helpful to an understanding thereof to provide definitions of certain terms to be used herein. Additional definitions are set forth throughout this disclosure.

Unless the context requires otherwise, throughout the present specification and claims, the word “comprise” and variations thereof, such as “comprises” and “comprising,” are to be construed in an open, inclusive sense, that is, as “including, but not limited to”. “Consisting of” shall mean excluding more than trace elements of other ingredients and substantial method steps disclosed herein, and in the case of an amino acid or nucleic acid sequence, excluding additional amino acids or nucleotides, respectively. The term “consisting essentially of” limits the scope of a claim to the specified materials or steps, or to those that do not materially affect the basic characteristics of a claimed invention. For example, a composition consisting essentially of the elements as defined herein would not exclude trace contaminants from the isolation and purification method and pharmaceutically acceptable carriers, such as phosphate buffered saline, preservatives, and the like. Similarly, a protein consists essentially of a particular amino acid sequence when the protein includes additional amino acids that contribute to at most 20% of the length of the protein and do not substantially affect the activity of the protein (e.g., alters the activity of the protein by no more than 50%). Embodiments defined by each of the transitional terms are within the scope of this invention.

In the present description, the term “about” means+/−20% of the indicated range, value, or structure, unless otherwise indicated.

It should be understood that the terms “a” and “an” as used herein include “one or more” of the enumerated components unless stated otherwise. The use of the alternative (e.g., “or”) should be understood to mean either one, both, or any combination thereof of the alternatives, and may be used synonymously with “and/or”. As used herein, the terms “include” and “have” are used synonymously, which terms and variants thereof are intended to be construed as non-limiting.

The word “substantially” does not exclude “completely”; e.g., a composition which is “substantially free” from Y may be completely free from Y. Where necessary, the word “substantially” may be omitted from definitions provided herein.

As used herein, the terms “peptide”, “polypeptide”, and “protein” and variations of these terms refer to a molecule, in particular a peptide, oligopeptide, polypeptide, or protein including fusion protein, respectively, comprising at least two amino acids joined to each other by a normal peptide bond, or by a modified peptide bond, such as for example in the cases of isosteric peptides. For example, a peptide, polypeptide, or protein may be composed of amino acids selected from the 20 amino acids defined by the genetic code, linked to each other by a normal peptide bond (“classical” polypeptide). A peptide, polypeptide, or protein can be composed of L-amino acids and/or D-amino acids. In particular, the terms “peptide”, “polypeptide”, and “protein” also include “peptidomimetics,” which are defined as peptide analogs containing non-peptidic structural elements, which peptides are capable of mimicking or antagonizing the biological action(s) of a natural parent peptide. A peptidomimetic lacks classical peptide characteristics such as enzymatically scissile peptide bonds. In particular, a peptide, polypeptide, or protein may comprise amino acids other than the 20 amino acids defined by the genetic code in addition to these amino acids, or it can be composed of amino acids other than the 20 amino acids defined by the genetic code. In particular, a peptide, polypeptide, or protein in the context of the present disclosure can equally be composed of amino acids modified by natural processes, such as post-translational maturation processes or by chemical processes, which are well known to a person skilled in the art. Such modifications are fully detailed in the literature. These modifications can appear anywhere in the polypeptide: in the peptide skeleton, in the amino acid chain, or even at the carboxy- or amino-terminal ends. In particular, a peptide or polypeptide can be branched following an ubiquitination or be cyclic with or without branching. This type of modification can be the result of natural or synthetic post-translational processes that are well known to a person skilled in the art. The terms “peptide”, “polypeptide”, or “protein” in the context of the present disclosure in particular also include modified peptides, polypeptides, and proteins. For example, peptide, polypeptide, or protein modifications can include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation, or ubiquitination. Such modifications are fully detailed in the literature (Proteins Structure and Molecular Properties, 2^{nd} Ed., T. E. Creighton, New York (1993); Post-translational Covalent Modifications of Proteins, B. C. Johnson, Ed., Academic Press, New York (1983); Seifter, et al., Analysis for protein modifications and nonprotein cofactors, Meth. Enzymol. 182:626-46 (1990); and Rattan, et al., Protein Synthesis: Post-translational Modifications and Aging, Ann NY Acad Sci 663:48-62(1992)). Accordingly, the terms “peptide”, “polypeptide”, and “protein” include for example lipopeptides, lipoproteins, glycopeptides, glycoproteins, and the like.

“Orthologs” of proteins are typically characterized by possession of greater than 75% sequence identity counted over the full-length alignment with the amino acid sequence of specific protein using an alignment algorithm, for example, the ALIGN program (version 2.0) set to default parameters. Proteins with even greater similarity to a reference sequence will show increasing percentage identities when assessed by this method, such as at least 80%, at least 85%, at least 90%, at least 92%, at least 95%, or at least 98% sequence identity. In addition, sequence identity can be compared over the full length of particular domains of the disclosed peptides.

The term “homologous” or “homolog” refers to a molecule or activity found in or derived from a host cell, species, or strain. For example, a heterologous or exogenous molecule or gene encoding the molecule may be homologous to a native host or host cell molecule or gene that encodes the molecule, respectively, but may have an altered structure, sequence, expression level, or combinations thereof. For example, the human CMV genes UL78, UL82, UL128, UL130, and UL146/UL147 are homologous to rhesus CMV genes Rh107 (UL78), Rh110 (UL82), Rh157.5 (UL128), Rh157.4 (UL130), and Rh158-161 (UL146/UL147), respectively.

As used herein a “(poly)peptide” comprises a single chain of amino acid monomers linked by peptide bonds as explained above. A “protein”, as used herein, comprises one or more, e.g., 1, 2, 3, 4, 5, 6, 7, 8, 9, or 10 (poly)peptides, i.e., one or more chains of amino acid monomers linked by peptide bonds as explained above. In particular embodiments, a protein according to the present disclosure comprises 1, 2, 3, or 4 polypeptides.

As used herein, the terms “nucleic acid”, “nucleic acid molecule,” “nucleic acid sequence,” and “polynucleotide” are used interchangeably and are intended to include DNA molecules and RNA molecules, including, without limitation, messenger RNA (mRNA), DNA/RNA hybrids, or synthetic nucleic acids. The nucleic acid may be single-stranded, or partially or completely double stranded (duplex). Duplex nucleic acids may be homoduplex or heteroduplex. A nucleic acid molecule may be single-stranded or double-stranded.

As used herein, the term “coding sequence” is intended to refer to a polynucleotide molecule, which encodes the amino acid sequence of a protein product. The boundaries of the coding sequence are generally determined by an open reading frame, which usually begins with an ATG start codon.

The term “expression” as used herein refers to any step involved in the production of the polypeptide, including transcription, post-transcriptional modification, translation, post-translational modification, secretion, or the like.

As used herein, the term “fragment” as applied to a protein or peptide refers to a subsequence of a larger protein or peptide. A “fragment” of a protein or peptide is at least about 10 amino acids in length (amino acids naturally occurring as consecutive amino acids; e.g., as for a single linear epitope); for example at least about 15, 20, 30, 40, 50, 60, 100, 200, 300, or more amino acids in length (and any integer value in between). Antigenic HPV polypeptides may comprise two or more fragments of an HPV protein linked together.

As used herein, the term “sequence variant” refers to any sequence having one or more alterations in comparison to a reference sequence, whereby a reference sequence is any of the sequences listed in the sequence listing, i.e., SEQ ID NO:1 to SEQ ID NO:24. Thus, the term “sequence variant” includes nucleotide sequence variants and amino acid sequence variants. For a sequence variant in the context of a nucleotide sequence, the reference sequence is also a nucleotide sequence, whereas for a sequence variant in the context of an amino acid sequence, the reference sequence is also an amino acid sequence. A “sequence variant” as used herein is at least 80%, at least 85%, at least 90%, at least 95%, at least 98%, or at least 99% identical to the reference sequence. Sequence identity is usually calculated with regard to the full length of the reference sequence (i.e., the sequence recited in the application), unless otherwise specified. Percentage identity, as referred to herein, can be determined, for example, using various methods of alignment known in the art, such as BLAST using the default parameters specified by the NCBI (the National Center for Biotechnology Information; http://www.ncbi.nlm.nih.gov/) [Blosum 62 matrix; gap open penalty=1 1 and gap extension penalty=1]. A “sequence variant” in the context of a nucleic acid (nucleotide) sequence has an altered sequence in which one or more of the nucleotides in the reference sequence is deleted, or substituted, or one or more nucleotides are inserted into the sequence of the reference nucleotide sequence. Nucleotides are referred to herein by the standard one-letter designation (A, C, G, or T). Due to the degeneracy of the genetic code, a “sequence variant” of a nucleotide sequence can either result in a change in the respective reference amino acid sequence, i.e., in an amino acid “sequence variant” or not. In certain embodiments, the nucleotide sequence variants are variants that do not result in amino acid sequence variants (i.e., silent mutations). However, nucleotide sequence variants leading to “non-silent” mutations are also within the scope, in particular such nucleotide sequence variants, which result in an amino acid sequence, which is at least 80%, at least 85%, at least 90%, at least 95%, at least 98%, or at least 99% identical to the reference amino acid sequence. A “sequence variant” in the context of an amino acid sequence has an altered sequence in which one or more of the amino acids is deleted, substituted, or inserted in comparison to the reference amino acid sequence. As a result of the alterations, such a sequence variant has an amino acid sequence which is at least 80%, at least 85%, at least 90%, at least 95%, at least 98%, or at least 99% identical to the reference amino acid sequence. For example, per 100 amino acids of the reference sequence a variant sequence having no more than 10 alterations, i.e., any combination of deletions, insertions, or substitutions, is “at least 90% identical” to the reference sequence.

While it is possible to have non-conservative amino acid substitutions, in certain embodiments, the substitutions are conservative amino acid substitutions, in which the substituted amino acid has similar structural or chemical properties with the corresponding amino acid in the reference sequence. By way of example, conservative amino acid substitutions involve substitution of one aliphatic or hydrophobic amino acids, e.g., alanine, valine, leucine, and isoleucine, with another; substitution of one hydroxyl-containing amino acid, e.g., serine and threonine, with another; substitution of one acidic residue, e.g., glutamic acid or aspartic acid, with another; replacement of one amide-containing residue, e.g., asparagine and glutamine, with another; replacement of one aromatic residue, e.g., phenylalanine and tyrosine, with another; replacement of one basic residue, e.g., lysine, arginine, and histidine, with another; and replacement of one small amino acid, e.g., alanine, serine, threonine, methionine, and glycine, with another.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include the fusion to the N- or C-terminus of an amino acid sequence to a reporter molecule or an enzyme.

Unless otherwise stated, alterations in the sequence variants do not abolish the functionality of the respective reference sequence, for example, in the present case, the functionality of an antigen or vector disclosed herein. Guidance in determining which nucleotides and amino acid residues, respectively, may be substituted, inserted, or deleted without abolishing such functionality can be found by using computer programs well known in the art.

The nucleotide sequences of the present disclosure may be codon optimized, for example the codons may be optimized for use in human cells. For example, any viral or bacterial sequence may be so altered. Many viruses, including HIV and other lentiviruses, use a large number of rare codons and, by altering these codons to correspond to codons commonly used in the desired subject, enhanced expression of antigens may be achieved as described in Andre, S et al. (Increased Immune Response Elicited by DNA Vaccination with a Synthetic gp120 Sequence with Optimized Codon Usage. J Virol. 72, 1497-1503 (1998)).

As used herein, a nucleic acid sequence or an amino acid sequence “derived from” a designated nucleic acid, peptide, polypeptide, or protein refers to the origin of the nucleic acid, peptide, polypeptide, or protein. In some embodiments, the nucleic acid sequence or amino acid sequence which is derived from a particular sequence has an amino acid sequence that is essentially identical to that sequence or a portion thereof, from which it is derived, whereby “essentially identical” includes sequence variants as defined above. In certain embodiments, the nucleic acid sequence or amino acid sequence which is derived from a particular peptide or protein is derived from the corresponding domain in the particular peptide or protein. Thereby, “corresponding” refers in particular to the same functionality. For example, an “extracellular domain” corresponds to another “extracellular domain” (of another protein), or a “transmembrane domain” corresponds to another “transmembrane domain” (of another protein). “Corresponding” parts of peptides, proteins, and nucleic acids are thus identifiable to one of ordinary skill in the art. Likewise, sequences “derived from” other sequences are usually identifiable to one of ordinary skill in the art as having its origin in the sequence.

In some embodiments, a nucleic acid sequence or an amino acid sequence derived from another nucleic acid, peptide, polypeptide, or protein may be identical to the starting nucleic acid, peptide, polypeptide, or protein (from which it is derived). However, a nucleic acid sequence or an amino acid sequence derived from another nucleic acid, peptide, polypeptide, or protein may also have one or more mutations relative to the starting nucleic acid, peptide, polypeptide, or protein (from which it is derived), in particular a nucleic acid sequence or an amino acid sequence derived from another nucleic acid, peptide, polypeptide, or protein may be a functional sequence variant as described above of the starting nucleic acid, peptide, polypeptide, or protein (from which it is derived). For example, in a peptide/protein one or more amino acid residues may be substituted with other amino acid residues or one or more amino acid residue insertions or deletions may occur.

As used herein, the term “mutation” relates to a change in the nucleic acid sequence and/or in the amino acid sequence in comparison to a reference sequence, e.g., a corresponding genomic sequence. A mutation, e.g., in comparison to a genomic sequence, may be, for example, a (naturally occurring) somatic mutation, a spontaneous mutation, an induced mutation, e.g., induced by enzymes, chemicals, or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making a mutation, e.g., in a nucleic acid sequence or in an amino acid sequence. A mutation includes substitution, deletion, and insertion of one or more nucleotides or amino acids as well as inversion of several successive nucleotides or amino acids. Some types of coding sequence mutations include point mutations (differences in individual nucleotides or amino acids); silent mutations (differences in nucleotides that do not result in an amino acid changes); deletions (differences in which one or more nucleotides or amino acids are missing, up to and including a deletion of the entire coding sequence of a gene); frameshift mutations (differences in which deletion of a number of nucleotides indivisible by 3 results in an alteration of the amino acid sequence). A mutation that results in a difference in an amino acid may also be called an amino acid substitution mutation. Amino acid substitution mutations may be described by the amino acid change relative to wild type at a particular position in the amino acid sequence. To achieve a mutation in an amino acid sequence, a mutation may be introduced into the nucleotide sequence encoding said amino acid sequence in order to express a (recombinant) mutated polypeptide. A mutation may be achieved, e.g., by altering, e.g., by site-directed mutagenesis, a codon of a nucleic acid molecule encoding one amino acid to result in a codon encoding a different amino acid, or by synthesizing a sequence variant, e.g., by knowing the nucleotide sequence of a nucleic acid molecule encoding a polypeptide and by designing the synthesis of a nucleic acid molecule comprising a nucleotide sequence encoding a variant of the polypeptide without the need for mutating one or more nucleotides of a nucleic acid molecule.

The term “recombinant”, as used herein (e.g., a recombinant protein, a recombinant nucleic acid, a recombinant antibody, etc.), refers to any molecule (protein, nucleic acid, antibody, etc.) that is prepared, expressed, created, or isolated by recombinant means, and which is not naturally occurring. With reference to a nucleic acid or polypeptide, “recombinant” refers to one that has a sequence that is not naturally occurring or has a sequence that is made by an artificial combination of two or more otherwise separated segments of sequence, for example a CMV vector comprising a heterologous antigen. This artificial combination is often accomplished by chemical synthesis or, more commonly, by the artificial manipulation of isolated segments of nucleic acids, e.g., by genetic engineering techniques. A recombinant polypeptide may also refer to a polypeptide that has been made using recombinant nucleic acids, including recombinant nucleic acids transferred to a host organism that is not the natural source of the polypeptide (for example, nucleic acids encoding polypeptides that form a CMV vector comprising a heterologous antigen).

As used herein, the term “vector” refers to a carrier by which into which nucleic acid molecules of particular sequence can be incorporated and then introduced into a host cell, thereby producing a transformed host cell. A vector may include nucleic acid sequences that permit it to replicate in a host cell, such as an origin of replication. A vector may also include one or more selectable marker genes and other genetic elements known in the art, including promoter elements that direct nucleic acid expression. Vectors can be viral vectors, such as CMV vectors. Viral vectors may be constructed from wild type or attenuated virus, including replication deficient virus.

As the term “operably linked” is used herein, a first nucleic acid sequence is operably linked with a second nucleic acid sequence when the first nucleic acid sequence is placed in such a way that it has an effect upon the second nucleic acid sequence. Operably linked DNA sequences may be contiguous, or they may operate at a distance.

As used herein, the term “promoter” may refer to any of a number of nucleic acid control sequences that directs transcription of a nucleic acid. Typically, a eukaryotic promoter includes necessary nucleic acid sequences near the start site of transcription, such as, in the case of a polymerase II type promoter, a TATA element or any other specific DNA sequence that is recognized by one or more transcription factors. Expression by a promoter may be further modulated by enhancer or repressor elements. Numerous examples of promoters are available and well known to those of ordinary skill in the art. A nucleic acid comprising a promoter operably linked to a nucleic acid sequence that codes for a particular polypeptide may be termed an expression vector. Promoters can be from CMV genes, including but not limited to UL82 and UL78.

As used herein, the terms “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included. Where distinct designations are intended, it will be clear from the context.

As used herein, the term “microRNA” refers to a major class of biomolecules involved in control of gene expression. For example, in human heart, liver, or brain, miRNAs play a role in tissue specification or cell lineage decisions. In addition, miRNAs influence a variety of processes, including early development, cell proliferation and cell death, and apoptosis and fat metabolism. The large number of miRNA genes, the diverse expression patterns, and the abundance of potential miRNA targets suggest that miRNAs may be a significant source of genetic diversity. A mature miRNA is typically an 8-25 nucleotide non-coding RNA that regulates expression of an mRNA including sequences complementary to the miRNA. These small RNA molecules are known to control gene expression by regulating the stability and/or translation of mRNAs. For example, miRNAs bind to the 3′ UTR of target mRNAs and suppress translation. MiRNAs may also bind to target mRNAs and mediate gene silencing through the RNAi pathway. MiRNAs may also regulate gene expression by causing chromatin condensation.

A miRNA silences translation of one or more specific mRNA molecules by binding to a miRNA recognition element (MRE,) which is defined as any sequence that directly base pairs with and interacts with the miRNA somewhere on the mRNA transcript. Often, the MRE is present in the 3′ untranslated region (UTR) of the mRNA, but it may also be present in the coding sequence or in the 5′ UTR. MREs are not necessarily perfect complements to miRNAs, usually having only a few bases of complementarity to the miRNA and often containing one or more mismatches within those bases of complementarity. The MRE may be any sequence capable of being bound by a miRNA sufficiently that the translation of a gene to which the MRE is operably linked (such as a CMV gene that is essential or augmenting for growth in vivo) is repressed by a miRNA silencing mechanism such as the RISC.

The term “vaccine” as used herein is typically understood to be a prophylactic or therapeutic material providing at least one antigen or immunogen. The antigen or immunogen may be derived from any material that is suitable for vaccination. For example, the antigen or immunogen may be derived from a pathogen, such as from bacteria or virus particles, etc., or from a tumor or cancerous tissue. The antigen or immunogen stimulates the body's adaptive immune system to provide an adaptive immune response. In particular, an “antigen” or an “immunogen” refers typically to a substance which may be recognized by the immune system (e.g., the adaptive immune system), and which is capable of triggering an antigen-specific immune response, e.g., by formation of antibodies and/or antigen-specific T cells as part of an adaptive immune response. Typically, an antigen may be or may comprise a peptide or protein which may be presented by the MHC to T-cells. Vaccines can be used prophylactically or therapeutically. Thus, vaccines can be used reduce the likelihood of developing a disease (such as a tumor or pathological infection) or to reduce the severity of symptoms of a disease or condition, limit the progression of the disease or condition (such as a tumor or a pathological infection), or limit the recurrence of a disease or condition (such as a tumor). In particular embodiments, a vaccine comprises a replication-deficient CMV expressing a heterologous antigen. In particular embodiments, a vaccine comprises a replication-deficient CMV expressing a fusion protein comprising HPV antigens. As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) the protein is able to evoke an immune response of the humoral and/or cellular type directed against that protein.

As used herein, “heterologous” or “exogenous” nucleic acid molecule, construct, or sequence refers to a nucleic acid molecule or portion of a nucleic acid molecule that is not native to a second nucleic acid molecule or to a host cell, depending on the context, but may be homologous to a nucleic acid molecule or portion of the second nucleic acid molecule or host cell. The source of the heterologous or exogenous nucleic acid molecule, construct, or sequence may be from a different genus or species, or may be synthetic. In certain embodiments, a heterologous or exogenous nucleic acid molecule is added (i.e., not endogenous or native) to a host cell or host genome by, for example, conjugation, transformation, transfection, electroporation, or the like, wherein the added molecule may integrate into the host genome or exist as extra-chromosomal genetic material (e.g., as a plasmid or other form of self replicating vector), and may be present in multiple copies. In addition, the term “heterologous” includes a non-native enzyme, protein, or other activity encoded by an exogenous nucleic acid molecule introduced into the second nucleic acid molecule or host cell, even if the second nucleic acid molecule or host cell encodes a homologous protein or activity.

As used herein, the term “heterologous antigen” refers to any protein or fragment thereof that is not derived from a vector to which it has been inserted. For example, in some embodiments, a “heterologous antigen” is any protein or fragment thereof that is not derived from CMV. Heterologous antigens may be pathogen-specific antigens, tumor virus antigens, tumor antigens, host self-antigens, or any other antigen.

As used herein, “antigen-specific T cell” refers to a CD8+ or CD4+ lymphocyte that recognizes a particular antigen. Generally, antigen-specific T cells specifically bind to a particular antigen presented by MHC molecules, but not other antigens presented by the same MHC.

As used herein, “immunogenic peptide” refers to peptide that comprises an allele-specific motif or other sequence, such as an N-terminal repeat, such that the peptide will bind an MHC molecule and induce a cytotoxic T lymphocyte (“CTL”) response, or a B cell response (for example, antibody production) against the antigen from which the immunogenic peptide is derived. In some embodiments, immunogenic peptides are identified using sequence motifs or other methods, such as neural net or polynomial determinations known in the art. Typically, algorithms are used to determine the “binding threshold” of peptides to select those with scores that give them a high probability of binding at a certain affinity and will be immunogenic. The algorithms are based either on the effects on MHC binding of a particular amino acid at a particular position, the effects on antibody binding of a particular amino acid at a particular position, or the effects on binding of a particular substitution in a motif-containing peptide. Within the context of an immunogenic peptide, a “conserved residue” is one which appears in a significantly higher frequency than would be expected by random distribution at a particular position in a peptide. In some embodiments, a conserved residue is one where the MHC structure may provide a contact point with the immunogenic peptide.

As used herein, the term “administration” means to provide or give a subject an agent, such as a composition comprising an effective amount of an antigen or pharmaceutical composition comprising an exogenous antigen by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes.

As used herein, a “pharmaceutically acceptable carrier” of use is conventional. Remington's Pharmaceutical Sciences, by E.W. Martin, Mack Publishing Co., Easton, PA, 19^{th} Edition, 1995, describes compositions and formulations suitable for pharmaceutical delivery of the compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol, or the like as a vehicle. For solid compositions (such as powder, pill, tablet, or capsule forms), conventional non-toxic solid carriers may include, for example, pharmaceutical grades of mannitol, lactose, starch, or magnesium stearate. In addition to biologically neutral carriers, pharmaceutical compositions to be administered may contain minor amounts of non-toxic auxiliary substances, such as wetting or emulsifying agents, preservatives, and pH buffering agents and the like, for example sodium acetate or sorbitan monolaurate.

Doses are often expressed in relation to bodyweight. Thus, a dose which is expressed as [g, mg, or other unit]/kg (or g, mg, etc.) may refer to [g, mg, or other unit]“per kg (or g, mg, etc.) bodyweight”, even if the term “bodyweight” is not explicitly mentioned. Alternatively, doses may be expressed in “unit dosage form” (or “unit dose form”), which is the form of a pharmaceutical product, including, but not limited to, the form in which the pharmaceutical product is marketed for use. Examples include pills, tablets, capsules, and liquid solutions and suspensions.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, “HPV” and “human papillomavirus” refer to the members of the family Papillomavirus that are capable of infecting humans. There are two major groups of HPVs defined by their tropism (genital/mucosal and cutaneous groups), each of which contains multiple virus “types” or “strains” (e.g., HPV 16, HPV 18, HPV 31, HPV 32, etc.). These include HPV types that are associated with genital infection and malignancy, as well as those that produce benign papillomas, both at mucosa and skin, resulting in morbidity to the patient. HPV subtypes associated with a high-risk of developing lesions progressing to invasive cancer include, but are not limited to, HPV16, HPV18, HPV31, HPV33, HPV35, HPV39, HPV45, HPV51, HPV52, HPV56, HPV58, HPV59, HPV66, HPV68, HPV73, and HPV82. The term “HPV-associated cancers” includes, but is not limited to, cervical, penile, vaginal, vulval, anal, rectal, oropharyngeal, skin, and head and neck cancer.

### II. Human Papillomavirus Antigens

Disclosed herein are fusion proteins comprising HPV antigens and nucleic acids encoding the same.

In some embodiments, the present disclosure provides a fusion protein comprising one or more of E6, E7, E1, and E2, or portions or fragments or variants thereof.

HPV E6 (also referred to as “E6”) protein plays a role in the induction and maintenance of cellular transformation, and acts by stimulating the destruction of host cell regulatory proteins. E6 associates with host cell E6-AP ubiquitin-protein ligase (E6AP) and inactivates tumor suppressors such as TP53 by targeting them to the 26S proteasome for degradation. A PDZ ligand on the C-terminal of the E6 protein interacts with cellular PDZ-containing proteins, which can alter differentiation of cells. In some embodiments, HPV E6 refers to a fragment of the amino acid sequence according to SEQ ID NO:4. In some embodiments, HPV E6 refers to a fragment of the amino acid sequence according to SEQ ID NO:1. In some embodiments, HPV E6 refers to an amino acid sequence of the HPV18 subtype according to UniProtKB—Entry P06463 (SEQ ID NO:5). In some embodiments, HPV E6 refers to an amino acid sequence of the HPV16 subtype according to UniProtKB—Entry P03126 (SEQ ID NO:6). In some embodiments, HPV E6 refers to a portion of an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:4. In some embodiments, HPV E6 refers to a portion of an amino acid sequence having at least 90%, at least 910%, at least 92%, at least 93%, at least 94%, at least 95%, at least 96%, at least 97%, at least 98%, at least 99%, or 100% identity to the amino acid sequence according to SEQ ID NO:1. In some embodiments, HPV E6 refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:5, or a fragment thereof. In some embodiments, HPV E6 refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:6, or a fragment thereof.

HPV E7 (also referred to as “E7”) protein plays a role in viral genome replication by driving entry of quiescent cells into the cell cycle. Stimulation of progression from Gi to S phase allows the virus to efficiently use the cellular DNA replicating machinery to achieve viral genome replication. E7 protein has both transforming and trans-activating activities. E7 induces the disassembly of the E2F1 transcription factor from RB1, with subsequent transcriptional activation of E2F1-regulated S-phase genes. E7 also interferes with host histone deacetylation mediated by HDAC1 and HDAC2, leading to transcription activation. E7 also plays a role in the inhibition of both antiviral and antiproliferative functions of host interferon alpha. In some embodiments, HPV E7 refers to a fragment of the amino acid sequence according to SEQ ID NO:4. In some embodiments, HPV E7 refers to a fragment of the amino acid sequence according to SEQ ID NO:1. In some embodiments, HPV E7 refers to an amino acid sequence of the HPV18 subtype according to UniProtKB—Entry P06788 (SEQ ID NO:7). In some embodiments, HPV E7 refers to an amino acid sequence of the HPV16 subtype according to UniProtKB—Entry P03129 (SEQ ID NO:8). In some embodiments, HPV E7 refers to a portion of an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:4. In some embodiments, HPV E7 refers to a portion of an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:1. In some embodiments, HPV E7 refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:7, or a fragment thereof. In some embodiments, HPV E7 refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:8, or a fragment thereof.

HPV E1 (also referred to as “E1”) protein is an ATP-dependent DNA helicase and the only enzyme encoded by papillomaviruses. It is essential for replication and amplification of the viral episome in the nucleus of infected cells. To do so, E1 assembles into a double-hexamer at the viral origin, unwinds DNA at the origin and ahead of the replication fork and interacts with cellular DNA replication factors (see Bergvall, M et al. The E1 proteins. Virology 445(1-2):35-56 (2013)). As used herein, “E1” may refer to the enzyme or an amino acid sequence encoding an E1 protein or peptide, or variant, or portions thereof, depending on the context. In some embodiments, HPV E1 refers to a fragment of the amino acid sequence according to SEQ ID NO:4. In some embodiments, HPV E1 refers to the amino acid sequence according to SEQ ID NO:2, or a fragment of the amino acid sequence according to SEQ ID NO:2. In some embodiments, HPV E1 refers to an amino acid sequence of the HPV18 subtype according to UniProtKB—Entry P06789 (SEQ ID NO:9). In some embodiments, HPV E1 refers to an amino acid sequence of the HPV16 subtype according to UniProtKB—Entry P03114 (SEQ ID NO:10). In some embodiments, HPV E1 refers to a portion of an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:4. In some embodiments, HPV E1 refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:2, or a fragment thereof. In some embodiments, HPV E1 refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:9, or a fragment thereof. In some embodiments, HPV E1 refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:10, or a fragment thereof.

HPV E2 (also referred to as “E2”) protein plays an accessory role in initiation of DNA replication by activating or repressing transcription. The E2 protein contains a transactivation domain (TAD) important for transcriptional activation/repression and replication; a flexible linker, and a DNA binding dimerization domain (DBD) that affects transcriptional activation/repression and replication. In some embodiments, HPV E2 refers to a fragment of the amino acid sequence according to SEQ ID NO:4. In some embodiments, HPV E2 refers to a fragment of the amino acid sequence according to SEQ ID NO:3. In some embodiments, HPV E2 refers to an amino acid sequence of the HPV18 subtype according to UniProtKB—Entry P06790 (SEQ ID NO:11). In some embodiments, HPV E2 refers to an amino acid sequence of the HPV16 subtype according to UniProtKB—Entry P03120 (SEQ ID NO: 12). In some embodiments, HPV E2 refers to a portion of an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:4. In some embodiments, HPV E2 refers to a portion of an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:3. In some embodiments, HPV E2 refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:11, or a fragment thereof. In some embodiments, HPV E2 refers to an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:12, or a fragment thereof.

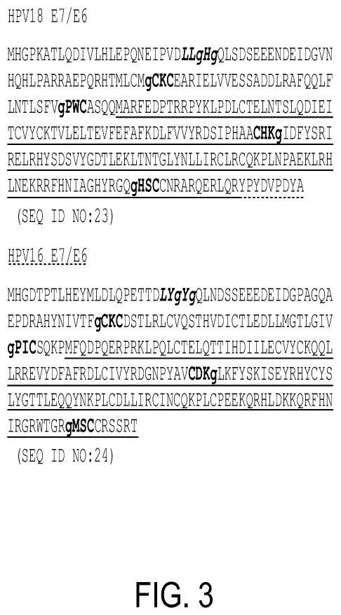

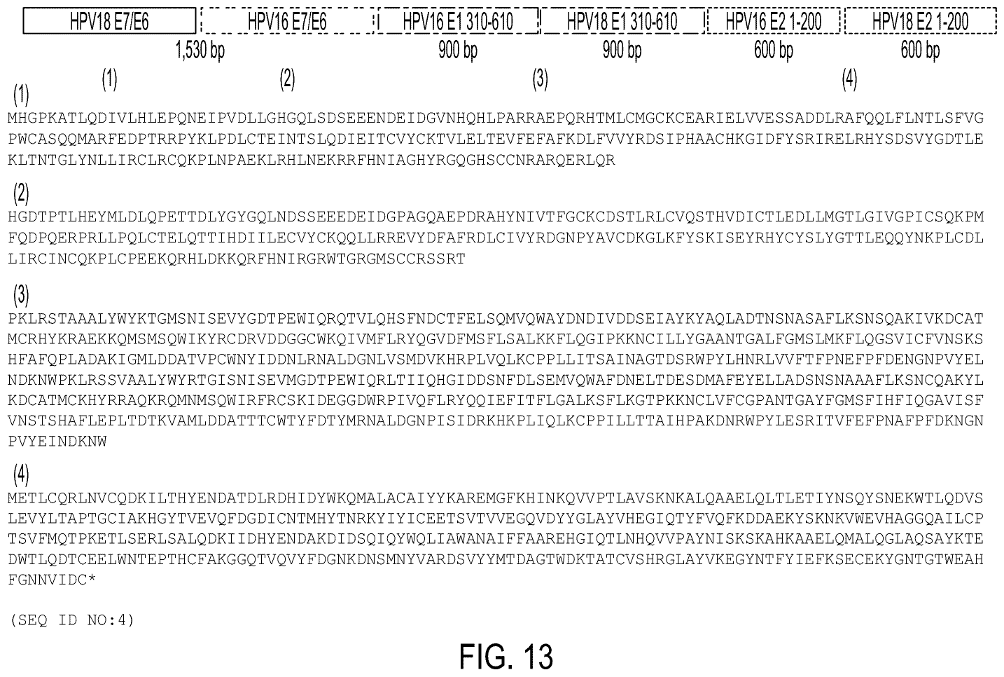

FIGS. 3 and 13 show non-limiting examples of fusion proteins and components thereof according to the present disclosure.

In some embodiments, the present disclosure provides a fusion protein comprising, consisting, or consisting essentially of E7, E6, E1, and E2, or fragments thereof. Unless otherwise specified, the individual HPV Early antigens can be present in the fusion protein in any order. Additionally, they may be connected in a C-terminus to N-terminus to C-terminus manner, with or without linkers as described herein. In some embodiments, the present disclosure provides an E7-E6-E1-E2 fusion protein. In some embodiments, the present disclosure provides an E6-E7-E1-E2 fusion protein. In some embodiments, the present disclosure provides a fusion protein comprising at least two E7, at least two E6, at least two E1, and at least two E2 proteins of HPV or fragments thereof, wherein the first protein is from one HPV subtype and the second protein is a from a different HPV subtype. Unless otherwise specified, the individual HPV Early antigens from different subtypes can be present in the fusion protein in any order. In some embodiments, the present disclosure provides an HPV18 E7-HPV18 E6-HPV16 E7-HPV16 E6-HPV16 E1-HPV18 E1-HPV16 E2-HPV18 E2 fusion protein. In some embodiments, the present disclosure provides an HPV18 E6-HPV18 E7-HPV16 E6-HPV16 E7-HPV16 E1-HPV18 E1-HPV16 E2-HPV18 E2 fusion protein. In some embodiments, the fusion protein comprises an amino acid sequence having 90%, 91%, 92%, 93%, 94%, 95%, 96%, 97%, 98%, 99%, or 100% identity to the amino acid sequence according to SEQ ID NO:4. In some embodiments, the fusion protein comprises the amino acid sequence according to SEQ ID NO:4. In some embodiments, the fusion protein consists of the amino acid sequence according to SEQ ID NO:4.

In some embodiments, the present disclosure provides a fusion protein comprising, consisting, or consisting essentially of E7 and E6, or fragments thereof. Unless otherwise specified, the individual HPV Early antigens can be present in the fusion protein in any order. Additionally, they may be connected in a C-terminus to N-terminus to C-terminus manner, with or without linkers as described herein. In some embodiments, the present disclosure provides an E7-E6 fusion protein. In some embodiments, the present disclosure provides an E6-E7 fusion protein. In some embodiments, the present disclosure provides a fusion protein comprising at least two E7 and at least two E6 proteins of HPV or fragments thereof, wherein the first protein is from one HPV subtype and the second protein is a from a different HPV subtype. Unless otherwise specified, the individual HPV Early antigens from different subtypes can be present in the fusion protein in any order. In some embodiments, the present disclosure provides an HPV18 E7-HPV18 E6-HPV16 E7-HPV16 E6 fusion protein. In some embodiments, the present disclosure provides an HPV18 E6-HPV18 E7-HPV16 E6-HPV16 E7 fusion protein. In some embodiments, the fusion protein comprises an amino acid sequence having 90%, 91%, 92%, 93%, 94%, 95%, 96%, 97%, 98%, 99%, or 100% identity to the amino acid sequence according to SEQ ID NO:1. In some embodiments, the fusion protein comprises the amino acid sequence according to SEQ ID NO: 1. In some embodiments, the fusion protein consists of the amino acid sequence according to SEQ ID NO:1. In some embodiments, the fusion protein comprises an HPV18 E7-HPV18 E6-HPV16 E7-HPV16 E6 fusion protein consisting of the amino acid sequence according to SEQ ID NO: 1.

In some embodiments, the present disclosure provides a fusion protein comprising, consisting, or consisting essentially of E1 or fragments thereof. In some embodiments, the present disclosure provides an E1 fusion protein. In some embodiments, the present disclosure provides a fusion protein comprising at least two E1 proteins of HPV or fragments thereof, wherein the first protein is from one HPV subtype and the second protein is a from a different HPV subtype. Unless otherwise specified, the individual HPV Early antigens from different subtypes can be present in the fusion protein in any order. Additionally, they may be connected in a C-terminus to N-terminus to C-terminus manner, with or without linkers as described herein. In some embodiments, the present disclosure provides an HPV16 E1-HPV18 E1 fusion protein. In some embodiments, the fusion protein comprises an amino acid sequence having 90%, 91%, 92%, 93%, 94%, 95%, 96%, 97%, 98%, 99%, or 100% identity to the amino acid sequence according to SEQ ID NO:2. In some embodiments, the fusion protein comprises the amino acid sequence according to SEQ ID NO:2. In some embodiments, the fusion protein consists of the amino acid sequence according to SEQ ID NO:2. In some embodiments, the fusion protein comprises an E1 fusion protein consisting of the amino acid sequence according to SEQ ID NO:2.

In some embodiments, the present disclosure provides a fusion protein comprising, consisting, or consisting essentially of E2 or fragments thereof. In some embodiments, the present disclosure provides an E2 fusion protein. In some embodiments, the present disclosure provides a fusion protein comprising at least two E2 proteins of HPV or fragments thereof, wherein the first protein is from one HPV subtype and the second protein is a from a different HPV subtype. Unless otherwise specified, the individual HPV Early antigens from different subtypes can be present in the fusion protein in any order. Additionally, they may be connected in a C-terminus to N-terminus to C-terminus manner, with or without linkers as described herein. In some embodiments, the present disclosure provides an HPV16 E2-HPV18 E2 fusion protein. In some embodiments, the fusion protein comprises an amino acid sequence having 90%, 91%, 92%, 93%, 94%, 95%, 96%, 97%, 98%, 99%, or 100% identity to the amino acid sequence according to SEQ ID NO:3. In some embodiments, the fusion protein comprises the amino acid sequence according to SEQ ID NO:3. In some embodiments, the fusion protein consists of the amino acid sequence according to SEQ ID NO:3. In some embodiments, the fusion protein comprises an E2 fusion protein consisting of the amino acid sequence according to SEQ ID NO:3.

In some embodiments, the present disclosure provides a fusion protein comprising, consisting, or consisting essentially of E7, E6, E1, and E2, or fragments thereof. Unless otherwise specified, the individual HPV Early antigens can be present in the fusion protein in any order. Additionally, they may be connected in a C-terminus to N-terminus to C-terminus manner, with or without linkers as described herein. In some embodiments, the present disclosure provides an E7-E6-E1-E2 fusion protein. In some embodiments, the fusion protein comprises (i) an amino acid sequence having 90%, 91%, 92%, 93%, 94%, 95%, 96%, 97%, 98%, 99%, or 100% identity to the amino acid sequence according to SEQ ID NO: 1; (ii) an amino acid sequence having 90%, 91%, 92%, 93%, 94%, 95%, 96%, 97%, 98%, 99%, or 100% identity to the amino acid sequence according to SEQ ID NO: 2; and (iii) an amino acid sequence having 90%, 91%, 92%, 93%, 94%, 95%, 96%, 97%, 98%, 99%, or 100% identity to the amino acid sequence according to SEQ ID NO: 3. In some embodiments, the fusion protein comprises (i) the amino acid sequence according to SEQ ID NO:1; (ii) the amino acid sequence according to SEQ ID NO:2; and (iii) the amino acid sequence according to SEQ ID NO:3.

In some embodiments, the present disclosure provides a fusion protein comprising, consisting, or consisting essentially of E7, E6, and E1, or fragments thereof. Unless otherwise specified, the individual HPV Early antigens can be present in the fusion protein in any order. Additionally, they may be connected in a C-terminus to N-terminus to C-terminus manner, with or without linkers as described herein. In some embodiments, the present disclosure provides an E7-E6-E1 fusion protein. In some embodiments, the fusion protein comprises (i) an amino acid sequence having 90%, 91%, 92%, 93%, 94%, 95%, 96%, 97%, 98%, 99%, or 100% identity to the amino acid sequence according to SEQ ID NO: 1; and (ii) an amino acid sequence having 90%, 91%, 92%, 93%, 94%, 95%, 96%, 97%, 98%, 99%, or 100% identity to the amino acid sequence according to SEQ ID NO: 2. In some embodiments, the fusion protein comprises (i) the amino acid sequence according to SEQ ID NO: 1; and (ii) the amino acid sequence according to SEQ ID NO:2.

In some embodiments, the present disclosure provides a fusion protein comprising, consisting, or consisting essentially of E7, E6, and E2, or fragments thereof. Unless otherwise specified, the individual HPV Early antigens can be present in the fusion protein in any order. Additionally, they may be connected in a C-terminus to N-terminus to C-terminus manner, with or without linkers as described herein. In some embodiments, the present disclosure provides an E7-E6-E2 fusion protein. In some embodiments, the fusion protein comprises (i) an amino acid sequence having 90%, 91%, 92%, 93%, 94%, 95%, 96%, 97%, 98%, 99%, or 100% identity to the amino acid sequence according to SEQ ID NO: 1; and (ii) an amino acid sequence having 90%, 91%, 92%, 93%, 94%, 95%, 96%, 97%, 98%, 99%, or 100% identity to the amino acid sequence according to SEQ ID NO: 3. In some embodiments, the fusion protein comprises the amino acid sequence according to SEQ ID NO: 1; and (ii) the amino acid sequence according to SEQ ID NO:3.

In some aspects, the present disclosure provides a nucleic acid molecule encoding any of the fusion proteins disclosed herein.

In some embodiments, the fusion protein is encoded by a nucleic acid comprising a sequence having 90%, 91%, 92%, 93%, 94%, 95%, 96%, 97%, 98%, 99%, or 100% identity to the nucleic acid sequence according to SEQ ID NO:16. In some embodiments, the fusion protein consists of an amino acid sequence encoded by the nucleic acid sequence according to SEQ ID NO: 16. In some embodiments, the fusion protein consists essentially of an amino acid sequence encoded by the nucleic acid sequence according to SEQ ID NO:16.

In some embodiments, the fusion protein is encoded by a nucleic acid comprising a sequence having 90%, 91%, 92%, 93%, 94%, 95%, 96%, 97%, 98%, 99%, or 100% identity to the nucleic acid sequence according to SEQ ID NO:13. In some embodiments, the fusion protein consists of an amino acid sequence encoded by the nucleic acid sequence according to SEQ ID NO: 13. In some embodiments, the fusion protein consists essentially of an amino acid sequence encoded by the nucleic acid sequence according to SEQ ID NO:13.

In some embodiments, the fusion protein is encoded by a nucleic acid comprising a sequence having 90%, 91%, 92%, 93%, 94%, 95%, 96%, 97%, 98%, 99%, or 100% identity to the nucleic acid sequence according to SEQ ID NO:14. In some embodiments, the fusion protein consists of an amino acid sequence encoded by the nucleic acid sequence according to SEQ ID NO: 14. In some embodiments, the fusion protein consists essentially of an amino acid sequence encoded by the nucleic acid sequence according to SEQ ID NO:14.

In some embodiments, the fusion protein is encoded by a nucleic acid comprising a sequence having 90%, 91%, 92%, 93%, 94%, 95%, 96%, 97%, 98%, 99%, or 100% identity to the nucleic acid sequence according to SEQ ID NO:15. In some embodiments, the fusion protein consists of an amino acid sequence encoded by the nucleic acid sequence according to SEQ ID NO: 15. In some embodiments, the fusion protein consists essentially of an amino acid sequence encoded by the nucleic acid sequence according to SEQ ID NO:15.

### III. Vectors

In some embodiments, the present disclosure provides vectors encoding a fusion protein as described above.

The vector may be any expression vector known in the art. For the antigens to be expressed, the protein coding sequence of the fusion protein should be “operably linked” to regulatory or nucleic acid control sequences that direct transcription and translation of the protein. A coding sequence and a nucleic acid control sequence or promoter are said to be “operably linked” when they are covalently linked in such a way as to place the expression or transcription and/or translation of the coding sequence under the influence or control of the nucleic acid control sequence. The “nucleic acid control sequence” may be any nucleic acid element, such as, but not limited to promoters, enhancers, IRES, introns, and other elements described herein that direct the expression of a nucleic acid sequence or coding sequence that is operably linked thereto. The term “promoter” refers to a group of transcriptional control modules that are clustered around the initiation site for RNA polymerase II and that when operationally linked to the protein coding sequences of the disclosure lead to the expression of the encoded protein. The expression of heterologous antigens and fusion proteins of the present disclosure may be under the control of a constitutive promoter or of an inducible promoter, which initiates transcription only when exposed to some particular external stimulus, such as, without limitation, antibiotics such as tetracycline, hormones such as ecdysone, or heavy metals. The promoter may also be specific to a particular cell-type, tissue, or organ. Many suitable promoters and enhancers are known in the art, and any such suitable promoter or enhancer may be used for expression of the transgenes of the disclosure. For example, suitable promoters and/or enhancers may be selected from the Eukaryotic Promoter Database (EPDB).

In some embodiments, the vector encoding the fusion protein is a plasmid, cosmid, phage, bacterial vector, or viral vector. In some embodiments, the vector is a viral vector, such a poxvirus, adenovirus, rubella, sendai virus, rhabdovirus, alphavirus, herpesvirus, lentivirus, retrovirus, or adeno-associated virus. In some embodiments, the vector encoding the fusion protein is a bacterial artificial chromosome (BAC). In some embodiments, the vector encoding the fusion protein is a CMV vector, e.g., a RhCMV or HCMV vector. In some embodiments, the vector encoding the fusion protein is a recombinant HCMV vector comprising a TR3 backbone.

In some embodiments, the recombinant CMV vector is or is derived from HCMV TR3. As referred to herein, “HCMV TR3” or “TR3” refers to a HCMV-TR3 vector backbone derived from the clinical isolate HCMV TR, as described in Caposio P et al. (Characterization of a live attenuated HCMV-based vaccine platform. Scientific Reports 9, 19236 (2019)).

As described herein, recombinant CMV vectors may be characterized by the presence or absence of one or more CMV genes. CMV vectors may also be characterized by the presence or absence of one or more proteins encoded by one or more CMV genes. A protein encoded by a CMV gene may be absent due to the presence of a mutation in the nucleic acid sequence encoding the CMV gene. In some embodiments, the vector can include an ortholog or homolog of a CMV gene. Examples of CMV genes include, but are not limited to, UL82, UL128, UL130, UL146, UL147, UL18, and UL78.

The human cytomegalovirus UL82 gene encodes pp71, a protein that is localized in the tegument domain of the virus particle. For example, the UL82 gene of the CMV TR strain is 118811 to 120490 for GenBank Accession No. KF021605.1.

Pp71 may perform one or more functions, including inhibition of Daxx repression of viral gene transcription, negative regulation of STING, and evasion of cell antiviral responses (Kalejta R F, et al. Expanding the Known Functional Repertoire of the Human Cytomegalovirus pp71 Protein. Front Cell Infect Microbiol. 2020-3-12; 10:95). Deletion of UL82 or disruption of UL82 by insertion of a foreign gene at the UL82 locus results in the absence of pp71 protein and consequently reduces replication in fibroblasts, endothelial cells, epithelial cells, and astrocytes (Caposio P et al., Characterization of a live-attenuated HCMV-based vaccine platform. Sci Rep. 2019-12-17; 9(1):19236). The effects of UL82 deletion or disruption are reversible by cell kinase inhibitors. The rhesus cytomegalovirus (RhCMV) gene RhCMV 110 is homologous to human CMV UL82 (Hansen S G, et al. Complete sequence and genomic analysis of rhesus cytomegalovirus. J Virol. 2003 June; 77(12):6620-36).

The human cytomegalovirus genes UL128 and UL130 encode structural components of the viral envelope (Patrone, M et al. Human cytomegalovirus UL130 protein promotes endothelial cell infection through a producer cell modification of the virion. J Virol. 79(13):8361-73 (2005); Ryckman, B J et al. Characterization of the human cytomegalovirus gH/gL/UL128-131 complex that mediates entry into epithelial and endothelial cells. J Virol. 82(1):60-70 (2008); Wang, D et al. Human cytomegalovirus virion protein complex required for epithelial and endothelial cell tropism. Proc Natl Acad Sci USA. 102(50):18153-8 (2005)). For example, the UL128 gene of the CMV TR strain is 176206 to 176964 for GenBank Accession No. KF021605.1 and the UL130 gene of the CMV TR strain is 177004 to 177648 for GenBank Accession No. KF021605.1.

The human cytomegalovirus genes UL146 and UL147 encode the CXC chemokines vCXC-1 and vCXC-2, respectively (Penfold, M E et al. Cytomegalovirus encodes a potent alpha chemokine. Proc Natl Acad Sci USA. 96(17):9839-44 (1999)). For example, the UL146 gene of the CMV TR strain is 180954 to 181307 for GenBank Accession No. KF021605.1 and the UL147 gene of the CMV TR strain is 180410 to 180889 for GenBank Accession No. KF021605.1.

The human cytomegalovirus UL18 gene encodes a type-I membrane glycoprotein that associates with β2-microglobulin and can bind endogenous peptides (Park, B et al. Human cytomegalovirus inhibits tapasin-dependent peptide loading and optimization of the MHC class I peptide cargo for immune evasion. Immunity. 20(1):71-85 (2004); Browne, H et al. A complex between the MHC class I homologue encoded by human cytomegalovirus and beta 2 microglobulin. Nature. 347(6295):770-2 (1990); Fahnestock, M L et al. The MHC class I homolog encoded by human cytomegalovirus binds endogenous peptides. Immunity. 3(5):583-90 (1995)). For example, the UL18 gene of the CMV TR strain is 24005 to 25111 for GenBank Accession No. KF021605.1.

The human cytomegalovirus UL78 gene encodes a putative G protein-coupled receptor (Chee, M S et al. Analysis of the protein-coding content of the sequence of human cytomegalovirus strain AD169. Curr Top Microbiol Immunol. 1154:125-69 (1990)) and may also have a role in viral replication (Michel, D et al. The human cytomegalovirus UL78 gene is highly conserved among clinical isolates, but is dispensable for replication in fibroblasts and a renal artery organ-culture system. J Gen Virol. 86(Pt 2):297-306 (2005)). For example, the UL78 gene of the CMV TR strain is 114247 to 115542 for GenBank Accession No. KF021605.1.

In some embodiments, the recombinant CMV vector expresses UL128 or UL130, or orthologs thereof. In some embodiments, the recombinant CMV vector expresses UL146 and UL147, or orthologs thereof. In some embodiments, the recombinant CMV vector expresses UL128, UL130, UL146, and UL147.

In some embodiments, the recombinant CMV vector (e.g., a recombinant HCMV vector or a recombinant HCMV vector comprising a TR3 backbone) does not express UL128 or UL130, or orthologs thereof, due to the presence of a mutation in the nucleic acid sequences encoding UL128 and UL130, or the orthologs thereof. In some embodiments, the CMV vector is deficient for one or more of UL146, UL147, UL18, UL78, and UL82, and orthologs thereof, due to the presence of a mutation in the nucleic acid sequence encoding UL146, UL147, UL18, UL78, or UL82, or the ortholog thereof. In some embodiments, the CMV vector is deficient for US11, and orthologs thereof, due to the presence of a mutation in the nucleic acid sequence encoding US11, or the ortholog thereof. In the aforementioned embodiments, the mutation or mutations may be any mutation that results in a lack of expression of active proteins. Such mutations include, for example, point mutations, frameshift mutations, deletions of less than all of the sequence that encodes the protein (truncation mutations), or deletions of all of the nucleic acid sequence that encodes the protein. In some embodiments, the recombinant CMV vector (e.g., a recombinant HCMV vector or a recombinant HCMV vector comprising a TR3 backbone) expresses UL40 and US28, or orthologs thereof.

In some embodiments, the recombinant CMV vector (e.g., a recombinant HCMV vector or a recombinant HCMV vector comprising a TR3 backbone) does not express UL78, UL128, or UL130, or orthologs thereof, due to the presence of a mutation in the nucleic acid sequences encoding UL78, UL128, and UL130, or the orthologs thereof. In some further embodiments, a nucleic acid molecule encoding a fusion protein as disclosed herein is replaces UL78. In some further embodiments, the fusion protein is operably linked to and is expressed by the UL78 promoter.

In some embodiments, the recombinant CMV vector (e.g., a recombinant HCMV vector or a recombinant HCMV vector comprising a TR3 backbone) does not express UL78, UL128, or UL130, or orthologs thereof, due to the presence of a mutation in the nucleic acid sequences encoding UL78, UL128, and UL130, or the orthologs thereof. In some further embodiments, the recombinant CMV vector expresses UL18, UL82, UL146, and UL147, or orthologs thereof. In some further embodiments, a nucleic acid molecule encoding a fusion protein as disclosed herein is replaces UL78. In some further embodiments, the fusion protein is operably linked to and is expressed by the UL78 promoter.

In some embodiments, the recombinant CMV vector (e.g., a recombinant HCMV vector or a recombinant HCMV vector comprising a TR3 backbone) does not express UL82, UL128, or UL130, or orthologs thereof, due to the presence of a mutation in the nucleic acid sequences encoding UL82, UL128, and UL130, or the orthologs thereof. In some further embodiments, a nucleic acid molecule encoding a fusion protein disclosed herein is replaces UL82. In some further embodiments, the fusion protein is operably linked to and is expressed by the UL82 promoter.

A challenge for manufacturing HCMV vectors having desirable properties for vaccines is that the vectors are often designed to have reduced viral replication or growth. For example, some live attenuated HCMV-HIV vaccine vectors are engineered to be growth deficient by deletion of the HCMV gene UL82 (which encodes the tegument protein pp71), resulting in lower viral yield. Pp71 is important for wild type HCMV infection because this tegument protein is translocated to the nucleus where it suppresses cellular Daxx function, thus allowing CMV immediate-early (IE) gene expression that triggers the replication cycle. Some manufacturing processes rely on functional complementation using transient transfection of MRC-5 cells with an siRNA targeting Daxx, which mimics one of the functions of HCMV pp71. Another approach is to use transfection of a mRNA encoding pp71, to enable the host cell to express the essential viral gene. Transfection of a mRNA for expressing the essential viral gene may be able to provide all of the functions of the gene that are likely to enhance the infection process, such as cell cycle stimulation, efficient virion packaging, and virus stability. In addition, protein present late in infection has the potential to be packaged in the progeny virus, which could lower the required dose of the vaccine by more efficient first round infection and establishment of persistent infection. Accordingly, in some embodiments, the present disclosure provides a method of producing a recombinant CMV viral vector, comprising: (a) introducing a mRNA encoding a pp71 protein to a cell; (b) infecting the cell with a recombinant CMV; (c) incubating the cell; and (d) collecting the recombinant CMV viral vector. In some embodiments, the nucleic acid encoding a pp71 protein is delivered to the cell using transfection. In some embodiments, the cell is a MRC-5 cell. In some embodiments, the recombinant CMV is a recombinant HCMV as described herein (e.g., a recombinant HCMV vector derived from a TR3 backbone). In some embodiments, the recombinant CMV and recombinant CMV viral vector comprises a nucleic acid encoding a heterologous pathogen-specific antigen, such as a HPV antigen as described herein. A CMV viral vector made by such a method is also within the scope of the disclosure.

In some embodiments, the recombinant CMV vector (e.g., a recombinant HCMV vector or a recombinant HCMV vector comprising a TR3 backbone) comprises a nucleic acid sequence encoding a microRNA (miRNA) recognition element (MRE). In some embodiments, the HCMV vector comprises a nucleic acid sequence encoding an MRE that contains target sites for microRNAs expressed in endothelial cells. Examples of miRNAs expressed in endothelial cells are miR126, miR-126-3p, miR-130a, miR-210, miR-221/222, miR-378, miR-296, and miR-328. In some embodiments, the recombinant CMV vector (e.g., a recombinant HCMV vector or a recombinant HCMV vector comprising a TR3 backbone) comprises a nucleic acid sequence encoding an MRE that contains target sites for microRNAs expressed in myeloid cells. Examples of miRNAs expressed in myeloid cells are miR-142-3p, miR-223, miR-27a, miR-652, miR-155, miR-146a, miR-132, miR-21, miR-124, and miR-125.

MREs that may be included in the vectors disclosed herein may be any miRNA recognition element that silences expression in the presence of a miRNA expressed by endothelial cells or a miRNA expressed by myeloid cells. Such an MRE may be the exact complement of a miRNA. Alternatively, other sequences may be used as MREs for a given miRNA. For example, MREs may be predicted from sequences using publicly available data bases. In one example, the miRNA may be searched on the website microRNA.org (www.microrna.org). In turn, a list of mRNA targets of the miRNA will be listed. For each listed target on the page, ‘alignment details’ may be accessed and putative MREs accessed. One of ordinary skill in the art may select a validated, putative, or mutated MRE sequence from the literature that would be predicted to induce silencing in the presence of a miRNA expressed in a myeloid cell such as a macrophage. One example involves the above referenced website. The person of ordinary skill in the art may then obtain an expression construct whereby a reporter gene (such as a fluorescent protein, enzyme, or other reporter gene) has expression driven by a promoter such as a constitutively active promoter or cell specific promoter. The MRE sequence may then be introduced into the expression construct. The expression construct may be transfected into an appropriate cell, and the cell transfected with the miRNA of interest. A lack of expression of the reporter gene indicates that the MRE silences gene expression in the presence of the miRNA.

In some embodiments, the CMV vector comprises a nucleic acid sequence that does not encode any MREs.

In some embodiments, the CMV vectors described herein contain mutations that may prevent host to host spread, thereby rendering the virus unable to infect immunocompromised or other subjects that could face complications as a result of CMV infection. The CMV vectors described herein may also contain mutations that result in the presentation of immunodominant and nonimmunodominant epitopes as well as non-canonical MHC restriction. However, in some embodiments, mutations in the CMV vectors described herein do not affect the ability of the vector to reinfect a subject that has been previously infected with CMV. Such CMV mutations are described in, for example, U.S. Patent Application Publication Nos. US2013/0136768A1, US2013/0142823A1; US2014/0141038A1; and International Application Publication No. WO2014/138209A1, which are incorporated by reference herein for teachings related to these mutations.

The CMV vectors disclosed herein may be prepared by inserting DNA comprising a sequence that encodes the HPV antigen (e.g., a fusion protein as disclosed herein) into an essential or non-essential region of the CMV genome. The method may further comprise deleting one or more regions from the CMV genome. The method may comprise in vivo recombination. Thus, the method may comprise transfecting a cell with CMV DNA in a cell-compatible medium in the presence of donor DNA comprising the heterologous DNA flanked by DNA sequences homologous with portions of the CMV genome, whereby the heterologous DNA is introduced into the genome of the CMV, and optionally then recovering CMV modified by the in vivo recombination. The method may also comprise cleaving CMV DNA to obtain cleaved CMV DNA, ligating the heterologous DNA to the cleaved CMV DNA to obtain hybrid CMV-heterologous DNA, transfecting a cell with the hybrid CMV-heterologous DNA, and optionally then recovering CMV modified by the presence of the heterologous DNA Since in vivo recombination is comprehended, the method accordingly also provides a plasmid comprising donor DNA not naturally occurring in CMV encoding a polypeptide foreign to CMV, the donor DNA is within a segment of CMV DNA that would otherwise be co-linear with an essential or non-essential region of the CMV genome such that DNA from an essential or nonessential region of CMV is flanking the donor DNA The heterologous DNA may be inserted into CMV to generate the recombinant CMV in any orientation that yields stable integration of that DNA, and expression thereof, when desired.

The DNA encoding the HPV antigen (e.g., a fusion protein as disclosed herein) in the recombinant CMV vector may also include a promoter. The promoter may be from any source such as a herpes virus, including an endogenous cytomegalovirus (CMV) promoter, such as a human CMV (HCMV), rhesus macaque CMV (RhCMV), murine, or other CMV promoter. The promoter may also be a nonviral promoter such as the EF1α promoter. The promoter may be a truncated transcriptionally active promoter which comprises a region transactivated with a transactivating protein provided by the virus and the minimal promoter region of the full-length promoter from which the truncated transcriptionally active promoter is derived. The promoter may be composed of an association of DNA sequences corresponding to the minimal promoter and upstream regulatory sequences. A minimal promoter is composed of the CAP site plus ATA box (minimum sequences for basic level of transcription; unregulated level of transcription); “upstream regulatory sequences” are composed of the upstream element(s) and enhancer sequence(s). Further, the term “truncated” indicates that the full-length promoter is not completely present, i.e., that some portion of the full-length promoter has been removed. The truncated promoter may be derived from a herpesvirus such as MCMV or HCMV, e.g., HCMV-IE or MCMV-IE. There may be up to a 40% and even up to a 90% reduction in size, from a full-length promoter, based upon base pairs. The promoter may also be a modified non-viral promoter. As to HCMV promoters, reference is made to U.S. Pat. Nos. 5,168,062 and 5,385,839. As to transfecting cells with plasmid DNA for expression therefrom, reference is made to Felgner, J H et al. (Enhanced gene delivery and mechanism studies with a novel series of cationic lipid formulations. J Biol. Chem. 269, 2550-2561 (1994)). As to direct injection of plasmid DNA as a simple and effective method of vaccination against a variety of infectious diseases reference is made to Ulmer, J B et al. (Heterologous protection against influenza by injection of DNA encoding a viral protein. Science 259, 1745-1749 (1993)). It is therefore within the scope of this disclosure that the vector may be used by the direct injection of vector DNA. Also disclosed is an expression cassette that may be inserted into a recombinant virus or plasmid comprising a truncated transcriptionally active promoter. The expression cassette may further include a functional truncated polyadenylation signal; for instance, an SV40 polyadenylation signal which is truncated, yet functional. A truncated polyadenylation signal addresses the insert size limit problems of recombinant viruses such as CMV. The expression cassette may also include heterologous DNA with respect to the virus or system into which it is inserted; and that DNA may be heterologous DNA as described herein.

It is noted that the DNA comprising the sequence encoding the fusion protein may itself include a promoter for driving expression in the CMV vector or the DNA may be limited to the coding DNA of the fusion protein. This construct may be placed in such an orientation relative to an endogenous CMV promoter that it is operably linked to the promoter and is thereby expressed. Further, multiple copies of DNA encoding the fusion protein or use of a strong or early promoter or early and late promoter, or any combination thereof, may be done so as to amplify or increase expression. Thus, the DNA encoding the fusion protein may be suitably positioned with respect to a CMV endogenous promoter, or those promoters may be translocated to be inserted at another location together with the DNA encoding the fusion protein. Nucleic acids encoding more than one fusion protein, or a fusion protein and additional antigens, may be packaged in the CMV vector.

In some embodiments, the present disclosure provides a recombinant HCMV vector comprising a nucleic acid sequence having at least 90%, 91%, 92%, 93%, 94%, 95%, 96%, 97%, 98%, 99%, or 100% identity to the nucleic acid sequence according to SEQ ID NO:17. In some embodiments, the recombinant HCMV vector comprises the nucleic acid sequence according to SEQ ID NO: 17. In some embodiments, the recombinant HCMV vector consists of the nucleic acid sequence according to SEQ ID NO:17.

### IV. Pharmaceutical Compositions

The present disclosure provides, in some embodiments, a pharmaceutical composition (e.g., an immunogenic or vaccine composition) comprising a fusion protein as described herein and a pharmaceutically acceptable carrier or diluent. The present disclosure also provides, in some embodiments, a pharmaceutical composition (e.g., an immunogenic or vaccine composition) comprising vector encoding a fusion protein as described herein (e.g., a recombinant CMV vectors disclosed herein). An immunogenic or vaccine composition containing the recombinant CMV virus or vector (or an expression product thereof) elicits an immunological response (local or systemic). The response can, but need not be, protective. In other words, an immunogenic or vaccine composition elicits a local or systemic protective or therapeutic response.

The present disclosure provides, in some embodiments, a pharmaceutical composition (e.g., an immunogenic or vaccine composition) comprising an HPV fusion protein antigen as described herein and a pharmaceutically acceptable carrier or diluent.

Such pharmaceutical compositions may be prepared in accordance with standard techniques well known to those skilled in the pharmaceutical arts. Such compositions may be administered in dosages and by techniques well known to those skilled in the medical arts taking into consideration such factors as the breed or species, age, sex, weight, and condition of the particular patient, and the route of administration. The compositions may be administered alone, or may be co-administered or sequentially administered with other proteins or peptides, with other vectors (e.g., other CMV vectors), or with other immunological, antigenic or vaccine or therapeutic compositions. Such other compositions may include purified native antigens or epitopes or antigens or epitopes from the expression by a recombinant CMV or another vector system.

Pharmaceutical compositions as disclosed herein may be formulated so as to be used in any administration procedure known in the art. Such pharmaceutical compositions may be via a parenteral route (intradermal, intraperitoneal, intramuscular, subcutaneous, intravenous, or others). The administration may also be via a mucosal route, e.g., oral, nasal, genital, etc.

Examples of compositions include liquid preparations for orifice, e.g., oral, nasal, anal, genital, e.g., vaginal, etc., administration such as suspensions, syrups or elixirs; and, preparations for parenteral, subcutaneous, intraperitoneal, intradermal, intramuscular or intravenous administration (e.g., injectable administration) such as sterile suspensions or emulsions. In such compositions the recombinant may be in admixture with a suitable carrier, diluent, or excipient such as sterile water, physiological saline, glucose, trehalose, or the like.

Pharmaceutical compositions disclosed herein typically may comprise or contain an adjuvant and an amount of the antigen (e.g., fusion protein) or vector encoding the antigen, to elicit the desired response. In human applications, alum (aluminum phosphate or aluminum hydroxide) is a typical adjuvant. Saponin and its purified component Quil A, Freund's complete adjuvant and other adjuvants used in research and veterinary applications have toxicities which limit their potential use in human vaccines. Chemically defined preparations such as muramyl dipeptide, monophosphoryllipid A, phospholipid conjugates such as those described by Goodman-Snitkoff, G. et al. (Role of intrastructural/intermolecular help in immunization with peptide-phospholipid complexes. J Immunol. 147, 410-415 (1991)), encapsulation of the protein within a proteoliposome as described by Miller, M D et al. (Vaccination of rhesus monkeys with synthetic peptide in a fusogenic proteoliposome elicits simian immunodeficiency virus-specific CD8+ cytotoxic T lymphocytes. J Exp. Med. 176, 1739-1744 (1992)), and encapsulation of the protein in lipid vesicles such as Novasome lipid vesicles (Micro Vescular Systems, Inc., Nashua, N.H.) may also be used.

The composition may be packaged in a single dosage form for immunization by parenteral (e.g., intramuscular, intradermal, or subcutaneous) administration or orifice administration, e.g., perlingual (e.g., oral), intragastric, mucosal including intraoral, intraanal, intravaginal, and the like administration. The effective dosage and route of administration are determined by the nature of the composition, by the nature of the expression product, by expression level if a vector is directly used, and by known factors, such as breed or species, age, sex, weight, condition, and nature of the subject, as well as LD50 and other screening procedures which are known and do not require undue experimentation. Dosages of expressed product may range from a few to a few hundred micrograms, e.g., 5 to 500 pg. The antigen or vector encoding the antigen may be administered in any suitable amount to achieve expression at these dosage levels. In nonlimiting examples: a CMV vector encoding the fusion protein disclosed herein may be administered in an amount of at least 10₂ pfu; or in a range from about 10₂ pfu to about 10₇ pfu. Other suitable carriers or diluents may be water or a buffered saline, with or without a preservative. The composition may be lyophilized for resuspension at the time of administration or may be in solution.

In nonlimiting examples: the CMV vectors may be administered in an amount of about 1×10³ ffu, about 3×10⁴ ffu, about 5×10⁴ ffu, about 5×10⁵ ffu, about 1×10⁶ ffu, about 5×10⁶ ffu, or about 1×10⁷ ffu. In nonlimiting examples: the CMV vectors may be administered in one dose, at least one dose, two doses, or at least two doses. As a non-limiting example, the CMV vectors may be administered in two doses. An initial dose may be referred to as a “prime” dose and any subsequent dose or doses may be referred to as a “boost” dose or “boost” doses. As a non-limiting example, a “boost” dose may be administered at about 84 days, or 12 weeks after administration of the “prime” dose. Other suitable carriers or diluents may be water or a buffered saline, with or without a preservative. The CMV vector may be lyophilized for resuspension at the time of administration or may be in solution. In nonlimiting examples: the suspended CMV vector may be administered as an injection having a volume of less than 1 ml, about 1 ml, about 2 ml, or more than 1 ml. In a nonlimiting example, the CMV vector may be administered subcutaneously, optionally, in the deltoid region.

### V. Methods of Treatment and Other Uses

The fusion proteins and vectors (such as recombinant CMV vectors) disclosed herein may be used in methods of inducing an immunological or immune response in a subject comprising administering to the subject a composition comprising the fusion protein, the vector, or the recombinant CMV virus or vector and a pharmaceutically acceptable carrier or diluent.

As used herein, the term “subject” refers to a living multi-cellular vertebrate organism, a category that includes both human and non-human mammals. The subject may be an animal, such as a mammal, including any mammal that can be infected with Human papillomavirus, e.g., a primate (such as a human, a non-human primate, e.g., a monkey, or a chimpanzee), or an animal that is considered an acceptable clinical model of HPV infection.

In some embodiments, the subject is human.

In some embodiments the subject has tested positive for HPV. In some embodiments the patient has tested negative for HPV. HPV testing refers to assays that determine the presence of the virus in cervical cells, or other cells from subject. Non-limiting examples of HPV tests include polymerase chain reaction (PCR), DNA:RNA hybridization, and lateral flow strip tests. The subject sample may be positive for any subtype of HPV infection such as, for example, HPV 16 or HPV 18 infection.

In some embodiments the subject has tested positive for CMV. In some embodiments the patient has tested negative for CMV. CMV testing refers to assays that determine the presence of the virus in the urine, saliva, blood, sputum, or other body fluids. Non-limiting examples of CMV tests include polymerase chain reaction (PCR), e.g., a CMV-salivary PCR test.

In some embodiments, the subject has a serological status with regard to HCMV infection. As used herein, the term “seropositive” refers to a subject or immune system that has been previously exposed to a particular antigen and thus has a detectable serum antibody titer against the antigen of interest. The phrase “seropositive for HCMV” refers to a subject or immune system that has been previously exposed to a HCMV antigen. A seropositive subject or immune system can be distinguished by the presence of antibodies or other immune markers in the serum that indicate past exposure to a particular antigen. As used herein, the term “seronegative” refers to a subject or immune system that has not been previously exposed to a particular antigen and thus has an absence of detectable serum antibody titer against the antigen of interest. The phrase “seronegative for HCMV” refers to a subject or immune system that has not been previously exposed to a HCMV antigen.

As used herein, the term “treatment” refers to an intervention that ameliorates a sign or symptom of a disease or pathological condition. As used herein, the terms “treatment”, “treat”, and “treating”, with reference to a disease, pathological condition or symptom, also refers to any observable beneficial effect of the treatment. The beneficial effect may be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed.

As used herein, the terms “preventing” or “prevention” refer to the failure to develop a disease, disorder, or condition, or the reduction in the development of a sign or symptom associated with such a disease, disorder, or condition (e.g., by a clinically relevant amount), or the exhibition of delayed signs or symptoms delayed (e.g., by days, weeks, months, or years). Prevention may require the administration of more than one dose.

As used herein, the disease, disorder, or condition to be prevented or treated may include an abnormal cytology, neoplasm, dysplasia, or any precancerous condition that may lead to the development of cancer. Abnormal cytologies, neoplasms, and dysplasias include, but are not limited to high-grade squamous intraepithelial lesions (HSILs), CIN (cervical intraepithelial neoplasia), HPV-associated high-grade squamous intraepithelial lesions (HSILs). HSILs include, but are not limited to cervical HSIL, cervical squamous intraepithelial neoplasia 2 (CIN2), cervical squamous intraepithelial neoplasia 3 (CIN3), anal HSIL, anal intraepithelial neoplasia 2 (AIN2), and anal intraepithelial neoplasia 3 (AIN3). As used herein, the disease, disorder, or condition to be prevented or treated may include anal intraepithelial neoplasia 1 (AIN1) or cervical squamous intraepithelial neoplasia 1 (CIN1).

As used herein, the disease, disorder, or condition to be prevented or treated may include a HPV-associated cancer. HPV-associated cancers include, but are not limited to cervical, penile, vaginal, vulval, anal, rectal, oropharyngeal, skin, and head and neck cancer. In some embodiments, the HPV-associated cancer is a HSIL. In some embodiments, the HPV-associated cancer is cervical HSIL, CIN1, CIN2, CIN3, anal HSIL, AIN1, AIN2, or AIN3.

As used herein, the term “effective amount” refers to an amount of an agent (e.g., a fusion protein or a vector encoding a fusion protein), that is sufficient to generate a desired response, such as reduce or eliminate a sign or symptom of a condition or disease or induce an immune response to an antigen. In some examples, an “effective amount” is one that treats (including prophylaxis) one or more symptoms and/or underlying causes of any of a disorder or disease. An effective amount may be a therapeutically effective amount, including an amount that prevents one or more signs or symptoms of a particular disease or condition from developing, such as one or more signs or symptoms associated with infectious disease or cancer.

The disclosed fusion proteins or vectors may be administered in vivo, for example where the aim is to produce an immunogenic response, including a CD4+ T cell/immune and/or a CD8+T cell/immune. For example, in some examples it may be desired to use the disclosed fusion proteins or vectors in a laboratory animal, such as rhesus macaques for preclinical testing of immunogenic compositions and vaccines using RhCMV. In other examples, it will be desirable to use the fusion proteins or vectors in human subjects, such as in clinical trials and for actual clinical use of the immunogenic compositions using HCMV.

For such in vivo applications the disclosed fusion proteins or vectors may be administered as a component of an immunogenic or pharmaceutical composition further comprising a pharmaceutically acceptable carrier. In some embodiments, the immunogenic compositions of the disclosure are useful to stimulate an immune response against the fusion protein, and may be used as one or more components of a prophylactic or therapeutic vaccine. The nucleic acids and vectors of the disclosure are particularly useful for providing genetic vaccines, i.e., vaccines for delivering the nucleic acids encoding the antigens of the disclosure to a subject, such as a human, such that the antigens are then expressed in the subject to elicit an immune response.

Immunization schedules (or regimens) are well known for animals (including humans) and may be readily determined for the particular subject and immunogenic composition. Hence, the immunogens may be administered one or more times to the subject. Preferably, there is a set time interval between separate administrations of the immunogenic composition. While this interval varies for every subject, typically it ranges from 10 days to several weeks, and is often 2, 4, 6, or 8 weeks. For humans, the interval is typically from 2 to 6 weeks. In a particularly advantageous embodiment of the present disclosure, the interval is longer, advantageously about 10 weeks, 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks, 50 weeks, 52 weeks, 54 weeks, 56 weeks, 58 weeks, 60 weeks, 62 weeks, 64 weeks, 66 weeks, 68 weeks, or 70 weeks. The immunization regimes typically have from 1 to 6 administrations of the immunogenic composition, but may have as few as one or two or four. The methods of inducing an immune response may also include administration of an adjuvant with the immunogens. In some instances, annual, biannual, or other long interval (5-10 years) booster immunization may supplement the initial immunization protocol. The present methods also include a variety of prime-boost regimens. In these methods, one or more priming immunizations are followed by one or more boosting immunizations. The actual immunogenic composition may be the same or different for each immunization and the type of immunogenic composition (e.g., containing protein or expression vector), the route, and formulation of the immunogens may also be varied. For example, if an expression vector is used for the priming and boosting steps, it may either be of the same or different type (e.g., DNA or bacterial or viral expression vector). One useful prime-boost regimen provides for two priming immunizations, four weeks apart, followed by two boosting immunizations at 4 and 8 weeks after the last priming immunization. It should also be readily apparent to one of skill in the art that there are several permutations and combinations that are encompassed using the DNA, bacterial, and viral expression vectors of the disclosure to provide priming and boosting regimens. CMV vectors may be used repeatedly while expressing different antigens derived from different pathogens.

Accordingly, the present disclosure provides, in some embodiments, a method of generating an immune response in a subject, comprising administering to the subject any of the aforementioned fusion proteins, nucleic acids, vectors, or compositions. Also provided herein is the use of any of the aforementioned fusion proteins, nucleic acids, vectors, or compositions in the manufacture of a medicament for use in generating an immune response in a subject.

In some embodiments, the present disclosure provides a method of treating or preventing HPV infection in a subject, comprising administering to the subject a fusion protein, nucleic acid, vector, or composition described herein. In some embodiments, a fusion protein, nucleic acid, vector, or composition described herein is used in the manufacture of a medicament for use in treating or preventing HPV infection in a subject. In some embodiments, the present disclosure provides a method of preventing HPV in a subject. In some embodiments, the present disclosure provides a method of preventing HPV in a subject, wherein the subject has had a negative result from an HPV test. In some embodiments, the present disclosure provides a method of treating HPV in a subject. In some embodiments, the present disclosure provides a method of treating HPV in a subject, wherein the subject has had a positive result from an HPV test.

In some embodiments, the present disclosure provides a method of preventing recurrence of HPV infection in a subject. In further embodiments, the prevention of recurrence occurs after a previous treatment for HPV.

In some embodiments, the present disclosure provides a method of treating or preventing an HPV-associated cancer (e.g. cervical, penile, vaginal, vulval, anal, rectal, oropharyngeal, skin, head and neck cancer, HSIL, cervical HSIL, CIN1, CIN2, CIN3, anal HSIL, AIN1, AIN2, AIN3, etc.) in a subject, comprising administering to the subject a fusion protein, nucleic acid, vector, or composition described herein. In some embodiments, a fusion protein, nucleic acid, vector, or composition described herein is used in the manufacture of a medicament for use in treating or preventing an HPV-associated cancer in a subject. In some embodiments, the present disclosure provides a method of preventing an HPV-associated cancer in a subject. In some embodiments, the present disclosure provides a method of preventing an HPV-associated cancer in a subject, wherein the subject has had a negative result from an HPV test. In some embodiments, the present disclosure provides a method of preventing an HPV-associated cancer in a subject, wherein the subject has had a positive result from an HPV test. In some embodiments, the present disclosure provides a method of treating an HPV-associated cancer in a subject. In some embodiments, the present disclosure provides a method of treating an HPV-associated cancer in a subject, wherein the subject has had a negative result from an HPV test. In some embodiments, the present disclosure provides a method of treating an HPV-associated cancer in a subject, wherein the subject has had a positive result from an HPV test.

In some embodiments, the present disclosure provides a method of preventing recurrence of an HPV-associated cancer in a subject. In further embodiments, the prevention of recurrence occurs after a previous treatment for an HPV-associated cancer.

In some embodiments, the present disclosure provides a method of treating an abnormal cytology, neoplasm, dysplasia, or precancerous condition (e.g., a HSIL, CIN1, CIN2, CIN3, AIN1, AIN2, AIN3, etc.) in a subject, comprising administering to the subject a fusion protein, nucleic acid, vector, or composition described herein. In some embodiments, a fusion protein, nucleic acid, vector, or composition described herein is used in the manufacture of a medicament for use in treating or preventing an abnormal cytology, neoplasm, dysplasia, or precancerous condition in a subject. In some embodiments, the present disclosure provides a method of preventing an abnormal cytology, neoplasm, dysplasia, or precancerous condition in a subject. In some embodiments, the present disclosure provides a method of preventing an abnormal cytology, neoplasm, dysplasia, or precancerous condition in a subject, wherein the subject has had a negative result from an HPV test. In some embodiments, the present disclosure provides a method of preventing an abnormal cytology, neoplasm, dysplasia, or precancerous condition in a subject, wherein the subject has had a positive result from an HPV test. In some embodiments, the present disclosure provides a method of treating an abnormal cytology, neoplasm, dysplasia, or precancerous condition in a subject. In some embodiments, the present disclosure provides a method of treating an abnormal cytology, neoplasm, dysplasia, or precancerous condition in a subject, wherein the subject has had a negative result from an HPV test. In some embodiments, the present disclosure provides a method of treating an abnormal cytology, neoplasm, dysplasia, or precancerous condition in a subject, wherein the subject has had a positive result from an HPV test.

In some embodiments, the present disclosure provides a method of preventing recurrence of an abnormal cytology, neoplasm, dysplasia, or any precancerous condition in a subject. In further embodiments, the prevention of recurrence occurs after a previous treatment for an abnormal cytology, neoplasm, dysplasia, or precancerous condition.

In some of the aforementioned methods, uses, or compositions for use, the subject has human immunodeficiency virus (HIV), i.e., is “HIV positive” or positive for HIV infection. In some embodiments, the subject has HIV and has received an antiretroviral therapy (ART). In some embodiments, the subject has HIV and is on an ART. In some embodiments, the subject is administered or is to be administered the fusion protein, nucleic acid, vector, or composition and an ART.

In some of the aforementioned methods, uses, or compositions for use, the vector is a CMV vector and the CMV vector is administered in an amount effective to elicit a CD4+ T cell response to an HPV antigen. In some embodiments, at least 10% of the CD4+ T cells elicited by the recombinant HCMV vector are restricted by MHC-II or an ortholog thereof. In some further embodiments, at least 20%, at least 30%, at least 40%, at least 50%, at least 60%, at least 75%, at least 80%, at least 85%, at least 90%, or at least 95% of the CD4+ T cells elicited by the recombinant HCMV vector are restricted by MHC-II or an ortholog thereof.

In some of the aforementioned methods, uses, or compositions for use, the vector is a CMV vector and the CMV vector is administered in an amount effective to elicit a CD8+ T cell response to a HPV antigen. In some embodiments, at least 10% of the CD8+ T cells elicited by the CMV vector are restricted by MHC-Ia or an ortholog thereof. In some further embodiments, at least 20%, at least 30%, at least 40%, at least 50%, at least 60%, at least 75%, at least 80%, at least 85%, at least 90%, or at least 95% of the CD8+ T cells elicited by the CMV vector are restricted by MHC-Ia or an ortholog thereof.

### VI. Example Embodiments

The following exemplary embodiments are also provided by the present disclosure:

1. A fusion protein comprising or consisting of:
(a) an amino acid sequence having 90%, 91%, 92%, 93%, 94%, 95%, 96%, 97%, 98%, 99%, or 100% identity to the amino acid sequence according to SEQ ID NO:4;
(b) the amino acid sequence according to SEQ ID NO:4;
(c) an amino acid sequence having 90%, 91%, 92%, 93%, 94%, 95%, 96%, 97%, 98%, 99%, or 100% identity to the amino acid sequence according to SEQ ID NO:1;
(d) the amino acid sequence according to SEQ ID NO:1;
(e) an amino acid sequence having 90%, 91%, 92%, 93%, 94%, 95%, 96%, 97%, 98%, 99%, or 100% identity to the amino acid sequence according to SEQ ID NO:2;
(f) the amino acid sequence according to SEQ ID NO:2;
(g) an amino acid sequence having 90%, 91%, 92%, 93%, 94%, 95%, 96%, 97%, 98%, 99%, or 100% identity to the amino acid sequence according to SEQ ID NO:3;
(h) the amino acid sequence according to SEQ ID NO:3;
(i) (i) an amino acid sequence having 90%, 91%, 92%, 93%, 94%, 95%, 96%, 97%, 98%, 99%, or 100% identity to the amino acid sequence according to SEQ ID NO: 1; (ii) an amino acid sequence having 90%, 91%, 92%, 93%, 94%, 95%, 96%, 97%, 98%, 99%, or 100% identity to the amino acid sequence according to SEQ ID NO: 2; and (iii) an amino acid sequence having 90%, 91%, 92%, 93%, 94%, 95%, 96%, 97%, 98%, 99%, or 100% identity to the amino acid sequence according to SEQ ID NO: 3;
(j) (i) the amino acid sequence according to SEQ ID NO: 1; (ii) the amino acid sequence according to SEQ ID NO:2; and (iii) the amino acid sequence according to SEQ ID NO:3;
(k) (i) an amino acid sequence having 90%, 91%, 92%, 93%, 94%, 95%, 96%, 97%, 98%, 99%, or 100% identity to the amino acid sequence according to SEQ ID NO: 1; and (ii) an amino acid sequence having 90%, 91%, 92%, 93%, 94%, 95%, 96%, 97%, 98%, 99%, or 100% identity to the amino acid sequence according to SEQ ID NO: 2;
(l) (i) the amino acid sequence according to SEQ ID NO: 1; and (ii) the amino acid sequence according to SEQ ID NO:2;
(m) (i) an amino acid sequence having 90%, 91%, 92%, 93%, 94%, 95%, 96%, 97%, 98%, 99%, or 100% identity to the amino acid sequence according to SEQ ID NO: 1; and (ii) an amino acid sequence having 90%, 91%, 92%, 93%, 94%, 95%, 96%, 97%, 98%, 99%, or 100% identity to the amino acid sequence according to SEQ ID NO: 3; or
(n) (i) the amino acid sequence according to SEQ ID NO: 1; and (ii) the amino acid sequence according to SEQ ID NO:3.

2. A fusion protein encoded by a nucleic acid comprising a sequence having 90%, 91%, 92%, 93%, 94%, 95%, 96%, 97%, 98%, 99%, or 100% identity to the nucleic acid sequence according to SEQ ID NO:16.

3. A fusion protein encoded by a nucleic acid comprising the nucleic acid sequence according to SEQ ID NO:16.

4. A fusion protein encoded by a nucleic acid comprising a sequence having 90%, 91%, 92%, 93%, 94%, 95%, 96%, 97%, 98%, 99%, or 100% identity to the nucleic acid sequence according to SEQ ID NO: 13.

5. A fusion protein encoded by a nucleic acid comprising the nucleic acid sequence according to SEQ ID NO:13.

6. A fusion protein encoded by a nucleic acid comprising a sequence having 90%, 91%, 92%, 93%, 94%, 95%, 96%, 97%, 98%, 99%, or 100% identity to the nucleic acid sequence according to SEQ ID NO:14.

7. A fusion protein encoded by a nucleic acid comprising the nucleic acid sequence according to SEQ ID NO:14.

8. A fusion protein encoded by a nucleic acid comprising a sequence having 90%, 91%, 92%, 93%, 94%, 95%, 96%, 97%, 98%, 99%, or 100% identity to the nucleic acid sequence according to SEQ ID NO:15.

9. A fusion protein encoded by a nucleic acid comprising the nucleic acid sequence according to SEQ ID NO:15.

10. The fusion protein of any one of embodiments 1-9, wherein the fusion protein comprises one or more proteins from at least one of HPV subtypes HPV16 and HPV18.

11. The fusion protein of any one of embodiments 1-10, wherein the fusion protein further comprises one or more linkers connecting one or more of SEQ ID NOs: 1, 2, and 3, wherein each of the one or more linkers comprises or consists of one or more amino acid residues.

12. A nucleic acid molecule encoding the fusion protein according to any one of embodiments 1-11.

13. A vector comprising the nucleic acid molecule of embodiment 12.

14. The vector of embodiment 13, further comprising a promoter, wherein a promoter is operably linked to the nucleic acid molecule encoding the fusion protein.

15. The vector of embodiment 13 or embodiment 14, wherein the vector is a viral vector.

16. The vector of embodiment 15, wherein the viral vector is a cytomegalovirus (CMV) vector.

17. The vector of any one of embodiments 12-16, wherein the viral vector is a RhCMV vector, a HCMV vector, or a recombinant HCMV vector.

18. A vector comprising a nucleic acid sequence having at least 90%, 91%, 92%, 93%, 94%, 95%, 96%, 97%, 98%, 99%, or 100% identity to the nucleic acid sequence according to SEQ ID NO: 17.

19. A vector comprising the nucleic acid sequence according to SEQ ID NO:17.

20. A vector consisting essentially of the nucleic acid sequence according to SEQ ID NO:17.

21. A vector consisting of the nucleic acid sequence according to SEQ ID NO:17.

22. The vector of any one of embodiments 16-21, wherein the promoter is operably linked to the nucleic acid molecule encoding the fusion protein and the promoter is a UL78 promoter, or an ortholog thereof.

23. The vector of embodiment 22, wherein the nucleic acid molecule encoding the fusion protein replaces all or part of UL78.

24. The vector of embodiment 23, wherein the vector comprises a nucleic acid sequence having at least 90%, 91%, 92%, 93%, 94%, 95%, 96%, 97%, 98%, 99%, or 100% identity to the nucleic acid sequence according to SEQ ID NO:17.

25. The vector of any one of embodiments 16-24, wherein the RhCMV vector or HCMV vector does not express UL128 or UL130, or orthologs thereof.

26. A recombinant HCMV vector comprising a TR3 backbone and a nucleic acid sequence encoding a heterologous antigen according to SEQ ID NO: 4, wherein:
(a) the vector does not express UL128 or UL130, or orthologs thereof;
(b) the vector comprises a nucleic acid sequence encoding UL146 and UL147, or orthologs thereof; and
(c) the nucleic acid encoding a heterologous antigen replaces all or part of UL78 and is operably linked to the UL78 promoter.

27. The recombinant HCMV vector of embodiment 26, wherein the recombinant HCMV vector comprises a nucleic acid sequence encoding UL18 and UL82, or orthologs thereof.

28. The recombinant HCMV vector of embodiment 26, wherein the recombinant HCMV vector comprises a nucleic acid sequence having at least 90%, 91%, 92%, 93%, 94%, 95%, 96%, 97%, 98%, 99%, or 100% identity to the nucleic acid sequence according to SEQ ID NO:17.

29. The vector of any one of embodiments 16-28, wherein the RhCMV or HCMV vector (i) comprises a nucleic acid sequence encoding UL146 and a nucleic acid sequence encoding UL147, or orthologs thereof; and (ii) does not express UL128 or UL130, or orthologs thereof.

30. The vector of any one of embodiments 25-29, wherein the vector does not express a UL128 protein or a UL130 protein, resulting from the presence of one or more mutations in the nucleic acid sequences encoding UL128 and UL130.

31. The vector of embodiment 30, wherein the mutation in the nucleic acid sequences encoding UL128 and UL130 is a point mutation, frameshift mutation, truncation mutation, or deletion of all of the nucleic acid sequences encoding the viral protein.

32. The vector of any one of embodiments 16-31, wherein the vector is a HCMV vector comprising a TR3 backbone.

33. A pharmaceutical composition comprising (i) (a) the fusion protein of any one of embodiments 1-11, (b) the nucleic acid of embodiment 12, or (c) the vector of any one of embodiments 13-32; and (ii) a pharmaceutically acceptable carrier.

34. An immunogenic composition comprising (i) (a) the fusion protein of any one of embodiments 1-11, (b) the nucleic acid of embodiment 12, or (c) the vector of any one of embodiments 13-32; and (ii) a pharmaceutically acceptable carrier.

35. A pharmaceutical composition comprising the fusion protein of any one of embodiments 1-11 or the vector of any one of embodiments 13-31, wherein the pharmaceutical composition is lyophilized.

36. A method of generating an immune response in a subject, comprising administering to the subject the fusion protein, nucleic acid, vector, or composition of any one of embodiments 1-35.

37. Use of the fusion protein, nucleic acid, vector, or composition of any one of embodiments 1-35 in the manufacture of a medicament for use in generating an immune response in a subject.

38. The fusion protein, nucleic acid, vector, or composition of any one of embodiments 1-35 for use in generating an immune response in a subject.

39. A method of treating or preventing an abnormal cytology, neoplasm, dysplasia, or precancerous condition in a subject, comprising administering to the subject the fusion protein, nucleic acid, vector, or composition of any one of embodiments 1-35.

40. Use of the fusion protein, nucleic acid, vector, or composition of any one of embodiments 1-35 in the manufacture of a medicament for use in treating or preventing an abnormal cytology, neoplasm, dysplasia, or precancerous condition in a subject.

41. The fusion protein, nucleic acid, vector, or composition of any one of embodiments 1-35 for use in treating or preventing an abnormal cytology, neoplasm, dysplasia, or precancerous condition in a subject.

42. A method of treating or preventing HPV infection in a subject, comprising administering to the subject the fusion protein, nucleic acid, vector, or composition of any one of embodiments 1-35.

43. Use of the fusion protein, nucleic acid, vector, or composition of any one of embodiments 1-35 in the manufacture of a medicament for use in treating or preventing HPV infection in a subject.

44. The fusion protein, nucleic acid, vector, or composition of any one of claims 1-35 for use in treating or preventing HPV infection in a subject.

45. A method of treating or preventing an HPV-associated cancer in a subject, comprising administering to the subject the fusion protein, nucleic acid, vector, or composition of any one of claims 1-35.

46. Use of the fusion protein, nucleic acid, vector, or composition of any one of embodiments 1-35 in the manufacture of a medicament for use in treating or preventing an HPV-associated cancer in a subject.

47. The fusion protein, nucleic acid, vector, or composition of any one of embodiments 1-35 for use in treating or preventing an HPV-associated cancer in a subject.

48. The fusion protein, nucleic acid, vector, or composition of any one of embodiments 1-35 for use in treating or preventing HPV infection in a subject, wherein the subject is CMV positive.

49. The fusion protein, nucleic acid, vector, or composition of any one of embodiments 1-35 for use in treating or preventing HPV infection in a subject, wherein the subject is CMV negative.

50. The fusion protein, nucleic acid, vector, or composition of any one of embodiments 1-35, 48, and 49 for use in treating HPV infection in a subject, wherein the subject is HPV positive.

51. The fusion protein, nucleic acid, vector, or composition of any one of embodiments 1-35, 48, and 49 for use in preventing HPV infection in a subject, wherein the subject is HPV negative.

52. The fusion protein, nucleic acid, vector, or composition of any one of embodiments 1-35 for use in treating or preventing an abnormal cytology, neoplasm, dysplasia, or precancerous condition in a subject, wherein the subject is CMV positive.

53. The fusion protein, nucleic acid, vector, or composition of any one of embodiments 1-35 for use in treating or preventing an abnormal cytology, neoplasm, dysplasia, or precancerous condition in a subject, wherein the subject is CMV negative.

54. The fusion protein, nucleic acid, vector, or composition of any one of embodiments 1-35, 52, and 53 for use in treating or preventing an abnormal cytology, neoplasm, dysplasia, or precancerous condition in a subject, wherein the subject is HPV positive.

55. The fusion protein, nucleic acid, vector, or composition of any one of embodiments 1-35, 52, and 53 for use in treating or preventing an abnormal cytology, neoplasm, dysplasia, or precancerous condition in a subject, wherein the subject is HPV negative.

56. The method, use in manufacture, or use of any one of embodiments 39-41 or 52-55, wherein the abnormal cytology, neoplasm, dysplasia, or precancerous condition is a high-grade squamous intraepithelial lesion (HSIL).

57. The method, use in manufacture, or use of embodiment 56, wherein the HSIL is a cervical HSIL or anal HSIL.

58. The method, use in manufacture, or use of embodiment 57, wherein the cervical HSIL is a cervical squamous intraepithelial neoplasia 2 (CIN2) or cervical squamous intraepithelial neoplasia 3 (CIN3).

59. The method, use in manufacture, or use of embodiment 57, wherein the anal HSIL is an anal intraepithelial neoplasia 2 (AIN2) or anal intraepithelial neoplasia (AIN3).

60. The fusion protein, nucleic acid, vector, or composition of any one of embodiments 1-35 for use in treating or preventing an HPV-associated cancer in a subject, wherein the subject is CMV positive.

61. The fusion protein, nucleic acid, vector, or composition of any one of embodiments 1-35 for use in treating or preventing an HPV-associated cancer in a subject, wherein the subject is CMV negative.

62. The fusion protein, nucleic acid, vector, or composition of any one of embodiments 1-35, 60, and 61 for use in treating or preventing an HPV-associated cancer in a subject, wherein the subject is HPV positive.

63. The fusion protein, nucleic acid, vector, or composition of any one of embodiments 1-35, 60, and 61 for use in treating or preventing an HPV-associated cancer in a subject, wherein the subject is HPV negative.

64. The method, use in manufacture, or use of any one of embodiments 45-47 or 60-63, wherein the HPV-associated cancer is cervical, penile, vaginal, vulval, anal, rectal, oropharyngeal, skin, and/or head and neck cancer.

65. The fusion protein, nucleic acid, vector, or composition of any one of embodiments 1-35 or 48-51 for use in treating or preventing HPV infection in a subject, wherein the subject is administered a second therapy.

66. The fusion protein, nucleic acid, vector, or composition of any one of embodiments 1-35 or 60-64 for use in treating or preventing HPV-associated cancer in a subject, wherein the subject is administered a second therapy.

67. The method, use in manufacture, or use of any one of embodiments 36-66, wherein the vector is a CMV vector and the CMV vector is administered in an amount effective to elicit a CD4+ T cell response to an HPV antigen.

68. The method, use in manufacture, or use of embodiment 67, wherein at least 10% of the CD4+ T cells elicited by the recombinant HCMV vector are restricted by MHC-II or an ortholog thereof.

69. The method, use in manufacture, or use of embodiment 68, wherein at least 20%, at least 30%, at least 40%, at least 50%, at least 60%, at least 75%, at least 80%, at least 85%, at least 90%, or at least 95% of the CD4+ T cells elicited by the recombinant HCMV vector are restricted by MHC-II or an ortholog thereof.

70. The method, use in manufacture, or use of any one of embodiments 36-69, wherein the vector is a CMV vector and the CMV vector is administered in an amount effective to elicit a CD8+ T cell response to an HPV antigen.

71. The method, use in manufacture, or vector or composition for use of embodiment 70, wherein at least 10% of the CD8+ T cells elicited by the CMV vector are restricted by MHC-Ia or an ortholog thereof.

72. The method, use in manufacture, or vector or composition for use of embodiment 71, wherein at least 20%, at least 30%, at least 40%, at least 50%, at least 60%, at least 75%, at least 80%, at least 85%, at least 90%, or at least 95% of the CD8+ T cells elicited by the CMV vector are restricted by MHC-Ia or an ortholog thereof.

73. The method, use in manufacture, or vector or composition for use according to any one of the preceding embodiments, wherein the fusion protein, nucleic acid, vector, or composition is administered subcutaneously or is for subcutaneous administration.

74. The method, use in manufacture, or vector or composition for use according to any one of the preceding embodiments, wherein the subject is positive for human immunodeficiency virus (HIV).

75. The method, use in manufacture, or vector or composition for use of embodiment 74, wherein the subject has received an antiretroviral therapy (ART).

76. The method, use in manufacture, or vector or composition for use of embodiment 74 or 75, wherein the subject is administered or is to be administered an ART.

### VII. Sequences

SEQ ID NO: 1-HPV18 E7-HPV18 E6-HPV16 E7-HPV16 E6 Fusion amino
acid sequence
MHGPKATLQDIVLHLEPQNEIPVDLLGHGQLSDSEEENDEIDGVNHQHLPARRAEPQRHTMLCMG
CKCEARIELVVESSADDLRAFQQLFLNTLSFVGPWCASQQMARFEDPTRRPYKLPDLCTELNTSL
QDIEITCVYCKTVLELTEVFEFAFKDLFVVYRDSIPHAACHKGIDFYSRIRELRHYSDSVYGDTL
EKLTNTGLYNLLIRCLRCQKPLNPAEKLRHLNEKRRFHNIAGHYRGQGHSCCNRARQERLQRHGD
TPTLHEYMLDLQPETTDLYGYGQLNDSSEEEDEIDGPAGQAEPDRAHYNIVTFGCKCDSTLRLCV
QSTHVDICTLEDLLMGTLGIVGPICSQKPMFQDPQERPRKLPQLCTELQTTIHDIILECVYCKQQ
LLRREVYDFAFRDLCIVYRDGNPYAVCDKGLKFYSKISEYRHYCYSLYGTTLEQQYNKPLCDLLI
RCINCQKPLCPEEKQRHLDKKQRFHNIRGRWTGRGMSCCRSSRT
SEQ ID NO: 2-HPV16 E1-HPV18 E1 Fusion amino acid sequence
PKLRSTAAALYWYKTGMSNISEVYGDTPEWIQRQTVLQHSENDCTFELSQMVQWAYDNDIVDDSE
IAYKYAQLADTNSNASAFLKSNSQAKIVKDCATMCRHYKRAEKKQMSMSQWIKYRCDRVDDGGDW
KQIVMFLRYQGVDFMSFLSALKKFLQGIPKKNCILLYGAANTGALFGMSLMKFLQGSVICFVNSK
SHFAFQPLADAKIGMLDDATVPCWNYIDDNLRNALDGNLVSMDVKHRPLVQLKCPPLLITSAINA
GTDSRWPYLHNRLVVFTFPNEFPFDENGNPVYELNDKNWPKLRSSVAALYWYRTGISNISEVMGD
TPEWIQRLTIIQHGIDDSNFDLSEMVQWAFDNELTDESDMAFEYALLADSNSNAAAFLKSNCQAK
YLKDCATMCKHYRRAQKRQMNMSQWIRFRCSKIDEGGDWRPIVQFLRYQQIEFITFLGALKSFLK
GTPKKNCLVFCGPANTGAYFGMSFIHFIQGAVISFVNSTSHAFLEPLTDTKVAMLDDATTTCWTY
FDTYMRNALDGNPISIDRKHKPLIQLKCPPILLTTAIHPAKDNRWPYLESRITVFEFPNAFPFDK
NGNPVYEINDKNW
SEQ ID NO: 3-HPV16 E2-HPV18 E2 Fusion amino acid sequence
METLCQRLNVCQDKILTHYENDATDLRDHIDYWKQMALACAIYYKAREMGFKHINHQVVPTLAVS
KNKALQAAELQLTLETIYNSQYSNEKWTLQDVSLEVYLTAPTGCIAKHGYTVEVQFDGDICNTMH
YTNRKYIYICEETSVTVVEGQVDYYGLAYVHEGIQTYFVQFKDDAEKYSKNKVWEVHAGGQAILC
PTSVFMQTPKETLSERLSALQDKIIDHYENDAKDIDSQIQYWQLIAWANAIFFAAREHGIQTLNH
QVVPAYNISKSKAHKAAELQMALQGLAQSAYKTEDWTLQDTCEELWNTEPTHCFAKGGQTVQVYF
DGNKDNSMNYVARDSVYYMTDAGTWDKTATCVSHRGLAYVKEGYNTFYIEFKSECEKYGNTGTWE
AHFGNNVIDC
SEQ ID NO: 4-HPV18 E7-HPV18 E6-HPV16 E7-HPV16 E6-HPV16 E1-HPV18
E1-HPV16 E2-HPV18 E2 Fusion amino acid sequence
MHGPKATLQDIVLHLEPQNEIPVDLLGHGQLSDSEEENDEIDGVNHQHLPARRAEPQRHTMLCMG
CKCEARIELVVESSADDLRAFQQLFLNTLSFVGPWCASQQMARFEDPTRRPYKLPDLCTELNTSL
QDIEITCVYCKTVLELTEVFEFAFKDLFVVYRDSIPHAACHKGIDFYSRIRELRHYSDSVYGDTL
EKLTNTGLYNLLIRCLRCQKPLNPAEKLRHLNEKRRFHNIAGHYRGQGHSCCNRARQERLQRHGD
TPTLHEYMLDLQPETTDLYGYGQLNDSSEEEDEIDGPAGQAEPDRAHYNIVTFGCKCDSTLRLCV
QSTHVDICTLEDLLMGTLGIVGPICSQKPMFQDPQERPRKLPQLCTELQTTIHDIILECVYCKQQ
LLRREVYDFAFRDLCIVYRDGNPYAVCDKGLKFYSKISEYRHYCYSLYGTTLEQQYNKPLCDLLI
RCINCQKPLCPEEKQRHLDKKQRFHNIRGRWTGRGMSCCRSSRTPKLRSTAAALYWYKTGMSNIS
EVYGDTPEWIQRQTVLQHSFNDCTFELSQMVQWAYDNDIVDDSEIAYKYAQLADTNSNASAFLKS
NSQAKIVKDCATMCRHYKRAEKKQMSMSQWIKYRCDRVDDGGDWKQIVMFLRYQGVDEMSFLSAL
KKFLQGIPKKNCILLYGAANTGALFGMSLMKFLQGSVICFVNSKSHFAFQPLADAKIGMLDDATV
PCWNYIDDNLRNALDGNLVSMDVKHRPLVQLKCPPLLITSAINAGTDSRWPYLHNRLVVFTEPNE
FPFDENGNPVYELNDKNWPKLRSSVAALYWYRTGISNISEVMGDTPEWIQRLTIIQHGIDDSNED
LSEMVQWAFDNELTDESDMAFEYALLADSNSNAAAFLKSNCQAKYLKDCATMCKHYRRAQKRQMN
MSQWIRFRCSKIDEGGDWRPIVQFLRYQQIEFITFLGALKSFLKGTPKKNCLVFCGPANTGAYFG
MSFIHFIQGAVISFVNSTSHAFLEPLTDTKVAMLDDATTTCWTYFDTYMRNALDGNPISIDRKHK
PLIQLKCPPILLTTAIHPAKDNRWPYLESRITVFEFPNAFPFDKNGNPVYEINDKNWMETLCQRL
NVCQDKILTHYENDATDLRDHIDYWKQMALACAIYYKAREMGFKHINHQVVPTLAVSKNKALQAA
ELQLTLETIYNSQYSNEKWTLQDVSLEVYLTAPTGCIAKHGYTVEVQFDGDICNTMHYTNRKYIY
ICEETSVTVVEGQVDYYGLAYVHEGIQTYFVQFKDDAEKYSKNKVWEVHAGGQAILCPTSVEMQT
PKETLSERLSALQDKIIDHYENDAKDIDSQIQYWQLIAWANAIFFAAREHGIQTLNHQVVPAYNI
SKSKAHKAAELQMALQGLAQSAYKTEDWTLQDTCEELWNTEPTHCFAKGGQTVQVYFDGNKDNSM
NYVARDSVYYMTDAGTWDKTATCVSHRGLAYVKEGYNTFYIEFKSECEKYGNTGTWEAHFGNNVI
DC
SEQ ID NO: 5-HPV18 E6, Human papillomavirus type 18, UniProtKB-
P06463
MARFEDPTRRPYKLPDLCTELNTSLQDIEITCVYCKTVLELTEVFEFAFKDLFVVYRDSIPHAAC
HKCIDFYSRIRELRHYSDSVYGDTLEKLINTGLYNLLIRCLRCQKPLNPAEKLRHLNEKRRFHNI
AGHYRGQCHSCCNRARQERLQRRRETQV
SEQ ID NO: 6-HPV16 E6, Human papillomavirus type 16, UniProtKB-
P03126
MHQKRTAMFQDPQERPRKLPQLCTELQTTIHDIILECVYCKQQLLRREVYDFAFRDLCIVYRDGN
PYAVCDKCLKFYSKISEYRHYCYSLYGTTLEQQYNKPLCDLLIRCINCQKPLCPEEKQRHLDKKQ
RFHNIRGRWTGRCMSCCRSSRTRRETQL
SEQ ID NO: 7-HPV18 E7, Human papillomavirus type 18, UniProtKB-
P06788
MHGPKATLQDIVLHLEPQNEIPVDLLCHEQLSDSEEENDEIDGVNHQHLPARRAEPQRHTMLCMC
CKCEARIKLVVESSADDLRAFQQLFLNTLSFVCPWCASQQ
SEQ ID NO: 8-HPV16 E7, Human papillomavirus type 16, UniProtKB-
P03129
MHGDTPTLHEYMLDLQPETTDLYCYEQLNDSSEEEDEIDGPAGQAEPDRAHYNIVTFCCKCDSTL
RLCVQSTHVDIRTLEDLLMGTLGIVCPICSQKP
SEQ ID NO: 9-HPV18 E1, Human papillomavirus type 18, UniProtKB-
P06789
MADPEGTDGEGTGCNGWFYVQAIVDKKTGDVISDDEDENATDTGSDMVDFIDTQGTFCEQAELET
AQALFHAQEVHNDAQVLHVLKRKFAGGSTENSPLGERLEVDTELSPRLQEISLNSGQKKAKRRLF
TISDSGYGCSEVEATQIQVTTNGEHGGNVCSGGSTEAIDNGGTEGNNSSVDGTSDNSNIENVNPQ
CTIAQLKDLLKVNNKQGAMLAVFKDTYGLSFTDLVRNFKSDKTTCTDWVTAIFGVNPTIAEGFKT
LIQPFILYAHIQCLDCKWGVLILALLRYKCGKSRLTVAKGLSTLLHVPETCMLIQPPKLRSSVAA
LYWYRTGISNISEVMGDTPEWIQRLTIIQHGIDDSNFDLSEMVQWAFDNELTDESDMAFEYALLA
DSNSNAAAFLKSNCQAKYLKDCATMCKHYRRAQKRQMNMSQWIRFRCSKIDEGGDWRPIVQFLRY
QQIEFITFLGALKSFLKGTPKKNCLVFCGPANTGKSYFGMSFIHFIQGAVISFVNSTSHEWLEPL
TDTKVAMLDDATTTCWTYFDTYMRNALDGNPISIDRKHKPLIQLKCPPILLTTNIHPAKDNRWPY
LESRITVFEFPNAFPFDKNGNPVYEINDKNWKCFFERTWSRLDLHEEEEDADTEGNPFGTFKLRA
GQNHRPL
SEQ ID NO: 10-HPV16 E1, Human papillomavirus type 16, UniProtKB-
P03114
MADPAGTNGEEGTGCNGWFYVEAVVEKKTGDAISDDENENDSDTGEDLVDFIVNDNDYLTQAETE
TAHALFTAQEAKQHRDAVQVLKRKYLVSPLSDISGCVDNNISPRLKAICIEKQSRAAKRRLFESE
DSGYGNTEVETQQMLQVEGRHETETPCSQYSGGSGGGCSQYSSGSGGEGVSERHTICQTPLTNIL
NVLKTSNAKAAMLAKFKELYGVSFSELVRPFKSNKSTCCDWCIAAFGLTPSIADSIKTLLQQYCL
YLHIQSLACSWGMVVLLLVRYKCGKNRETIEKLLSKLLCVSPMCMMIEPPKLRSTAAALYWYKTG
ISNISEVYGDTPEWIQRQTVLQHSFNDCTFELSQMVQWAYDNDIVDDSEIAYKYAQLADTNSNAS
AFLKSNSQAKIVKDCATMCRHYKRAEKKQMSMSQWIKYRCDRVDDGGDWKQIVMFLRYQGVEFMS
FLTALKRFLQGIPKKNCILLYGAANTGKSLFGMSLMKFLQGSVICFVNSKSHFWLQPLADAKIGM
LDDATVPCWNYIDDNLRNALDGNLVSMDVKHRPLVQLKCPPLLITSNINAGTDSRWPYLHNRLVV
FTFPNEFPFDENGNPVYELNDKNWKSFFSRTWSRLSLHEDEDKENDGDSLPTFKCVSGQNTNTL
SEQ ID NO: 11-HPV18 E2, Human papillomavirus type 18, UniProtKB-
P06790
MQTPKETLSERLSCVQDKIIDHYENDSKDIDSQIQYWQLIRWENAIFFAAREHGIQTLNHQVVPA
YNISKSKAHKAIELQMALQGLAQSAYKTEDWTLQDTCEELWNTEPTHCFKKGGQTVQVYFDGNKD
NCMTYVAWDSVYYMTDAGTWDKTATCVSHRGLYYVKEGYNTFYIEFKSECEKYGNTGTWEVHFGN
NVIDCNDSMCSTSDDTVSATQLVKQLQHTPSPYSSTVSVGTAKTYGQTSAATRPGHCGLAEKQHC
GPVNPLLGAATPTGNNKRRKLCSGNTTPIIHLKGDRNSLKCLRYRLRKHSDHYRDISSTWHWTGA
GNEKTGILTVTYHSETQRTKFLNTVAIPDSVQILVGYMTM
SEQ ID NO: 12-HPV16 E2, Human papillomavirus type 16, UniProtKB-
P03120
METLCQRLNVCQDKILTHYENDSTDLRDHIDYWKHMRLECAIYYKAREMGFKHINHQVVPTLAVS
KNKALQAIELQLTLETIYNSQYSNEKWTLQDVSLEVYLTAPTGCIKKHGYTVEVQFDGDICNTMH
YTNWTHIYICEEASVTVVEGQVDYYGLYYVHEGIRTYFVQFKDDAEKYSKNKVWEVHAGGQVILC
PTSVFSSNEVSSPEIIRQHLANHPAATHTKAVALGTEETQTTIQRPRSEPDTGNPCHTTKLLHRD
SVDSAPILTAFNSSHKGRINCNSNTTPIVHLKGDANTLKCLRYRFKKHCTLYTAVSSTWHWTGHN
VKHKSAIVTLTYDSEWQRDQFLSQVKIPKTITVSTGEMSI
SEQ ID NO: 13-HPV Fusion (HPV18 E7-HPV18 E6-HPV16 E7-HPV16 E6)
nucleic acid sequence
ATGCACGGCCCCAAGGCCACACTGCAGGATATCGTTCTGCACCTGGAACCCCAGAACGAGATCCC
CGTGGATCTGCTGGGCCATGGCCAGCTGTCTGACTCCGAAGAAGAGAACGACGAGATCGACGGCG
TGAACCACCAGCATCTCCCTGCTCGAAGAGCCGAGCCTCAACGGCACACCATGCTGTGCATGGGA
TGTAAATGCGAAGCCAGAATCGAGCTGGTGGTTGAGAGCTCGGCCGACGACCTGAGAGCCTTCCA
GCAGCTGTTTCTGAACACCCTGAGCTTCGTGGGCCCTTGGTGCGCCAGCCAGCAGATGGCCAGAT
TTGAGGACCCTACACGGCGGCCTTACAAGCTGCCTGACCTGTGCACCGAACTGAACACCAGCCTG
CAGGATATTGAGATCACCTGCGTGTACTGTAAAACCGTGCTGGAGCTGACAGAGGTTTTTGAATT
CGCCTTCAAGGACCTGTTCGTGGTGTACAGAGATAGCATCCCTCACGCCGCTTGTCACAAGGGCA
TTGACTTCTACAGCAGAATACGGGAACTGCGGCACTACTCTGATTCTGTGTACGGCGACACCCTA
GAAAAGCTGACCAACACAGGCCTGTACAACCTCCTGATCAGATGCCTGCGTTGCCAGAAGCCACT
GAACCCAGCCGAGAAGCTGAGACACCTAAACGAGAAGCGGCGCTTCCACAACATCGCCGGCCACT
ACAGAGGACAGGGCCACTCTTGCTGCAACAGAGCTAGACAGGAGAGGCTGCAGAGACACGGCGAC
ACACCTACTCTGCATGAGTACATGCTGGACCTGCAGCCTGAAACCACCGATCTGTACGGATACGG
CCAGCTGAACGACAGCAGCGAGGAGGAAGACGAAATCGATGGCCCTGCCGGCCAGGCCGAGCCTG
ATAGAGCTCACTACAACATCGTCACCTTCGGGTGTAAGTGCGACTCTACACTCCGGCTGTGTGTG
CAGAGCACACACGTGGACATCTGCACCCTGGAGGATCTTCTGATGGGCACCCTGGGAATCGTGGG
CCCTATCTGCAGCCAGAAGCCTATGTTCCAGGACCCTCAGGAGAGACCCCGGAAGCTGCCTCAGC
TGTGCACCGAACTGCAGACGACAATCCACGACATTATCCTGGAGTGCGTGTACTGCAAGCAACAA
CTGCTGCGGAGAGAGGTGTATGACTTCGCTTTCAGAGATCTGTGCATCGTGTACAGAGATGGCAA
CCCCTACGCTGTGTGCGACAAGGGCCTCAAGTTCTACAGCAAGATCTCTGAATACCGGCACTACT
GCTACAGCCTGTACGGCACAACCCTGGAGCAGCAGTATAACAAACCCCTGTGTGACCTCCTGATC
AGATGCATTAACTGCCAAAAACCACTGTGCCCAGAAGAAAAGCAGCGACACCTGGACAAGAAGCA
GCGGTTTCACAACATCAGAGGCAGATGGACCGGGCGGGGAATGTCTTGTTGCAGAAGCTCTAGAA
CT
SEQ ID NO: 14-HPV Fusion (HPV16 E1-HPV18 E1) nucleic acid
sequence
CCAAAGCTGAGATCTACAGCCGCTGCACTGTACTGGTATAAGACCGGCATGTCCAACATCAGCGA
GGTTTACGGCGATACCCCTGAGTGGATTCAGAGACAGACCGTGCTGCAGCACTCCTTCAACGACT
GTACATTTGAACTGAGCCAAATGGTGCAGTGGGCTTACGACAATGACATTGTGGATGATAGCGAG
ATCGCCTACAAGTACGCCCAGCTTGCCGACACCAACTCCAACGCTTCTGCCTTCCTGAAAAGCAA
CAGCCAGGCCAAGATCGTGAAGGATTGCGCCACAATGTGCAGACACTACAAGAGAGCTGAGAAGA
AGCAGATGTCCATGTCGCAATGGATCAAGTACCGGTGCGATAGAGTGGACGACGGCGGAGATTGG
AAACAGATCGTGATGTTCCTGCGGTACCAGGGCGTGGACTTCATGAGCTTCCTGTCTGCTCTGAA
AAAATTTCTGCAGGGCATCCCTAAGAAAAATTGCATCTTGCTGTATGGCGCCGCTAACACCGGCG
CCCTGTTTGGCATGAGCCTGATGAAGTTCCTGCAGGGCAGCGTTATCTGCTTCGTGAACTCCAAA
AGCCACTTCGCCTTCCAGCCCCTGGCTGACGCCAAGATCGGCATGCTGGACGACGCCACAGTGCC
GTGCTGGAACTATATCGACGACAACTTGCGGAACGCCCTGGACGGCAATCTGGTCAGCATGGACG
TGAAGCACCGGCCTCTGGTGCAGCTGAAATGTCCTCCACTGCTGATCACGTCCGCCATCAACGCC
GGCACCGATAGCAGATGGCCTTACCTGCACAATAGACTGGTTGTCTTCACCTTCCCTAATGAATT
CCCTTTCGATGAGAACGGCAACCCCGTGTACGAGCTGAATGATAAGAACTGGCCCAAGCTGAGAA
GCAGCGTGGCCGCTCTGTACTGGTACCGGACCGGCATTAGCAACATCAGCGAGGTGATGGGCGAC
ACACCCGAATGGATTCAGAGACTGACCATCATCCAGCACGGCATCGATGACAGCAATTTCGACCT
ATCTGAGATGGTCCAGTGGGCCTTTGACAACGAGCTGACAGACGAGAGTGATATGGCCTTTGAGT
ACGCCCTGCTGGCCGATTCTAACAGCAATGCCGCCGCCTTCCTCAAGAGCAATTGCCAAGCCAAG
TACCTGAAGGACTGCGCCACCATGTGCAAGCACTATAGGAGAGCCCAGAAGCGGCAGATGAACAT
GAGCCAGTGGATCAGATTCAGATGTAGCAAGATCGATGAGGGCGGCGACTGGAGACCTATCGTGC
AGTTCCTGCGGTACCAGCAAATCGAGTTCATCACCTTCTTGGGCGCCCTGAAGAGCTTCCTGAAG
GGCACCCCTAAGAAAAACTGCCTGGTGTTCTGCGGCCCCGCCAATACAGGAGCCTACTTCGGCAT
GAGCTTTATCCACTTTATCCAGGGAGCCGTGATCTCCTTCGTGAACAGCACCAGCCACGCCTTTC
TGGAACCTCTGACCGACACCAAGGTGGCCATGCTGGACGACGCTACCACAACCTGCTGGACCTAC
TTCGATACATACATGAGAAACGCCCTGGACGGAAATCCTATCAGCATCGACCGGAAGCACAAGCC
ACTGATCCAGCTGAAATGCCCCCCCATCTTGCTGACCACCGCCATCCACCCCGCTAAGGACAATC
GGTGGCCTTACCTGGAAAGCAGAATCACCGTGTTCGAGTTCCCTAACGCTTTCCCCTTCGACAAG
AACGGCAACCCTGTCTACGAGATCAACGACAAGAATTGG
SEQ ID NO: 15-HPV Fusion (HPV16 E2-HPV18 E2) nucleic acid
sequence
ATGGAAACTTTGTGCCAGAGACTGAATGTGTGTCAGGACAAGATCCTGACCCACTACGAGAACGA
CGCCACCGATTTGAGGGATCACATCGACTACTGGAAGCAGATGGCACTCGCCTGTGCCATCTACT
ACAAGGCTCGCGAGATGGGCTTCAAGCACATCAACCATCAGGTGGTGCCCACGCTGGCCGTTAGC
AAGAACAAGGCCCTGCAGGCCGCTGAACTGCAGCTGACACTGGAGACCATCTACAACAGCCAATA
CAGCAACGAGAAGTGGACCCTGCAAGATGTGAGCCTGGAAGTGTACCTGACAGCTCCTACCGGCT
GCATCGCCAAGCACGGCTATACCGTGGAAGTGCAGTTCGACGGCGATATCTGTAACACCATGCAC
TACACAAACAGAAAGTACATCTACATCTGCGAGGAAACCTCCGTGACCGTGGTGGAAGGCCAGGT
GGACTACTACGGCCTGGCCTACGTGCACGAGGGCATTCAGACATACTTCGTGCAGTTCAAGGACG
ACGCCGAGAAGTACTCCAAAAACAAAGTGTGGGAAGTGCATGCTGGAGGACAGGCTATCCTGTGT
CCTACAAGCGTGTTCATGCAGACACCTAAAGAGACACTGAGCGAGCGGCTGAGCGCCTTGCAGGA
CAAGATCATCGACCACTACGAAAACGACGCCAAAGACATCGATAGCCAGATCCAGTACTGGCAGC
TTATCGCCTGGGCCAACGCGATTTTCTTCGCCGCGCGGGAACACGGCATCCAAACCCTGAACCAC
CAGGTGGTGCCTGCTTATAATATCTCTAAGTCCAAGGCCCACAAGGCTGCCGAGCTGCAGATGGC
CCTGCAGGGCCTGGCACAATCCGCCTACAAGACCGAGGACTGGACACTCCAAGACACATGTGAAG
AACTGTGGAACACAGAGCCTACACATTGTTTTGCCAAAGGAGGCCAGACCGTGCAGGTGTATTTC
GACGGCAACAAGGACAACTCCATGAATTACGTGGCCCGGGATTCTGTGTACTACATGACCGACGC
CGGCACCTGGGACAAGACCGCCACCTGCGTGTCCCACAGAGGACTGGCCTACGTAAAGGAAGGTT
ACAATACCTTTTACATCGAGTTCAAAAGCGAATGTGAGAAATACGGCAACACCGGAACGTGGGAG
GCGCACTTTGGCAACAACGTGATCGACTGCTGA
SEQ ID NO: 16-HPV Fusion (HPV18 E7-HPV18 E6-HPV16 E7-HPV16 E6-
HPV16 E1-HPV18 E1-HPV16 E2-HPV18 E2) nucleic acid sequence
ATGCACGGCCCCAAGGCCACACTGCAGGATATCGTTCTGCACCTGGAACCCCAGAACGAGATCCC
CGTGGATCTGCTGGGCCATGGCCAGCTGTCTGACTCCGAAGAAGAGAACGACGAGATCGACGGCG
TGAACCACCAGCATCTCCCTGCTCGAAGAGCCGAGCCTCAACGGCACACCATGCTGTGCATGGGA
TGTAAATGCGAAGCCAGAATCGAGCTGGTGGTTGAGAGCTCGGCCGACGACCTGAGAGCCTTCCA
GCAGCTGTTTCTGAACACCCTGAGCTTCGTGGGCCCTTGGTGCGCCAGCCAGCAGATGGCCAGAT
TTGAGGACCCTACACGGCGGCCTTACAAGCTGCCTGACCTGTGCACCGAACTGAACACCAGCCTG
CAGGATATTGAGATCACCTGCGTGTACTGTAAAACCGTGCTGGAGCTGACAGAGGTTTTTGAATT
CGCCTTCAAGGACCTGTTCGTGGTGTACAGAGATAGCATCCCTCACGCCGCTTGTCACAAGGGCA
TTGACTTCTACAGCAGAATACGGGAACTGCGGCACTACTCTGATTCTGTGTACGGCGACACCCTA
GAAAAGCTGACCAACACAGGCCTGTACAACCTCCTGATCAGATGCCTGCGTTGCCAGAAGCCACT
GAACCCAGCCGAGAAGCTGAGACACCTAAACGAGAAGCGGCGCTTCCACAACATCGCCGGCCACT
ACAGAGGACAGGGCCACTCTTGCTGCAACAGAGCTAGACAGGAGAGGCTGCAGAGACACGGCGAC
ACACCTACTCTGCATGAGTACATGCTGGACCTGCAGCCTGAAACCACCGATCTGTACGGATACGG
CCAGCTGAACGACAGCAGCGAGGAGGAAGACGAAATCGATGGCCCTGCCGGCCAGGCCGAGCCTG
ATAGAGCTCACTACAACATCGTCACCTTCGGGTGTAAGTGCGACTCTACACTCCGGCTGTGTGTG
CAGAGCACACACGTGGACATCTGCACCCTGGAGGATCTTCTGATGGGCACCCTGGGAATCGTGGG
CCCTATCTGCAGCCAGAAGCCTATGTTCCAGGACCCTCAGGAGAGACCCCGGAAGCTGCCTCAGC
TGTGCACCGAACTGCAGACGACAATCCACGACATTATCCTGGAGTGCGTGTACTGCAAGCAACAA
CTGCTGCGGAGAGAGGTGTATGACTTCGCTTTCAGAGATCTGTGCATCGTGTACAGAGATGGCAA
CCCCTACGCTGTGTGCGACAAGGGCCTCAAGTTCTACAGCAAGATCTCTGAATACCGGCACTACT
GCTACAGCCTGTACGGCACAACCCTGGAGCAGCAGTATAACAAACCCCTGTGTGACCTCCTGATC
AGATGCATTAACTGCCAAAAACCACTGTGCCCAGAAGAAAAGCAGCGACACCTGGACAAGAAGCA
GCGGTTTCACAACATCAGAGGCAGATGGACCGGGCGGGGAATGTCTTGTTGCAGAAGCTCTAGAA
CTCCAAAGCTGAGATCTACAGCCGCTGCACTGTACTGGTATAAGACCGGCATGTCCAACATCAGC
GAGGTTTACGGCGATACCCCTGAGTGGATTCAGAGACAGACCGTGCTGCAGCACTCCTTCAACGA
CTGTACATTTGAACTGAGCCAAATGGTGCAGTGGGCTTACGACAATGACATTGTGGATGATAGCG
AGATCGCCTACAAGTACGCCCAGCTTGCCGACACCAACTCCAACGCTTCTGCCTTCCTGAAAAGC
AACAGCCAGGCCAAGATCGTGAAGGATTGCGCCACAATGTGCAGACACTACAAGAGAGCTGAGAA
GAAGCAGATGTCCATGTCGCAATGGATCAAGTACCGGTGCGATAGAGTGGACGACGGCGGAGATT
GGAAACAGATCGTGATGTTCCTGCGGTACCAGGGCGTGGACTTCATGAGCTTCCTGTCTGCTCTG
AAAAAATTTCTGCAGGGCATCCCTAAGAAAAATTGCATCTTGCTGTATGGCGCCGCTAACACCGG
CGCCCTGTTTGGCATGAGCCTGATGAAGTTCCTGCAGGGCAGCGTTATCTGCTTCGTGAACTCCA
AAAGCCACTTCGCCTTCCAGCCCCTGGCTGACGCCAAGATCGGCATGCTGGACGACGCCACAGTG
CCGTGCTGGAACTATATCGACGACAACTTGCGGAACGCCCTGGACGGCAATCTGGTCAGCATGGA
CGTGAAGCACCGGCCTCTGGTGCAGCTGAAATGTCCTCCACTGCTGATCACGTCCGCCATCAACG
CCGGCACCGATAGCAGATGGCCTTACCTGCACAATAGACTGGTTGTCTTCACCTTCCCTAATGAA
TTCCCTTTCGATGAGAACGGCAACCCCGTGTACGAGCTGAATGATAAGAACTGGCCCAAGCTGAG
AAGCAGCGTGGCCGCTCTGTACTGGTACCGGACCGGCATTAGCAACATCAGCGAGGTGATGGGCG
ACACACCCGAATGGATTCAGAGACTGACCATCATCCAGCACGGCATCGATGACAGCAATTTCGAC
CTATCTGAGATGGTCCAGTGGGCCTTTGACAACGAGCTGACAGACGAGAGTGATATGGCCTTTGA
GTACGCCCTGCTGGCCGATTCTAACAGCAATGCCGCCGCCTTCCTCAAGAGCAATTGCCAAGCCA
AGTACCTGAAGGACTGCGCCACCATGTGCAAGCACTATAGGAGAGCCCAGAAGCGGCAGATGAAC
ATGAGCCAGTGGATCAGATTCAGATGTAGCAAGATCGATGAGGGCGGCGACTGGAGACCTATCGT
GCAGTTCCTGCGGTACCAGCAAATCGAGTTCATCACCTTCTTGGGCGCCCTGAAGAGCTTCCTGA
AGGGCACCCCTAAGAAAAACTGCCTGGTGTTCTGCGGCCCCGCCAATACAGGAGCCTACTTCGGC
ATGAGCTTTATCCACTTTATCCAGGGAGCCGTGATCTCCTTCGTGAACAGCACCAGCCACGCCTT
TCTGGAACCTCTGACCGACACCAAGGTGGCCATGCTGGACGACGCTACCACAACCTGCTGGACCT
ACTTCGATACATACATGAGAAACGCCCTGGACGGAAATCCTATCAGCATCGACCGGAAGCACAAG
CCACTGATCCAGCTGAAATGCCCCCCCATCTTGCTGACCACCGCCATCCACCCCGCTAAGGACAA
TCGGTGGCCTTACCTGGAAAGCAGAATCACCGTGTTCGAGTTCCCTAACGCTTTCCCCTTCGACA
AGAACGGCAACCCTGTCTACGAGATCAACGACAAGAATTGGATGGAAACTTTGTGCCAGAGACTG
AATGTGTGTCAGGACAAGATCCTGACCCACTACGAGAACGACGCCACCGATTTGAGGGATCACAT
CGACTACTGGAAGCAGATGGCACTCGCCTGTGCCATCTACTACAAGGCTCGCGAGATGGGCTTCA
AGCACATCAACCATCAGGTGGTGCCCACGCTGGCCGTTAGCAAGAACAAGGCCCTGCAGGCCGCT
GAACTGCAGCTGACACTGGAGACCATCTACAACAGCCAATACAGCAACGAGAAGTGGACCCTGCA
AGATGTGAGCCTGGAAGTGTACCTGACAGCTCCTACCGGCTGCATCGCCAAGCACGGCTATACCG
TGGAAGTGCAGTTCGACGGCGATATCTGTAACACCATGCACTACACAAACAGAAAGTACATCTAC
ATCTGCGAGGAAACCTCCGTGACCGTGGTGGAAGGCCAGGTGGACTACTACGGCCTGGCCTACGT
GCACGAGGGCATTCAGACATACTTCGTGCAGTTCAAGGACGACGCCGAGAAGTACTCCAAAAACA
AAGTGTGGGAAGTGCATGCTGGAGGACAGGCTATCCTGTGTCCTACAAGCGTGTTCATGCAGACA
CCTAAAGAGACACTGAGCGAGCGGCTGAGCGCCTTGCAGGACAAGATCATCGACCACTACGAAAA
CGACGCCAAAGACATCGATAGCCAGATCCAGTACTGGCAGCTTATCGCCTGGGCCAACGCGATTT
TCTTCGCCGCGCGGGAACACGGCATCCAAACCCTGAACCACCAGGTGGTGCCTGCTTATAATATC
TCTAAGTCCAAGGCCCACAAGGCTGCCGAGCTGCAGATGGCCCTGCAGGGCCTGGCACAATCCGC
CTACAAGACCGAGGACTGGACACTCCAAGACACATGTGAAGAACTGTGGAACACAGAGCCTACAC
ATTGTTTTGCCAAAGGAGGCCAGACCGTGCAGGTGTATTTCGACGGCAACAAGGACAACTCCATG
AATTACGTGGCCCGGGATTCTGTGTACTACATGACCGACGCCGGCACCTGGGACAAGACCGCCAC
CTGCGTGTCCCACAGAGGACTGGCCTACGTAAAGGAAGGTTACAATACCTTTTACATCGAGTTCA
AAAGCGAATGTGAGAAATACGGCAACACCGGAACGTGGGAGGCGCACTTTGGCAACAACGTGATC
GACTGCTGA
SEQ ID NO: 17-TR3 dUL128-130 dUL78-HPV
SEQ ID NO: 18-HPV strain 16 E7 Rb binding domain amino acid
sequence
LYCYE
SEQ ID NO: 19-HPV strain 18 E7 Rb binding domain amino acid
sequence
LLCHE
SEQ ID NO: 20-HPV strain 16 E7 Rb binding domain Mutant amino
acid sequence
LYGYG
SEQ ID NO: 21-HPV strain 18 E7 Rb binding domain Mutant amino
acid sequence
LLGHG
SEQ ID NO: 22-D6 PDZ binding domain amino acid sequence
RRETQV
SEQ ID NO: 23-HPV18 E7/E6 amino acid sequence
MHGPKATLQDIVLHLEPQNEIPVDLLgHgQLSDSEEENDEIDGVNHOHLPARRAEPQRHTMLCMg
CKCEARIELVVESSADDLRAFQQLFLNTLSFVgPWCASQQMARFEDPTRRPYKLPDLCTELNTSL
QDIEITCVYCKTVLELTEVFEFAFKDLFVVYRDSIPHAACHKgIDFYSRIRELRHYSDSVYGDTL
EKLTNTGLYNLLIRCLRCQKPLNPAEKLRHLNEKRRFHNIAGHYRGQgHSCCNRARQERLORYPY
DVPDYA
SEQ ID NO: 24-HPV16 E7/E6 amino acid sequence
MHGDTPTLHEYMLDLQPETTDLYgYgQLNDSSEEEDEIDGPAGQAEPDRAHYNIVTFgCKCDSTL
RLCVQSTHVDICTLEDLLMGTLGIVgPICSQKPMFQDPQERPRKLPQLCTELQTTIHDIILECVY
CKQQLLRREVYDFAFRDLCIVYRDGNPYAVCDKgLKFYSKISEYRHYCYSLYGTTLEQQYNKPLC
DLLIRCINCOKPLCPEEKQRHLDKKQRFHNIRGRWTGRgMSCCRSSRT

### Example 1

### Generation and Immunogenicity Testing of a HPV16/18 E6/E7 Fusion Protein Antigen Expressed by RhCMV or HCMV in Rhesus Macaques

The immunogenicity of a HPV16/18 E6/E7 fusion protein comprising HPV18 E6, HPV18 E7, HPV16 E6, and HPV16 E7 was tested in a pre-clinical study in rhesus macaques. CD4+ and CD8+ T cell responses were elicited in response the HPV16/18 E6/E7 fusion when expressed by a RhCMV or HCMV vector (data not shown). The frequencies of HPV-specific CD8+ T cells detected in the CD8+ memory population were quantified. The RhCMV-HPV16/18 E6/E7 vector 20 elicited a frequency of approximately 0.5%-2.0% at 196 days (data not shown). The RhCMV vector was modified to inactivate the UL128 and UL130 genes and these modifications cause the vector to elicit unconventional MHC-II and MHC-E-restricted CD8+ T cell responses, as was observed. The HCMV-HPV16/18 E6/E7 vector elicited a frequency of approximately 0.2%-0.5% (low range) to approximately 2%-5% (high range) at 112 days (data not shown).

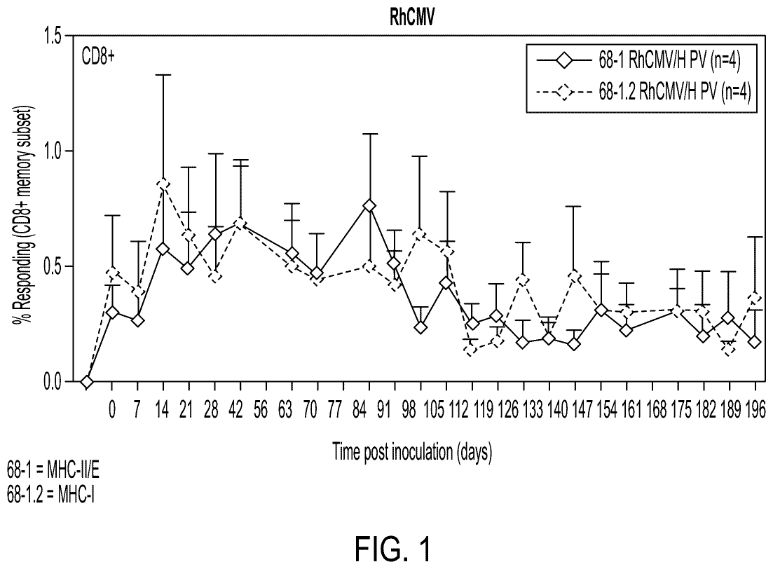

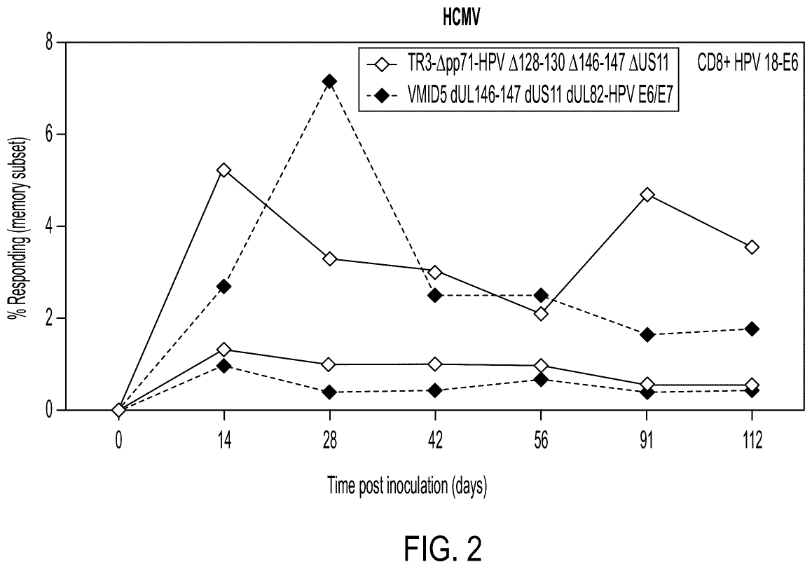

The HPV16/18 E6/E7 antigen was evaluated for its ability to elicit CD8+ T cell responses when expressed by a RhCMV or HCMV vector. For evaluation of the response from an RhCMV vector, rhesus macaques were administered either 68-1 RhCMV (n=4) or 68-1.2 RhCMV (n=4) expressing the HPV16/18 E6/E7 antigen. The 68-1 vector is a well-known laboratory strain of RhCMV (Hansen S G, et al. Complete sequence and genomic analysis of rhesus cytomegalovirus. 30 J Virol. 2003 June; 77(12):6620-36; Gill R B, et al. Coding potential of UL/b′ from the initial source of rhesus cytomegalovirus Strain 68-1. Virology 2013 447(1-2):208-12). The 68-1 vector does not express active UL128, UL130, UL146, and UL147 genes, and elicits unconventional MHC-II and MHC-E-restricted CD8+ T cell responses (Picker L J, et al. Programming Cytomegalovirus as an HIV Vaccine. Trends in Immunology 2003 44(4):287-304). The 68-1.2 vector is a version of the 68-1 vector, wherein the UL128 and UL130 genes have been repaired so they are active. The 68-1.2 vector elicits a conventional MHC-I-restricted CD8+ T cell response. The frequency of HPV18 E6-specific CD8+ T cells in the CD8+ T cell memory population was evaluated weekly (FIG. 1). For evaluation of the response from an HCMV vector, rhesus macaques were administered either a HCMV vector expressing inactive (TR3-Δpp71-HPV Δ128-130 Δ146-147 ΔUS11) or active (VMID5 dUL146-147 dUS11 dUL82-HPV E6/E7) UL128 and UL130 genes, wherein the former elicits an unconventional MHC-II and MHC-E-restricted CD8+ T cell and the latter elicits a conventional MHC-I-restricted CD8+ T cell response. The frequency of HPV18 E6-specific CD8+ T cells in the CD8+ T cell memory population was evaluated every two weeks (FIG. 2).

HPV-specific CD8+ T cells persisted in vaginal tissue at least approximately 3 years after administration of RhCMV vaccine.

The HPV genes E7 and E6 have been definitively associated with the transforming capacity of HPV. Mechanism of action studies have detailed the points of contact between the viral gene products and cellular genes that mediate transformation. In an effort to eliminate these interactions without sacrificing the antigenic potential of the construct, selective mutations were made to disrupt the transformation binding domains within E7 and E6 of HPV-16 and HPV-18 to generate a HPV16/18 E6/E7 fusion protein.

The sequences of HPV strains 16 and 18 were modified to disrupt the E7 Rb binding domains from their native L-Y-C-Y-E (SEQ ID NO:18) and L-L-C-H-E (SEQ ID NO:19) to a non-transforming L-Y-G-Y-G (SEQ ID NO:20) and L-L-G-H-G (SEQ ID NO:21), respectively. In addition to these changes, zinc binding domains known to be involved in transformation capacity were disrupted in E7 of HPV 16 and 18 by modifying the CXXC motifs by glycine substitutions C58G and C91G, and C65G and C98G, respectively.

Similar zinc finger binding motif modifications were made for the HPV-16/18 E6 genes at C66G/C136G and C68G/C140G, respectively, disrupting the E6 known functions in the binding and degradation of p53. Further transformation safety was engineered into the constructs by deletion of the PDZ binding domain RRETQV (SEQ ID NO:22) at the carboxy terminus of high-risk human papillomavirus E6 genes. Binding of PDZ domain-containing cellular partners leads to their degradation and transformation while elimination of this domain prevents such interactions.

A fusion protein was constructed using the modified E7 and E6 proteins of human papillomavirus. FIG. 3 shows sequence components of the HPV16/18 E7/E6 fusion protein. The conserved residues of the Rb-binding motif (FIG. 3, bold, italicized font) and cysteines were replaced with glycine residues (FIG. 3, lowercase “g”) in order to disrupt zinc-finger domains (FIG. 3, bold font). Additionally, the N-terminal p53, E6/AP binding domains, and C-terminal PDZ motifs (FIG. 3, dashed underlined font) were deleted.

### Example 2

### Addition of HPV E1 and E2 Sequences to Enhance Immunogenicity of the HPV16/18 E7/E6 Fusion Protein

Additional antigens were contemplated for incorporation into the HPV16/18 E7/E6 fusion protein in order to further enhance the immune response. HPV E1 helicase, which is involved in initiation and elongation of viral DNA synthesis, and HPV E2 helicase, which is auxiliary to E1 in forming initiation complex at origin, were evaluated to for inclusion with the HPV16/18 E7/E6 fusion protein. Inclusion of E1 and E2 has the potential to increase the breadth of T cell responses to HPV16 and HPV18 and expand antigenic coverage to cervical carcinomas caused by HPV31 and HPV45 strains.

Conservation of the E1 and E2 proteins among alphapapillomaviruses was profiled to inform design of E1 and E2 immunogens. A portion of the conservation profiling was performed among HPV types associated with cervical cancer, including alpha(α) 9 species (types HPV16 and HPV31) and alpha(α) 7 species (types HPV18 and HPV45). HPV16 (alpha(α) 9) and HPV18 (alpha(α) 7) are most commonly associated with cervical carcinoma, whereas HPV31 (alpha(α) 9) and HPV45 (alpha(α) 7) are most strongly associated with cervical carcinoma.

### E1 Sequences

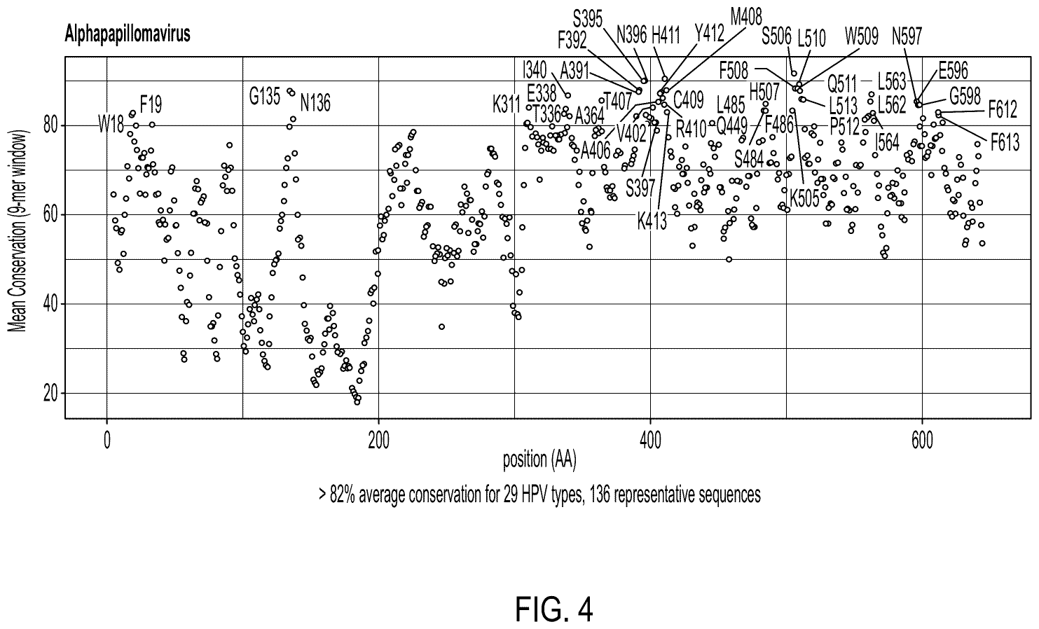

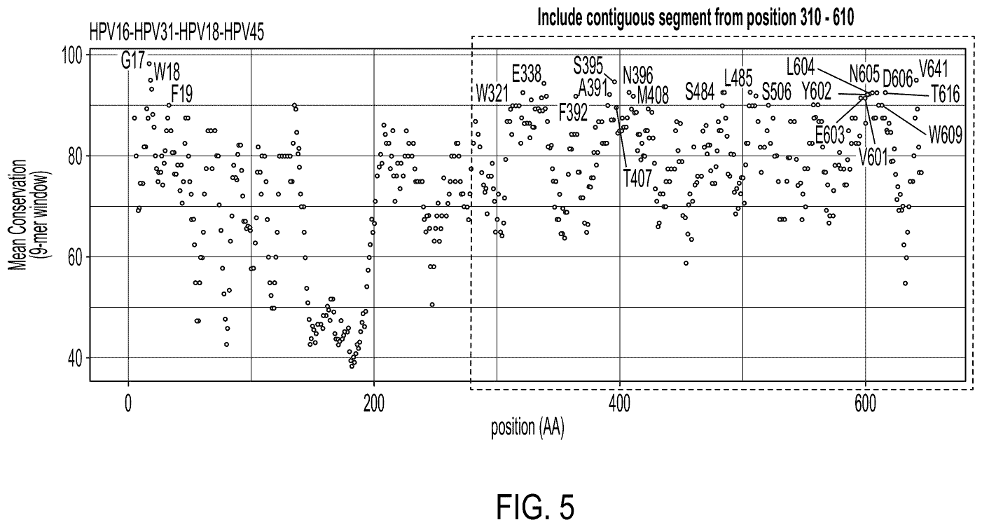

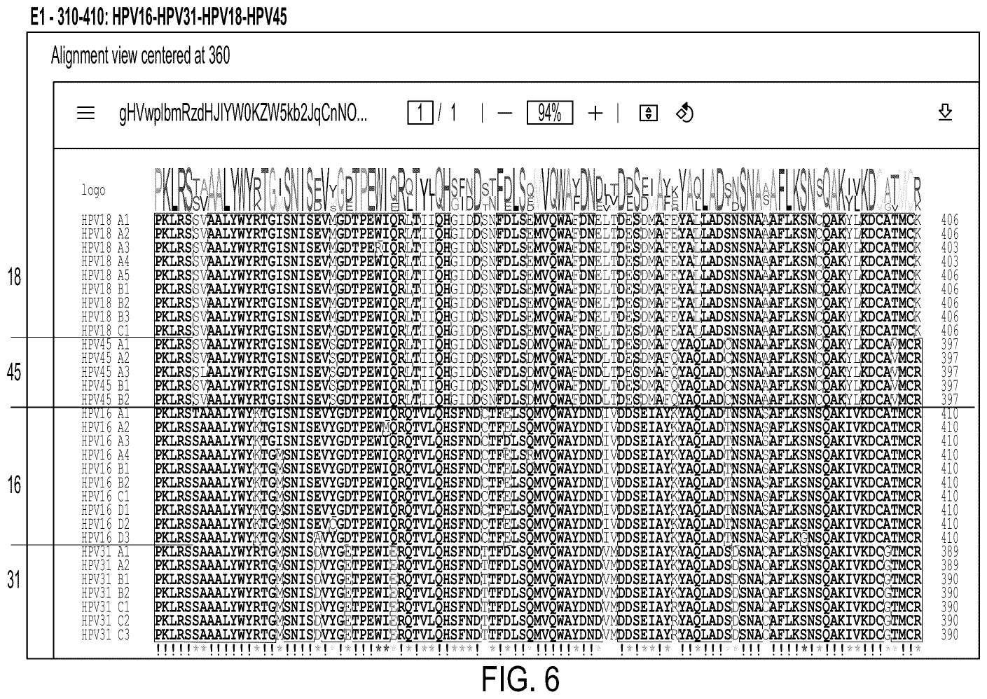

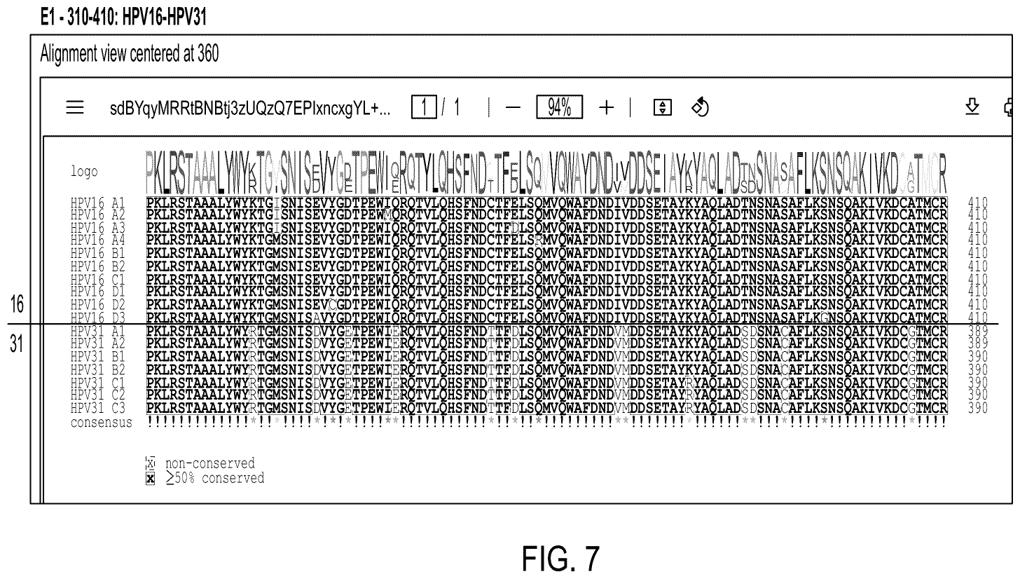

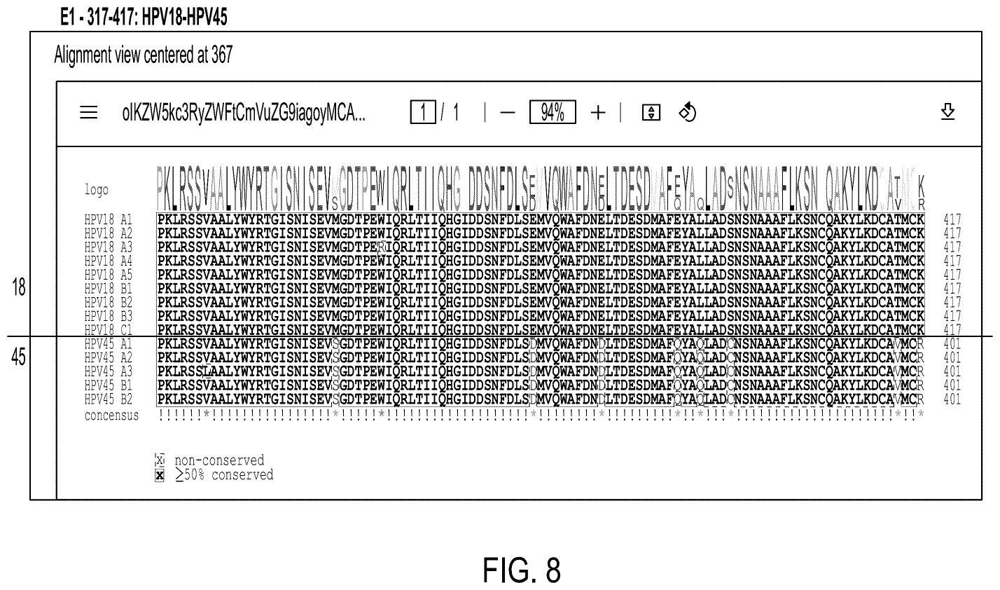

Amino acid conservation of the HPV E1 helicase was profiled for 29 HPV types and 136 representative species. More than 82% average conservation was found across the 29 HPV types and 136 representative species (FIG. 4). Profiling was also performed for HPV E1 among HPV types associated with cervical cancer, including alpha(α) 9 species (types HPV16 and HPV31) and alpha(α) 7 species (types HPV18 and HPV45). More than 92% average conservation was found between HPV16, HPV31, HPV18, and HPV45 (FIG. 5). Sequence-level conservation of the HPV E1 helicase from amino acid positions 310-410 between HPV16, HPV31, HPV18, and HPV45 and between HPV16 and HPV31 is shown in FIGS. 6 and 7, respectively. There was good homology between HPV18 and HPV45, and between HPV16 and HPV31. Sequence-level conservation of the HPV E1 helicase from amino acid positions 317-417 in HPV18 and HPV45 is shown in FIG. 8.

E1 helicase is required for initiation and catalysis of viral DNA synthesis (Bergvall, et al., The E1 proteins. Virology. 445(1-2):35-56 (2013)). E1 activity is ori-dependent (papillomavirus-specific) and E1 must recognize the “origin of replication” (or “ori”). E1 functions as a hexamer in concert with E2, which is required for targeting to the ori. Conservation profiling was used to inform targeted mutation of residues 310-610 of the E1 helicase ATPase domain to eliminate both helicase and ATPase activity (FIG. 5). To eliminate helicase activity, mutations were made in the conserved hydrophobic loop (FWL) (see Castella, et al., Common determinants in DNA melting and helicase-catalysed DNA unwinding by papillomavirus replication protein E1. Nucleic Acids Res. 34(10):3008-19 (2006)). Briefly, the phenylalanine (F) residue of the FWL loop was mutated to tryptophan (W) or alanine (A), and/or the tryptophan (W) was mutated to phenylalanine (F). Additionally, to eliminate ATPase activity, conserved lysine (K) residues of the Walker A motif and asparagine (N) residues of the conserved Sensor 1 motif were mutated to alanine (A) (see Castella, et al., Common determinants in DNA melting and helicase-catalysed DNA unwinding by papillomavirus replication protein E1. Nucleic Acids Res. 34(10):3008-19 (2006)).

### E2 Sequences

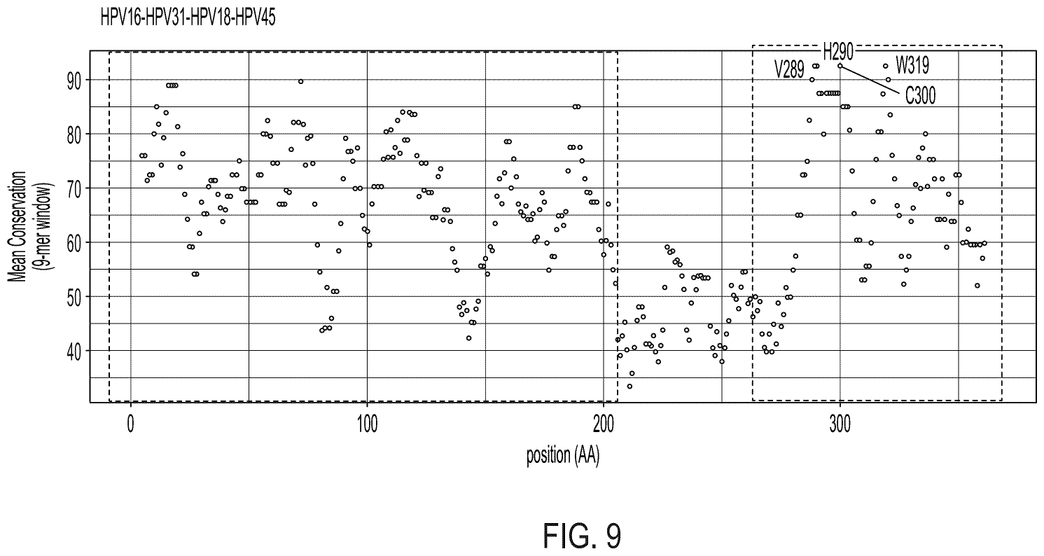

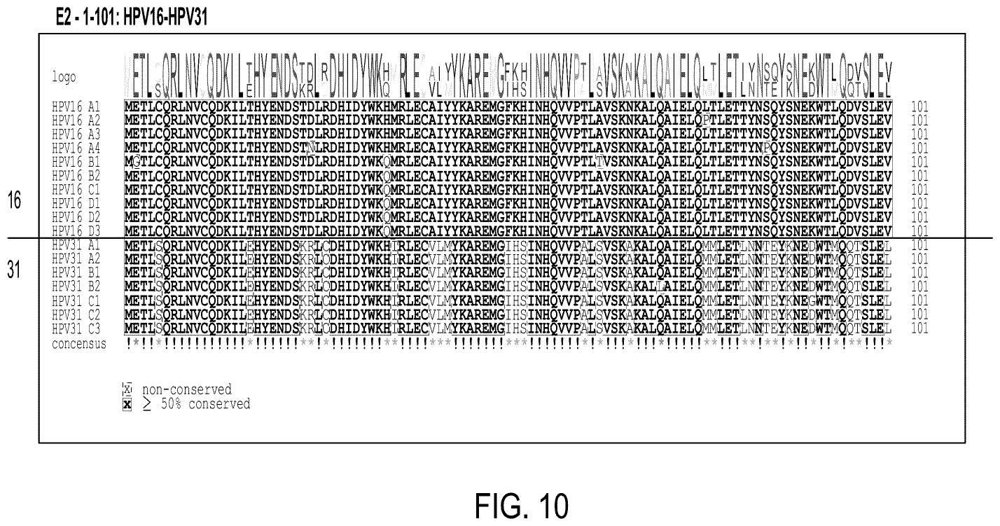

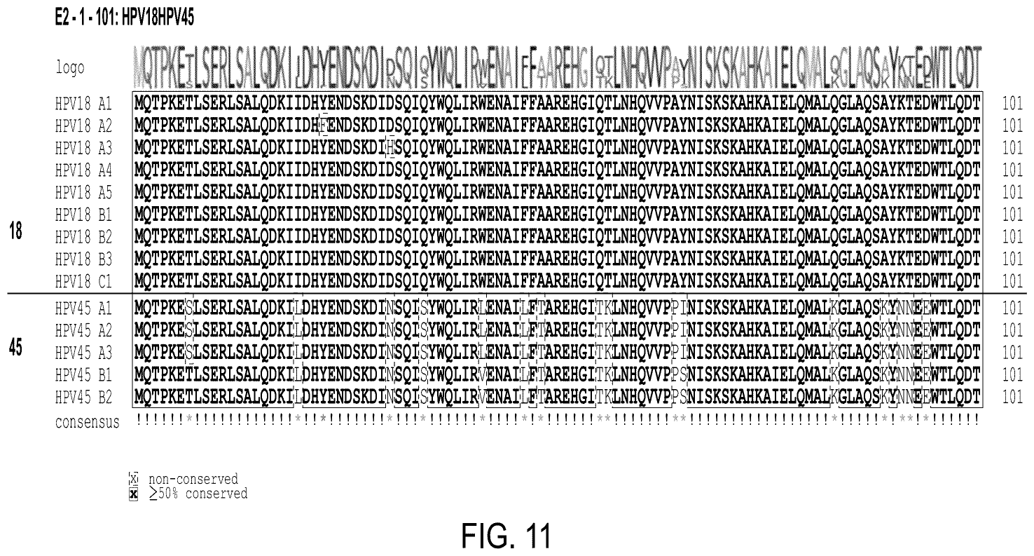

E2 helicase functions in transcriptional regulation, initiation of DNA replication and partitioning the viral genome. The E2 transactivation domain is important for both transcriptional activation and repression, and replication. The R37 and I73 residues are important for transcriptional regulation. The E39 residue is important for replication and interaction with E1 helicase (McBride, AA. The papillomavirus E2 proteins. Virology. 445(1-2):57-79 (2013); Giri I and Yaniv M. Structural and mutational analysis of E2 trans-activating proteins of papillomaviruses reveals three distinct functional domains. EMBO J. 7(9):2823-9 (1988); McBride, A A et al. E2 polypeptides encoded by bovine papillomavirus type 1 form dimers through the common carboxyl-terminal domain: transactivation is mediated by the conserved amino-terminal domain. Proc Natl Acad Sci USA. 186(2):510-4 (1989); McBride, A A et al. The carboxy-terminal domain shared by the bovine papillomavirus E2 transactivator and repressor proteins contains a specific DNA binding activity. EMBO J. 7(2):533-9 (1988)). Amino acid conservation of HPV E2 helicase was profiled in HPV types associated with cervical cancer (HPV16, HPV31, HPV18, and HPV45; FIG. 9). Sequence-level conservation of the HPV E2 helicase from amino acid positions 1-101 between HPV16 and HPV31, and between HPV18 and HPV45 is shown in FIGS. 10 and 11, respectively.

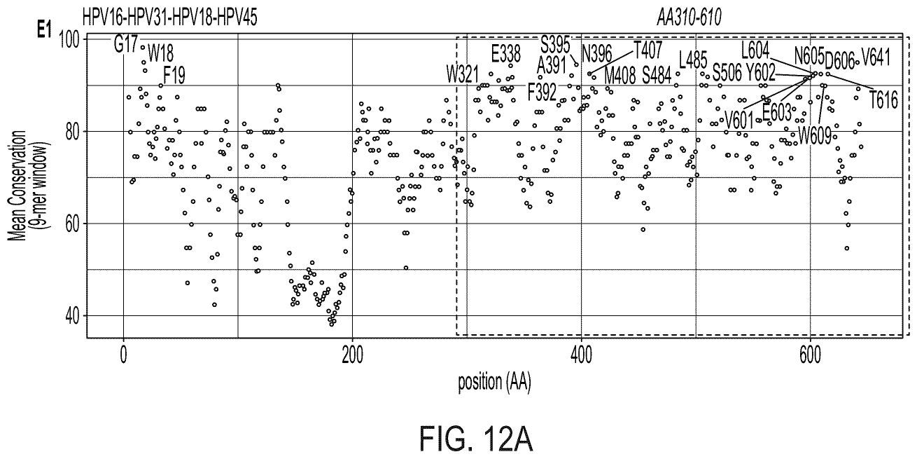

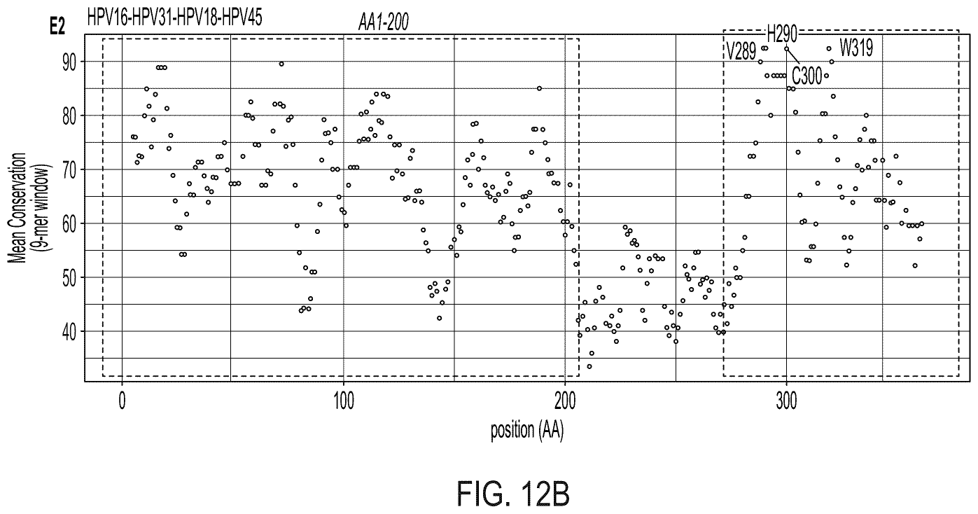

The E2 transactivation domain important for both transcriptional activation and repression, and replication. The R37 and I73 residues are important for transcriptional regulation, and the E39 residue is important for replication and interaction with E1. The E2 sequences of HPV16 and HPV18 included in the fusion: (1) retain the conserved N-terminal 600 bp sequence, (2) minimize E2 transcriptional regulatory activity and interaction with E1 by alanine substitutions of R37, 173, and E39, and (2) include alanine substitutions at K111, S23, and W134 and in the β-sheet region to minimize potential interactions with cell proteins. The regions of conserved sequence regions of HPV E1 helicase (amino acids 310-610) and HPV E2 helicase (amino acids 1-200) used for the construction of a HPV16/18 E7/E6 E1/E2 fusion protein are shown in FIG. 12A and FIG. 12B, respectively.

### Example 3

### Design of a CMV-HPV Vaccine

A fusion protein was constructed using the E7, E6, E1, and E2 antigens described in Examples 1 and 2. The E7 and E6 proteins contained disrupting mutations to prevent oncogenic cell transformation. For E1 and E2, conserved regions were used in the fusion, wherein ATPase- and helicase-disrupting mutations were included. The fusion was codon optimized for expression and to avoid internal insert homology. The resulting HPV16/18 E7/E6 E1/E2 fusion protein gene is a total of 4,527 bp and is diagrammed in FIG. 13.

A CMV vector was selected for expression of the HPV16/18 E7/E6 E1/E2 fusion protein in order to construct the CMV-HPV vaccine candidate. The UL128 and UL130 genes are inactivated in the selected CMV vector and UL146 and UL147 are intact. The vector design facilitates programming of an immune response by conventional MHC-I-restricted CD8+ T cells and CD4+ T cells. The HPV16/18 E7/E6 E1/E2 fusion protein replaces the UL78 gene and is expressed under the control of the UL78 promoter.

### Example 4

### T Cell Epitope Prediction in the HPV16/18 E7/E6 E1/E2 Fusion Protein

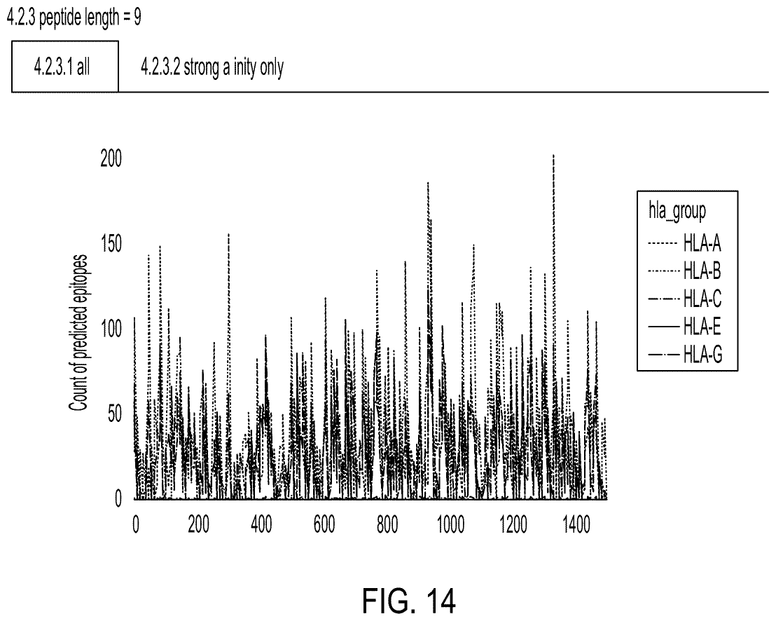

The HPV16/18 E7/E6 E1/E2 fusion protein was analyzed to predict MHC-I-restricted epitopes using MetMHCpan 4.1, a publicly available online MHC-I prediction tool provided by the Immune Epitope Database (IEDB) (https://services.healthtech.dtu.dk/services/NetMHCpan-4.1/). Predictions were made using all known HLA-A, HLA-B, HLA-C, HLA-E, and HLA-G alleles and multiple peptide lengths, including nine (9) amino acids (“9-mers”). Predicted affinity was ranked compared to a set of 400,000 random natural peptides. FIG. 14 shows the mean count of predicted MHC-I-restricted epitopes for 9-mer peptides across the CMV-HPV fusion protein sequence.

The predicted MHC-I-restricted epitopes in the HPV16/18 E7/E6 E1/E2 fusion protein sequence were compared with confirmed epitopes identified in subjects infected with HPV (data not shown). The confirmed epitopes from subjects infected with HPV aligned with the predicted epitopes for E6 and E7 sequences from HPV16 and HPV18 strains. Of the predicted E6 and E7 epitopes, the HPV16 epitopes aligned at 100% identity. Lower match percentages were present in some segments, likely due to modification in the fusion protein. A similar pattern was observed for HPV18 epitopes but significantly fewer epitopes were identified. Of the predicted E1 epitopes, twelve of twelve predicted HPV16 epitopes aligned at 100% identity to the confirmed epitopes and two of two predicted HPV18 epitopes aligned at 100% identity to the confirmed epitopes.

### Example 5

### In Vivo Immunogenicity Testing of the HPV16/18 E7/E6 E1/E2 Fusion Protein

The HPV16/18 E7/E6 E1/E2 fusion protein (SEQ ID NO:4) was expressed in a CMV vector (HPV-CMV) to evaluate the ability of the fusion to induce an immune response in vivo. The vector had a TR3 backbone with the UL128 and UL130 genes inactivated and the UL146 and UL147 genes intact. The vector design facilitates programming of an immune response by conventional MHC-I-restricted CD8+ T cells and CD4+ T cells. The HPV16/18 E7/E6 E1/E2 fusion protein replaces the UL78 gene and is expressed under the control of the UL78 promoter.

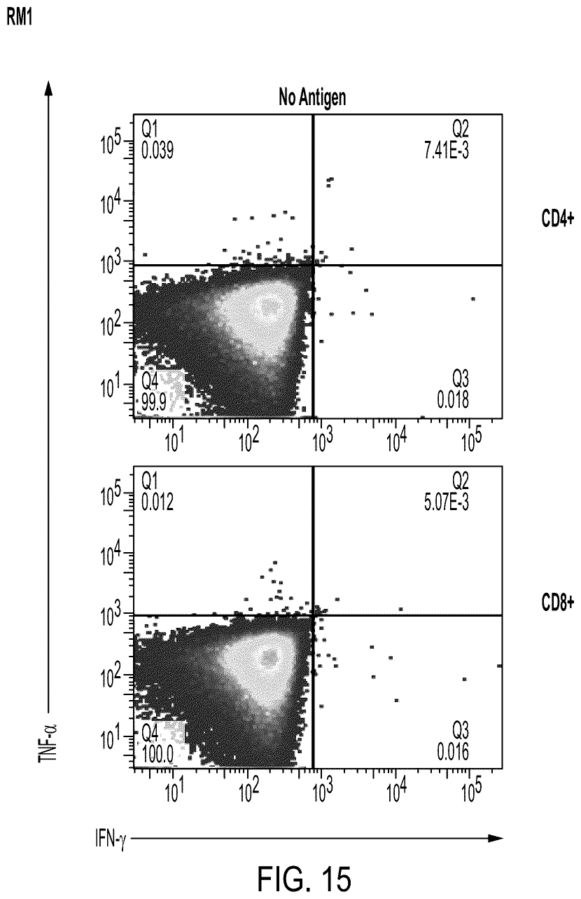

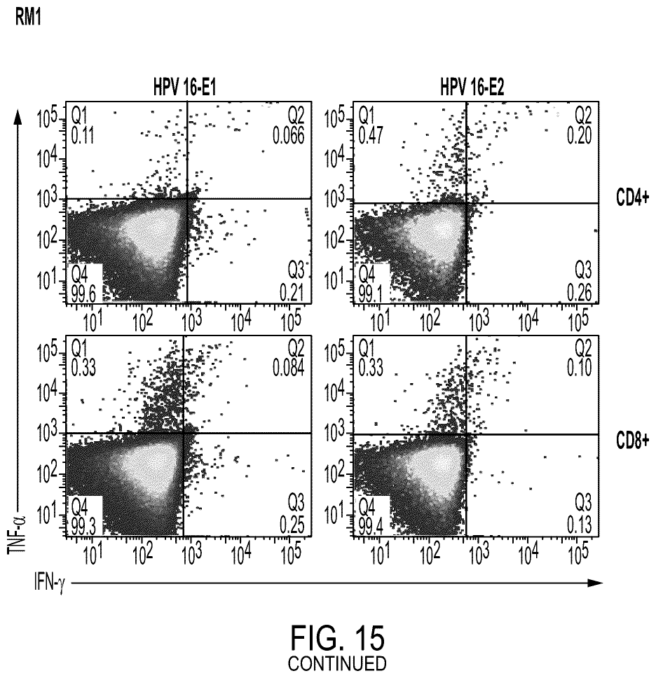

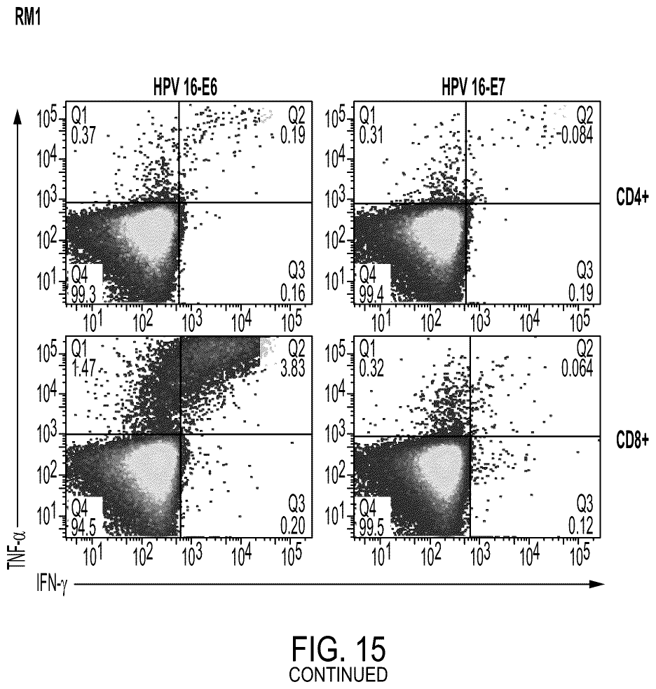

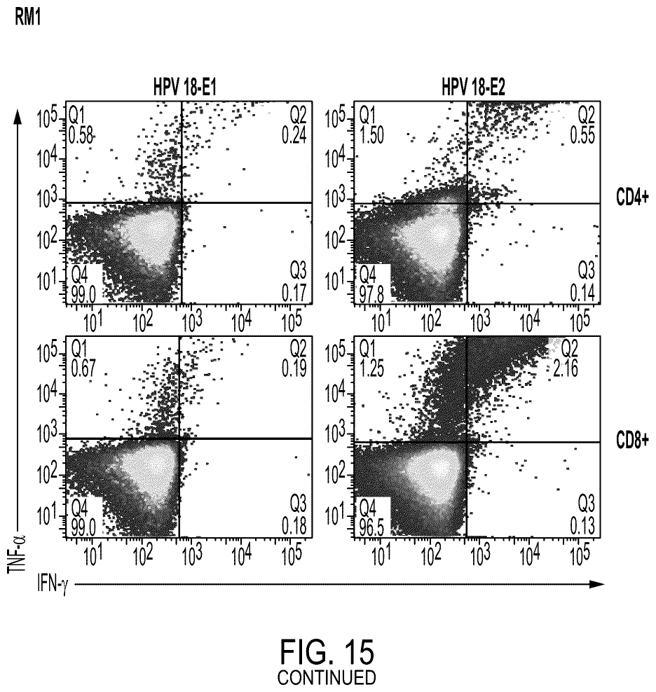

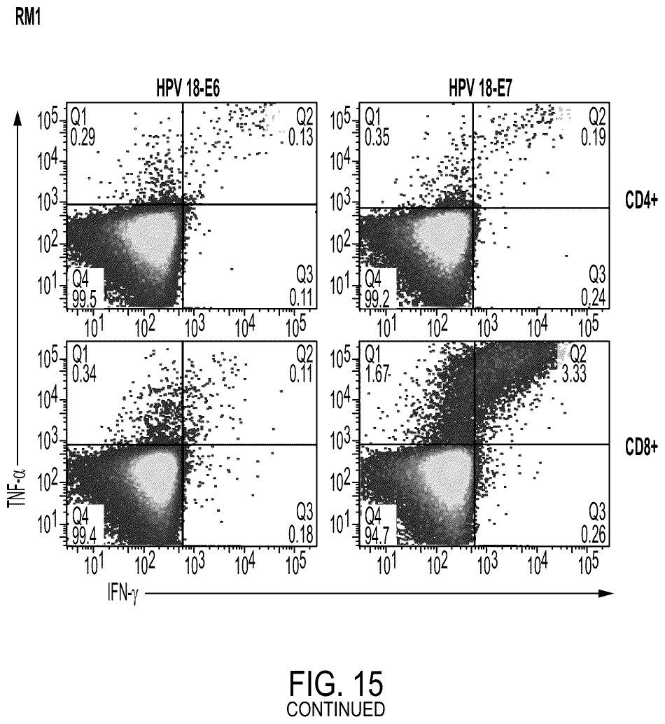

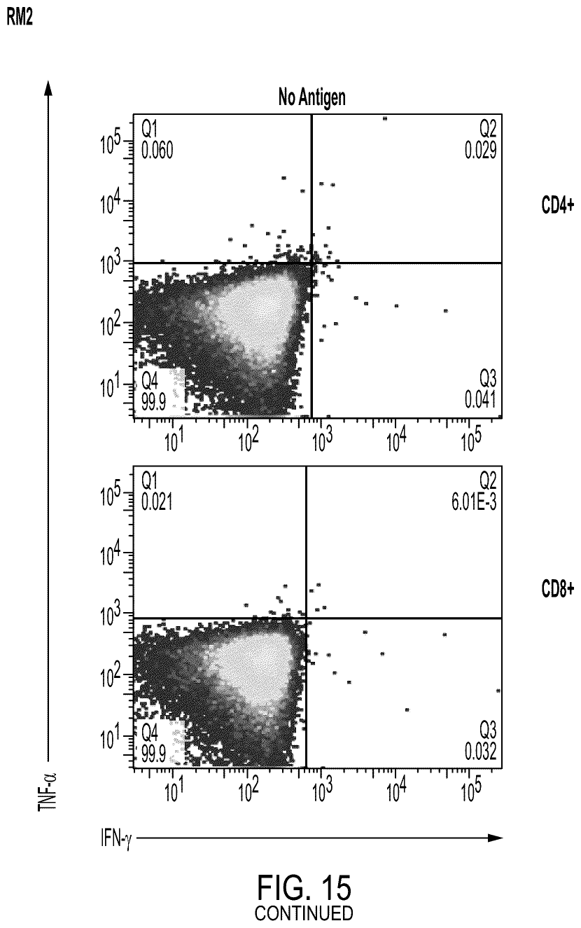

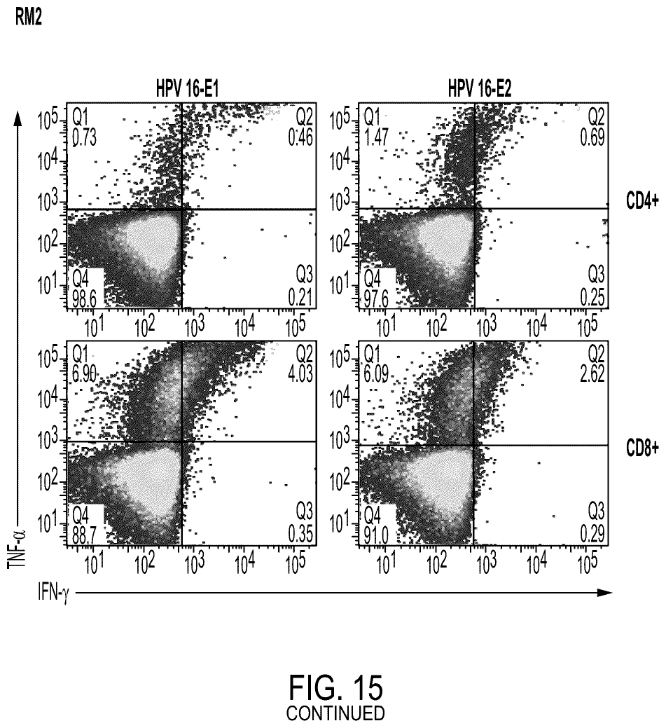

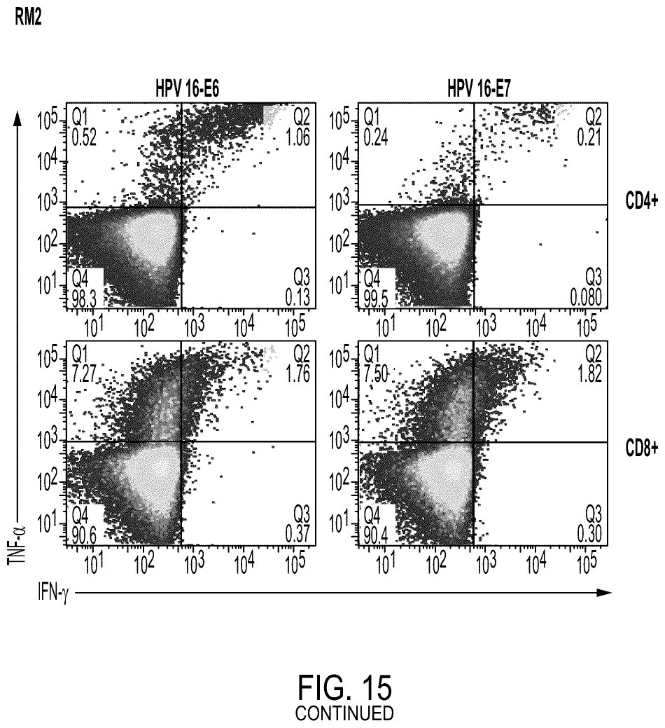

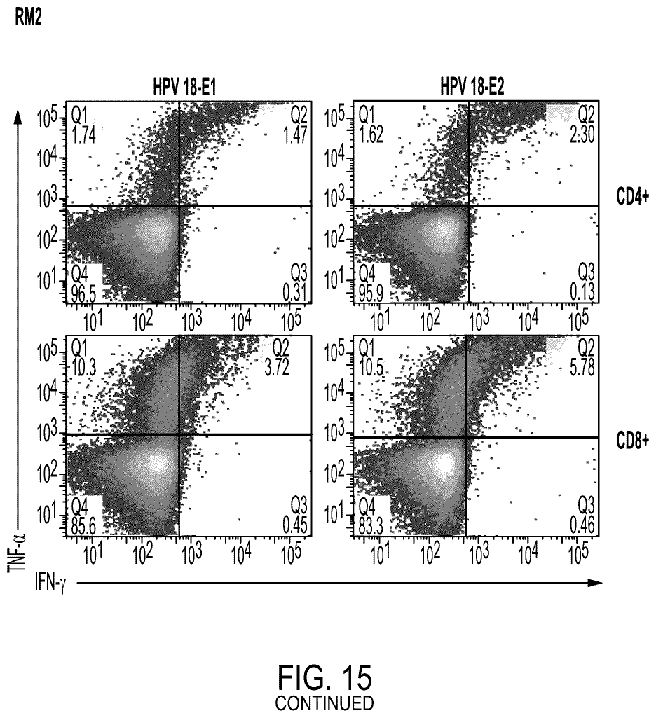

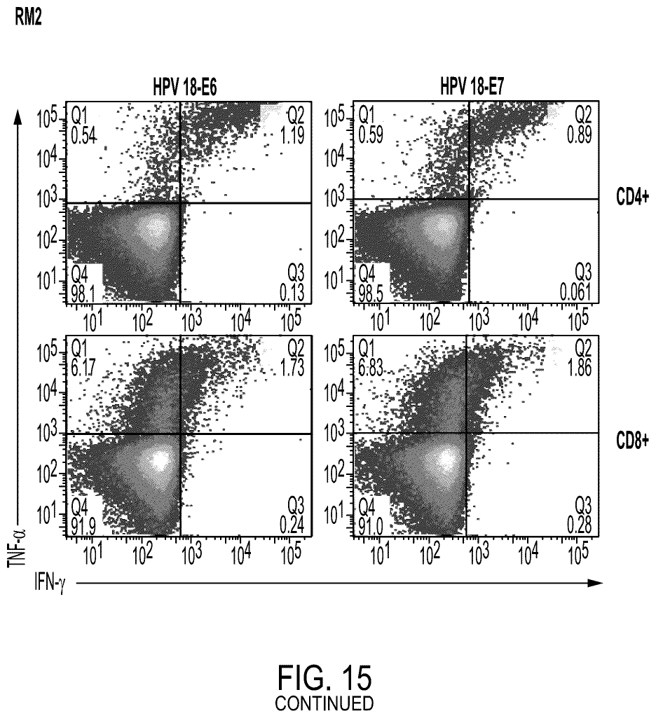

Rhesus macaques (n=2) were administered CMV-HPV. PBMCs were isolated on day 35-42 post-inoculation and CD4+ and CD8+ T cell responses were detected by intracellular cytokine staining (ICS) of IFNγ and TNFα after stimulation with peptide pools corresponding to each component of the HPV fusion protein (HPV 16-E1, HPV16-E2, HPV16-E6, HPV16-E7, HPV 18-E1, HPV18-E2, HPV18-E6, and HPV18-E7) (FIG. 15).

The various embodiments described above can be combined to provide further embodiments. All of the U.S. patents, U.S. patent application publications, U.S. patent applications, foreign patents, foreign patent applications, and non-patent publications referred to in this specification and/or listed in the Application Data Sheet, including U.S. Provisional Patent Application No. 63/517,310 filed Aug. 2, 2023 and U.S. Provisional Patent Application No. 63/574,128 filed Apr. 3, 2024, are incorporated herein by reference, in their entirety unless specified otherwise. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A fusion protein comprising or consisting of:
(a) an amino acid sequence having 90%, 91%, 92%, 93%, 94%, 95%, 96%, 97%, 98%, 99%, or 100% identity to the amino acid sequence according to SEQ ID NO:4;
(b) the amino acid sequence according to SEQ ID NO:4;
(c) an amino acid sequence having 90%, 91%, 92%, 93%, 94%, 95%, 96%, 97%, 98%, 99%, or 100% identity to the amino acid sequence according to SEQ ID NO:1;
(d) the amino acid sequence according to SEQ ID NO: 1;
(e) an amino acid sequence having 90%, 91%, 92%, 93%, 94%, 95%, 96%, 97%, 98%, 99%, or 100% identity to the amino acid sequence according to SEQ ID NO:2;
(f) the amino acid sequence according to SEQ ID NO:2;
(g) an amino acid sequence having 90%, 91%, 92%, 93%, 94%, 95%, 96%, 97%, 98%, 99%, or 100% identity to the amino acid sequence according to SEQ ID NO:3;
(h) the amino acid sequence according to SEQ ID NO:3;
(i) (i) an amino acid sequence having 90%, 91%, 92%, 93%, 94%, 95%, 96%, 97%, 98%, 99%, or 100% identity to the amino acid sequence according to SEQ ID NO: 1; (ii) an amino acid sequence having 90%, 91%, 92%, 93%, 94%, 95%, 96%, 97%, 98%, 99%, or 100% identity to the amino acid sequence according to SEQ ID NO: 2; and (iii) an amino acid sequence having 90%, 91%, 92%, 93%, 94%, 95%, 96%, 97%, 98%, 99%, or 100% identity to the amino acid sequence according to SEQ ID NO: 3;
(j) (i) the amino acid sequence according to SEQ ID NO:1; (ii) the amino acid sequence according to SEQ ID NO:2; and (iii) the amino acid sequence according to SEQ ID NO:3;
(k) (i) an amino acid sequence having 90%, 91%, 92%, 93%, 94%, 95%, 96%, 97%, 98%, 99%, or 100% identity to the amino acid sequence according to SEQ ID NO: 1; and (ii) an amino acid sequence having 90%, 91%, 92%, 93%, 94%, 95%, 96%, 97%, 98%, 99%, or 100% identity to the amino acid sequence according to SEQ ID NO: 2;
(l) (i) the amino acid sequence according to SEQ ID NO:1; and (ii) the amino acid sequence according to SEQ ID NO:2;
(m) (i) an amino acid sequence having 90%, 91%, 92%, 93%, 94%, 95%, 96%, 97%, 98%, 99%, or 100% identity to the amino acid sequence according to SEQ ID NO: 1; and (ii) an amino acid sequence having 90%, 91%, 92%, 93%, 94%, 95%, 96%, 97%, 98%, 99%, or 100% identity to the amino acid sequence according to SEQ ID NO: 3; or
(n) (i) the amino acid sequence according to SEQ ID NO:1; and (ii) the amino acid sequence according to SEQ ID NO:3.

2. A fusion protein encoded by a nucleic acid comprising a sequence having 90%, 91%, 92%, 93%, 94%, 95%, 96%, 97%, 98%, 99%, or 100% identity to the nucleic acid sequence according to SEQ ID NO:16, 13, 14, or 15.

3-9. (canceled)

10. A nucleic acid molecule encoding the fusion protein according to claim 1.

11. A vector comprising the nucleic acid molecule of claim 10.

12. (canceled)

13. The vector of claim 11, wherein the vector is a viral vector, optionally a cytomegalovirus (CMV) vector, a RhCMV vector, a HCMV vector, or a recombinant HCMV vector.

14. The vector of claim 13, wherein the vector comprises a nucleic acid sequence having at least 90%, 91%, 92%, 93%, 94%, 95%, 96%, 97%, 98%, 99%, or 100% identity to the nucleic acid sequence according to SEQ ID NO:17.

15. (canceled)

16. The vector of claim 13, wherein the vector comprises a promoter that is operably linked to the nucleic acid molecule encoding the fusion protein and the promoter is a UL78 promoter, or an ortholog thereof.

17. The vector of claim 16, wherein the nucleic acid molecule encoding the fusion protein replaces all or part of UL78.

18. The vector of claim 13, wherein the RhCMV vector or HCMV vector does not express UL128 or UL130, or orthologs thereof.

19. A recombinant HCMV vector comprising a TR3 backbone and a nucleic acid sequence encoding a heterologous antigen according to SEQ ID NO: 4, wherein:
(a) the vector does not express UL128 or UL130, or orthologs thereof,
(b) the vector comprises a nucleic acid sequence encoding UL146 and UL147, or orthologs thereof; and
(c) the nucleic acid encoding a heterologous antigen replaces all or part of UL78 and is operably linked to the UL78 promoter.

20. The recombinant HCMV vector of claim 19, wherein the recombinant HCMV vector comprises a nucleic acid sequence encoding UL18 and UL82, or orthologs thereof.

21.-23. (canceled)

24. A pharmaceutical composition comprising (i) the recombinant HCMV vector of claim 19; and (ii) a pharmaceutically acceptable carrier.

25. A pharmaceutical composition comprising the recombinant HCMV vector of claim 19, wherein the pharmaceutical composition is lyophilized.

26. A method of generating an immune response in a subject, comprising administering to the subject the recombinant HCMV vector of claim 19.

27.-28. (canceled)

29. A method of treating or preventing an abnormal cytology, neoplasm, dysplasia, or precancerous condition in a subject, comprising administering to the subject the recombinant HCMV vector of claim 19.

30.-31. (canceled)

32. The method of claim 29, wherein the abnormal cytology, neoplasm, dysplasia, or precancerous condition is a high-grade squamous intraepithelial lesion (HSIL).

33. The method of claim 32, wherein the HSIL is a cervical HSIL or anal HSIL.

34. The method of claim 33, wherein the cervical HSIL is a cervical squamous intraepithelial neoplasia 2 (CIN2) or cervical squamous intraepithelial neoplasia 3 (CIN3).

35. The method of claim 33, wherein the anal HSIL is an anal intraepithelial neoplasia 2 (AIN2) or anal intraepithelial neoplasia (AIN3).

36. A method of treating or preventing HPV infection in a subject, comprising administering to the subject the recombinant HCMV vector of claim 19.

37.-38. (canceled)

39. A method of treating or preventing an HPV-associated cancer in a subject, comprising administering to the subject the recombinant HCMV vector of claim 19.

40.-41. (canceled)

42. The method of claim 39, wherein the HPV-associated cancer is cervical, penile, vaginal, vulval, anal, rectal, oropharyngeal, skin, and/or head and neck cancer.

43. The method of claim 29, wherein the subject is CMV positive.

44. The method of claim 29, wherein the subject is CMV negative.

45. The method of claim 29, wherein the subject is HPV positive.

46. The method of claim 29, wherein the subject is HPV negative.

47. The method of claim 29, wherein the subject is positive for human immunodeficiency virus (HIV).

48. The method of claim 47, wherein the subject has received an antiretroviral therapy (ART).

49. The method of claim 47, wherein the subject is administered or is to be administered an ART.

50.-51. (canceled)

52. The method of claim 29, wherein the recombinant HCMV vector is administered in an amount effective to elicit a CD4+ T cell response to an HPV antigen.

53. The method of claim 52, wherein at least 10% of the CD4+ T cells elicited by the recombinant HCMV vector are restricted by MHC-II or an ortholog thereof.

54. The method of claim 29, wherein the recombinant HCMV vector is administered in an amount effective to elicit a CD8+ T cell response to an HPV antigen.

55. The method of claim 54, wherein at least 10% of the CD8+ T cells elicited by the recombinant HCMV vector are restricted by MHC-Ia or an ortholog thereof.

56. The method of claim 29, wherein the recombinant HCMV vector is administered subcutaneously.

57. The method of claim 29, wherein the recombinant HCMV vector is administered intramuscularly.


SEQUENCE LISTING (24 sequences)

SEQ ID NO: 1 - protein, 499 aa, synthetic construct
MHGPKATLQDIVLHLEPQNEIPVDLLGHGQLSDSEEENDEIDGVNHQHLPARRAEPQRHTMLCMGCKCEARIELVVESSADDLRAFQQLFLNTLSFVGPWCASQQMARFEDPTRRPYKLPDLCTELNTSLQDIEITCVYCKTVLELTEVFEFAFKDLFVVYRDSIPHAACHKGIDFYSRIRELRHYSDSVYGDTLEKLTNTGLYNLLIRCLRCQKPLNPAEKLRHLNEKRRFHNIAGHYRGQGHSCCNRARQERLQRHGDTPTLHEYMLDLQPETTDLYGYGQLNDSSEEEDEIDGPAGQAEPDRAHYNIVTFGCKCDSTLRLCVQSTHVDICTLEDLLMGTLGIVGPICSQKPMFQDPQERPRKLPQLCTELQTTIHDIILECVYCKQQLLRREVYDFAFRDLCIVYRDGNPYAVCDKGLKFYSKISEYRHYCYSLYGTTLEQQYNKPLCDLLIRCINCQKPLCPEEKQRHLDKKQRFHNIRGRWTGRGMSCCRSSRT

SEQ ID NO: 2 - protein, 598 aa, synthetic construct
PKLRSTAAALYWYKTGMSNISEVYGDTPEWIQRQTVLQHSFNDCTFELSQMVQWAYDNDIVDDSEIAYKYAQLADTNSNASAFLKSNSQAKIVKDCATMCRHYKRAEKKQMSMSQWIKYRCDRVDDGGDWKQIVMFLRYQGVDFMSFLSALKKFLQGIPKKNCILLYGAANTGALFGMSLMKFLQGSVICFVNSKSHFAFQPLADAKIGMLDDATVPCWNYIDDNLRNALDGNLVSMDVKHRPLVQLKCPPLLITSAINAGTDSRWPYLHNRLVVFTFPNEFPFDENGNPVYELNDKNWPKLRSSVAALYWYRTGISNISEVMGDTPEWIQRLTIIQHGIDDSNFDLSEMVQWAFDNELTDESDMAFEYALLADSNSNAAAFLKSNCQAKYLKDCATMCKHYRRAQKRQMNMSQWIRFRCSKIDEGGDWRPIVQFLRYQQIEFITFLGALKSFLKGTPKKNCLVFCGPANTGAYFGMSFIHFIQGAVISFVNSTSHAFLEPLTDTKVAMLDDATTTCWTYFDTYMRNALDGNPISIDRKHKPLIQLKCPPILLTTAIHPAKDNRWPYLESRITVFEFPNAFPFDKNGNPVYEINDKNW

SEQ ID NO: 3 - protein, 400 aa, synthetic construct
METLCQRLNVCQDKILTHYENDATDLRDHIDYWKQMALACAIYYKAREMGFKHINHQVVPTLAVSKNKALQAAELQLTLETIYNSQYSNEKWTLQDVSLEVYLTAPTGCIAKHGYTVEVQFDGDICNTMHYTNRKYIYICEETSVTVVEGQVDYYGLAYVHEGIQTYFVQFKDDAEKYSKNKVWEVHAGGQAILCPTSVFMQTPKETLSERLSALQDKIIDHYENDAKDIDSQIQYWQLIAWANAIFFAAREHGIQTLNHQVVPAYNISKSKAHKAAELQMALQGLAQSAYKTEDWTLQDTCEELWNTEPTHCFAKGGQTVQVYFDGNKDNSMNYVARDSVYYMTDAGTWDKTATCVSHRGLAYVKEGYNTFYIEFKSECEKYGNTGTWEAHFGNNVIDC

SEQ ID NO: 4 - protein, 1497 aa, synthetic construct
MHGPKATLQDIVLHLEPQNEIPVDLLGHGQLSDSEEENDEIDGVNHQHLPARRAEPQRHTMLCMGCKCEARIELVVESSADDLRAFQQLFLNTLSFVGPWCASQQMARFEDPTRRPYKLPDLCTELNTSLQDIEITCVYCKTVLELTEVFEFAFKDLFVVYRDSIPHAACHKGIDFYSRIRELRHYSDSVYGDTLEKLTNTGLYNLLIRCLRCQKPLNPAEKLRHLNEKRRFHNIAGHYRGQGHSCCNRARQERLQRHGDTPTLHEYMLDLQPETTDLYGYGQLNDSSEEEDEIDGPAGQAEPDRAHYNIVTFGCKCDSTLRLCVQSTHVDICTLEDLLMGTLGIVGPICSQKPMFQDPQERPRKLPQLCTELQTTIHDIILECVYCKQQLLRREVYDFAFRDLCIVYRDGNPYAVCDKGLKFYSKISEYRHYCYSLYGTTLEQQYNKPLCDLLIRCINCQKPLCPEEKQRHLDKKQRFHNIRGRWTGRGMSCCRSSRTPKLRSTAAALYWYKTGMSNISEVYGDTPEWIQRQTVLQHSFNDCTFELSQMVQWAYDNDIVDDSEIAYKYAQLADTNSNASAFLKSNSQAKIVKDCATMCRHYKRAEKKQMSMSQWIKYRCDRVDDGGDWKQIVMFLRYQGVDFMSFLSALKKFLQGIPKKNCILLYGAANTGALFGMSLMKFLQGSVICFVNSKSHFAFQPLADAKIGMLDDATVPCWNYIDDNLRNALDGNLVSMDVKHRPLVQLKCPPLLITSAINAGTDSRWPYLHNRLVVFTFPNEFPFDENGNPVYELNDKNWPKLRSSVAALYWYRTGISNISEVMGDTPEWIQRLTIIQHGIDDSNFDLSEMVQWAFDNELTDESDMAFEYALLADSNSNAAAFLKSNCQAKYLKDCATMCKHYRRAQKRQMNMSQWIRFRCSKIDEGGDWRPIVQFLRYQQIEFITFLGALKSFLKGTPKKNCLVFCGPANTGAYFGMSFIHFIQGAVISFVNSTSHAFLEPLTDTKVAMLDDATTTCWTYFDTYMRNALDGNPISIDRKHKPLIQLKCPPILLTTAIHPAKDNRWPYLESRITVFEFPNAFPFDKNGNPVYEINDKNWMETLCQRLNVCQDKILTHYENDATDLRDHIDYWKQMALACAIYYKAREMGFKHINHQVVPTLAVSKNKALQAAELQLTLETIYNSQYSNEKWTLQDVSLEVYLTAPTGCIAKHGYTVEVQFDGDICNTMHYTNRKYIYICEETSVTVVEGQVDYYGLAYVHEGIQTYFVQFKDDAEKYSKNKVWEVHAGGQAILCPTSVFMQTPKETLSERLSALQDKIIDHYENDAKDIDSQIQYWQLIAWANAIFFAAREHGIQTLNHQVVPAYNISKSKAHKAAELQMALQGLAQSAYKTEDWTLQDTCEELWNTEPTHCFAKGGQTVQVYFDGNKDNSMNYVARDSVYYMTDAGTWDKTATCVSHRGLAYVKEGYNTFYIEFKSECEKYGNTGTWEAHFGNNVIDC

SEQ ID NO: 5 - protein, 158 aa, Alphapapillomavirus 7
MARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPLNPAEKLRHLNEKRRFHNIAGHYRGQCHSCCNRARQERLQRRRETQV

SEQ ID NO: 6 - protein, 158 aa, Alphapapillomavirus 9
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 7 - protein, 105 aa, Alphapapillomavirus 7
MHGPKATLQDIVLHLEPQNEIPVDLLCHEQLSDSEEENDEIDGVNHQHLPARRAEPQRHTMLCMCCKCEARIKLVVESSADDLRAFQQLFLNTLSFVCPWCASQQ

SEQ ID NO: 8 - protein, 98 aa, Alphapapillomavirus 9
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 9 - protein, 657 aa, Alphapapillomavirus 7
MADPEGTDGEGTGCNGWFYVQAIVDKKTGDVISDDEDENATDTGSDMVDFIDTQGTFCEQAELETAQALFHAQEVHNDAQVLHVLKRKFAGGSTENSPLGERLEVDTELSPRLQEISLNSGQKKAKRRLFTISDSGYGCSEVEATQIQVTTNGEHGGNVCSGGSTEAIDNGGTEGNNSSVDGTSDNSNIENVNPQCTIAQLKDLLKVNNKQGAMLAVFKDTYGLSFTDLVRNFKSDKTTCTDWVTAIFGVNPTIAEGFKTLIQPFILYAHIQCLDCKWGVLILALLRYKCGKSRLTVAKGLSTLLHVPETCMLIQPPKLRSSVAALYWYRTGISNISEVMGDTPEWIQRLTIIQHGIDDSNFDLSEMVQWAFDNELTDESDMAFEYALLADSNSNAAAFLKSNCQAKYLKDCATMCKHYRRAQKRQMNMSQWIRFRCSKIDEGGDWRPIVQFLRYQQIEFITFLGALKSFLKGTPKKNCLVFCGPANTGKSYFGMSFIHFIQGAVISFVNSTSHFWLEPLTDTKVAMLDDATTTCWTYFDTYMRNALDGNPISIDRKHKPLIQLKCPPILLTTNIHPAKDNRWPYLESRITVFEFPNAFPFDKNGNPVYEINDKNWKCFFERTWSRLDLHEEEEDADTEGNPFGTFKLRAGQNHRPL

SEQ ID NO: 10 - protein, 649 aa, Alphapapillomavirus 9
MADPAGTNGEEGTGCNGWFYVEAVVEKKTGDAISDDENENDSDTGEDLVDFIVNDNDYLTQAETETAHALFTAQEAKQHRDAVQVLKRKYLVSPLSDISGCVDNNISPRLKAICIEKQSRAAKRRLFESEDSGYGNTEVETQQMLQVEGRHETETPCSQYSGGSGGGCSQYSSGSGGEGVSERHTICQTPLTNILNVLKTSNAKAAMLAKFKELYGVSFSELVRPFKSNKSTCCDWCIAAFGLTPSIADSIKTLLQQYCLYLHIQSLACSWGMVVLLLVRYKCGKNRETIEKLLSKLLCVSPMCMMIEPPKLRSTAAALYWYKTGISNISEVYGDTPEWIQRQTVLQHSFNDCTFELSQMVQWAYDNDIVDDSEIAYKYAQLADTNSNASAFLKSNSQAKIVKDCATMCRHYKRAEKKQMSMSQWIKYRCDRVDDGGDWKQIVMFLRYQGVEFMSFLTALKRFLQGIPKKNCILLYGAANTGKSLFGMSLMKFLQGSVICFVNSKSHFWLQPLADAKIGMLDDATVPCWNYIDDNLRNALDGNLVSMDVKHRPLVQLKCPPLLITSNINAGTDSRWPYLHNRLVVFTFPNEFPFDENGNPVYELNDKNWKSFFSRTWSRLSLHEDEDKENDGDSLPTFKCVSGQNTNTL

SEQ ID NO: 11 - protein, 365 aa, Alphapapillomavirus 7
MQTPKETLSERLSCVQDKIIDHYENDSKDIDSQIQYWQLIRWENAIFFAAREHGIQTLNHQVVPAYNISKSKAHKAIELQMALQGLAQSAYKTEDWTLQDTCEELWNTEPTHCFKKGGQTVQVYFDGNKDNCMTYVAWDSVYYMTDAGTWDKTATCVSHRGLYYVKEGYNTFYIEFKSECEKYGNTGTWEVHFGNNVIDCNDSMCSTSDDTVSATQLVKQLQHTPSPYSSTVSVGTAKTYGQTSAATRPGHCGLAEKQHCGPVNPLLGAATPTGNNKRRKLCSGNTTPIIHLKGDRNSLKCLRYRLRKHSDHYRDISSTWHWTGAGNEKTGILTVTYHSETQRTKFLNTVAIPDSVQILVGYMTM

SEQ ID NO: 12 - protein, 365 aa, Alphapapillomavirus 9
METLCQRLNVCQDKILTHYENDSTDLRDHIDYWKHMRLECAIYYKAREMGFKHINHQVVPTLAVSKNKALQAIELQLTLETIYNSQYSNEKWTLQDVSLEVYLTAPTGCIKKHGYTVEVQFDGDICNTMHYTNWTHIYICEEASVTVVEGQVDYYGLYYVHEGIRTYFVQFKDDAEKYSKNKVWEVHAGGQVILCPTSVFSSNEVSSPEIIRQHLANHPAATHTKAVALGTEETQTTIQRPRSEPDTGNPCHTTKLLHRDSVDSAPILTAFNSSHKGRINCNSNTTPIVHLKGDANTLKCLRYRFKKHCTLYTAVSSTWHWTGHNVKHKSAIVTLTYDSEWQRDQFLSQVKIPKTITVSTGFMSI

SEQ ID NO: 13 - DNA, 1497 bp, synthetic construct
  source 1..1497: mol_type = other DNA
atgcacggccccaaggccacactgcaggatatcgttctgcacctggaaccccagaacgagatccccgtggatctgctgggccatggccagctgtctgactccgaagaagagaacgacgagatcgacggcgtgaaccaccagcatctccctgctcgaagagccgagcctcaacggcacaccatgctgtgcatgggatgtaaatgcgaagccagaatcgagctggtggttgagagctcggccgacgacctgagagccttccagcagctgtttctgaacaccctgagcttcgtgggcccttggtgcgccagccagcagatggccagatttgaggaccctacacggcggccttacaagctgcctgacctgtgcaccgaactgaacaccagcctgcaggatattgagatcacctgcgtgtactgtaaaaccgtgctggagctgacagaggtttttgaattcgccttcaaggacctgttcgtggtgtacagagatagcatccctcacgccgcttgtcacaagggcattgacttctacagcagaatacgggaactgcggcactactctgattctgtgtacggcgacaccctagaaaagctgaccaacacaggcctgtacaacctcctgatcagatgcctgcgttgccagaagccactgaacccagccgagaagctgagacacctaaacgagaagcggcgcttccacaacatcgccggccactacagaggacagggccactcttgctgcaacagagctagacaggagaggctgcagagacacggcgacacacctactctgcatgagtacatgctggacctgcagcctgaaaccaccgatctgtacggatacggccagctgaacgacagcagcgaggaggaagacgaaatcgatggccctgccggccaggccgagcctgatagagctcactacaacatcgtcaccttcgggtgtaagtgcgactctacactccggctgtgtgtgcagagcacacacgtggacatctgcaccctggaggatcttctgatgggcaccctgggaatcgtgggccctatctgcagccagaagcctatgttccaggaccctcaggagagaccccggaagctgcctcagctgtgcaccgaactgcagacgacaatccacgacattatcctggagtgcgtgtactgcaagcaacaactgctgcggagagaggtgtatgacttcgctttcagagatctgtgcatcgtgtacagagatggcaacccctacgctgtgtgcgacaagggcctcaagttctacagcaagatctctgaataccggcactactgctacagcctgtacggcacaaccctggagcagcagtataacaaacccctgtgtgacctcctgatcagatgcattaactgccaaaaaccactgtgcccagaagaaaagcagcgacacctggacaagaagcagcggtttcacaacatcagaggcagatggaccgggcggggaatgtcttgttgcagaagctctagaact

SEQ ID NO: 14 - DNA, 1794 bp, synthetic construct
  source 1..1794: mol_type = other DNA
ccaaagctgagatctacagccgctgcactgtactggtataagaccggcatgtccaacatcagcgaggtttacggcgatacccctgagtggattcagagacagaccgtgctgcagcactccttcaacgactgtacatttgaactgagccaaatggtgcagtgggcttacgacaatgacattgtggatgatagcgagatcgcctacaagtacgcccagcttgccgacaccaactccaacgcttctgccttcctgaaaagcaacagccaggccaagatcgtgaaggattgcgccacaatgtgcagacactacaagagagctgagaagaagcagatgtccatgtcgcaatggatcaagtaccggtgcgatagagtggacgacggcggagattggaaacagatcgtgatgttcctgcggtaccagggcgtggacttcatgagcttcctgtctgctctgaaaaaatttctgcagggcatccctaagaaaaattgcatcttgctgtatggcgccgctaacaccggcgccctgtttggcatgagcctgatgaagttcctgcagggcagcgttatctgcttcgtgaactccaaaagccacttcgccttccagcccctggctgacgccaagatcggcatgctggacgacgccacagtgccgtgctggaactatatcgacgacaacttgcggaacgccctggacggcaatctggtcagcatggacgtgaagcaccggcctctggtgcagctgaaatgtcctccactgctgatcacgtccgccatcaacgccggcaccgatagcagatggccttacctgcacaatagactggttgtcttcaccttccctaatgaattccctttcgatgagaacggcaaccccgtgtacgagctgaatgataagaactggcccaagctgagaagcagcgtggccgctctgtactggtaccggaccggcattagcaacatcagcgaggtgatgggcgacacacccgaatggattcagagactgaccatcatccagcacggcatcgatgacagcaatttcgacctatctgagatggtccagtgggcctttgacaacgagctgacagacgagagtgatatggcctttgagtacgccctgctggccgattctaacagcaatgccgccgccttcctcaagagcaattgccaagccaagtacctgaaggactgcgccaccatgtgcaagcactataggagagcccagaagcggcagatgaacatgagccagtggatcagattcagatgtagcaagatcgatgagggcggcgactggagacctatcgtgcagttcctgcggtaccagcaaatcgagttcatcaccttcttgggcgccctgaagagcttcctgaagggcacccctaagaaaaactgcctggtgttctgcggccccgccaatacaggagcctacttcggcatgagctttatccactttatccagggagccgtgatctccttcgtgaacagcaccagccacgcctttctggaacctctgaccgacaccaaggtggccatgctggacgacgctaccacaacctgctggacctacttcgatacatacatgagaaacgccctggacggaaatcctatcagcatcgaccggaagcacaagccactgatccagctgaaatgcccccccatcttgctgaccaccgccatccaccccgctaaggacaatcggtggccttacctggaaagcagaatcaccgtgttcgagttccctaacgctttccccttcgacaagaacggcaaccctgtctacgagatcaacgacaagaattgg

SEQ ID NO: 15 - DNA, 1203 bp, synthetic construct
  source 1..1203: mol_type = other DNA
atggaaactttgtgccagagactgaatgtgtgtcaggacaagatcctgacccactacgagaacgacgccaccgatttgagggatcacatcgactactggaagcagatggcactcgcctgtgccatctactacaaggctcgcgagatgggcttcaagcacatcaaccatcaggtggtgcccacgctggccgttagcaagaacaaggccctgcaggccgctgaactgcagctgacactggagaccatctacaacagccaatacagcaacgagaagtggaccctgcaagatgtgagcctggaagtgtacctgacagctcctaccggctgcatcgccaagcacggctataccgtggaagtgcagttcgacggcgatatctgtaacaccatgcactacacaaacagaaagtacatctacatctgcgaggaaacctccgtgaccgtggtggaaggccaggtggactactacggcctggcctacgtgcacgagggcattcagacatacttcgtgcagttcaaggacgacgccgagaagtactccaaaaacaaagtgtgggaagtgcatgctggaggacaggctatcctgtgtcctacaagcgtgttcatgcagacacctaaagagacactgagcgagcggctgagcgccttgcaggacaagatcatcgaccactacgaaaacgacgccaaagacatcgatagccagatccagtactggcagcttatcgcctgggccaacgcgattttcttcgccgcgcgggaacacggcatccaaaccctgaaccaccaggtggtgcctgcttataatatctctaagtccaaggcccacaaggctgccgagctgcagatggccctgcagggcctggcacaatccgcctacaagaccgaggactggacactccaagacacatgtgaagaactgtggaacacagagcctacacattgttttgccaaaggaggccagaccgtgcaggtgtatttcgacggcaacaaggacaactccatgaattacgtggcccgggattctgtgtactacatgaccgacgccggcacctgggacaagaccgccacctgcgtgtcccacagaggactggcctacgtaaaggaaggttacaataccttttacatcgagttcaaaagcgaatgtgagaaatacggcaacaccggaacgtgggaggcgcactttggcaacaacgtgatcgactgctga

SEQ ID NO: 16 - DNA, 4494 bp, synthetic construct
  source 1..4494: mol_type = other DNA
atgcacggccccaaggccacactgcaggatatcgttctgcacctggaaccccagaacgagatccccgtggatctgctgggccatggccagctgtctgactccgaagaagagaacgacgagatcgacggcgtgaaccaccagcatctccctgctcgaagagccgagcctcaacggcacaccatgctgtgcatgggatgtaaatgcgaagccagaatcgagctggtggttgagagctcggccgacgacctgagagccttccagcagctgtttctgaacaccctgagcttcgtgggcccttggtgcgccagccagcagatggccagatttgaggaccctacacggcggccttacaagctgcctgacctgtgcaccgaactgaacaccagcctgcaggatattgagatcacctgcgtgtactgtaaaaccgtgctggagctgacagaggtttttgaattcgccttcaaggacctgttcgtggtgtacagagatagcatccctcacgccgcttgtcacaagggcattgacttctacagcagaatacgggaactgcggcactactctgattctgtgtacggcgacaccctagaaaagctgaccaacacaggcctgtacaacctcctgatcagatgcctgcgttgccagaagccactgaacccagccgagaagctgagacacctaaacgagaagcggcgcttccacaacatcgccggccactacagaggacagggccactcttgctgcaacagagctagacaggagaggctgcagagacacggcgacacacctactctgcatgagtacatgctggacctgcagcctgaaaccaccgatctgtacggatacggccagctgaacgacagcagcgaggaggaagacgaaatcgatggccctgccggccaggccgagcctgatagagctcactacaacatcgtcaccttcgggtgtaagtgcgactctacactccggctgtgtgtgcagagcacacacgtggacatctgcaccctggaggatcttctgatgggcaccctgggaatcgtgggccctatctgcagccagaagcctatgttccaggaccctcaggagagaccccggaagctgcctcagctgtgcaccgaactgcagacgacaatccacgacattatcctggagtgcgtgtactgcaagcaacaactgctgcggagagaggtgtatgacttcgctttcagagatctgtgcatcgtgtacagagatggcaacccctacgctgtgtgcgacaagggcctcaagttctacagcaagatctctgaataccggcactactgctacagcctgtacggcacaaccctggagcagcagtataacaaacccctgtgtgacctcctgatcagatgcattaactgccaaaaaccactgtgcccagaagaaaagcagcgacacctggacaagaagcagcggtttcacaacatcagaggcagatggaccgggcggggaatgtcttgttgcagaagctctagaactccaaagctgagatctacagccgctgcactgtactggtataagaccggcatgtccaacatcagcgaggtttacggcgatacccctgagtggattcagagacagaccgtgctgcagcactccttcaacgactgtacatttgaactgagccaaatggtgcagtgggcttacgacaatgacattgtggatgatagcgagatcgcctacaagtacgcccagcttgccgacaccaactccaacgcttctgccttcctgaaaagcaacagccaggccaagatcgtgaaggattgcgccacaatgtgcagacactacaagagagctgagaagaagcagatgtccatgtcgcaatggatcaagtaccggtgcgatagagtggacgacggcggagattggaaacagatcgtgatgttcctgcggtaccagggcgtggacttcatgagcttcctgtctgctctgaaaaaatttctgcagggcatccctaagaaaaattgcatcttgctgtatggcgccgctaacaccggcgccctgtttggcatgagcctgatgaagttcctgcagggcagcgttatctgcttcgtgaactccaaaagccacttcgccttccagcccctggctgacgccaagatcggcatgctggacgacgccacagtgccgtgctggaactatatcgacgacaacttgcggaacgccctggacggcaatctggtcagcatggacgtgaagcaccggcctctggtgcagctgaaatgtcctccactgctgatcacgtccgccatcaacgccggcaccgatagcagatggccttacctgcacaatagactggttgtcttcaccttccctaatgaattccctttcgatgagaacggcaaccccgtgtacgagctgaatgataagaactggcccaagctgagaagcagcgtggccgctctgtactggtaccggaccggcattagcaacatcagcgaggtgatgggcgacacacccgaatggattcagagactgaccatcatccagcacggcatcgatgacagcaatttcgacctatctgagatggtccagtgggcctttgacaacgagctgacagacgagagtgatatggcctttgagtacgccctgctggccgattctaacagcaatgccgccgccttcctcaagagcaattgccaagccaagtacctgaaggactgcgccaccatgtgcaagcactataggagagcccagaagcggcagatgaacatgagccagtggatcagattcagatgtagcaagatcgatgagggcggcgactggagacctatcgtgcagttcctgcggtaccagcaaatcgagttcatcaccttcttgggcgccctgaagagcttcctgaagggcacccctaagaaaaactgcctggtgttctgcggccccgccaatacaggagcctacttcggcatgagctttatccactttatccagggagccgtgatctccttcgtgaacagcaccagccacgcctttctggaacctctgaccgacaccaaggtggccatgctggacgacgctaccacaacctgctggacctacttcgatacatacatgagaaacgccctggacggaaatcctatcagcatcgaccggaagcacaagccactgatccagctgaaatgcccccccatcttgctgaccaccgccatccaccccgctaaggacaatcggtggccttacctggaaagcagaatcaccgtgttcgagttccctaacgctttccccttcgacaagaacggcaaccctgtctacgagatcaacgacaagaattggatggaaactttgtgccagagactgaatgtgtgtcaggacaagatcctgacccactacgagaacgacgccaccgatttgagggatcacatcgactactggaagcagatggcactcgcctgtgccatctactacaaggctcgcgagatgggcttcaagcacatcaaccatcaggtggtgcccacgctggccgttagcaagaacaaggccctgcaggccgctgaactgcagctgacactggagaccatctacaacagccaatacagcaacgagaagtggaccctgcaagatgtgagcctggaagtgtacctgacagctcctaccggctgcatcgccaagcacggctataccgtggaagtgcagttcgacggcgatatctgtaacaccatgcactacacaaacagaaagtacatctacatctgcgaggaaacctccgtgaccgtggtggaaggccaggtggactactacggcctggcctacgtgcacgagggcattcagacatacttcgtgcagttcaaggacgacgccgagaagtactccaaaaacaaagtgtgggaagtgcatgctggaggacaggctatcctgtgtcctacaagcgtgttcatgcagacacctaaagagacactgagcgagcggctgagcgccttgcaggacaagatcatcgaccactacgaaaacgacgccaaagacatcgatagccagatccagtactggcagcttatcgcctgggccaacgcgattttcttcgccgcgcgggaacacggcatccaaaccctgaaccaccaggtggtgcctgcttataatatctctaagtccaaggcccacaaggctgccgagctgcagatggccctgcagggcctggcacaatccgcctacaagaccgaggactggacactccaagacacatgtgaagaactgtggaacacagagcctacacattgttttgccaaaggaggccagaccgtgcaggtgtatttcgacggcaacaaggacaactccatgaattacgtggcccgggattctgtgtactacatgaccgacgccggcacctgggacaagaccgccacctgcgtgtcccacagaggactggcctacgtaaaggaaggttacaataccttttacatcgagttcaaaagcgaatgtgagaaatacggcaacaccggaacgtgggaggcgcactttggcaacaacgtgatcgactgctga

SEQ ID NO: 17 - DNA, 237532 bp, synthetic construct
  source 1..237532: mol_type = other DNA
caccattccgggccgtgtgctgggtccccgaggggcgggggggtgttttcatcgggggggtgaaatgtgggaatttgggaagttggcggtggcgggggacggcgacggcgaataaaagcgccgtgcggcgcgcacggcgaaacccagacgcgcgtgtgtcttgcgtctctttcagtcgcgcgtctgtcttgcgtgtctttgagtccccggggaaaagaggaagcggtcccgaggggacggcggcgcgactccctggggacgcgaagatcggacgcggaaaagaggaagtccccggggacggccacaggcggaaaaaaaaaacgaggaagccgcagcccagtctgccgggaccggcaggcaggcgcacagacccgcgtcgaggacacacgcagaagaagcgcccgcgccggggcggggggggggatcgcgggccccggggcacactgcttccatctggccgcgcgcacaccccgccgacacacccctgacacacccgcgacacacccggcacacgcccgcgacacacccggcacgcgcccgccacacagccagccgacacacccgcgacacacccgccacacccagccgcacccggcacacacccacccagccgcacccccgccacacccagaccgacgccggtgcgggaccgggctgaggggggtgtaagcgccttgacgcgcggcgctatggcactgcgtgccacggtattggttggcgggacgaggctgaggggggtttgcgggacgtaagcgcgacgccgagtttgcggcgtgctgtggcgcgctcaaggctagggacgggggccgcgcgtagcttttcggcggcgacggggaacaaaacaggcacgcacacgcagctcgcttcagcggtcgccctatcggcaccgccgctattctttattaacgtcgttctccccccgcttctacacgcggaccgctgcagacggctatgctcagactgtttctcttgacggcgacatgtgtttgcgtctgtgcggcgcggcgacggccgcaagcggacgacgccgcctgccaccacacgtggcccacgcgctgcgccggcccgcgtcccgcgccgtcggctcacaacacggcgctttcttcctcgcacgccatctccatcgcacaccgtgtgctgcattcgccgtcgcctccacgagagaacatccgccacagcatgcgctgtcgccgtcgcgccatggcctcctcggcttgcacacccgtttcgcacacccagcctcgggccgccaaccacagccgttcgaggatgacgtacgcgacgagcgagcccaccaattctcccacggcctcgcccgccaagtcagacggtggtgttgcctgcggcggggacgggggttgcgctgggatcgggggtggcgccggggacggggggctttcgcagcggggaacacacaccgcctatttaacctccacccgctacaacacacacatgccgcacaatcatgccagccacagacacaaacagcacccacaccacgccgcttcacccagaggaccaacacacgttacccgtacaccacagtaacacacaaccgcaagtccaaacctcggacaaacgcgccgccgaagaccaccgcacgcagatggagctcgacgccgcggactacgctgcttgctcacaggcccgccaacacctctacggtcaaacacaaccccaactacacgcataccccaacgccaacccacaggaaagcgctcattttttcacagagaatcaacatcaagtcacgcatctacttcacaacattggcgagggcgcagcgctcggctaccccgtcccccgcgcggaaatccgccgtggcggtggcgactgggccgacagcgcaagcgacttcgacgccgactgctggtgcatgtggggacgcttcggaaccatgggccgccaacctgtcgtgaccttactgttggcgcgccaacgcgacggcctcgctgactggaacgtcgtacgctgccgcggcacaggctttcgcgcacacgattccgaggacggcgtctctgtctggcgtcagcacctggtttttttactcggaggccacggccgccgtgtacagttagaacgtccatccgcgggagaagcccaagctcgaggcctattgccacgcatccggatcacccccctctccacatctccacgcccaaaaccaccccagcccaccacatccaccgcctcgcacccacatgctacggctcggccagatcacacgctctttcctgtcccttccacaccctcaaccacggttcacaatccccgaaactacgccgtccaacttcacgccgaaacgacccgcacatggcgctgggcacgacgcggtgaacgtggcgcgtggatgccggccgagacatttacgtgtcccaaggataaacgtccctggtagacggggtagggggatctaccagcccagggctcgcgtatttcgccgccacgctgcttcaccgatatccaataaacccatcccctcgccacgacgtctccgcgtatctttgtagcctcaggaatccgtccccacgtccacccatcccgagcactccacacgctataacagaccacggacacggcaaatgcatgcaaacttctcatttattgtgtctactactctgtgttgctacagggggtgaaggcaaagaaaaaaaaaaggaacaaaataatagattagcagaaggaataatccgtgcgaccgagcttgtgcttcttttcttataaggaggcaaatatactagggaaaacataagaataggaagaaaccgaggtttgggagaaaagctgagataaaatagcgcattttccatacagaggttgttgtttttgtggatcctaagaggtttcaagtgcgaatcttaaagttctcacgagaatattgtcttcaagaatcgacaactgtggtccaagatttttttttggtctttttaggttctgcgagggacatcacgatggatcgttgcgatgaagtcacgcgtacgcctctggtgtggcgcggtgtcgtgacaggagagtgtgttttcagtgcagagctgtcttgattcctatatccgagtatctgttttctcgtaaggacggtaatcttctttggtgtaagtacatctaaaagctgcaaactatattttaagggctgtctctaggtgtactttgatgctggagtttttcgctgtgttgatgtgaataaatctactactactattatatgcagaaagagtgattatgccgagacaagattgcattggctgaactgtttcaaaaacgcctacactctacttatccgtaaacctaaggtaatactatgtgtaagttgttttttttttctttttgtagtaaaatggtgatacgtgcaattaaaactgtattccatgtttccatcctttcatttcaactttaaaggcggctttgagagcgaagaagtgcgaggataaaaatggatgactccttcgtgtccagggagtcgactactgcaacgctgattgattaaaagatggtctccgatgatgatgttgttattgatcgaatcatggtgcagaacggcgacggagaggagcgtgtccgccgccgggaaggtggtctctttctcttttcttttttcaagaaatcttccatgtgtttatcgtagtgatcgaaatcgactgatctcgggttctttttgttggtttcttttcggttaatcatgtattgttttctttttttacagaaagatacttttttcatgagcaattcctcgcccggcgccggcatgccgaggtggggccactgcgatcagcggcatgccgacgccgacccggggatcttggattcaccgttttctctcttctctctctacatacagaccgggtggcaggagcggtaaggaatcatcgtcgtctttcattcttcgatgattatggtaatactaaatcttatctaggagcatatacatctaagattggagtactagtagtcgtttgtggtttctatttttttttatatttatctatgacagtttttctgtttttcgttttgataataatataataaaaactcatggacgtgaaatctggcttggttgtggtgatttcattctcattattgttgttttctttccgtcttgcggatgaagatgttgcgatgcggttgttgttggtgttgctatacaccgagagagatgatctttttgttcttctggttcatttcctatgattgtttggctgctgaccgacgcgtcaggatgtgcagggcatgcggggaatcaggaccggacacgggataatttcatctacctatacggagatcgcggtcctcgccatgaggatcgcgacaggcgcgtcgagggggcaggaacacccttgcggattgacattcttggtggtgtttcgttgttgtcggtagttgttgttgacgatgaggataaataaaaatgaccttgtttttgttctgttttctcttgttgggaatcgtcgactttgaattcttcgagttatcggaaagctgaggtacccaaatgtctgtagcttttttctttttaccctcttgtttatcatctgcgattcgtggtaggtaggagagggaaatgataatccgagattaaggaaaggagaagataaaataaaaaaaaaataaaacagaagccgaccggccgccgacccgttccccaggaccagcctacgaggaacggataacgcggtggcgacggcagcggtggtggcgctgggggtggcggtagtggtactgctgatggtagtcgggacggaggagagacgatgcatacatacacgcgtgcatgctgcatgggtggatggtccgaccgggagacgcggaagagaaactcacataaaaaggtgacaaaaagagcggttgaaaaaagaaaacgagattcgaccagacagaagaggaggaccggggcttggcgacccttccacgactgctgttgtcatctcggctcctccgtcttctcccggccacgggcggctaagtcaccgccgttctccccatccgtccgagcgccgaccgaccagccggccgattcgcccgccggggcttctggagaacgccggggcagcagcgatctggagaagccgctaaacccctgcgtttttatatggtagctctgccgagcgcgggctgacgcgttaagtaagcggaaagacgtgtgtgacgaaaaggggtcccatggtatttcacgtgacgatgaggagatgcggtttggagcacatacggtttagaaaaagggagttgtcgtgacaagggctgagggacctctgtctccatgtgtgtataaaaagcaaggcacgttcataatgtaaaaaagaacacgttgtaaacaagctattgctgtatcattcggctgactatgcttcattcggactgattttcttttcctaacggcgtaacttaaagtgattaacgtatgatatttgttccccagagttatactatagtcatcatcctaaaattcagatataaatgaacacatgtcgtatgagattattaagaaaccgaaaccacccatagttcaccatcctcttcatcattcagccgatgacccactccgtacaacgactcagtctgcttcgtcatattgcaaagcacaagcgacgtatgtgaacaacttgaaacacagactgtgttattaatgaccgttgtaccattactagtcacattgcataaagatcctccgccgtcgtcccatcttttccactcggtggaaaaccggtcgctatcatcaactatggtgagattttcaccctgcgtggtattcagtttcttcatattcataccttggattccattattaaaccccaatattaagcacgttattagtaccccccccccccaccaaggaatgtgactggaccggttcctagcagctctgggagccatgttcaggttgaaccacagctacagcgaaaccgagtccagtgaccggtaaccacgtccagcccctgcgtatgtaccagtccaagcacgtccggtcattgttctacacaggaaatctaactaggtcaacgtaattttattccaccgttacgcagaatactaacaaaaaactacacaaatgtaacggattacacataatttattacgtgaaaactgtaagaaagccaattcaccaagcgatacatttatttgacttccaagtgccacacatcaccactatattcatccatgttttcaccgaaccaacgagacagatcgaagaagccagaatcttccgactttaaattacataaatccaacgtattatgaccacagctcgacacacaaatagttgcgttactattcacagtggcattacctatacccgtaacgttgcacaaccactgatcaccattgtcaccaaaaacggttttccacttagttgtcaacggatctttcctatgcgtaatggtaaaattactaccagtcgtcgcttttagctcattacgagtattatccgcatccacatatatcaacgtcatagctaggcacgctataagtaccccccccccacaatggaatgttgccaaaccggttctttcccgttatagccatagcgttcccaggcaaaagcaaacgccgaacctaatgcagtaaaaagcgcttgcagccagaaccagcttatgtaccagccacgataacatccggtcactgtttccacaggaaaccctaccagggtagagccccgcttgttttttcctgtctatcttgtttagcaactcgtaaactgtcagtctagccacgtccgtttagatcaaaagtcacgtatactgcgacgttgtttccacccgtttccccgtcccgccgtttccgaacaacccacccgggttcagacaaccgaccaccaacagaaatatacacacagaccaccgggagttcagttaaagatttcatcaggtttattttggctgctgctagtcttttgcttcttagaaaaaaaatacccatatagagaaataatgatagtttgacaacacatatggcagggatttcttcttcatcaataagatatgcaattcccccagggagagactttcaacaattgaatttacaaaaacaaaattacatcaggagaaagagaggatacattaataaatatattatatctggtgtatatactgaatgctgctggttcataaggtaacgatgctactttttttaattccaagatggtttttctttgttagtcttttgttgacttgctggttcctaaaagttctcaaaaacgattgtgtgaagattttatgacgttggttgactagttcatgagattctgctgtacgtgtgatggttattcgctggttcgttctaagatgagtatcgtactgtgtctgcgatggtcgtctcttactggcattctctcggctgcctcttgctttcatgattgaaaaggaaaaaaggactccgagggcgcggtcatcttttacttttcggttttttcgttggcgggtcagaggtagtcagatcatgagactgtcgtggtcgatgaaactgtgtctgctcaagtgacgtccatttcttgtacggagaaaaaagtcatcgggataaataaggctatacaaggcgttgtcaagcgtgcggctctaaacaaattaagcgatacaaaattacagtaatacgaataataagttaccccctccccctgtggtccccccgagacgagagccacccatcgtgtactctcgcaccacccacgaccacagagggagacgggacgaagagacgacgcagagcgccatctccttctggaggccggcggcgttaactgctacagctgcggcggcgacaacagctgcgatttgtcggccgacatgccgatggtatgggcggcggcggcagtggccgcggcagcggggaggagaggagagagaagaggagcggggcgtccgaaggcgaggatggcatggtctcgccggagcgcccggcttttatggaacgctcgcgtccggtcgggcagcgcccacaggaagatgagtcaaaacttttaaaccatcctgagacccgagtagcggtttacaggccgcacgccagtcttagctaaaaacagcggacagtcccacgctgtttctgttgtggctctctccagtttcctcatcgccgtcccgatctccgtcgtcatcggaagaataccacccgctctcatgcggcagtcgatcgacctcgacgaacgagacgcggcgacgcctctctacggccgactggttgtggtggtgaaagaagagcaccagcaatcccaggaggagcaacaagccctcacatgtccaggaggtcggggagagggcctgtcggagatggccgtgaggcatcacgtacggcagctgaggagaaacggagaagaaaggaaaattaccgtcaggggccggggttcttattagagaaacagcacgtaggtcaggatccagatgctaatggcaatcatgatgacgatgatcatgcaggccaagacgcggcgcaccaatgccgaatccaatagccgccgtgcctccggttggtggccggcggcatctagagacatgatttgggggggaccggcggcgcgaaaagacagggagatggacagtgtcacggtgttttgttatgattaggacatggggaccggaagccgagacagagtactacagggtgttgaagggtaacgtgagggagatcatgtcatgggcgggctgaagaccgtgcggggaggattggcgtgtgcggtgcttgtggaacacggtgttttaatatgtatccgcgtgtaatgcacgcggtgtgctttttagcactcggcttgataagctacgtggccgtttgcgccgaaaacacggttaccaccaactgtctcgtgaaaacagaaaatacccacctaacatgtaagtgcaatccgaatagcacatctaccaatggcagcaagtgccacgcgatgtgcaaatgccgggtcacagaacccattaccatgctaggcgcatactcggcctggggcgcgggctcgttcgtggccacgctgatagtcctgctggtggtcttcttcgtaatttacgcgcgcgaggaggagaaaaacaacacgggcaccgaggtagatcaatgtctggcctatcggagcctgacacgcaaaaagctggaacaacacgcggctaaaaagcagaacatctacgaacggattccataccggccctccagacagaacgacaactccccgttgatcgaaccgacgggcacagacgacgaagaggacgaggacgacgacgtctgataaggaaggcgagaacgtgttttgcaccatgcagacctacagcacccccctcacgcttgtcatagtcacgtcgctgtttttgttcacaactcagggaaatttatcgaacgccgtcgaaccaaccaaaaaacccctaaagctcgccaactaccgtgccacctgcgaggaccgtacacgcacgctggttaccaggcttaacactagtcatcacagcgtagtctggcaacgttatgatatctacagcagatacatgcgtcgtatgccgccactttgcatcattacagacgcctataaagaaaccacgcatcagggtggcgcaactttcacgtgcacgcgccaaaatctcacgctgtacaatcttacgattaaagatacgggagtctatcttctacaggatcagtgtaccggcgatgtcgaggctttctacctcatcatccacccacgtagcttctgccgagctttggaaacgcgtcgatgcttttatccgggaccagggagagttgtggttacggattcccaagaggcagaccgagcaattatctcggatttaaaacgccagtggtccggcctctcactccattgcgcctgggtttcgggactgatgatctttgttggcgcactggtcatctgcttcctgcgatcacaacgaatcggggaacaggacgctgaacagctgcggacggacctggatacggaacctttgttgttgacggtggacggagatttggaataaaagatgcgcgtcaaccgtcaaagacgcaacaaccttacgtaccgacaaacggtatatgtaattctgaccttctacattgtacataggggcatatgtaacagcaccgataccaacaattctacatctacttcaaatagcaccgtctctgatactaatgtatattctactccaaatcctcccagtgtatcttctacaactcttgatacatctaccgactcacagatatcaatcgcctcaaacaccatatccagcactacaaatacattgaccgcatattctataactacgttaaacacctcgacttcatcttcaactcttactgctgtctctagtacccatacaagatcctcaatactctccaacaacgcatcatacaccacatcattggataatacaactacagacataacgtccagcgaaagttcaatcaacgtgtcgacagtttacaacaccacgtacattcctgtaacatcgcttgctattaattgtactgctacaattaacggaacaaataattctagttcaaaaacttgccaacaagacattgaaacaatacctgtgaaatcaactccactaacggcagaagaaggaacaaatattacaatacatggcaatgacacgtgggattgtcccgacgtggtttggtatcgacattataattggtctacacatggacaccacatttatcccaatacacattacaaaactttgatacatcgacgcaagatcctaacgtcacatcctatatgttattctgatcgctcatcacctaccgcgtatcatgatctatgccgttcatgtaataaaacagaactacgcctttacgatttaaacaccaccaattctggtagatatagccgacggtgttacaaacagtaccatcaccagggaccacacgaggatgaaaatttcggactaactgtaaatcccaggaacaacactgacaattataccatcccagtatgtcccagatacgtagaaacacaatcacaggaagatgaacaagacgacgattatacactaagcactaccataaataataatcttatgcgcaaaacaggtcactatgacatctcacatggcacgcacactacatgggctcttatactaatttgcatagcctgcatgcttcttttttttgttcgacgagccctcaataaaaaatatcgtccactacgagatgatattagtgaatctagccttgttgtgcaatatcatcctgaacatgaagactaacgtttccggacatgcaacacataaaattaagtaacatatctaccatgaagtacagcaaatatctactaatgtctatccatccaacagtgataccatgcactggcatcttgcgattacatggacggtaatcatatccacgttttcggaatgttgtaaccaaacttgtccgtgttcctgcgtttgtgtcaattctacaacagtcaacatatccacaaatgaaacaacgtctaaagccatcactccaactgctacgacaaataccgcaaaaacaacgtcaagccttgttattactacaccgtcatcagtaacgattagcaaagccgtgtctactgcagcttcatcaaccatactatctcaaaccaatcgcagtcatacaagtaatgtcatcacaaccccaaaaacgcggtttgaatataatatcacgggatatgttggccaagaagtgactttcaacttcagtggatcattttggagctacattgaatggttccggtacagttctccaggctggctttattcctcggaaccaatatgcaccgttaccaacagttatcatcatactttccctcgtggtaccttatgtttcgattgtaacatgacaaaatttgttatttacgatctaacgttaaacgattctggaaagtacgttgtcaagagaacacgtcatgacaatcaatacgaagaagcatgctacaatctcacggtaatttatgccaacacgacagccatagttaccaacaggacgtgtgatagaagacaaacaaaaaatacagacactactaaccatggaatcgggaaacatattattgaaactattaaaaaagccaacattcccctggggattcatgctgtgtgggcgggcatagtggtatcagtggcactcatagcactatatatgggtaaccgtcgtaggcccaggaaaccgcgttataccagacttcctaaatacgacccggatgagttttggactaaaacctgatatgcacatcaataaacttttttgtatctttagttattaatgtctgtgtgtttattcagaataactcatttataatataagacggaatattcatatacattaaaaacatgggtgtacaatataacactaaactgttattagccgtattagcaattatcccagctggcattctagtacaggcaatttcacatgagcaaaaaacatcctaccggcaacttttgctgcaaagtgaacgtgtgcaaatacccatcacaacagtcgagggagatacaatttgctttaacgttagtaacaacccctgcaacttttccagttattggaatcacaataattgtgaactttgcggttggacaccgtttttctttgaatatgctggatatactgaaaacacgtcgtgtcacccacgatttacctgtattcatgatactaaaggtctaaaactatacaatgtaaccatgaatgactcgggaatttatacacaacacgtttatcactgtgatattccatgtaacatcagcgatgatcgtaaatataacgtagatgacattgataactgcaacgctactataaatgtaaccgactatattattaccgtgttgtcttcacgttattctaaacgcaccgattaccacgtagatacttacattggttatgcaaccactgtggtgacaatagtatttatctgtgttttaacttgcattaacgtctcagcaactctaaggcacagactacgaactagaaacaacgttaacagcataacgtgattacaaagtatcaacgctagtttatccaagagaaactttcatgaaggatcgcaataaagcattgctatgtatcatctttattttaatcatgtacctcatttatatttattttaaacgtcgttgtattcctactccgtccccagacaaagcggatctgcgagtggaatttccctcattacccccgtgtgtcggcatacagtgcgctgcataagaacacgcatgacacatagcgtacctctggacggtacagtatatgataacatgattcaaggaaagtatggattcctaccgacatgttatgacagaacacacaggttttctgcgtgttttataaaagagcgtctcgaagcagcttgagccacactacggtccagataacgagcgttgcaaaaaatatgccgcgcagtagtcgaaagccgtactgagcgtgcgaagcgggtagggtgccgaacgacggatatgcgccgttgtcatcttcgactataaggatcgcgaccgagtcttcgggcatggtaaaagccacacggtgtggttgatatgtagcgtatccggtttggaatcgttcggctccggctcaggggatagtgaggaattctcaggggacgatatgggacccaatgactggataaaagaagggtttttcccagtaagatgatccccgtatcacatgaggtctggatatatataaatgaggagtgaaataggcaaagggtatcagacaccagcctcgtcatgcagccgttggttctctcagcggaggaactatcgtctctgctaatttgcaaatacatcccaccttaagcgacgagtccataaagcaccgttgtccgggtacggtgaaagtgacccggattgtagcacgtcccttttttgtttttgcatcgtttatcgtcaccactagtgcaatattttgatcgtaaggctgaaagagtattgttatgatgcttagaacgtggagattattacagatggtactgcttgccacgtactgttattatgtttttgcgaattgttcaatcagcacgacgactgctcctgtggaatggaagtctcccaaccgtcagattcccaagaatattacctgcgctaattactcagggaccgtcggcggtaacgttacttttcagggtctcaagaataaaacggaagactttttatcttggctactcgggtctggctataagtccatttgctcgttcttcccacaactccctggtgattctaatgagcagcattacagatatgaagtaaccaacctcacgtacaattgtacctatgaccgcctaacgttactgaatctgacaatggaaaacagcaggaattactatttcagaagagaagatgcgaattccaccttctactactcttgttacaatctgaccgtgtcctagagaacgcacgtgaagttccacagagccgcgtggctgtagctattgtttacgttgcttttgaaatgttaagcgtccctacggcgctaacatgtttctaggctactctgactgtgtagatcccggctttgctgtatatcgtgtatctagatcacgcttgaagctcgtgttgtcttttgtgtggttggtcggtttgcgtctccatgattgtgccacgttcgaatcctgctgttacgacatcaccgaggcggagagtaacaaggctatatcaagggacgaagcagtattcacctccagcgtgagcacccgcacaccgtccctggcgatcgcgccgcctcctgaccgatcgatgctgttatcacgggaggaagaactcgttccgtggagtcgtctcatcatcactaagcagttctacggaggcctgattttccacaccacctgggttaccggcttcgttttgctaggactcttgacgcttttcgccagcctgtttcgtgtgccgcaatccatctgtcgtttctgcatagaccgtctccgggacatcgcccgtcctttgaaataccgctatcaacgtctcgtcgccaccgtgtagctagttagccagctgtgtatagtttgttgtgttttgcttttgcgtatttgttttcagtcagagagtctgaaacggggtgggagggacttttgcgggtaatgcatgctaaaataaacgggtgggctggggtgtgcttggtaactcactgtttgaatacgcgctcacgcacatatgtagcactcaacatgttagcttttgcccgcacgccccggggcgtgccgagctgcctttttaataaagtctgggtttccagatacgcgctggttctgattttgatgatttgtgcctctgaaagctctacgagctgggccgtgacatccaatcgactgcctaactgtagcacggtaactacaacagcgggtcaagacgctgaattgcacggtccggcaccgttaagctgtaatgtgacccagtggggacgttacgagaatgaaagcacacccgtattatggtgcactttatggggatcacgcatgcgagtctcattaggacaccgtgtagcgtttggctgttcttggaaaacattttttatttataacgtttctgaaagtagcggtggcacttactatcaaaaaggttacaactgcaccgacaaacatataacactatcttgtttcaacctaacggtggttcctcgagcggttcaaagcacaaccaccgtaatgacacccacggtggttacaaactccacattcagtgtgtcacttattgcgttgagactgacgacaaattccagcgcggttggacacgctagttatcaacgacaacagcgtgttgaaaacgggacgttatccaagaacataactaacttggcattcacctatggcagctggggcgttgcgatgctgctgtttgccgccgtgatggtgctcgttgatttgggtttgcctcaatcggcttggcgacgctggcaaagccacgtggacgatgaagaacgtggtttgttaatataggaagtaaaaggcactgttttagcatgactgtttccaaaccgtaacgtggtaaataaatcatggcttccgacgtgggttctcatcctctgacagttacacgattccgctgcagagtgcatcatgtgtacaataaactgttgattttagctttgtttgcccccgtgattctggaatccgtcatctacgtgtccgggccacagggagggaacgttaccctgatatccaacttcacttcaaacatcagcgtacggtggtttcgctgggacggcaacgatagccatctcatttgcttttacaaacgtggagagggtctttctacgccctatgtgggtttaagcttaagttgtgcggctaaccagatcaccatcttcaacctcacgttaaacgactccggtcgttacggagcagaaggttttacgagaagcggcgaaaatgaaacgtttctgtggtataatttgaccgtgaaacccaaacctttggaaactactccagctagtaacgtaacaaccatcgtcacgacgacatcgacggtgaccggcgcgaaaagtaacgttacggggaacgccagtttagcaccacaactacgtgccgtcgctggattctccaatcagacgcctttggaaaacaacacgcacatggccttggtaggtgttgtcgtgtttctagccctaatagttgtttgtattatggggtggtggaagttgttgtgtagtaaaccagagttatagtaatgtgttttttatcagggagaaggttttgtaccaacaatgactacatcggggctatctgtgtcggaaaattatgacggaaattatggactcacggaaaccgccaatacaacgcgtacaaatagcagtgactgggtaacgttaggaaccagtacgccactgttgggaagcacggagactgcgatcaatttcggcaacgcaactacggttattccacaacctgtggaacacccggctggagaagtacagtaccaaagaacgacaacgcattactcttggatgctgattatcgttatcattttcatcatttttattatcatctgtctacgagcacctcgaaaagtttatgatcgttggaaagacagcagagagtacggacaagtgtttatgacggatacagaactgtaatatactatgatgtctaagaagtgtttgcggttatttccatggatgacaattttgttttgcataccaaaagcacaacattggaactatatgacaataccatgcgttcttaaaattggacgcggcggtcaaaatatgagtttgcctccccttaacaattcattgtacggaaacgatatttttcaatggtatacagacagaccgacagtcaccaacacgttatgtctttatcaaaacaatgagtactacacacaatccaatgaagatatttcaaacatcaaatggcaatgtacaaaaaaccatacgttaattcttattaacctaaccgcaacatatagtaggaactattactttcaatctcttaaaactcttgggcaaggaataccgagaccgagcagcttatgttataatgttagtgtacaccttacccaccaaacacattgtcatacaaccacattatccctgtatccacctacacctgtacacaattcattaacaatatcaccgtcattagcttcaaccaactttacacatgttgcggtccatcatgccgcaggtaacgttgaagcacaacacaacactgccactccacatacaacgtggatcatacccctggttatcattataacaatcatcattttaatttgtttcaaatttccccagaaagcttggaataaattcacacaataccgatacaacagtatgctcgccgccgcttaaagaatcaccgtcgaggaaactaaaagctatgtacgtttatttttcagctcactgtttgaataccgtaaacataatgacgtacatatacgtggttatacaacaggtgtttgtgttatgcggcgactgattaaccatatcgtgaaccatgatcttttccgatggtctgtcgtgaccgcaatgatattttacaagtattccgaaacctgtatggaggtcactgtcagagtaggtgatccagttaccctcggtagtggacatggttatcatccaggacaaaaagtacactggtataaccagtcatgcgtcggcatcagcaacggcgaaaatacgcatcctatctgcacctacgaccctcctaaacctggtagacaaaagacaatgaaaaccactccgttgccatcaccactgttgtatgaatgtcacaattccacattaagcattcttcatgtaaacgtctcagatcccagaaactattgcaggcgaaaatgtccaccaaagggtaactgtgagtttcccacatgttttacattatcgctgatttctagaacgacaaccaccagaagacccggacaaaaaactacgctgtcgcgattaaaaactacgccaaataaacatacgcagcacaaaagatccacgcgaagaacgtcacttaaagattacaatgtaacgggtctgccgaaaggctttgcggactcgtttaccggtaacgtagaggcacatagagccaaagatgccgcacacagcgcatggattctcattgtcatcatcattatcatagtcgtcattctgtttttcttcaagattcctcaaagactccgagagaaatgggacaccaagggatacctttacaaagggaccgatggcctgcccactacggactaattatcgtgagcggacggatatgtccggtttcaaactcactgtttgaatatagggacagtccctacggaacctgagaacatgtggaaatcacctgtggtagaatgctgctcaggtacattacctttcatcgcgaaaaggtactttacctaacggctgcatgcatctttggtgtctacatcagcctccatgatgcctgcataccggtggttggcaagataggtaccaacgttacgttgaacgcggtagatgttctttcccctcgcgatcaagttcgttggtcatacggtccaggcgggcaaggctacatgttatgcattttcactggcacatcaacaacaacgtttaacagcacgcgctttaatttttcatgtctgagtaattacagcctcctcctcattaacgttaccgcgcagtatagtactacctatcgtactatgacatcgctagacgattggcgtcaccaaaaacataaccatggttttcgatggactttagacacatgttacaatctgacagtgaacgaaaacggtacattccccactaccaccaccaaaaagcccactacgactacgagaacgacaactaccacaacacaaaaaacaaccaccacgagaacaaccaccaccgccaagaagacgacgataagcactacccatcataaacactccagtcccaaaaaatccaccacccctaacagtcacgtagaacatcacgttggttttgaagccacagcagcggaaacaccgttacaaccaagcccacagcaccaacacgtggctacacacgccctctgggttttagcggtcgtaatcgttattatcatcattatcattttctactttcgaataccgcaaaagctgtggctgctctggcagcatgacaagcacggcatcgtgctcatcccccaaaccgatctgtgagcaagtcgcgtaggaaacgattgcatgaaatcactgtgaaacgccaactccgtgccaactggcacggcggacaggcctttgacgtatttgaagccaggcgcgctcttgataccgaaaggatccgagggggctttccaaagccgacgtccctgattcccttcataaagctgttgaccggccctagaaagaccaagagcatgctgtgggcccactgcggtcgcttcttgcgttatcatctgctcccgctgctgctgtgtagactgccattcttactcctttttcagcggccgcagtgggcccacggcttggacattgtcgaggaggacgagtggctacgggagatacaaggagcgacgtaccagctgtccatagtgcgccaagctatgcagcacgccggattccaagtcagagcggcgtcggtcatgacacggcgaaacgccgttgacctggaccgaccgccgctttggtcgggatcgctcccgcatttgcccgtctacgatgtgcgttccccgcggccgttgagaccgccgtcatcacagcatcacgccgtatcacccgaactgccgtcgcgaaacgggatacgttggcagtaccaagagttgcagtatatggtggaagaacaacggcggcgaaatcagtcgcgtaatgcgattccgagaccctcgttcccccccccggatccaccatcgcagccggcagaggatgcacgagacgcggacgcagaacgtgccgaatcaccacatagtgcagaaagcaccgtcaggcacgacgcgagtgagaacgcagtgcggcaacggcgcgaaagacggcgctataacgctctgacggtccgcagccgggactcgctgctcctgacgcgaatacgcttctccaaccaacggtgtttcggacgcgggcgtttgagacatcccgcgggaagtggtcccaacaccggcggaccgcgacccggcggtgcgggactccgtcaactacgccaacaactgacggtccgctggcagctgttccgcctacggtgccacggttggacacagcaagtctctagccagatcagaacccgctgggaggaaagcaacgtcgtgagccagacggccacgcgagtacgtacgtggtttgtggaaagaaccacgttttggcgtcgcacgtggattccgggacagaacccggcggccgaagcgcaagaactggccgtcataccgctggcacccacggtgctccagcagaacgaggaaccacgtcaacagcttacgggagaggagacaagaaattcaacgcacactcaacgtgaagaagtggaggacgtttcgagagaggacgcgagagaagggaatgatgggagccgagcaagtggaaacgacgagagaaggaataatgcgggaagatatgatgatcacgaggttcaagagccgcaggtcacttatccagcgggacaaggagaactgaacaggaggtcacaggaggagaacgaggaaggtggaccgtgtgaatcgccgccaatgacgacaaatacgctgaccgtggcctgtccgccccgcgaacccccgcatcgtgccctgtttcgtctatgcttaggactgtgggtctcgagctacctggttcgacggcccatgacgatttagaatacaccgagccattcctttatttcccccccccccatccccggtcgcttatgcgtgtcaaacactaccaataaagataatctgccaatagcaccttatatataatatgtggtcgcgtgtggtctttttaaggagccctgaaacacagacaggtatgggcggtggccggctgccgccgctgtggctgccgctactgatcgcctggagcgagtggggcaactgctgcctcgatgcgcctccggtggtgcgttcgccctgtctgcagccggtgcgcgaccgcaaccgcgagcggaacccgggctcaccgcagttgctgccttacggcgaccgtctggaggtggcctgcatcttccccgcgcacgactggccagaggtctctatccgagtccacctctgctactggcccgagatcgtgcgttcgctggtggtggacgcacgcagcggtcaggtgttacacaacgacgccagctgttacatcgccggcgggcgctggcgttttgaggacggcggcgcggcgcagcggctgagcctctcgtttcgtctcatcaccgagaccgcgggcacctacacctgcgtgctgggcaacgagacccacagcctggcgaccgagaccacggcgctggtggccgacgtgcacgacctgcgccactcggaccgctcctgcgacctggctttcggatcgcgctcacagacgcggtacctgtggacgcccgatccctccaggttgcgcagtataaactgcggttgggagggtgaacggcaccgcgtagtccactacatccccggcacctcgggtctgctgccctcgtgcgaggaggacgagcgcgaactgtgcgtgcccttcatcagccatagcatcgccgacaacaactgcagccgccggcatcgagtagacggcgctaggcggcgctatcatctgcggagggattactggctgacggatccgaagatcgggctgctggccgcgggatcggtggccctgacctccctctgccacctgctgtgctactggtgttccgaatcgtaccggcgcctgaacaccgaagaggaaaacgaggcggcggaggaaactgccgcgggagaagcctctgcggtagcggcggcggccgtctctgaggaagagcagcagcgggagtaaacggggagagccatgaagcggatgattcgcagtcacggcaggaaaacggaatgtcagatgacgggcgccggcgagcgacgcggctccgccgtcggtgcgctcatctgcgacagcggtacccgacgcggcagcggcgccaacgaacgccgcgactccgacgtcggtcccatcgcccacagtagcggtaccagacgcggttcggcaaatgaaacgtccgcctgtacgcggaccgatcaccagaaggcggacattgggctgtggttcatgtttctgttttttggactgtgttcgtggttagcgatgcggtatcgcgcacaataaattttgaatccatatcaaggaacgcgtgttttgtattttattgggaatattggcggggataaaccggtttcggatgtttacccttaatcttaccggggacctcgttgtcctctcccccttcttcctcggacaccgggcttcatgctgacgtaggtaccgactggggtcaaaagcctgggtacttatggggagcgcgcacaaaggaccgtcaggcgccggcatggagcgtcgccgaggtacggtaccgctgggatgggtgttttttattctttgcttatctgcctcttccccgtgtgctgttgacctgggtagcaagtcctcaaactctacctgccgcttgaatgtgacggagttggcctcgatccgtcctggggaaacgtggacgttacacggaatgtgtatctctatctgctactacgagaatgtgaccgaggacgagatcatcggcgtggcttttacttggcagcataacgagtctgtggttgacctgtggttgtaccagaacgatacggtgatccgcaatttcagcgacatcaccaccaacatcttgcaagacggactgaaaatgcgaaccgtccctgtgactaaactgtacaccagccgcatggtcactaatcttaccgtgggccgttatgactgtttacgctgcgagaacggtacgatgaaaataatcgagcgcctccacgtccgattgggctcgctatatccgagaccgcccggatccgggctcgccaaacacccctccgtaagagccgacgaggaactgtccgcgaccttggcgagagacatcgtgttggtctcggccatcactctgttcttcttcttgctggccctacggatcccccagcgactgtgtcagcggctgcgcattcgcctgccgcatcgataccagcggttacgcaccgaggactgaacggataaccgcaaaggccacgtgcaacgttcacgctgctataagaaggccatgtcccccgtggacgggtctctttgacacgagcgcggcacgccgttgccacgagcatggatcacgcgctcctcacacacttcgtcggccggccccgtcactgtcggttggaaatgttgattctggacgaacaggtgtctaagagatcctgggacaccacggtttaccacaggcgccgcaaacatctacctcgacgtcgcgctccgtgcggcccccagaggcccgccgagattcccaaaagaagaaaaaaggcggccgtccttctgttttggcacgatttgtgctggctgtttcgacgacttttctttcctcgggaggactcggagccactgatgtcggatccggcacggtctcccgaagaggaggagtaaacaacacacggctaagaggatacatcatcaaagaagataggaggggtcaaaacgcggactgaaagtatataacgccgatcatgtccgaggaactgttaataaaacgccatgatgacaatgtggtgtctgacgttgtttgtgctgtggatgttgagagtggtgggaatgcacgtgttgcgttacgggtacacggggattttcgatgatacatcgcatatgacgttgaccgttgtggggatttttgacgggcaacacttttttacctatcacgttaattccagcgataaagcgtcaagtcgggccaacggtaccatttcttggatggctaacgtctcggcggcctaccccacctacctggacggggaaagagccaaaggtgaccttatttttaaccaaaccgagcaaaacctgttagagctggaaattgcgttgggttaccggtcacagagcgtgctgacgtggacgcacgagtgtaataccacggaaaacggtagttttgtagccggttacgagggatttgggtgggacggggaaactttaatggagctcaaggataacctgacactatggacgggccccaattacgaaattagttggttgaagcaaaacaaaacgtacatcgacggtaaaattaaaaacatcagcgagggggatactacaatacaaaggaactatctcaagggtaattgcactcaatggtccgtcatttatagcgggtttcaaacccccgtcacccacccagtggtaaagggcggtgtccgaaaccagaatgacaacagagctgaagcattctgtacatcttacgggttctttccaggggaaattaatattacttttatccattacggtaataaggcgcccgatgatagcgagcctcaatgcaatccgctacttcccaccttcgatgggactttccatcagggatgttacgtagccatcttttgcaatcaaaactacacctgccgcgttacacacggtaattggacggtggaaatccccatcagcgttacctcacctgacgacagttcctcgggggaggtccctgatcacccgacagctaacaaacgctataacaccatgaccatcagcagtgtcctcctagccctgcttttatgcgctttgctattcgcgttcctgcactactttaccaccttgaaacaatacctacgtaacctggcctttgcgtggcgctatcgcaaggtccggtcgtcatgaccagcaacgccctgtatgagctgtttcgacgtcggttaccgcgtgcccccgtcaacacggtcatgtttctcacgcgacgcactcgtgatgggttctgcggtcggttgacgtccatcgccacgaattcccactacactatgttcgtgttagatcacggatccgtgcgcatcgagcgaccgagtcagtcagaagtggattgcgccagtttaatggaaacgctgaagcggattcggttacgaaattcgtgggtagcgtcagaagacgagctagatgtgagtcgcagggacgcgtgacacgaaacgcgttcaggattaacgtaggttttcgaaataacctacgtccgtgagtgacgcggtttcgtgttgaaacccgcgcccgcttctcacggtggtttatgatgaaaccggcgttgcggatccacgcgggttcctcattcaacctgcgaaaagaggaagttgcggtaaaaccacgtcaataaagacgtcaatgacacctcaatgttgcgttggaacggtctttatatatacaaacgccgttatgatcagtgtccggcaagatgctcgggatacgggctatgctggtgatgctggattactactggatacagttgataacgaacaatggcactcgaagcaacaataccgataccatctttgtatctctccttaccgggcccaacggagttactcgcacagccatcggaggtctgtattcaaactacaccaacttaactggagcatttggcttcacttcaacaaatatgtcagcaaccaactcttccgctgaggataattggagcgtaaccaacctgacggagagttgcatcaaccgcggtgagtcctatgtgactaccatctggcttctggactgcactaaaaacgatacttattggtactatggaaatgcctacaatcatacatgtgaaggtacaatttcgggatatctcctgggcatgtgcaagctatggaaaagttgggtcaataatattacttcttataacactgtcagagtcgaatcgctgggaaatgaaacacggtgcatgctgctccctagacagtatactctcaacgccacggtggaatggtacaacaaatctgaaggtgacgtaccagaagaattcatggactatgttatcctgacccccttggctgtgcttacatgcggactgcaggaagcttatatactcgacaaaggtcgtagatacatgtatttgttttccgtgtcctgcgtgggaatcacaggtaccgtatctattatactcgtctccctatcgctgctcatcctcatctgttactatcgctgtggccggcttctgatatgcccacgcggctttgaactcttgccagaattcactgaggaagaggaggaaaaagaaaaattgttaacgcataatgacattgaagtccaagtgcctattcgcacgcggcgactactcgtcccttggatccgggagagcaaaatgtgggtactaccacccccgttgcctccacgacctccccacttaatagaattcccgccgtctcctccgtcatcgcctgggcccatgcacatggtggtctgcatgccagcatgacgaactttggactctgagccccaagcggtacgaactacatattttccataaatctacactgaacttgagcacaaagatactgacaatagactggatatacagacttttatatgatccctgtacagatgtaaataaaatgcttttatttaaaactggtcccaatgttcttcgggaatcatggggtggggacgggggacgcggtagggagcaaaaccgggtacatgggggggaacatcgtccaacagtagcaccagcggattgggtaggggttgctgcggaggtcggtcgatgacgatgtcgatctccatcggcagatccggcaacatctcttcatctccctcaccgaccagcactcggcgctgttctggatgtatatgattctggaaaagcctccgacgagctcgcggcgcgtagaaagccaagcggcgcaagggccggcgagcccgaaagtccatgcgcacagatggcatgagtccttgagtgacggtggtgagctggggaacagggctacctcccatcgcgacggtgacagtggatccatgagagaggcgccgcacgctgcatggctaaataccgtgaatcccctgacgtcgtctttcgtcccgaacgcgtcatgttgggggcgaggcgtaaaccgtcgaggttgaaaaaccgcgtatctgcgacccgtccggactacgttgttttttagaagcggccacatgacctcgagatgtcgtcacccaaggtatttaacggcacacagccagacgcgttcgtcagcagcgacgccgacaagacctcagcatggctcggaggctatggatcttgagcttactagccgtgaccttgacggtggctttggcggcaccttctcagaaatcgaagcgcaggtaaacggaatctggggaattcaacacaggtaagaaatataaaaaaataacgtgattgtgaacgcggttatcgtgtttttgcagcgtgacggtggaacaacccagtaccagcactaactccgatggtaataccacccccagcaagaacgtaactctcagtcagggggggtccaccaccgacggagatgaagattactccgggggagactatgacgttttgattacggatacagatggaggtaaccatcagcaaccacaagagaagaccgacgaacacaagggagaacacaccaaagaaaatgaaaagacccagtagcagcagcagatcccaagggttaaagaccatgttgactattttgttttttattaaaaagctgtaaggttttgctctaaaaacaccccgcctccggtcttttttcttttgtattcggcacgcgaaacacggtttcttcccatagcctgtctaactagccttcccgtgagagtttatgaacatgtatctcaccagaatgctagtttgtagaggctatgcgggatgctgcggcggcgcgaccttccctctccacccagccccgtcaaaacacacgcgactcgagcggttcgtatgaaaaataaaaaacagctttttatttacaggaacggggaaaaaaaaggcacacggtccgtgggagacgcgggttcacgcgtcgtcaaaaagttggtggtccactccgtaaggacaggtaggcttatttagcttccgcatgctcctggttccgtaataaatgccgttttcgtggcagcgtgtcatgccgcgagtcacaaactccatcaaactgtcggccacgatgcaaacgtgctgattgttggcagcaaagacgcgcatacagtcgtccacgaagaggttgatcacgtcgtaggggctcaccaaccagcctaaaggttccacgtggttactgccgaccatgaccctccagtcgttaatctcgctccagtcgtacagccgaatcgtggagacgcgaatgacgctgtaatcacccatgaccatgagtcggccgcgatacgtagcacgccactgcgcgaacgcgtggatgtgcatgcagccggccagcgctctaagcgaggcggtgtgcggcagctcctctgggacggtgatgaagttgcagcgtcgcaaaccgatgttgagaaattcagtgatgctctcggccacaaaggtcaacgagtcagagtagatgtggtcggtccacaggtacatggcgcccgaggcgcccaggtacagttcagacggcacgttgtgatcgcccttgtgtttaagaaagttgtaggtgcagatgctgccgacgaaacgcagcggctcggggcagcagaggtagctggccagacgctgtgcatcccgtccttcgtcgcgcaccaagcgccagcgacgccggataacgaggcagcggtctttgggccagaccagggccacgcgttgcccgggtttccacggtcgcgacgtcttaggaggcctccagcggtcgagcagattgagaaaacagtccttgattaccgacatcgcggtcgcgcgtcggtggacaaaaagaaatcgggccgatccagaaaaaaaaaaaacgacagcgaaacaccgccgtgctcgagcgaagggtggcggagggccagaagaggcggccttgacgacgttggcagcgaaaaaattggcacgcgagtcaaacgggaagtagcgtcggtgttttatgccccaagcagcgtcgtcgtcactcgtggcgtcacagtcaacggtgctgacgtcctttggggcagtcgggcacgcgatcgtagatgccgttgtggccgctgaaacgtcggttttcaaacagcaggttaagtcccagacacatgaacgtgttcagattatctcccacccggatgtagcggtcgtcgcgcacgtcgcaggcgtagacggccccggtataggcgacgacgatggggataaggtcgacgggccagcgcaggtgaggaaagggcgcgttctcgcccttgaggctgacggttcccaggccgagaacgcgcattccgaaagcggttttgatgttgcgcagcaagtgaccgccttccacgctgttttcgaaacacctgaggttgcatagacgcagttccgttcccggcgggaacgtcaatggcatgaactgcccgtggtggcggatgatgaatcgtgccatggtatccaaaccgaggctccaggcgcgcaacagcgggcgaaagtagcgcttaaccaacgacgaggtcaggtagcgcatgcagtgcagggtttcgacggcgcgcagcccgacgcgcgcaaactccatgaggttgcgggccaggtagtagacggcggtgtcctcgcgtacatagcaaaagacatagccctcgtccgagatgaggcacacggcggtcttcttctgctgatccggcgacaacacggcctcgttcacgaagcgacccacgaaggccaggcgcgtctcgcagcacaggtagtgactccaagctttcacgtcctccggtttgaagtcctcgtccgtctcgatctcctgcagcactaggttccagcccggcggccagaccacgggcaacacctggcctgcgttgatgcgcacgtaagcttccagacagcccaggccgaactcggccgtgagcgccaggctagccagatcgctcatgtgacgcgccgagtcggtgggcgagcccgggggcccgtcgcacaccacgctccgtcttcttgtcctcaccgcggccagcgtggcgaggacactttccgcgcccgaggctgtatcttcggtttgcccgccggagccggccctcactatataacgtcccgcccgggtctcctccatgtatgcaggtaagcaactgagccgaacgcacctcagcagacgagaggatgtcgtcgcggcgtcgcagctcgtcacgtcgctctggcgaaccctcgacggtgatttatatcccctcgagcaacgaggacacgccggcggatgaggaggcggaggacagcgttttcacgagcacgcgggcgcgcagcgccacggaagatctggatcgcatggaggccggtttgtcgccctacagcgtctcctcggacgctccgtcgtccttcgagctcgtgcgcgagaccggcggcaccggcgccgccaagaaaccgagcgaaaagaaacgatcgtcgtcacgtcggcaaccgcagatcgcagcgggcgcgcctcggggctcgccggcgacacccaaggccggcaagtcgcctaaagtctcgcgaccgcctagtgtgccctcgctgcccgagaacggcgccggcggcggtggcgacgataacagcagcagcggcggtagcagcagtcgcaccaccagtaacagtagcagaagcaccagtcccgtggcgccaggtgagccgtccgctgccgagggcgatgagttttccttctgtgacagcgacatcgaagactttgagcgcgaatgttaccgggtcagcgtggccgacaatctgggcttcgagcccagcgtggtcgcgccgcagcacgtcgagtatctcaaattcgtgctgcaagactttgacgtgcagcacctccgccgcctcaacgaatgcatacccatgccggccttcgcgctcaccagcctcgtcgaccccgtcttaaacaacgtagcgcctggcgagcgcgatctcacgcgtcggataatcacgcacgcggtgatcatcaactattactacgtggcgcaaaagaaagcgcgccacatggtggaggccatacggaccaccgtgcgggacgacacggtacgccgggtagccgcgcaggtcaacaaccagagccgttcggggcgtgcggccgcgctagcgcttcactttctcacgtcacgaaaaggagtgacggacggtcagtacgccacgtctctgcggcggctggacgaagagctgcggcatcgcggcacgcccgaatcgccgcggctcaccgaggtctaccagacgctacgcgattacaacgtgctcttctataccgcccactacacctcgcgcggcgcgctctacctctatcggcaaaacctgcagcggctcaacgaaaaccaccggggcatgctccggctgctttcggtcgaagagatatgcgaagagcacacgctcaacgatctggcgttcctagtaggcgtcgagcttatgatcacgcactttcaacgcaccattcgcgtgctgcgctgctatctccagcaccagctgcagagcatctcggagctgtgttacctcatctatgtacaactgccgtcgctgcgcgaagactacgcgcagcttagtgacgtgctctactgggccgtcagtcaaaactacgactacgcgctctacgcgagcacgccggcgttgtttgactttttacgcgtcgtgcgtcagcaggacgccttcatttgcaccgactacgtgtactgcgccctgcgtctgctggcctgtcccgacagacctattatcggtgacaccggcggcagcagtagctcccaacgcctcgtaggcgagtttatggtgcgcgatccgctgttgcgcgacccgcgcgccacccacctgcgccagaaactcatcacccgcgacatatgcgtggcgcggttgcaagcgcagccctcgagtcgacacattccggtcgaacacacgggtgtctcctccgtcaccctgctcaagatctttagccaggtcccccccgacgaacgcgaagaagacacgttacgcgagatggctcttaaagcgtttatggaagcgaacggtaatcaccccgaacaaatctgccgatccccaccacccccgctgccaccgcgcgactatcctcaacgcgacgagcgggaccgtcaccgtcgcgaccgccgcgacagcggggaatactgttgctgatggtgggacgaaacagcagggcggaacagtttatgatagaaagtcacaggaaagtatgtgttgttttttttttaatgtaccaagaataaaaagtgcgtctacgaccaaagcggtgtgtggacgctcgtcctctgtcttctccggtttttttttatgtgtgtgtttttcttttccttcctattttgttacggcaacagcgctgatggcacgttgccggcttcgaacatcgcgtcggtgatttcttgcttgcccggcgtcacacggtgacgcagcagcgcgcggctcacgtagcaggccgactcgcggatgacctggccgtcggcgtcgcgtcgcaggcccgagcggttgccgtgacgcagtcggccctgcgcggcgcgctccacgtcttcaaagtagctgtgtagcaggccgcgctccagcagctgcggcagcgagtcggcggcgcgcaccacaaagttctcacggctgatctcgtagcacagcacgctgccgtcggctgccacgccggccacgctgcggtcccaactgaagaggttggcgagtccgatggtgccgatgacgcgcaactgaccctgggtcaccaccagcagcttccagtattctacgtcgcgcggggtgaggatggtctcctccacgtcgcagacaaacaacgtgtagccgcgcggatagggcagatccaggtggcgaccgcgctggcggcgcataaaatcgtctaaattcaaaccgccgtcgggtgcgcgcctgctcgtcatcgccgcgcctcgtcggtcgatgaccccacggtgcttataacgcgccgccgcggcttcatgtggcgtgacctccgacctcgtgaggccgaaaacggcgtacatgaagacgctcaaacttttgaatgtgggcccggtagcgcaccgagggccccggggcggcgacgacggcgggtccgagttccagcggggccttgcggcggcagcggttggcgtggttgctcagctcggcgtccgagagcgccgagctgaactgcggcagccgcgtgcgatcctgcggcgcgtccccgtgtcgcagcgagtgccagagcaggcgctggacgcgcgccgtctcgggcgtcggcggcgcgcgacagccccggcgcagcttgaaaacgtgcaggcacagcagctcgcgcttgatgcgcagcgacacgctgcggtagtcgggaatccgctgcaccagctcgagaaagtcgcagaaggtctccacgaacgtgtcctcggtgaagcgaatgcgcttcagatcgtggacgtgtttgcgaaaccgcgacagttctcgacgttgcacggggttctgagcgagtcccttgcgcagcagcgcagcctcgcctttaaacagcctgatgagccgctgcacgtccccgctcaacatacgtatacacgccgtgtactcgtgacgtatactggcgcgcagcagccgaatgatacgcagggccagcacggcgttggaggccaggtacatggcgtagccgcgacgcgggttggcacaggcccagcccgcggggagcagaaagtagtcgtcgaccagcgtctgcgaccagtcggcgaagcccaggtcacgtgatacgctgtcctggacgcgggccacgtcgccggctgtgaggtggcggatcgccggcaggtgaaacgcgcccaggtgtcgattgcgctccagcctcagctcggcgtgctccaaacgggaatggtgggacgccaccgcggagggcgacaaagaggagtggtcgccgccgccgtagttaccgttgtgattaccgccgtcgtcgcgcccgtcgccgcactcgcaaaaggccgcgtagaggtccttcaacgccgcttcggctcgcgccataaacgtggcgtggaaaaaaacggcggcgcggtgcgtccggtacttgacgggcaacccgcggcacagggccgccggcaggcagcggccgatgagttcgcgctcctcgggctccagaaacaggcacagggtgccgtccaggcgcaggtacagctcctcggtcatcgagcatagctgccgcaagtaatgggtgcgcgtcccaaaggtcttgtaatcgagcaacgtgcacaccacgtattgccccgtggccacggccagagcgatgcgtttggcggcgcgactgatctctggcaagtactgcgcctcgtgcaccagacggcggaaagcgccggcgttgagccagcgaaaatgctgcggatcgggcggcaagggcacgcctcgaagcgcggcccagacagcgaggtccgactcgagcgtcagaccgcggatgtcgtacttgccgtgcgccgtagcgcaggctgaatggaccagacagctgcggcgaatgtacaccatggcgtgcttgggatgtttgggcgccggcgttttctttttctgaccgccggcggccgccagatcctcgggcgtgcgacacaacaggccggcgcgcacagcctcctgtcgattacgaatcggcgtcaggtaggcgcgcaggaactggtgacaaaactcctcatcatcacgacagtcgtcgagatactcgtacgtggtgagcggatcgcgaaataggcgctcgtcaccgtcgtcatggtcttctttagcctgctcctccggctgctgggttggcggtggaggcggcggctgatccacggggttcatgactgagaggaagaagaaggtggcggcgaagcgacgcggagcgacggcggtaaagccagacaccggctatatagctagtcatcacagtctcctccttcacgacgcccccgtgccgctcacgctatccagcacgctacggcccgaaaacacgtactcgctgacgtcgtacgcgggcgatgtatggctgctcaccggtttcgcggcgacggttgcgctcgagtccaacggcgagaagcaaaaacgccgtgggcaacgaaaccagaaggagccctgacggataaaaccgcgcagcgtctcggccaacttaaccagcatcgtaccgtacagcagtacgtgaatgccgccgtgcgcgtccataaatacggctttgtttacgggttccatccatccgatgactacaaagtgagcctgttctagcacgccgatcacaaaattgttggcctcgtcggcctcggccacattccacgagccgaaagtgaaagtacaagcgggcgagccgcccaggcggattttgctaccggcgtggagctgacatacgcgcagcagattggcgcggtcgtgcagtatctgggagagttcgtacatgcccgcgaaggtgtgcttaaaccacgcgccctctacgatttcatccacgtaatcgcgctcaaagaagctatacacggcaaagaggccgttctcaaaaaactcgccgaacgagagccccagcacgtacaccttgtcctcgccgggcaggtacgcaaaggcgtgcccgtgcccggagacccagatctcgggcgccgtgtttgcgtccggcacgcattcgtacacactgacgaggccgataaagtacaagcggccagcctggcgcaggcacgagaagcgccggtaggtcttgtgatcgcgcaccaccccaaagtactgagtgtcgcccagcatgatgccgtgcagcggcggccagcacagcgggagccaacgacccgccgtggcgcgcacgtagcgctgcaggtgaaccccgctcgcacgctcgcgcggcttcgggcgcttgtgggtccaggcatcacgcagaccgcgccagatgctgctgaacttgggctgcccgcgcagatagagcgacgagagcgagtcaaagtagcccacgacgagcctgtcgggagacacaagagcgcgaaaatcaaacctagagcgacgacggtgaaaaaaccgattataagcgcgtgtctcaaacacgctactttcggttataaaaacaccgtcgccctatttctgggcgtgtgtacactgatgactcacctacgctttttgaacggcagtctcagctcgggattggcctcgtacagcgagctgcggtccacggggccgatgctctcgtagcgaaagtcgtcgatgagcagcgccagccccacgcgcacgaagcccctgaggtcgcgcgccagccgcaccaacttatcctgccccaccagcgccgcgtacacggtacccgtgtcgccgcagagaatccgcacgcggtgaaagaaggtcttgtcctcggcgccctcgatctcgcccagcggcatgacgggctcgcgcgtgtacaacgaacgttgaaagcggcgcagcatcgaggccgagagccccagatcgcgcgccgtgcgcagcaccagggaatgcttctcgggccagatgagggtcagctgcgcctcgcgatgcgcctctacgtaggcgcagcgagcggcggtgtcctcgcaagccagcaactcgcggaaagccagcagcgaacgtaggtagcggccgcgagcggaggcgcgcgagcggcggcacagctcggcccgatgatcgggatgcaccaagggcacgttgggttgcagacgcgcgcagatggattcgtgcaccgggtcgcagcggatcatgcccttggcaaaaaatccggccagatccgaggccaactcgtacaggcagtcctcttgcgcgtcgtaggcgaacacggcgccgtacgcgtccacgaacacctggtaccggcaggtggcgtgcgagaccgtgccaatgagatgcagagctcggaattcgccgaaaaagtcgttctggcagtgctccagatcgatctcggtcagcgagtgcggcgaatgctcgcccccgaccacgtagatgcactgcgagggccagcccagcgacacgcacgagccctcgaagcgccgcaagtaacgccgcaggccctcatagtcgcgtcgcacgcacaggtcggccaagtcgcgcgtgcaaaagacctcgggtaccaagcagcgtttgcgacgcggccgacgcgcgtgcccgggcagaggaggaaggcgcgacggcggcgacgacgaggaggaagacgccgtggccgccgagcagcccttgcgacggccggacatgccggcagtccgcgacgatccacaggagacaaaaaagcagaagcagcagtagcctcggcgacccgctccaccccgtcctccacacgctcagccgcgactgaacgccggggcgcgccgctacttgggtttttatagccatctgccccccgtctcgggcacccgggagcgatctacggagacctgacagcagttgggcaacacaagatagggaaatacaaagacacttttaataaaaaacgagactactttgtgtgtgtgctccgtaaactgtttattctccccctccgcttcgctctggatgggctccgggcccgtcaacacgcgactcgcgcggcaaaaggcacgctgttgacggcgcgagagcccgtcgtgatagtccatcatgccccggagatcgtgcacaaagcagctgtcgccgcgcagaaaccgacgcagcgtctccacgtgctgcagctgccggcgcgtatcaggagccgtcatcgctgatgtcgtcatcgccctgacaggcgcgtagatggctccgcgagatcacgcgcgttttcaaccgccgtgacacatcaggtccatcttgagctggcgccgggcctcgcgcaggtgtcgcacgcgttgtgagcgggaggcgagttcggcttcttgctcgaactcctgctgctcactgtccgagagggtgcgataaaaggcggcaaagtcctccaagtcggctacatgcgccctgggtctgacgctccaaagcgtacgcagtctgatgaagcggacccatcgagcgtcacggcacgccgtcttgaacgcggggcccgggaagaggttcttctccccggcgcgctcgggccggcgaggccgacgcggtttatatacaccgtctcggacggcgggacgccgagcccgcgccgcggccgctcatccggagacggcggaaaccgcggcgccggaggaaacggggaccggcaacgacggcggtggcggcgaccagattatgggggaaaaacccacgcttgtaaccctgttgaccgtcgccgtgtcgtcgccgccaccgtcgtcgccgctgccgcttgtcagcttcacggagctgctgttaccgccgccgtccgtcgccgccgccgcggtggcggcgacagcgacgagcgaggtgggcgagaaaaccgcggagcaagaggtagcggctgcgggtccggagaccgggaatgagagaagagaaaacagggagaacgaaggaggggagacgaggacgacagacaccaccgcggtcaaaaggtcgcacgacggtatccctcgccaactagcagagcgcctgcggctgtgccgccacatggaccccgagcaggactatcgtctgccggcgcaggacgtggtgacctcgtggatcgaagcgctacgcgacgcggaccgcgataactacggtcgctgcgtgcgccacgctaagattcaccgttcggcctcgcacctgacggcctacgaatcgtacttggtgtccatcaccgagcagtacaacacggcctcgaacgtgacggagaaagcttcgtacgtgcagggctgcatctttctctcgtttcccgtcatttacaacaacacgcagggctgcggctacaagtacgactggtccaacgtggtgacgcccaaggcggcgtacgccgagcttttctttctgctctgctccaccagcgagagctccgtggtgctgcaaccgctcatcaccaagggcgggctctgctcgtccatggcggtttacgacgaggaaaccatgcggcagtcgcaggcggtgcagatcggttttctgcacacacaactggtcatggtgcccttcgtgccgcacgcctgcccgcattacgccgtgcctttcacgacgccgggaaagccgggctgcggcggtgctccgagcggcgttgcggggttggaggaggcggcgccctttggacgggtcagcgtcacgcggcatggcgcgacgctgctgtgtcgcgtggaccatctgacctggatcagtaagcgcgtaaccacgtacggacacaaaaaaattacgcgctacctcgcgcagttccgcggcacgatggacgacgacgaggcggcgctacccggtgaggacgaagcgtggatcgcgtccaaaaacgtgcagtacgaattcatgggtctcattttcaccgtcaacgtggattcactatgcgtggacgcggaacagcgccaactgctgggcaccgtggccacctccttctgtcaccgcgtctcggacaagatcacggcgcgcaacatgccgcgcgccttttccttctacctgctgacgagcgcgcagcgcgggtacgacctgcgatttagccgcaacccgtcactcttttttagcggcgacgcgctcaactgtccgcttctcaatgagcccaacgtgttttcgctcacggtgcacgcgccttacgatatccacttcggggtgcaaccgcggcagacggtggagttggacttgcgctacgtgcagatcacagaccggtgtttcttggtggccaacttgccacacgaggacgccttttacacggggctcagcgtgtggcgcggtggcgagccgctcaaagtcacgctgtggacgcgcacgcgttccatcgtgatcccgcagggcacccccatcgccacgttgtatcaaatcaccgagggcgacggtaacgtgtactcgtacaaccaccacacggtgtttcggcagatgcacgccgccggagcaaccacgttctttctgggcgacatgcaattgcccgcggacaactttctcacgtctccccatccctgaccctccgtccgtcctcctttcccgacacgtcactatccgatggtttcattaaaaagtacgtctgcgtgtgtgtttcttaattattcctccgtgttcttaatcttctcgatcttttggaggatgttctgcacggcgtccgacggcgttttggcgccccccatgccggcagaacccggttgcggccccgtaccgctcttctggggcgacgataggtcgaaagccaccgttttcatgcccgtcgtgctcttgacgggggaacctacggcggcggttcccgtcgagcggcgtgattgcaaagccgcgctcgcccccggtttcaggatggagggagaggccacaggcggcgcattcgatacgctgcttttggccgtagacgacggtgggtaaacggtggttaccgcgggatacgtcggcgtggtcgaggcggcccggctggtgccggacaggcgacccggcgcgctaccgctcacggggaccgagggcggtcgacctaccaccgctttgccgcccaaagtaggtttcaaggaaggaacaacaccgacacggccgccccggcctttcaccggagacgggggggcactcttggccggggacggagaggctgacgaaagcatggacagcggcgatgtggcgggggacacgacatcatcctccgtgggcgacaaaacggacgccgaagctgacggctgtcgagccgaagcggaagaggttcccgcgccagaagtcacgttccttgatgacgtcgttttagacgaagccggttgaggttgcaacagcgtggcgggtaccgtcgacggcgtgcccgacacctgtttctctagccttccctgaaccggtgtcgacgtcaccgtctgcgctcgggcggacgcgtgcggcgtcgcgactcgcttgcccagcaccggtttctggctcgtggatgtcgtcgtcattggagacgataacttagctttacgtattctggacggcgtcgactgctcgggcgtctgactgggaggcgaaatgacgtcgttgtaatcggacgacggtgttgtgtgtcccaggctgacgacggagccggtgtccgaggagtcgtcgtcttcctcctcgctgtcttcgaccggtgactctgcagtttggtcccttaaagcccaaacctcatcagcggcgttctgagacgctgtttgtgtcaccgcggcgcgtggagtcgacggcctccgaggggtggtggacacggtgttttgagaagccgtggaagtcgtaggcatcctgaagggattgtgagccaggtgaggattcttgagggcccacgcgcgttcgcgcggccagttggcggggttcatatccccgggcaacggcgccgtcggagcccagggcgagttaccgttgaccggggtttgggtacccgcgaaggtaggtgtcggggccggagcgggggccgtggaaggattgacaggcgtcggcgtgaggatggcagtgccggcgccagcagggacgttaactccggcgccgaacgtcaacgtcggttgctcgaacttgtacgcggtggtgacgggcggtttggcactcgtctcggtatccgtgatgtccaccagcgtgtcggtgaaacgcggatcttgacggttggggggatagccatccgagctgtcggaatcctcgtcgcccgagaaaagatcccctctggtctccgtgagcggcctcacgtcccacgcgctgtcccgacggacccttcccgggctggccttggtcacctgcggggagacgagactgaaagccgcgtgacgctgttgttgctgcgggatgttcaagggaccgctggtcggtttctgactgcccgaggataacaggccgctgaaaacgctggaaacaccgccaccactagcgacgcccttgccgctagttcccggtttcttgatgggcgtaaagatgtttttctcgtcatcgtcatcgtcgtcgtcctcatcggcactggagccaaagagcctccgggaggcgctcggtttacgtgccgggggcggtggttgctgttgacgttgctgcaggttctgctgcctctcctcccaagccttcagctgctgtttctcacgctgcaccacctcgtcgtccacccgtttctgccgctcgcgacgcttttcctcttcgtcgtaatagccgacggccgccgaacgggcggcgtgggcgtcggcggccggtgccagagaaccatgggcctcgaagcggaacggtttgtgtcccttccagggactggcgatccagctccagccgtccagcggctgcgtggggacatgtttcttgggtaccgacgagaaggccgaaccgccgccgagcgagaggagattggcgtcatcatcaaactccaacgacggcgagcgcgcgcccaaaaaggtgtgcgccgactgcgggaagctgtccacgtagatgtcaaagtcctcgatgagcagctccagcagcgtgtcggccgagtcgccgttttccacggcgtgcttgaggatattgcgacagtagttggaatcaaaggaaaggcacatacgcagctccttgaccagcagcttgcagcgctcctgaatgcgcgccagacatttgcgctccagctcctcccaagacctacgcacgttcatgatgagacggcccgtgtacacgagcttgttgacggcgttgaccagcgccgtgttggcgtgccggtccaggttaaggtcgagcggtttcacacagaacatgttacggcgcacaccctccaggttttcttcaatgcgctgcacctccgtatccttgaggtgcacaaaggcgatgggttccgtctggccgatggctgtgaccagcgtctcgcgcaccgacatcttggccagaatgaccgcgcttacgagcgcgcgctcgacgatctcggcatcgtggcgcacgtccgtatcgaattcggtatggtctagcacagccaggtgatcgcgcgccttaccacgatcaccgaacgggtaagtgtagccgcgacgcgccacggccgcgcaacgcacctcgaactcctcgagcaccgaggagaggtcggggttgtggaaacgcagctcgcggtagtatcccaaccaaagcatgagctcgttgaacagcaccgtacgccggtgcaggcgtttttcgccacattttttcaggatcttggggtgtgcctcgagatccacgtcgggcttttgcgtgagatggcgcagaaagttgaccagggctaccacatcgcgccgctgtagaccgataaactgcaaactcatgctggcttttctccagaacccggaagcgtcgtcgccccggactgcgcccgcggtctgctattcgcccgcgatggacaccatcatccacaactcggtgagcgtcccacccaaagggagggggggtagtttaatagcggaggcggatacgcggttttcttttaagcgccgctgacttgtttcttctgttttttcgccccgtgtgctgttccgcccagacccgcaacaacactcctccgcacatcaatgacacttgcaacatgacagggccgctattcgccattcgaaccaccgaagccgtactcaacacattcatcatcttcgtgggcggtccacttaacgccatagtgttgatcacgcagctgctcacgaatcgcgtgcttggctattcgacgcccaccatttacatgaccaacctctactctactaattttctcacgcttactgtgctaccctttatcgtactcagcaaccagtggctgttgccggccggcgtggcctcgtgtaaatttctatcggtgatctactactcaagctgcacagtgggctttgccaccgtagctttgatcgccgccgatcgttatcgcgtccttcataaacgaacatacgcacgccaatcataccgttcaacctatatgattttgctattgacatggctcgctggactaattttttccgtgcccgcagctgtttacaccacggtggtgatgcatcacgatgccaacgataccaataatactaatgggcacgccacctgtgtactgtacttcgtagctgaagaagtgcacacagtgctgctttcgtggaaagtgctgctgacgctggtatggggtgccgcacccgtgataatgatgacgtggttctacgcattcttctactcaaccgtacagcgcacgtcacagaaacaaaggagtcgtaccttaacctttgttagcgtgctactcatctccttcgtggcgctacagactccctacgtctctctcatgatcttcaacagttatgccacaaccgcctggcccatgcagtgtgaacacctcacactgcgacgcaccattggcacgctggcgcgtgtggtgccccacctacactgcctcattaatcccatcctgtacgcactgctgggtcatgactttctgcagcgcatgcggcagtgtttccgcggccagttgctggaccgccgcgctttcctgagatcgcagcagaatcagcgagctacagcggagacaaatctagcggctggcaacaattcacaatcagtggctacgtcattagaccccaatagcaaaaactgcaatcagcacgccaaacgcagcgtgtcttttaactttcccagcggtacgtggaaaggcggccagaaaaccgcgtccaacgacacatccacaaaaatcccccatcgactctcacaatcgcatcataacctcagcggggtatgagctttcctgttactttattcagaaagcaccagaacccgtcgccatttcccctcatatacggtacacgtccccctgatctgtcatcacggtacacagatttcgcccgactgcggacgccgacggccaatcgcgtggcgtaggagtggcgccccggcttcattataacgccacgtcggagcccctgcgcgccacaacgccgtccggcgcaacttctgtctcggcacggtacgataaaaacgacgtcccccgtcgacgttgttttctccgagcggtgatcgttcccgtccctatcctccctccgcggcccccacggcggcggcctgctcgcacggacctatactattaccgccccaccgccgtcgtcgtcatgaacttcatcatcaccacccgagacttctccaacgacgattcagtcctgcgagccgccgagatgcgtgacaacgtggcaggctcgatttccaaagcgtacaagggcacggtacgcgccgaaggcaagaagaagctgctgctgaagcacttgcccgtgccgcccggcggctgctcgcgccgcaacagcaacctcttcgttttctgcaccgaacgcgactaccgcaagttccaccagggcatcgcacagctcaagcgcgcgccggccgaactggacccccacgagatccagcaagtcacggccagtatccgctgccgcctgcagcccagtctccgcgagccgcccacgccggccgacgagctgcagacggctgtgtcgcgcgtgtgcgcgctcttcaaccagctggttttcacggcccagctgcgccactactgcgagcaccaggacaaggtggtgagctacgcgcgcgacgagttgaccaaacgctgcggcgaaaaatcggcgctgggcgtggaggtgcatcaactggtagccttgctgccacacgagcgccaccgcgaactgtgccacgtcctcatcggcttgttgcaccagacgccgcacatgtgggcgcgctccatccgtctcatcggacacctgcgccactacctgcagaacagcttcctacacctgttgatgaactcaggtttggatatcgcgcaagtcttcgacggctgttaccacagcgaggcctaccgcatgctcttccagatcggtcatacggactcggtgtcggcggccctggaactctcacacagcgcggcggccgggctgcccgaggccgatgagaacaacgacgagggagaggaggacgacgacgagctccgtcacagcgacccggcgccgcttcacgagtccaagaagccccgcaacgcccgtcgtccccgcacacgcatgccgcctcacgagcaaaagcccgaagaaaacgaggaggaagaagaggagctgtttccctcctgcaaggcaaccgcagcattcctgcgggcagaaccctccgtctccaacgacgacggcaacggcggcgaacgctgcgacacgctagcgaccgccctgcggcattgcgccgacgaagaagacggacctctagccagccagaccgctgtgcgggtcgccgcgaccccctcaccttcagtcaccccagcccttacccccgtcacgtcccccataaccccgttgtgtatttaacgtcactggagaacaataaagcgttgatttctcaagttccgctctggttttggtttcgttttcaaagggagccccatcatggcccaaggatcgcgagccccatcgggcccgccactgcccgttctccccgtggacgactggctcaactttcgggttgacctgtttggggacgagcaccggcgcctgctgctcgaaatgttgacccagggctgctccaactttgtggggctgctcaacttcggcgtgcccagccccgtatacgcgctggaggccctggtggacttccaggtgcgcaacgcttttatgaaggtaaagcccgtggcccaggagattatccgtatctgcatcctcgctaaccactaccgcaacagccgcgacgtgttgcgggacctgcgcacgcagctcgacgtgctgtactcggagccgcttaagacgcggctgcttagagggctcatccggctctgccgcgctgcgcaaaccggcgtcaagcccgaggacatcagcgtgcacctgggcgccgacgatgtgacattcggcgtgctaaaacgagcgctggtccggctgcaccgggtacgcgacgcgctggggctgcgcgcgtctcccgaggccgaggcgcgctatccgcgcctcaccacctacaacctgctgttccacccaccgcccttcaccacggtcgaggcggtggatctgtgcgccgagaacctgtccgacgtaacacaacgtcgcaaccgaccgctgcgctgcctcacctccatcaaacgcccgggctcacgcaccctggaggacgcactaaacgatatgtatctgttgttgacgctgcgacacttgcagctgcgacacgcgctggagctacaaatgatgcaggactgggtagtggaacgctgcaaccggctttgcgacgcgctttacttttgttacacgcaagcccccgagacgcggcagactttcgtcacgctggtgcgtgggctggaacttgcgcggcaacacagcagtccggccttccagccgatgctgtacaatctgttgcagctactgacgcaactgcacgaggccaacgtgtacctctgcccgggatatttacatttcagcgcgtacaagctgctgaaaaagatccaatcggtctcggacgcccgcgagcgcggcgagttcggggacgaggacgaagagcaggagaacgacggcgagccgcgcgaggcccagctcgatctcgaagccgatcccacggcgcgcgagggcgagctttttttcttctccaagaacctgtacggcaacggtgaggttttccgcgtgccagaacagcccagccgctacctgcgccgacgtatgttcgtggaacggcccgaaaccctgcagatcttctataacttccacgaaggcaagatcaccaccgagacgtatcacctccagcgcatctatagcatgatgatcgagggcgcctctcggcagacgggcctgacacccaagcgcttcatggaactcctcgacagagcgcctctgggccaggagtcggaacccgagatcacagaacatcgcgatttatttgccgatgtttttcgccgtcctgtgaccgacgcggcttcttcgtcgtccgcgtcttcgtcgtcgtcctcagcatctccgaattctgtttcgctgccgtctgccaggtcgtcatccacacgaaccaccacgcccgcgtccacgtacacctcggccgggacttcttctaccacgggtctcttgctctcctcttcttccttgtcgggatcgcacggcattagctccgcggacctggagcagccgccccggcaacgacgccgcatggtcagcgtgaccctcttttcgccctactcggtagcctacagccaccaccgacgtcaccgaagacgacgcagcccgccacccgcaccccgagggccggcccacacacgcttccagggacccgacagcatgccgagcactagctacggcagcgacgtcgaagacccgcgggacgatctggccgaaaacctacggcatctctgaacgcggtttttcctctttttctacgtgtctgtctcaggacgagacgtcgatatcaataaaaataccgtcgacgtggtttttttaacagtgtggttttctttattgactagcggagtacacagtttacgagtaaaaaagacagggaaaggttatataaaatgctgtattatatacaaaaacatgcacataaacagacgggaccaccgtgctcgtcatcctctcctcaatcagttgttcatgtaggcgtgtggcggggtgaggggcggcatgccgttggcggcgccgggaataatgtgccgtcgaccgacgtcgcacaccttgaaacgccgtcggcgcacgcagcggtcgcaggacgggatatcccagaggaagcccatgtaggtctcggggtcctcgtcgtgaaagcggtaggagagttcaaagtggtgcaacgagcccgtccgagctcgcagcttctggcgaacaccctccacgtcatcggtgcacaacgacagtgctgggctctcacacagggcctgaagctcctgcggccacaggtgcgtggccaggggcgagtccgtcgtcaccagtttgacgcagtgcatcaggttctcggtgatggcgtcgtacaggcgactctcagcctcctcgtgcgtcatcacgtttcgaggcagcgacagctcgtcgtcgtcatcctcgtcaaacatgatcatggggtcaggggtttttttgggatgttgacaggtgggtgtcttttccagacgcacgatggcctcacgccggccgctgaaacggtggtttcggtgtcccttctttcccatgacgcaggtgaacataaccacgtcctcggccaaacggtagacggcgtccatggcggggtcgtagccgtagacgacgccgaaagtgtccaccaagacgtactggcgtacgaggaactctttgcgttctggcacctcgtggcccagcgcgcccaacaactggtggtaacaggtgatgcgcggcacggtacggatcatgagctccatggtctggatgctgccgcccgcgcggacgacgctgaaggatgtttccttgaacttcataacctctgtgttgtgggtccagaaggcgaaatgggtgtcgggacactcatcgaaagggtcgtcgatggtgtaggaagcgtagccccgcttggtcacctcggccgacaggctctccacgtcaccgcggtagagcatgacggcgttccagtaatcgtcgtactgcaccatgggccgctggtagtcgcgcatagtgtggaagtggtcgcagtgacgaaagccatgccgcagaaagtccttcatggtggatgccagctcgtagacgcagtcgcgcaggtcatcgtagcagtagatgccgccgcgctgcccgatgagcacgatgagttggtagcgcataaagcccggaccctcgacgaagccaaaggggtgcaggtattcctgacagcacacgtaagcacctggtggagaaataagaaaaatccacgcacgttgaaaacacctggaaagaacgtgcccgagcgaacgtcctctttccaggtgtcttcaacgacgtggggcttaccttgcgaacagacggtgcccatcttgcccacgaagggccccagggcgttgcgcgaacggagctggatgaagcagcgttcgggccaggccacgtgcagccgggtgccgcattcctgctccagaaagtcgttgagaccgttaaagtccccggctcgaatggcgatgcagccgtaggccatcagcgtgtcccgtaggtcgtccatgacggactcctctaccttcgctcgccgacgctgcgcttctccagccaccgctgcggtcgacagactccttcgtccgccttcggagaactacggcgcggcggcacggcctttatagacactatcagcgttgacgtcagacgatccgatgaacgtcgttttttgtgctggaacttccctcgtcccgacaaatgtagcggaaatcttcaagcaaatcgcgacgaagtccgatgaggaggatgcaaaagaggctgagcaacgcgatgctgcccgccgccacagtacatatgctcaacaacgcccagtgtcccaacgcgcgacttttggctcggagcagagccgaacggcggtttctccacatgacagataacgtggtccagtacgtccatcctttgcattccggcgtccagacgggaagcgttgtcatgttagttcccgtaaaggtcgtgttttgtcttgttttgtttctcatgagtttaacagtcctttttagaaaccgcgggcacatgtcttgtagaaagatgtaatcactctccgcgtatgtcgctagggttgacatcacagtggtagtgttttccgaagaagtgacgttgtcagtgatattgtcagtgacgttaatttcttcccagtgtacggataactcgaacggtgtcgtatgcgccaccgctctcaacacgtaactacggccggtgaggttaagtgttagttgtcccacggtcacattggtgtcatttgtaaaacacgcgatttctccgcgaacttccgtgacgttggtttcacgggtctcgttgagaacacgcagaggaaaccagccttccagatgatactggaaaccaaacgtaagcatgacgctatgccattgtctccgtggttgccgaaacgttacgttcagaggcagtttggcttcggctcctgcgcaaggcccgttatagatttgcgtgtcattgcgcgtacagtttaaccggcagttcatactcgtggtgttagaagtgatgttaacacccgtgccgtggtacgtgcatcggaccgaaacaccgtgtcccgtgctccaaaacagcgtcaacaacagccacacagacacctacgtggagacgacacgggactttttattgacggagactcacgtttctaccctcccctttcccgtaggtaaaaacccacgtttatcacacacgttgtttttacctgaaacccgcgcagcccgtggacgcgacaaaaaaccgcggcactagaaagaaaatgaaacaagtatgtttattaagcagcatgtggggctaataggggggataactgaggtatagcaactatgaaaaaatactacaaaaaaaaaagctgaacatggtcatctagcagcaaagttctccttctagaccacgaccaccatctgtaccacgtcgccctccccggtcgtgtacatcacatccttcaccacgaccggtggcaacggcggcgacgaggacaactcgctctcgacggaggccgggacgacagaggacgggggggtggtggcggcggaggacggaggggtggcggcgacagcggggtcttcttccgacacgggcgacggcaggctcggcggcgcggacagcacccgttgcgccggggcgtgagaaggctgagccccggtggcctggatgtgggccaacgaattggctcgcagcgagtcgcgatccacgaaggtcataggaattttcccttcgcggatccgccgctcagattccaggatggcgcgcacgtagctgttcaccgacttggcaaaagtgcgcggcccttccgtattcttgtcgcgacgcgcttccagcacctgcttttcgtagtccagctggtggaagaccatcaccaggtcgtccatagtgtgcgcgtgctgacggacgtgggagcgcacctccaccgggaacaaagcgttccaatactccagcacgatggcaccgtgccagaactgcgccatgctgggcgccaggaaaaacaggataccggagtcgtaggcgaacacgtcccacttgggcgtcatgaacaacaccagctgacgcgtgggccgcaccgaagcttcctcccaggcctcgatgaccccgaacatgatgagctcctggtccaacggggggcagtgtcgctccagccaactgatcttgctcaggttcatctgcagaaactcgtaagaggggtcgcagatgcacacgtagagacccgagtcgtgccgcagcctggctccgcgcttcatcagtttcctcaccgcgtagcgaagcgccaccttgcccaacgccgacgcctggatcagtccccccacgtccatctgcgtctgtcgccactcggcctcgtccagcaggctcgtgatagcggaagtgctatgcgtggtcgtagtcatcctttctatccttctctatgaatagcagcaatagcggtaaagtcccttcttatactatcccggagtctgtggttttttttgtttacccctgcttactggtgagactgctgggggccgttgtgctgcagcagctgagctcgtcgccgccgttgccacaggaaccggtgcctccgcagggcctttttgagggcctcgcaggcttctcgcgcaagtcctgagaggccctcggcgtcgatggggttcacctcgggcgtccgagcctcgttttcttcttcttcatcctccctttcctcctccgtgtcctcccgctctgtgtcctccgttacgctctcctccccggcctcggccaagagcgcagccaccaagtccacggaccgctcggtctccgagttctcaccgtcaattacgccatgttggcggcgtaaccggtgccgagaacgccgggtgagcgcacatgcttttttctttcttaaccaaggcgggagaggatcttcaaggcgttttcgctggatccagcggtagctaaagtaccaaaaggccagcaggcccacgctacctaacagattcacgtagactggagacataattaaagaaagaagtgaaacccgcgtgtgggtctcacgtcgtcttgaaacaccgtcttatatacatgaagatgccggacatgacgcgcccaagacacgtggggtttcccccttaggggacccggtttcttaagatgtttttcatcttcgcacgcgatgtactacatcaaagggtcggctgaccgaccgcattgacgcacagtttccgagtacgcgcgtctcggagcacctgacggtgagccacccagctcacgcggataggggacaacactgacgtgaggggcgattcacgtcactgacggctgacgggaataagacgggtgagggatttccacctttttcttaagtgtgactctccttacggtaaatcgcacctgtgacctcttaacccctcctccctggtacccaataacagtgaaaaacacacaccacacgtcacgacaccgatcgattttctttattcttagtgtgatgataggtaagggcactcgtgaggatgtgcagttatcattatcaagccttcttcaaggcgtagtgatgatcgttgggcagaacccccaagctcctagcgatctgggaatagaaggaggagaacgaccccagggccagaatgcccacagtgtacatggcccaggtctccagaccgaacgtggcgggtcgcagcttcagatggtaggccacccgctccgagagttgtgaatgctcgttcaggcaacaggactgcaggtgggtgagcccaaaagcgctttcgtttacgccgcgcacgtgcaccgtctgggccgggcaatcctggtgttgcgcgcgaaaatggtcctgacaggagattccgtctacgtggcggcgcgtgttgttacccacttcgatcagcaacgtgttatcggcaggatgatgcgagaacgcgacgacggtgttgctggaggtctggcggcagcagtacacgtcgagcgtcatgagggccatgtcgccttggtggtacacggcgtacgcccaaccctggaacacgagcggacatcgcggaccgtgagcggacatcgcgccggcggttgttaccgtcgtctcggcaggagaacacaataaactcctgatcctcatacacaggagtccaagcgtcagaattaaagtccgcggagccataaccgcgcaagtgaagccgatacgagtgttgctgaatttgttcattctgccgactgttgctcacgagcgttcggaggcggtgccacaggctgttggccattaaaaagtcctggcccgaatgacgacgagacagagcccgaggcgaagaaaaaggcgcccgtcatgaagacgtaggcaggggaattcccatatttttatggcttcttttaaaagtctgtatccgactccatccggcgcttttcccaaaccgtggtctcctcgtcgtccgactcggtacccaggaggtggtaagtcttttgccgcacgtagaaagctttcaacgtggagcaaaaaatgagaataaagaccccgaaaacgaaacaaaccacgccgatcatgccgatgcagacgttcatgtcgacgtagccggcggtgctgttggcggtgcggcaaaagagtgtcatgtcgtgcgtgcacaaaaaacaacacacaccacaggccaggtcgtagcgtagttattattccgtagcagcaatgatggtacagtcaagcacatgctctatttcccgttaccccgatgatgatgatgatgttgtccccgttgcagtggaattgtcccggttaatcaccacggtgaacaccacggccaagaaaatgatccctaatatagcgaccactaagagagcaaaagtccatttccagccgttgtcaaagtacgcccccgtggtgggatgcatggtggcgggcatttccatcatatccatgtcgaacgtgtgtcgcggcgacggcgaactaaccaggcagtacgggggtcgatagggcggtgggctgcagtcgggtggtggcggcggtggcgtggaaaccgtcgtcgggcacagacccatggcctgctcgtaggtggggggcgcgtcgtcgtgatcccggtcgcggagcatcggcgtgggctccatgtcggtggcagtgacggcgacggtggtaactgtggtggagacggtaccgacggcgtccgcggctcaccttcgagcaaagagccccttctttttgcgcaaacgacggcaaaacagttctctgggacagccggtggcgcggtaagcgggtgccacgctttcagggtgggtaaaacagtcgcgggcgaagcagtagttgttgcagaaccgcaaaaacccgacgcgaaagaagcccaggagtccgcgcgccagaaagtgcgcctgccgcgtctcgggatgcacgccgaagacggcgccgctctcgttcaccagtatggagatgtccaggcgctgctgcgactccaccggcacggcccgcaccacaaatacctgcagcacgttcagcgagcacgtctcttttaaccagttgccgtgggccggatcctcgtaagtctggctcccgttcaagacgaccgtcgtcagcgcctcattaccgtctcgccagctgaagatggaaccctcgcgcttcatgcacaggcgccacagggccagcaggtcgcgcgccaacatgaactcgcgacccacgtcgccgccggtctcgaagcggacatagcccagttcttcgcgcagcggcgcgtagttgcgcaggccctcctgcacgaagccgcggaaaccggaccgcgacaccaggtacagcgattccaccacgggcgagtagacgtagacgcggccgccctcgccgatgagtacgggtagcggtgggcggccgatggcttcgcaacgactcacagtgcccaccggcagcaggaacttgtcgcagcacaggaaggtcttctccaaacctttaatattgagatgtccaaagtagccgacgcgtaacaggtcgcagtaggtgaaaaaccaaccgttcggccagctgagacgcagcaccgtgccgctgacgcgacgaaccagcttctgcaggtccttgcgggcgtcggcggtgacagagcagcggaaggtctcgttgaccagctcgacagccagcgcgtcctccagcgtacgttccttcatctcgtcgttgatgctctggcggcgccgccggatttcgtcgaaacgagccgcggaggcggcgaccgacgcggaggtcgtccgaacgccctctgtgacgctgtcgtccggccagtcaagaaagctaaggctggcgctgcgccgcctaaagtgtccgatccgcgcgggacgtcgctgagggacggtggctggtctgctggggcgggtacggccgcgggtgtccgcggacacgttagttatacacggaattgagtcacgtggcacgttgccagctgaaaccgccgtcgtctccgccggcgttttctccatcacgggaccgcgccgtgcgcgcgttcccaggcacgcggcccacgctctacccgcacttttgcttcttggtgttagggacgaactcgaacgttacagaatcctcgctgtcgctctcctctttcgcgtcgttaaagtaattgccggagttgcgatccaaaccgccgcctcctcctcctccgccgccgcccgatccacctttggacgtcaggtagctggtgatcttgtgctgctcgtatttttccttggaggaaagaccgtggtcgtgatcaccgccgccgccaccgctgctcattttccgcgtaccggaaccaccgccaccaccgcggtcgtgcttcttgccgccaccgccgccacctcctcccagaccgccgagacccatgggttcgttcatgagatcgttatccagacccgggccgtcgtcgtgcagaccgccggcattggccagcgaagagaggctgccgccaccaccgccgccgccacgcgacttgccgctgttcccgacgtaatttttatcgaagggatcgccacgctggaaaggttcctcggtgagaaaattctccacggcgaacagaccgttgcgactggccacgtacaacagcgtgtcgtgctccgtaactatacgcaacgtgcacggcagtttggtgacggcgcaattgagcagcgtctggtagaagttcttcagctgcacgttgatacgcatgttttttacgccgtggaaactgacgcggttattggccgtgaattccagctcgctgccgttggtcaggatgaatttgatggccggcggaccggcgtgcaccagaatctgcacggtgcccgtagggcagggcgcttttttaacgttacgcttgacgcgggtatgcggcccgatccacttaagcaggtcggccaccacgccgaaatctagatccacgtgcacggccgaattctcgctttcgcgcacaatgtcttggccgtgcacgcaggccgagctgaactccatattgaaatcgggcgcgcacatggagatcttggccgaaaggtccgagatgtcctgcacgtagaacttggtcaggtccttgctggaagtcaggtacatgaaattaccgagcagcggcgtggaattgttaatggtcttgggctgaaacgacttgtcagtgatgtagagacatgagctgttaaaagtgatttttgacacgcagtgactgcgtaccgtttgcaagataagcgacggcgtgggcaagaaggtaaccgtggtgttctccttgagcgcacggatcacagatcgcagctgctggatagccgtcttgtacggcttcagccgcagcgccagcgtcggcggctccgagaggcgcgtcttgcgatccatcccggacagcgtgcaagtctcgactaaggagcgggcgcgagcgagcgaaagttttatagagagcacacacgacgaccgggaacgctgcgaagacgcccggcgtctaataatacagccgcgccgagccagcgggcccccgactaagaggcacagtacttatatactccgaccttaaagcgccagtggtaccacttgagcatcctggccagaagcacgtcgggcgtcatccccgagtcatagtagaaaaccagggccacgcactggtccacaaacacgctcaggttcacggccgccatttccacgtcgttttggatcgccggcgccgcctggaacagacactgcgtcgccttaccctcctcctggtgctgctccaaccacgcgtaattcaccacgggcacgcgcagcggcctccgcaccacggtggggaagtaacactcacggttgggcgggcacaatgaccacaccgtctcctcctcgaacacggtgccgcgcgaagcccacactgacggcgtcacgccccacagatgcgccacctcgtcgtcgggacccaccgccagaaactgacagttgcgcaatccgaactcgagcatgtcggcgcgcagcgcttcccagcgcgcgctggcgatagagagccgcggcaaccgatacaattcgaaaatgaatttgccctcttgatagatggtgcgttcgaaccactcgcagcgcggcaaacccgacttgcacaaatcgacgctagcgcgcaccgcggcaaagtacatgtgctcaaagatgcgctcgatcaagtcccaagaggcaaagtacgtgaaccctaaccgcatgagcgccgtgtgcaagccggccacgccgatgtgcagcggacgcagtttttccagcgcgctctctacccaccattcggacgctgacattagcgcgtccaggcgcgcgttgccccaaaccaccgcctcggtcaccaactcgcgcagcacgctcaaatcaaagtaacgtcgcgtgttccccaaaaccacgtcgggtagatgcagcttctgctcgtcgctacgcgcaaacacgcagcgagccacgttcaccgtcagccgctgcaccggcatgtcacactcgccaaagtggcacgacgccatatcgggactcaagcacggcggcaggcacacgctgtcggccataatcgagtacttgactacgtgatggacaaagaccaccgaggcacggcccttgagcgcgcacagcaacatctttttcagaaaatcgtccgtgttcacgatcaccttggggcacgattgctcgcagcgcgaatactctttctcgaaagccgactcctgacccaggtccgagagccgccgggagacaggccgcccgaacagcgagtagcgctgctcacgcgcacggtatcgcttcattaacacgctaggcacgttgaaagcgtagcaaacccccgtcaactccgacgtgctttctttgagaataaagttaatcacgcggatagcggccacgtcccacatgtccacaaacacacgtaccacgggtcgatgcacctccttctcgcgtatcaaatcgcagtatccccccaggcaacgaatcacgctgttcacatcggcgttaagtcgcgttacgttcaccgacacagaaacgccgcaactcaaggtgctcatccatttgcacatagccgcccaactggcgtcacgcgaaaaagggtcggccgagatcagaaagtcgtactgcggcacgcgatcgaaacccacggtagacatggtgaaggtggacagcgacagctgcccatcgcgacagcgcttcaacaccgattccaacacctcgccttcgaaacgcgcatccagatggaaacgatagatgcgcgagtgcctactgttctcgatagccgccgtcaacgccacggcgatgcgcaaaaacacgccgcccggactctcgtcctgtccgtgcagttggcgacacaccttatccaaacacaaaatggccgcgtacaagccccagcaaccggccaattccacaaaacgcgccgtctcctcggccagcttgggtagatcctccatgtgacgcagcacaaaacggcgcaccgactcatcgcacagctccgaagcgtaacacagtggcgtgcggctttcacgcgcccagttggctttgaaataaaagcgacccaacagcagatcgcaacgcggcgagtgacgaattagacagggaccgtggcgcatgataagctgaaacagcctgaaactgcccaaaccggcactgtgccgcgacacggtgtccatctcgcgccacagcgcgttcctgtcggacggcagctcccgtgccggctcctgtacgccgcaaaagcgaaacttgccccaatagccgtgacaatgacactttttgcccatcaacatgcgcgtagcctgtatcggcggcgatactttgcagagcgaagccccgaaatcgtcctcctcctcgacactgtccagctccatcctggtcgcgccggtcggattgaaggtgctcaaaccgctactcacgcgtccaccgcgactgggcacggcggaaccgctgtcacgcgtcaacgacagcacagacggcgtgccgtcaggagacggcgactcgggacgccaactgacgacgccgccaccactcgtaaaacccgctacacacgctacgccgctcgacatgttagtattttcagcggatgcttccttgtcacccccgggcagcggcccttcctcgagctcgctgtcatctcccccggtagtatcagcgacggcctctgccgacgattcctccgtctcggtttccgcgccgcggctcggaatcctacctggccggcaccgatgtgcgggcaccgaggacacccgctgttcctcgtccgcgtcagccggagtcataagtttacgaggaaaagaacaaagaaatcaggtagatttcaataaagtgagtctagatggcgccgataactacggtttataaagtctgtgtgcgatgtgtttatttttttcttctgtgtctcctccccgtatgctgtcagcgccgctcagacgaattctcgaaagtctcccaattcgacgctaaagttgtccaaacggacgacggacagtttgagttctttgtgtaccaggaacgaggtgtgaatgtcgtcagccaggcaccagcccagcttttgtatgaccccggtacacagagggatctggcgtgggcgcgtgatgcgacggttgacaaagctacagcgctcgcgggcgaactttccgcgtgcaacgtcgaccagggtctgccagtgtgcgatgctggaggtgagcacgtagatgccgggacgtgtttcgggcccgtcatagtcatagacgatgattaaatacacgtattgcagccgtccccgggtctcttcccacgtcagatacatgtctttcggtatcatcaacgcgaacacctccgttttgagcgtgttgtaaaggtagccgcgcatgacgcaggtgagcaacgaggtgatgcccagcgagacggtcttgacgcagcccagcgtctcgaggcggcggtgcagcagatgcgggcccagatccagccactgcagcgcggcgcgcgcggccgaggccgtgtacacgctttcgagcaggcagcgcgtgctggccgagacgttggaggcgcgaatgcctaacaggtagaggctaatgtagaggtgtcgcggcgagtcgcaacccgtctccatgcggatgagcagcgcgcccggctgcgcctcgaactctaccaggccctcgggcacgaagaaacgcgccgtgagcgcctggtgatcggcgtggtagaggtagcgcaccgatatagtatttacctcgcgtttggctttgagcgccgtcactagttcattgtcctcgtcggccgggtcgcgcggccgtttggccaccgcgcgcgcgtccatgatggcgaggcgcacggtagatttcaaaaagttgatagagcagctgcgggcacgggccacggacaaagcggaggcgttaaataccgtgagccaattggagatcggcgcggtggatgcccaggacgtgaccgcgagcgccgtgcgcgccttcgtgggtgcgttgccgagctcgggctaccactttggcttcgtgcgtcagaacgtggtcttttacctcctaagccacgccacggtacagacggcgcgcgacccgctgtacgccgccgagcagttgcacgaacagctggaccgcttcctgcgacaccagcacgacggcggcggggacgaggaccggttgccgttctaccacaacggggccacactgacggctttccagaagctgttgcagaccctgcgcgagatccagaccgtaatagccgaacagagcggcggcaccgcggcagcggcggacttgatcgccagtaacaacgcgtcgaccgagcgccgcggcaagaagggcggttcgagttccgggggccagcagccgctggtccgccgggtgatcacgcagctggaaacggctgccacggaggcgcggccctacgtcaattgtcgcgccgtggccgaactcctggacctgacctaccagcggctcatctactgggcctgcacgctcatgccctacgtgttgtttcggcgcgacaccgacaccgaactggacacggtgcttctgatgcattttttttacacacactaccgttcggttaacggcgatttggccgtggagtttcaaaactacgtcaagaacagcgtgcggcacatgagctctttcgtcagttccgatatcgacggcgaccagaagcccggtgccgaacacatgcgtgacgtcagctacaagctgttcgtgggtaatctgcaggcgcgtgacgccagcggcctcatgtttcccatcattagcacgcgcatctccaccgtgaacctttacctgtcgcccgaacgtatgtttttccacccgggtctgatctcgcgtctgttgagtgaggaagtttcgccacgcgccaacctagacgcttacgcgcgcgtgtgcgatcgcgtgctggaagaccacttgcatacgccgcgacgcgtgcagcggctactggatctgacgcagatggtaacgcgactggtggaactgggtttcaatcacgatacctgcgcggcctacgcacaaatggcgctgatccagccggccagtcagaagagctcgctctttgtcagcgagattcgcgagaaactcatacagatcatctacaatttttacacgtttttcatgtgcctctatgtgtacagccccacgttcctgttcgaccaccggcggcggttgattttggagcagcatcgatccacgttgatcggctccaaggaggaactacagcacgtctggagcaacgtgatactgaacgtcaatacgcactttgcggttcagtacacggaagaagactttgaggcacatacgaagggtgccacggaggcggagcgcgagtacctgtatcgggacctgcacagcaagtggggcgtgcacctgtttaccttgcgtccgtctcgcggcgcggccggcgcggcctcgcctttgcctccgcttgacggcgtcacacgctccgacatcttacgcgaatgcgcgctcgttaatctgaacgaaggccgcgtcaactacgcctccctgctagccttcagtcatcatcccgagttccccagcatcttcgcgcagttggtggtggtaactgaattttcggagatctttggtatcccgcagggcctgtttcaagccgtgggttcgccgcgtctttttgcgctcattcagctgtgtcgtgtattgttgcccgagcaggtgacgctgtaccagaacctggtctccatctacaacctgaccacctttgtcaagcacatcgacgccgcggtttttaagacggtacgcgattgcgtcttcgacatcgccacgaccctcgagcacctcagcggtgtacccgtcacgcccaatgtggacctgctggccgagctcatggcgcgctccgtagcgcataacctgtacaccaccgtcaacccgctgatcgaggacgtgatgcgcagcagcgccggcagtctgagaaactatctgcgacacacgcgactctgtttcggtctggcgcgtgggcgggcgcgcctctcggaggacggcgtgacggtgtacgtggaggtacagggtcagtacggactgcgcgtacctaccacgcgtttcgtagaacagttgcgcgagctggttcgccgcgatcggctgttggccgagaatctgcgcggcttgaatgagcgcctgctgagtgttcgcgtgcgcgtacgtcagatcagcagcgacacagaggaagtaagccgacacgccaagggtcaccgcacggtggcccagatgagcaaggcgctcaaaaagacggcctccaaaatcaaagtgttggaaacacgcgtgacattggcgctcgagcaggcgcaacgttccaatggcgccgtcgttaccgcggtgcaacgcgcgctagccgtctttgacgtactaagtcgcgagaacttggaacgccgcggcgcacagctctgtctgacggaagcgacgagcctactgcaccgacatcgcgcgctagcgccgatgacctggcccgcgggcacgggcgttgcggcggcggccgaagcggatcgcgccttacgcgagttcttggaggcgccctgggaatcggcgccccaaccgccgcgactccgcatgacgcccgacaccgatcacgaagaatcgacggcaggcgcgacgtccgtaccggaggtcctgggtgcgcgctacgaacccgcacacctggccgcgagcgacctattaaactggtacatcgtccccgtaagccaggcgcagcaggacatcttgtcttcgatcgacccgcccgccggctcgacatcggtgtccctgccgccggcctcgccatgaaagtcacgcaggccagctgccaccagggcgacatcgctcgctttggagcgcgagcgggcaatcaatgcgtctgcaacggcatcatgttcctacacgccttgcacctgggtggaacgagcgccgtcctgcagaccgaggcgctggacgccattatggaagagggcgcgcgtctggacgcgcggctagagcgcgagttgcaaaagaagctgcccgccggcgggcggctgccggtctaccgactgggcgacgaagtgccgcgccgcctggagtcgcggttcggccggaccgtgcacgcgctctcgcggcccttcaacggcaccaccgagacgtgcgacctggacggctacatgtgtccgggcatcttcgactttctgcggtacgcgcacgccaaaccgcggcccacctacgtactcgtcaccgtcaactcgttggcgcgcgccgtggtcttcaccgaggaccacatgttggtctttgatccgcacagctccgcggaatgtcacaacgccgccgtgtatcactgcgagggtctccatcaggtgctgatggtgctcacgggcttcggcgtgcagctgtcgcccgctttctactatgaggccctttttctctacatgctggatgtggcgaccgtgccagaggctgagatcgccgcgcgtttggtctccacctatcgcgaccgcgatatcgacctcaccggcgtcgtccgggaaagcgcggacacggcggcgacaacgaccaccgccgcaccttccttacctccgctgcccgaccccatcgtcgacccgggctgccctcctggcgtggcgcccagcattcccgtctacgatccctcgtcctcacccaaaaaaacacccgagaaacgccgcaaggacctcagcggtagcaaacacggaggcaaaaagaaacccccgtccacgacgtccaaaacactggccaccgcctcctcctcctcctcagcgatagcggcggcctcttcttcgtccgcggtaccaccgtcctacagctgcggcgaaggggccctgccggccctgggccgctaccaacagctggtcgacgaggtagagcaggagttgaaggctctgacgctgccgccgttgcctgccaacaccagcgcctggacgttgcacgcggcgggtaccgaaagcggcgctaacgcggcaacggccacggcgccgtccttcgacgaagctttcctcaccgatcgtctccagcagctcatcatccatgccgtcaatcaacgctcgtgtctgcgtcgcccctgcggcccgcaatcggcggcgcagcaggcggtacgcgcctatctgggcctatccaagaaactggatgcctttctgctcaattggctgcaccacggcctggatctgcggcgcatgcacgactacctgagccacaagaccaccaaaggcacgtactcgacgctggatcgcgcactgctggagaagatgcaagtcgtcttcgatccctacggacgtcagcacggcccggcgctcatcgcctgggtggaggagatgctgcgctacgtggaaagcaagcccactaacgaactgtctcaacgactgcaacgtttcgtaaccaagcgaccgatgcccgttagcgacagcttcgtctgcctgcgacccgtagactttcagcgtctgacgcagatcatcgaacagcgacgtcgggtgttgcaacgtcaacgcgaggaataccacggcgtttacgagcacttggccggcctcatcaccagcatcgacattcacgacctagacgccagcgatctgaaccgacgcgaaattctgaaagcgctgcagccgttggacgacaacgccaagcaggaactctttcgcctgggcaacgccaaaatgctagagttgcagatggacctggaccgtctgagcacgcagctgctgacgcgcgtgcacaatcacatcctcaacggctttttgccggtagaggacctgaagcagatggaacgcgtcgtcgagcaggtactgagactcttttacgacctgcgcgacctgaaactgtgtgacggcagctacgaagagggattcgtcgtcatacgagaacaactgagctacctcatgacgggcactgtgcgcgacaacgtaccgctactgcaagagatcctgcagctgcgacacgcgtaccagcaagccacgcagcaaaacgagggtcgcctcacgcagatccacgacctgcttcatgtcatcgagacgctggtgcgcgacccgggcagccgcggctcggcgctgacactggccttggtacaggagcagctagctcaactggaagcgctaggcggcctgcagctacccgaagtgcagcagcgcctacagaacgcgcaactcgcgctaagccgcctctacgaagaggaagaggaaacgcagcgtttcctcgacggactctcgtacgacgatccgcccaccgaacagaccatcaagcgacacccacaattacgcgagatgttacgtcgcgacgaacagacgcgtctgcgactcatcaacgccgtactgagcatgttccacacattagtgatgcgactggcgcgcgacgagtcgccgcgaccgacgttttttgacgccgtcagtctgttgttgcagcaactgccacccgactcgcatgaacgtgaggatctgcgtgccgccaacgccacgtacgcgcagatggtcaagaaactggagcagatcgagaaagccggtaccggcgcatccgaaaaacgcttccaagcgttacgggaattggtttactttttccgtaaccatgaatatttctttcaacatatggtcggacgactgggcgtcggacctcaggtaacggaactctacgagcgatatcaacacgagatggaagaacagcacctggaacggctagaacgtgaatggcaagaagaggccggcaagctcacggtaacttctgtggaggacgtgcagcgtgtcttggcccgggcaccgagccatcgtgtcatgcatcaaatgcaacaaacgttaaccaccaagatgcaagactttttagacaaggagaaacgtaaacaggaagaacagcaacggcagctactggacggctaccaaaaaaaggtgcagcaggatttgcaacgcgtggtggacgccattaagggcgagatgctctccaccatcccgcaccaaccactggaggccacactcgagctgctcttgggcctagatcaacgcgcccaaccgttactggacaagttcaaccaggacttgctgtcggcgctacagcagctgagcaaaaaactagacgggcgaatcaacgagtgtctgcacggcgtgctgacgggtgatgtagagcgacgctgtcacccgcaccgagaagcggctatgcaaacccaagcctcgctaaaccacttggaccaaattttgggtccacaactcctgatccatgagacgcagcaggccctgcaacacgccgtccatcaagcgcagttcatcgagaagtgtcaacagggcgatccaactacagccatcacgggcagcgagttcgagggcgactttgcacgctaccgcagcagtcaacagaagatggagggacaattacaagagactagacaacagatgaccgaaactagcgagcggctggatcgctcgctgcgccaggatcccgggaacagctccgtcacgcgtgtacccgaaaaacccttcaagggtcaggagctggcgggtcgaatcacgccgccacccgccgacttccagcggcccgtcttcaaaacgctgctagatcagcaggccgacgcggcccggaaagcgctcagcgacgaggccgatctgctgaatcagaaagtacagacgcagttgcgacaacgcgacgagcagctgagcacggcgcagaacctgtggactgatctggtcacgcgccacaaaatgagcggcggactggacgtgaccacccccgacgccaaggcgctgatggaaaagccgctggagacacttcgcgagctgttgggcaaagccacgcaacaactgccgtacctgtcggcggagcgcacggtgcgctggatgctggcctttctggaggaagcccttgcgcaaatcaccgcggaccctacgcacccgcatcacggaagcaggacccactaccggaacctacaacagcaagccgtcgagagcgccgtgacgctagcgcatcaaatcgaacaaaacgcggcctgtgaaaattttattgcacagcatcaagagacgactgccaacggcgcgtccacgccgcgggtcgacatggtccaggcggtggaagcggtctggcagcgactggaacccggacgcgtagccggcggcgccgcgcgtcatcaaaaagtgcaggaactgttgcagcgcttgggtcagacgctaggcgacctagaactgcaggaaacgttggcgacggaatactttgcgctgttacacggaatccagaccttcagctacgggctggactttcggtcgcagttggaaaagatccgcgatctgcggacccgttttgcggaactggccaagcgacgcggtacgcgtctctccaacgagggagccctgcccaacccccggaaaccgcaggcgacgacttcgctgggcgcctttacacgcgggttgaacgcgctggaacgacacgtccagctgggtcaccagtatctgctcaacaagctcaacggctcatcgctagtctataggctggaagacattcctagcgtgcttccgccaacgcacgagaccgatcccgcgctgataatgcgcgaccgcctgcgtcgcctatgcttcgcgcgtcaccacgacaccttccttgaagtggtagacgtcttcggcatgcgacaaatcgtcacgcaagccggcgaacccattcacctggtcaccgattacggcaacgtagcctttaagtacttggcgctgcgagacgatggccggcccctggcatggcggcgccgctgtagcggcggaggactcaagaacgtcgtcaccacacgttataaagccatcacggtagccgtggccgtctgtcagacattgcgcactttctggccgcagatctcgcagtacgacctacgaccctacctcacgcagcatcagagccacacgcaccccacggagactcacacgttacataaccttaagctcttttgttatctggtgagcaccgcctggcaccagcgcatcgacacgcagcaggagctgacggccgccgatcgcgtaggaagcggcgagggtggtgacgtaggggaacaaagaccgggccgcggtaccgtgctgcgcctgagtctgcaagagttttgtgtactcatagcagctctgtaccccgagtacatctacaccgtcctcaagtacccggtgcaaatgtcactaccctccctcacagctcacctacatcaggatgtgatacacgcggtagtcaataacacacacaaaatgccccccgaccacctccccgaacaggtcaaggccttctgtatcacccccacccaatggcccgccatgcagctcaataaactgttttgggaaaataaactggtacagcaactgtgccaggtaggcccgcaaaaaagcacaccacccctaggcaagctatggctctacgccatggccacgctggtctttccacaagacatgctgcaatgtctgtggctagaactgaaaccccagtacgccgagacctacgcctcggtgtccgaattggtacagacgttgtttcagattttcacgcaacaatgcgagatggtgaccgaggggtacacgcaaccgcagctccccaccggagagccggtgcttcagatgatccgcgtgcgacaccaggacacaaccaccacagacacaaacacgaccacagagccaggacttttagatgtttttattcaaacagaaaccgccctagactacgcgttgggctcctggcttttcggcatacccgtgtgtctcggcgtgcacgtagccgacctgctgaaaggccaacgtgtactagtagcgcgccacctcgaatacacgtcgcgagaccgcgacttcctccgcatccaacgctcccgggacctcaatctcagtcaactgctccaggacacgtggaccgaaacgccgctggagcactgctggctacaagcccaaatcagacggctacgcgattacctgcgtttccccacccgcttagagtttattcccctagtcatttacaacgcacaggaccacaccgtcgtacgcgtgctgcgaccgccctccacgttcgaacaggaccacagtcggctggtgttggacgaggccttccccaccttcccgctgtatgaccaagatgataactcatccgcggacaacgtcgctgcgtctggcgccgctccaacaccgccggtacctttcaaccgcgtgccagtcaatattcagtttctgcgtgaaaacccgccacccatcgcgcgagttcagcagccgccgcgccgacatcgtcatcgagcggccgcggccgcagacgacgacggacagatagatcacgtacaagacgatacatcaaggacagccgactctgcattagtctctaccgcctttggcgggtccgtctttcaagaaaaccgactgggagaaacaccactatgccgagatgaacttgtggccgtggcgcccggcgccgccagcaccagtttcgcctcgccgcctatcacggtgctcacgcagaacgtcctcagtgctctagaaatactgcggctagtgcgattggacctgcgacaactggcgcaatctgtgcaggacactattcaacacatgcggtttctctatcttttgtaaccgacactgacagtagcgggtaataaaaacaagaggattgttatcgtttttttatgataaaaaaacaacgtgtcattttcacggtgatttattcttgctattatttttccccatgggctgtcagcgtcgggtgcgcgacactgctaccatgcgcaacaggtccagtttaaaggcgcacttgtcgttaaacaggctggacatgcgtgtatatttgctcagcatggtggccagcaccgggtgggtggcctctgagatctcggtcggcaactccaaaacgacgttgacgacgtgacggtgtttttcgtcccgcttgttggccaccgtgggtcccggcgcggtgttagacatggggcaggccgtggggggaggacgaggaggaagtcgctgctaaaccgccacgcgcctgctgcacaatgtggccgccgacgtggcaggcggtctgtttaaccagcgcgcagccccgacacagcggggcgccgtcttcgctttccaaacagctgtcgcggtactcgcccgtctgacagcgcgcgcacagcaggccgtgcccgtgcgaagtgaggcgcaggagacgcgggaccgtcacgccgcgtaccaccacagtggagtcgcaggtgcgtgccgcgcagggcagaatgacgtcgaaagccagccggtgatcgtacacggcgcaagccgcgttgaggcccagcacggctttccagcccacgcgtacgcagcgctgtccaaagagcgtctcggagacgagctcgtagacgcgctgccgcaccacccgctgactgccgcagagcgagcagtgtacgagctcggcgtgcgtgttgaagatgacgctcttttcttgacggtcccgataatagaacatcgagttgagcggaaaattttgctggcagtgtagcttttccttacccaggttgaggcagtgtccgcactgccgacagaccacggccaccagcgagcgcgcgtccagatggcgctcgcacttgagtcgacacagacaccagagcggcaggtcgatgacgctgccgatgaggccgccgcgcagcgcggcgctgagtgcaaagaggacgatcttggtgggctctacgtgacgcgcctgctgtccggcgcccgcgtgtcctaccgccgcagctgccgccgtcgagcctcctccgcgcgtctcgtcgtgcagacccagtgcccgcaacggcaccaggtatcgcggacacgtgtcgcaaaacgtctgcaccgcttgtcgggccagtacgtagagcgggtttccgcagggtaccttcccagcgtgccggcgcaaggctgcgatgaggccccgcagctgcggcgaccgcggctgccgttggtgacaccactggttacggtggtatacggccaaatcagcgcgggcgtcgaagcgcttggcgcgtagtagtgctaggcacggcgagctggtggggtgaagcacgggcagccgaaggtccaccccgaaaaggaaacggtgaaggtcacctagcagcgaggcggtgacaccgtccaacaacgcgtgcagccgctcgggcgggtagagccgcagacggcgcagcaggtagtcggtgtcgtagcgttcgaaacgcagaaaggccatcgtgcggacggccacggtgtgcagacagtccatgctgtagacgtaagcgagaaacacaaagtagggcttggtcataaccatacgctgaaagagcgccgtcaccgcctcccgctcggcctgccgacacaccagccattcgcgcaggaagcgttggtagagacggtcgcccagctcccgattcagaaagcgcttatccgtcacgaagagatgaaggacgcaagaacgtggcacgtgatgcaccagctgctgctggaggaccgccgacgtctgcgccgcaaactgcgccggtggctgcgacgtttctaccgccgcttcctccggctgcagcgcaccgcggccgatcaccagctgcacatggaaatggtcctcgtgaacgcagaggggcgcgaagagacggcgcagagcctggtggaactcatcagtcgcggtgtgcggagcgtgtcggagacgacgactggccatgaccgcgccacagcagagccagcaccagcagaagagccagcaccagcgggcccagagtcgcaaagcgcgcgggcagccacggcccagactgcggtcgcgatggcccggagcgcgctcgccaccacgatgacggtgcccaacgataaccagtccgctccaaggacggcgcgcacggcggagacggcggatgacggtgatgggtcgacacccctcgccgacgactcacgtgctcctccagaggccgacgcgcggaccctccgacgtcctggcccgccgctgccgccgccgccttcccttctcccgccagagccagcaactcctcctcctcttcatcagcgtctccctcgcttgcgcatccgcatcgtcccatacaggcctcacaacgacacagccgccacgaccccgccgccatgggtggcggcggcggccgaggcccggcagcggcgccgccagcggcgaccatggtgggagagcaactcggatgacgaggaggaggaggaggagggggagatgcggtccgagaggaccgctttcccgccgttcgcgtgagcgcggccgacatgcgggcgcgccacagggacggaccgctgccgctgtgactgcttacggtgacgtggttccggaccgccaacgacgtcgacgcggctttcttggcgtacagctcgcgcagcagattctcgtactcgccctcgttttcgggtccgaaggcgatgagctcgatgttgaagaccgacgccgaattggatttgcgcaccacgcacttcgtcagcactccgtaggccgagggcttgatctcctcgatgtccttgagcgtgacgatgagcgactcgttcaccttaagcacattgaactcacctacgtggcgcgccggcgagacgagcttgacgggcgctcgcacaaaacagcagagggagacggcgcagccagtgtttttaaagataaaacaaggcacgtggtctgtgcggctctcccagtagctgagcagatactcgacacaatagaccgtgtctgtcttgagcatggcgtcgcacaccgagtaattgggatttttacagataaggccggcgtcggtgacgcgcagctcgctgggacccaacttgaggatacgccgcgtggcctgcaccagatcctgatggagaaccttgttcatctccatcgcaccgacgccaccgccgatttatttacccggcgccggctcgtcttttccctccaggattccgttaatgtccatgagcttgctgacgatcgccgttaatagttgcgtcttctcacggaggatctctccgtgactgcaggtcgcgcagtcgccgtgcacgtacttgaggaaggcggcgtacttctgacccgcgttcacgaaatttaagcgcgcgtccagggagggcagcaacagatcgtagacgcgcggcagcatcggctcgaactgtaatagcagatcgtcgtcaagatcgggtagcgcgtgcccgtcttcaccgtcctcgtcgtcaccacctcccccctcgagcccaccgctcgtaccagccgcgggctccgcgtcctcgtcgatcaccagcggtcgcgtcggcaccggagaatccacgtcatcctgcacgtcgttttcctcctctccgtcgtcatcgtccagaaacggcacccgctgcttagcccaggacattctttctccgcgtcctcaatcagcggcgccgatcgccatgaatccgagtacccacgtgagcagtaacggcccaacgactcccccccacggggcccacaccacgcttcttcccccgaccagcccggccccgtccaccagctccgtcgccgccgctaccttgtgcagtccgcaacgacaggccgtttcgcgttacagcggctggagcaccgagtacacccagtggcactcggacttgacaactgagctgctatggcacgcgcacccgcgtcaagtacctatggacgaagcgctggccgccgcggcggccgcctcataccaggtgaatcctcaacaccccgccaaccgttaccgtcattacgaattccagacgctcagcctcggcacctcgggggtagacgaactgctcaactgctgtgcggaagaaaccacgtgcggcggcacgcaatccaccgtactcaccaatgcgaccaacaccaccaactgcggcggagccgtcgccagcagtagcaacgcaggacctgccggcgcttcggccgcctgcgacctagatgcggaactggccggcctcgaaacctcggcggccgactttgaacagctgcggcgactgtgcgcgccgctggccatcgacacgcgctgtaacctatgcgccatcatcagcatctgcctcaaacaggactgcgaccaaagctggctcctcgagtacagcttactgtgcttcaagtgcagctacgcaccccgtgcggcgctcagcacgctcatcatcatgtccgagtttacgcatctgctgcagcagcacttttccgacctgcgcatcgacgacctgttccgacaccacgttctcacggtcttcgatttccacctgcactttttcataaatcgttgctttgaaaaacaagtgggcgacgcggttgataacgagaatgtcaccctgaaccatctggccgtggtgcgggccatggtcatgggcgaagacacggtgccttacaacaagcctcggcgccacccgcaacagaagcaaaaaaacaacccttatcacgtcgaagtgccgcaagaactgatcgacaactttctagaacacagctcacccagccgcgaccgcttcgtgcagcttcttttctatatgtgggccggcaccggcgtcatgagcaccacgccactcacggaactcacgcacactaagttcgcgaggctagacgcgttatccacggcctcggaaagagaagacgcaaggatgacgatggaagaagaggaggatgaagaaggggaagaaaaaggaggagacgatccgggccgtcacaacggcagtggcaccagcggggggttcagcgagagcacgctaaagaagaacgtgggtcccatttacctatgtcccgtacccgccttttttaccaaaaaccaaaccagtaccgtgtgtctgctgtgcgaactcatggcctgctcctattacgataacgtcgtcctgcgcgagctgtaccgccgcgtcgtctcgtactgtcagaacaatgtgaagatggtggaccgcattcaactggtattggccgatctgttgcgcgaatgcacgtcgccgctcggcgcggcgcacgaggacgtggcgcgctgtggactcgaagcacccacctcgcccggaggcgactcggactatcacggcctgagcggcgtcgacggcgcactggcgcgacccgacccggtattttgccacgtcctgcgtcaggcgggcgttacgggcatctacaagcactttttctgcgacccgcagtgcgccggcaacatccgcgtcaccaacgaggccgtgctcttcggacgcctgcacccccaccacgtccaggaggtgaaactggccatctgccacgacaattactatataagtcgacttccgcgacgtgtgtggctctgcatcacactcttcaaggcctttcagattacaaaacgcacctacaaaggcaaagtgcacctggcggactttatgcgcgatttcacgcagctgttggagagttgcgacatcaagctggtggaccccacgtacgtgatagacaagtatgtctagcgtgagcggcgtgcgcacgccgcgcgaacgacgctcggccttgcgctccctgctccgcaagcgccgccaacgcgagctggccagcaaagtggcgtcaacggtgaacggcgctacgtcggccaacaaccacggcgaatcgccgtcaccggccgacgcgcgcccgcgcctcacgctgcacgacctgcacgacatcttccgcgagcaccccgaactggagctcaagtatctcaacatgatgaagatggccattacgggcaaagagtccatctgcttacccttcaatttccactcgcatcggcagcacacctgcctcgacatctcgccgtacggcaacgagcaggtctcgcgcatcgcctgcacctcgtgcgaggacaaccgcatcctgcccaccgcctccgacgccatggtggccttcatcaatcagacgtccaacatcatgaaaaatagaaacttttattacgggttctgtaagagcagcgagctactcaagctctccaccaaccagccgcccatcttccaaatttattacctgctgcacgccgctaaccacgacatcgtgccctttatgcacgccgaggacggccggttgcacatgcacgtcatcttcgaaaactccgacgtgcacatcccctgcgactgcatcacgcagatgctcacggcggcgcgcgaagactacagcgtcacgctcaacatcgtgcgcgaccacgtcgttatcagcgtgctgtgtcacgccgtctcggccagcagcgtcaagatcgacgtgactattttgcaacgcaagattgacgagatggacattcccaacgacgtgagcgagtcctttgagcgctacaaagagctcattcaggagctgtgtcagtccagcggcaacaacctatacgaggaggccacgtcatcctacgcgatacggtctcccctaaccgcgtcgccgttgcacgtagtttccaccaacggctgcggcccctcctcctcctcgtcccagtccacgccgcctcatctccacccgccgtcgcaggcgacgcagccccaccactactctcaccaccagtctcagtctcagcagcatcatcaccgtccccagtcaccaccgccgccgctgtttctcaacagcattcgtgcgccttgacactgtacggcagaaaagccggctccaagtgcaagcgccgcggcagcaccatgtgcaaaaacttgtccttgcgcgcggtttcgccgccgggaaagacgggcgacagcacgttggttacagccttgagaacctgctcaaagtacttgtcggtgtgaatgggcacgccgtgctcgcgcacgtagctcggatcttcggctacctcgtagttgcacacggccgacggtggtttccgcgccctcttctttgccggctctcctcctctcctgttgctctcctctaccccgccgccgtcagcgtcgtcgtccgtgccatcaatcgcgtccgaccgggaaaccacgccggcggttacagaatcaccgttgtcggaggaaccctgcggcgccgtccggacaccgggcgccgtcaggacgtaaaagacccgatccccgaccgagggtagctcctcagaacgggccgccagtcgcttaatgacggcaatgtgcggcaggttagattgacggtacaacgagatgtccttagaaagcaccgacgaaagcaccaggtcctcgacacgcacacggtgcaggtacagatcgtcgcgggcctgcaccaggcggcgcaagatacgccagaaaccgcgtggcacgccgtatttcttgacttcatcgagtgagaggcgcgacaggcgcacggctgcttccgagacctcgcgatcctcaaagagcagcgagaggacgtcacgcgtgacgcccttgacgaactcgcaggccgtcttgcgcaccagatccacgcccttcatgctcagacccgaggcgccctccactttgccgatgtaacgtttcttgcagatcatcataagagagacgaagaccttttcaaactccagcttgacgggctccacaaaaagacaggccgtcacgtagtgcgccaggctgggcccacgcgccaccagagcctgcggcgtcaggccacgaaagcggacaaacacgctgtccgtgtccccgtagatgacccgcgcctccacccgccgttcgtccgagccccctgacgatgtttcgagcccctccggtaacgtgctgctctcctccgaatccccctctcgcgttctcactacatagtcttcctgattaaaaaaattgtgcaaaaaacacggctctgaaaaattgtctttgatgaaccgcgccgtgcgctctagcatgtcgcgaccgatgcgcgtgatgctggcggcgatgggcagacacggcatcatgccgttgaccacgccggtaaaaccgtagaaagcgttacacgttactttgagtgccatctgttccttatcgagcagcatacggcgcacggggtcttgacactcgcgcatgcattcgcgcacggcacggcgctgcgaaacccacttgttgagcagttccgaaagcaccgagacgcgcaccgaagcacgcacaaagcggtgagtcacgccgttctctagcgtgacgctgtatacgtcggcggggtccacggggtactcgccacccggcaccagcagggtggagtagcagaggttgtgagccatgatgatggaagggtagaggctggcaaagtcgaacacggccacggggtcgttgtaataacccacctcgggctcaaacaccgtggcaccctggtacgaaaccgccgcagtaccgccggcgccgtgactgtcgttggaaacgccgacgctgccactactgccggagccgacgctgaaaacgccgacgctgctactactgttactgccagagccgggtaaaacgccgtcctgactcgacggcgcagattgcaagggcggcgacatctgaaacatggccgccacagaacccgcgtcgccgggcacggcagcggtagagatgatagcggcgttaggtgacacggcaacgctattcgtttcgggcaccgtcgtacctttgctgtagtggttgggcaggataaaatcgcggcaggcgcactcgtccagcagcgaggtgtagatacggatctgctgtccgtcaaagatgacacgccgcaacggaattttagccagccgcgcgatggccccggcctcgtagtgaaaattaatggtgttgaacagatcgcgcaccaatacggcgtcctgcagacagtaacggcctacctgggcgcggccctcggcattagccacgaaacaacgcgggatgtccttgtaggacaggtcatccttgcgttgccgcaggtaaagctcggccatagtgttgagcttatagttgggcgagttagtcttggccatgcatacggggtacatgtcgataaccaccgaacccgcaatatacaccttggtggcggccgtgctggccggattgttgtgagaagccgagggaaaggcggcggcgtactgccgcttaaaacccacggcggggctgtgtaaaaagaaacggccgccctgcgccgtgggcaacttgcagaagcgctgcgagtccaccttatacaggtactcgaggcgcgtgaggatgtacttcaagtcaaaagagttgatgttgtaaccggtcacaaaggccggcgcgtaccgttgaaagaaaagcataaagcccagcagcagctcgtattcggaagggaactcgtagacgtccacgtctgggcccacctgcccgcaggtgccgatcgtaaagagatgaagacccgagtgcccaaagatcacaccctccgaagtgcagccccgaccatcgttcccgtttgggatcccctgatccacggcggtgtttccccccgtctcgtagcacacgcacgagatctgaatgacaatgtcatcggacttctcggcgcagggaaaaccaccctcgccgctcatgcactcgatatcgaaggacaggcatcgatagcgcggccacgagctgtcgtcgggcacggccaccaggtcagagacatcgcagtcgacctcgatatcacaagtcgacgcgcgaccctgctgccgccagtcgtaacgattcacggagcaccagccgaacgtggtgatccgccgatcgatgaccaaacgcgtcagcggatccacacggacctcgtacacgggaaaaccctgctccagcagatactcgccgatttttctggccatggtccagttgctgatagacacacactgcaaatcgggcacgggtcgcgtcccgtacccgtagatggaggtcttggtggccggcgtgacagacacggcgtatggcgtccgcggctcgggcactagttcgcccacgctggcaatgacctcacgcagcctatcggtgtcgctgtactcacagtaaaagtagctgcgctgcccgaaaacgttgacgcagatactgtagccgtgttctgtggccccgaagaaacgcaacacgttccccgaaggcaccagatgctgacgatagcgcggcgacacgttttcgggcgagtcgaagaagagcacggcgtccgtctgatcgtaggtgtgaaaacgaataggtcccaccacgcgacccaccagggtctcgcgccaaggacacggccaaaccatgtcatgactcaacaaatgtttaatctctcgatagaacatgagaggcagccgtcccgtcttatgcttgatcaaccccgtctgaccgtcgaacatgacgcctcgcggcacgatctgcaaaaactgtttctgtggcggccgcttgcccgagccctgcgcggagccgggctgcgaacgctgacgccggccacccgcgaccgcaccgccggtcacgccgccgctcagatacgggttgaaaaacatagcggaccgtgagaggctgacagcttacgaagcacaatcacaaagaaaatacacatgcagcacctagatatccagtttgaccccgtatatcacaagtctctgtgtcactttttttgtctgttttttttttcttctcctggttcagacgttctcttcttcgtcagagtctttcaagtgtcggtagccgtttttgcgatgtcgcagtcggtctagcaggttgggcttctgtcccttgtcctgcgtgccagtctgtccgtccaaagaatctgtaccgttctgctgcgctcgctgctctgcgtccagacggaccagggccagaagcatctggtaagcctgctcgttggtgtaaggcggagccgccgtggatgcatcagacgacggtggtcccgatcctttgcgaccagaattataaacactttcctcgtaggaaggcggagcctgtaacgacgtgtctttggtgttgcccgacgtcacggtggtcccgttggcggacaccagatagggaaagaggttctgcagcggctgcatgcagagacgccgctgtcgagtatagatcaaataagtgataatgactacggctatggccacgaggatgatggtgaaggctccgaaggggtttttgaggaaggtggcaacgccttcgaccacggaggccaccgcgccacccacggccccaatggctacgccaacggcctttcccgcggcgcccaggccgctcatgaggtcgtccagacccttgaggtagggcggtagcgggtcgactaccttgtcctccacgtactttacccgctgcttgtacgagttgaattcgcgcatgatctcttcgaggtcaaaaacgttgctggaacgcagctctttctgcgagtaaagttccagtaccctgaagtcggtattttccagcgggtcgatatccagggcgatcatgctgtcgacggtggagatactgctgaggtcaatcatgcgtttgaagaggtagtccacgtactcgtaggccgagttcccggcgatgaagatcttgagactgggaagctgacattcctcagtgcggtggttgcccaacaggatttcgttgtcctcgcccagttgaccgtactgcacgtacgagctgttggcgaaattaaagatgaccacgggtcgtgagtagcagcgtcctggcgaatccttcacgttcatatcacgcagcaccttgacgctggtttggttgatggtcacgcagctggccaggcccaagacatcacccatgaaacgcgcggcaatcggtttgttgtagatggccgagagaatggctgacgggttgatcttgctgagttccttgaagacctctaggctgcgccgttgatccacacaccaggcttctgcgatttgcgccagcgcccggttgatgtaaccgcgcaatgtgtcataggtgaactgcagctgggcgtagaccagattgtgcaccgattccatgctggataaatgagttgcattgttgccatctgcacttcttttggttctactatgagtaagattcagactggagcggttggccaaacgttcgagttccaccagagatttttgcttgataccttgccagaacaccaccaaaccaccagtggtttcaaacacggacacgtttccatatttttcatatgtttgattgtatgaagtattgaaaatctgctgtaacttatttatggcctcatcacgtacgcagtccagcgcagagtcggacatgttcacctcttgcttcttagataagaaagtggcggtcattttggcagaagaaaagtgatacgagtcctcggcttcggaacgaatggtgcgttccgaggcttcccagaaagtgagttgacaagtaacattcttctcgtcctgtatatcccaggagatcactgagtccgcacgttcaagaaaagccaccaacctgtgggtctctaacgcagaattcggtcttccaaagtcggagacgatagtgtagttcggaaaaatgaaaaacttgtcggcgttttctccaaaatagctggcattgcgattagttccgttgtagaaaggagaaatgtcaaccacatcacccgtggaagttgcgaaaaaatgatagggatacttggagcgcgcagtagtgatggtcaccatacaattcagattacaggtctcacgatagagccaggtgctgccgcggctgtgccattgatccttgaccgtcacgtaacgggtactgtgggtgttggaataatcgtcgggcattaattgcatggttttgttttcatagctgtccctatgataagccacgaaaaccgtgcctgctataacgcggctgtaggaactgtagcactgactgtggctgttgatatgatgaatctcccacataggaggcgccacgtattccgtgttgctgcccagcagataagtggtgtggatgtaagcgtagctacgacgaaacgtcaaaaccttctggtagactcgtaccttaaaggtgtgcgcgacgatgttgcgtttgtagaccaccatgatgccctcgtccaggtcttcattgataggcttcatcgaggtgcagacgatattacgttcaaagcgaataagatccgtaccctgtgccatagaacacacgcgataggggtacttggtggtattgacccccaccacatctccgtacttgagggtagtgttgtagatggtctcgttaacaccatggctgaccgtttgggaagaagttacgcgttgagagactgaaccggatcgagagtgagcagcagacgtcgtatgagaggaatggtgactgtgagtagcagaagttccacgagaagtagaagatgaggaaaccgcagcacccagacagacgatacacaagttaacgcagactaccaggcaccagatcctggattccatgttcgtcgcgggccaaatccagcagcgatgaggcgcgtcgtggtctcttgcgtgttgcgcggaccctccgggaaacgcccgcggtcgaggaggaggggtacggacttggcagccaaggtcggtccggctccctgaaggcacccgagacggccgcggcggccgttagggtggagggcttggccacgggagctgttggcacgtcgccactctcatccggtctggacagatgcctgtagaggaggagatatagatctttggacttataaagacttccttcgtgacgaagcagcagcggccactctttgttatacgtgagaatcacatctctgtccgggtgcagttcgtcgcgcaggcacgcgatcgagagttgtttcccgaaagtttcattatatagtgcgacggagagcacgagctcccgcacgtgcatccacatctccttctgcagcacgtttaggtcctgacagtccgaaaaattgaaaaaacccatgtacttcaccaccatccactcactgggatacacggtaccttccgcgcatttgaccaaatcgtccttgacgtggggtagtacgcccgcgttgtcgcaggcataggccatgtccacattgtgagagaggggatagcgatcggtacagtgtgtgaagaggggcccgttacacaactcgtagatctgctgacccagtagcgggagggattccacaggcagactcttgtggatcaggttattgaccacatacaggtgctcatcgtacgtgaactgatcccccacgtccaccacgtcttggtcctggtggtattggctgcggtatagaaacccattcatgagcttagagataaagtccagacacaagggccccactaggttgacatcgatgagtttgctagtcagacgctcctgcgttttgatgcaacggatcaccttgccatagcccacctctgagaccttctgcaggtaggcgcgtttgcgcacgttcacctcgcgggtgacgttgtggatgcgggaacgcgcgtccaccaagtcgagagcctcgtgttcgtcgcagttgcgcacccgtaagccgttctcgccgccgtcgccgtcctgcccattcgcccctccccctaccgctttcttgcctcctccacgggcccggccgccgccaccgttattcctctgactgtgagtactgctgttgctgctgttgctggccgtcatcaaagtcgtacccgtccccgacatcgcctcccgtccacgcaggtgaatagcctcgccctcggggccgtcgccccccgtgccatcgggcagcggacgtcgaatctcctcgagaatatgcttgattttggtgtacatctcgttgctttcgtggagcttgttgaacaccgggttgtcctcgaaagcttgaatgctgagggatgtgatgaggtcgatgatcctgttgggggcggcaaagaccgaccccacgaacatgcgctcctccccgtccaacgccttttccccgagcacgaagatgtcctccacgtcctccccgtacagatggcgactgatgccgttcatgagcgcccggcacagctggtgatacacatttagctgctggatggtgatgcccacccgcttgacgataacctccgaggtacgggaccagtaggtaaaatccgacaaggaatatattcgttccggtatatccgtaaacaggttgtactccctcagcgcctcctccgcctcctggatgtagctgtggtaggccgatgaagaagagaataggcttttgagggccgaaaggactccagccaagtgggggatgcgcgttgtcaggtccagcaggtcctgctccaccgtctggatattcacatcggactggcttgacggacggtggaccgctatatggttgcacagcaagccctgcagccgcttgttcagcgagcggccctgattcgggatgatggtcagctcctcgtagcattgggcgcatgtcgtcccttcgacgtacacttcctgacgcgccaccggcgagatgccgcataggcgacggaggagctccagcagctgcgcgcagacctccaggccggcctccggcgccaggatcccgtacacgtagttcattttgcacaggaagcgctcgatgtcgttgagtgtggccagactgacgctgaaacggacgttgtccgtaaactggagctccacggtgtgatggcgatcgcagcgatccaaacggaggacggtacggtagaaggccgcccggtccggctggcgcgagtaggccatcagcgcccgatccagcaaagccgtatcctcgtgcagcgccttcagcagcatctccagatagagcgtcagcagcgaactctgcgtacgattctgcgccaccacctccgggtagatcttccggtacagatacactatagccgccgcgtttctcttgaacggcgtggactccgccagtaacacgttcggatcgcagtactttagacactccagctccatggcgtattcgttgcatttcgaacacactacgcatagtttctgtaacaaattcatctccatgactcgactcgctcacgtacgagacgctgtcgtccggtctggcgccggccagagacatggagtcggtgcacaaataactcgcgggccgctcgctatgccgactgacgttgacgttaatatataacgacgtcgtcgacgacgcgggttctgctcccgaagctgttgccgccgcttgcggcgcaacctcctccaccaccgccgccgccggctcctccgcctcgggcgacgggggctcggagatgaccggctgtgtctgacactcctccccttcctcaggcggcccgggcgccgacgcgaatgtcggagtttgccagcgcggcggcggtctctgtctctggtgccgcggcgctaaccttcggggctgttgctgctgttgatgatgcgacgccgtctgtcgccgctgttgcggcggtagctgatacggtgtcgcctggtgctgctgtgtcggtggctgctgttgctgctgttgttgcggtctgaaaagcggccacgggggctgcgactgttgctgctgttgttgcgatgctcgtggctgcggcggccgttgtcgcggcgtttgctggcggttacaaccggctgcgtttggccggcaataacccgctgcccccgccgcccccgctgctcccgccgacgccgccagcctcgtcttcgccggcgttcacgagaaagcagccacctcccgtctcgccgggcacgccgaagcaaatggagttgcccgcgacggactcgccgagaagaagaccgccacccccgacgccggacgccgcgccgacgccactgggcgcgaagagcgccgacaggtcgtgcacctcccccccggcggcgtccgttaatcgctgggcgtcggcgtccagcacgcgtcgcaagttctccagcgaaaagtcctccacgccctgctcctgcaacgcggcaaacttgtccatcagcgacgcggccagcgcctcgcagccatccacgaagaagagcacatcgtcggacgcggggatctcctcgcgcacgctcagaatctcgtacacggccatcacttcggggtcgcaatccaagttctcggcgtccagcgccagcatgacgcggttttttataagatccgcgtcaaaaagcacgttctcgcggcgcgagcgtttgatgagcacgtcggccagacgcgtagccaagaggtagcgctggcgcatgaaacgataatcttggccgctcatagagctcacgttaaggctgcgttccacaccgttgcccgaaaagtagccgatctgcccaaactgatagatctccttgctgttgttgatacccgcatacttttccacgctcacgggcacggtcaccaaggaacgatgctcaaaaacgctccgtaccaacgattcacgcgccacagtggcggccatgggcgccggcacgcctgcggtcttcaagcccttgacatgcaacgcaaattcggcgggcgacgagaaacgcggactagcacctaacacgtgaggaaactgcgcgtggttctgcgtcgttaagcgcgtcgtcaacccgtgcagcgagccaatgtagtctttgaagccgtagtagcagaggaatttgttatggaaacggctttccacgtaactcagcacacagtctggcgccacatccagcagatcgtgctcctgatagtcagccgtcacagccaccagaaatttgacgaaagcattgaactcgcccatgtcacctatgggcacattcttgggcaacgcgttggaacagaccttctgccaaaactgtaagcaggggagaccacattcaggaaagagtcgctcgtgatgtcgatacagcagaaatcccaagcagcccttagccggattacgacgcggaacgtgatcgcggcgaaaaaacacgctacccgcgttgcccttgcccgcgcggtagatgggtcggtttttcactcgcaccatgatcaacgtgggtaccgacagccgcgagagcttgatctccatgggcaccacggcgtacgtgccctgcgcgtacagcctaaagtccagcaggcggtcgtgatccgaattcttggacgacttgatctgcttggtgaagagaaagcccttgcgcgacgacgtggtggagaacgcgccgtgaatggattgaaaatgctgcgtcatccatttggataccaagttggtggtcaacggattgtccacaatgtatgaggtagcggtaataagcgccacgttctggatcacgtaaaagacggatctgaaataggcgtaggccagcagcggctggaaggccacggcgtagggattcagatccaggttgaaggcctgcgtggcgcccgccacctcgtcgcggctgctcttgaggcgcacctccgaaacgaaacccagggcctcgtcgtccacaaacttgttgagcgccgaaaagacggccacaaagtcgcttttgccgtgcgcgctaaaggtatcctcgcccgtcacggggtcgatgagccgcatcttgcggcagtaatccaagatgcgattgagccgataggtacggtccacgctagcgcccagcatgcgaccgccgcgccccatcattcccccggaatccccgccacccccaccaccacgaccgccgcccagaccgtcgctcgggcccccgctcacgtcccgtccaccacccccgccagcaccgccgcccggaaccccgtcgtcacctttgccgtccaaacccccgtccttggcgtcgacgttgtaacgccgaccgaagctgcccaaaatatccacgtcgttgagaaaacgcgactgcacggtgatcacgcagggctccttcttgggctgcttgggcaccacgggcaagcgggtgcgcacccgcacgaaggccgtctgataacacgtgtggcaacaagtacccccacaggcctcgcacagccccgcggcgcagcccaccaggtgattcgtgagcgtcgacgaacccgacaagcccgtgttatacaccgagacacgatttagataccagacgaagcccgaaactagctgcggacacgtgccacacaccaacgccaaatgctgcggcccatagcgttcgtccttgagcggcgcgccttgaaatttgagcaccttgcgcgcgtcgttgtagacgtcttcgcaggccgccgacaacccgttggtgaactgaatagccttgagcaacgtctcctgactggccgtaccgccggcgctgggatgccgcgccgacgactggagatacaccagcctgtgctggtagagcaccgaattagcgctgaagaccaaggcggccacgtgcgtcgagagatgcaacttgagctcggtcagcgcgcggatcagatcgcggtgatcggttgcgttggtcactaaaggccactcggaaaagagcatagactcggcaggttggtaggccgaatcgaaaaataccgaggcaaaactgaaggccaactcgcaaaccaccgcgtcactcagcatcagatgatccttttccagactgctgagtcgctggctcatgtaccccaagtagcgcttatgtggcgccagcttcaccgactgctgactgtcgtgcacaaactgccgcaacgccgcctcgatcagcacacgcggctccgagaagcgcagcgattgacaccatgacgtgtacacgtagtagaaaagcgtctcgcttacggccggcacgtagagccctcgcgcctccacaaaagcgctgcgcgcatccagcgagacctcgtcggcttcggcgtcaagctgcagcgaattaaagagcgtaggcgggtacaacggcacgcgcaccgcctcgccgccgtacagtcgcaccgtggtcgcctcctccacgcatggaatcagctgaccggcaaagagaaactccttcaagccgttgcccaccaccacgtgcacagtcgtctcggacgcctgacagcccaccgccgcgcacaacgccgacagatcggtaggcacgcgatccgcctcgggcgtgtaggcctccaacgcgtacttctggcgggcgtcctcgcacaaccgatgcacgtctccgtgatcctcggtaaaagccacgatgccttgcgtatgatgaaagtagagcgcaaaaggacagaaggacgtgactttcgtgagcaccccgccgtcgtaacaaagcacaggcgtgcgcacagagacgccgaaatccgcctccaccgtcagccccgccaacagaggagcgatcaccacgctcgaggaacggtcgcatagcgagagagtggccagaatctcctgcgtttctgcgttcaacctgctgaagtagagaaaagccgcgggccccaccggcgctagcgcggttagttcctcgtggctcatggtggatgaacggaagacaatggctacgccgccactgagtgaattttataccaaggaaaagttcagcacgtcatgtttgacgcacgacgtctgatacaccaccgtggccaccactgcggtctggctgcggttgcggaccaccaaaggcgacaaccgcaacgatcccagcaattcgtaagaaaagctaaccgttacggtcgggcagcctctcgcagccagaccgctagccgacgcacccgcccgcgaaaatagcgtgatgttcgggacggcttcgcgtcaccgcaaactaacgtcggtagtcgcgcacgtcgtttatcctcagcacaccgtccgatcacaacccgttttcccactcagtcgcacaagcagcacataaaaaccccacacagtgcacgtgaaaacaacgtccctagaaaacggtgttttctgtcctaccgtcaccgggccacacaggcaaatcccgagcccgatccccgaaaacaccgtacggtgtttgtggcctccaaaatcacatcagataacaaaccgtgaaaagtcacgtttcacgaacacggtgtttttaaatcacaaagaaccacctgacggtttacaagcagaaacaccgcaccacggtggtacaagcgcggtggatctggtctcgcaacctcaatcgccgctatcaccaccgactttcgctgcgctccgccgacaaaacgccgtataagctacacaccccaaaaacccgcacgcctatgggcgccaaacgtgtgtattatctcaacgtcacgacacgacacaaacagcgtaacgtggtttcccgaacacgtacgcggcacagacccccgacacgtactcgaagaccttacagtttacgagtcaataaaacaggaaaagatccgaactttaaaattgtgtatttttattttcccatccccctctttttaccaaaaaacacatttttcgtcttgtaaaaagtaactttcgcccattgccatgaaacaccgtgatggggaacggtgttgtgtgtcgactgacgtcactacggcgatcagtatcgacgtcgtgtatacataacggtgcccggtgtttttattcggggcgttgtcgcgtcttgatgtaatgtaacctgaaaccgccgtgtccaagaatgcggaagccagcgtgtaatcataacggggttttgggtacaatctgacgacatctggcggcgagcgtacaccatcgaatgtggcgatcgccggctctacgtcacaatgacgcaaaaacacactgtaaaacacgcgtagacagctttcctggtcaacgagcgccatctggtgtcggcataagaacaggcatcaaccccgtggccggcgaggcggtgagcacttttgctggtcacgtgaccatcagcgcaggaagcgaggcccgtagaaccgcccaagaggcggtgccagatgccaacgtcataatcacaaggtgatttgttacgtcacgcgcgcgcacgcacgcgcgcgcggtagaatacagcgatccctagtgaagccacacccattacgtgtagccatatccgcttacgtatacagccacacccctaggtacgccaccttatctaccaatcatagaaacggatatacaatgacccctccctagactccaccccttgtacggaaatttcagataggtggaacccgttagggttccaccgtcctcggtgtacgtacaggcttctccgtctaccggaaatatacacctgctgacgtagacgctactcccggatacgcgtcataagctactggaccctaggggggagtgtctacagggctacgtgcacgcccccttacttagggtatccgcccccttcctctgttttggcctagtaaacttaacgccgccgcttctcacgtgacccctggcaagcctacgtcacactcgcgtgaccacacccactccggatatacgtcatcctgtggaattccggacatacggtgacgtagcgagcgtagcgagctacgtcacgtatgcgtacgtcacctccggcggaaatcatctctgatgacgtagcgagcgaagcgagctacgtcatcagtccgttttacgtataccgggtgctaggcgacgccccgtaggggcggagcctagcttccacccctaggatgcataccctatatagcataattcttctaacgaaacgttctacgaaaacggactggcggaacgggaaccaccgtaacccccccccctcacccccccccttctcctccggaaccggggggggcaaatttttaccaaatttgggcaaccatgttttccaatgggacggcgtttccgtgcgcatgcgcagtcggggcgaatttttggttgtcagggcgttgccacgcggattatgggatgggggctcgagtgcgcatgcgccggggatgccgcatggaaagcctatatataaggaggggtgaaccaggggccccggtgcgcatgcgcggaccaggccccgcgggagggtcgccctgcgcatgcgccggtaaaattccactgggtgtgtgtcgtgcgcatgcgccagtatttttccactggaggcggtcagtgcgcatgcgtcggtaaaattccactggatgtgcaccgtgcgcatgcgccggtatttttccactgggcggccgcacctagggagcgcgagccccgtgccgggcatgggccgcggcggtggaaaattaccgctccgcccacctaggcggggcatcttaaaacctataaaacccggcgtacccgccgccccccggcgcagtccgcggcagggttccggccgtgctgcggtccgcacgctgcgcccgctcccgcctgcctcccgccctaccccccaccctccccggccgaggcccggcgccggtccgtccgcgggcccgtcccaccgccctggagcaccatccggggccgtgggccgggcaccgggcgcggcccgctccggacctcggccgggggtccctcccctccccccgctcgacccccccatccgacggcccggccgggctgggacccccgcaccggggtcccggttcccgtccgcggcccggggggacccgagcgggggcttcccacccccaccccgctcctccccgggctccggcccgggatccctcgctgctcccggcgacctccgccggcttcccggtccacccgccgcggaacggacgggacccggggtccgcgcccttcccctccccccacggggggctgggtcacggaccccggttcctaggctcgttccgcggtgggcgaccggggatcccccacccagctccccttcccggcccgccccgctggcttttgggcccctccgggctttttttccggctgggggtcgcggcgctcggccgacgacgacggtaggtgggccgggtggacggtggtggggacgggcgacgccccggctcgacggcagtcggtcccggaaggttgggggctgggggcccggtcaggagctccgggagcggggtcgaccgcgacggcttccgggtctcgcggcggctccctctcggcggctccggttgggctcccctcccccctctcgagggtccggccgccagtcgtgaccgggggtccctcggcctagccgccggctctcggtccgccttatcctgggcgttggccggtcccgtgacgctcccctcccccgctgctccccaaaaaactccgcccgaaccgtcgcggcttgctggccctgggcgtggtcccccactcccctccccccatcggccgcccagccggggtcggcgcctcggaccccaccaggctgtggcgtgtgtgctggccgatgcggcggcgaggttgggtgtggccggaagcgctcggggtcgacggtgggccgtcatgacacctcaattgccgtcagtacgcccctccacaatcaccgtccctacacgatgggcccggcaggtcacccaacgttggttcaggcccagtcgggtttttccccggcacaaacgcacgtccccgtgggctccacgcgttttccaccctttcctggaggggtccggaacaccgtgaatccgcggggagggtctcggcacgggccgaggagaccacgaccgtcccacccggcgtgtcgacccgtccgagacccgggaagggaacaggccccacccttttttttcccttctccgatttgccgtggaaaacccgtgaaccgatacgggtacagacggccgaaaaaattcgagacgacaatacgacggcagggcgtgattttcttccccatccgacaaaaccgtgtccctcaaaattccccacctttctctgttcaaatggccccgaaactgtaaaacaccgttcgaccgcaccccaaccggcgccatcttggtgaccttctcgacggttctctcgctcgtcatgccgttctgagctccgacatggcggacgagagaaaatggcgtcgagagcataggagcgttttcgctccaggcgggtaaaagaatagcacgataacttttctgtgctttttttgagacgttttagaagagctttttttctgctcagagcgaaaaaatgatagccctgaaaatctcgacgagtctggccgagcggcgccatcttggaggaggggcgagtcgcgggcaccgcctcggtaccccctggccgaggcgagtccgcggtcgccgcctgttccgtgatgctacctagagggcgccgtcgaggcgactcttcctgttttcgccctgagggctaacggtcgctgacgtcaaaccatctcgtgctcgctgagtcacatccggttgttgacaagcgatggaggaccgcacccaaagtgcgccctctagtcatcgcgcctgaccccttttataaactgctcgaagaaaagaacaccttatgtgaaaaaatacagaatgatgacaagttcatccaacacaaccgctcaacaacgccatatctatcagtgtccaaaaactatcttctatcctttgaaactataaatgctgcctatatacatatttagtatccaagactcttaccacgtagacgaaaagaagtgatacaatgatcttgacgtgtatcgtctatatcgtgctagatatattcagataagacgcgcaaaccatagatttctcatcagtatcatgaaagacctatagctctatatacgaacctagtcattttaggacagccgccggagaagccgacgagggatcgggcgggtgcagccagaacctcacgcccgatcccgcctccggtaggcgatttgcatctgtttggtaaaaagctcataagtctgtatgtgacctatatatatattatacgctatgtacaccgaactgtcgctgttgtataagaagaaaaaactctccatatttatatcgtctgaatttttgcttgatagacacgtgtttggaactctgtccccccacgttttcactgtgtataacaaaaatatgtgtttctcaaaagatcttgaggtgtttgaaaacgggggaaacccgcgtttgggtgcgctaagccccggactgggacgtagccggcgtccggcacctatatttttctattttttttacaaaatatatgatgaaccaagaataaaactctagctctcgtctatttttaatatgctctacttagaacctttttaatgacagaatgaactccatgttatacgctctttatatagtttctctgcactaacctttaaaatcgtatccttccctgttgtacaaatcatcttttgatacacaatgatgacctgatatccctccatatatatgatcggatattattccgttagacttgtcctccttttttttcctcatctcctgtatctggagatatatgttgaccaccaccgccatgaccaccaaaaagctagccgtcacgactagaaatgtgtaggattcggactttccgttcgagaagaaagagaccgcgtctctggacgctctttttgtcggtctgaatcgacccgggatacgtaagagagcggccctacatcggggggcgctcgagaccgacgacgttccatctgaccagaaaaaaaaaaggcacccctcggtagcgacctctcaccatcgtttgcccgtccgcccgtccttcgtagccatcatcatcatctcaggctctatcggtaccatcgttgtcatctgaaaaaaaaaaactgcctcacccacctgcgtaaaaacaccatctttccggaggtgcggtaagacgggcaaatacggtcgtgccgaggcaaaaaaacgcaccatcgacaccacaccctcatgagcaccacctgtcggtgttggtcgtcctccatcgttctctacgaacatctcgacgcccgggtgacggacgacggcaagacgtcccggagaagacggtgttctctcgggcggtacgctctctggatctataatatctatagtagctaaacgagactgtgagtacgacgaaccacatcatcttttttttatgttgctcctttagaaaatgacttatgtcgacgacactcggcatcagccatctcgtgaaacacgctcgcttttcgtctctccaaggaacactgggtccgctgaaagggaccgtgtaccgaccaaagcaaaaaacacacacgtagtaacatgatcaaccacgtctgaatgacacgaaaacacaatcgtataacgctctattcatggaacgaacttggaataaaaaaaccatcgcaggccagaggctaagccgaaaccgtccggggaagcgggcgcgagttttccgacttagtctctggtgctcgttgagcctctttttttttcctgattctctgaagaatcaccgtcacagccctatgacgcgaaatcaattgctagaacataaacgttctcaacaggtatgaaatgaacaaactagatgatgctataaccttatattgtgtgtatatagataggtgtgaaatttgtaggataaaaagtgtcgttgtatgatgcacaacgatcgtgaaactggagactgtagctctctaccgaatgcaaatacacaaatgacatcgattcccgtccccacataaagaaatgtgctttactgtgaaagaatgaagaagattcttgttcctcgtacgacggggccctcgctcgtcgtacctcttcccccctccgggagaggggacgtcggggccctccgtcgcaccgggccgaagccagtgaaatgtttactacactgtcatcagaatatatgatgtatattatttcctccaaactcctcaccatagccaccaattcgcatcacttaagaaagtagtagcaaccgcggcggcggcgaccggccggtcgtcgtctcctcgtcctcaaatgttgtacatgtgcagaaaaatgtgtaaatacgtgttatttatcccatgcgtcttgtacatagatatatgtttttatatacgctatttatactttatatatccttttgcataaccatagacagtcaaggattttaatgatttgctcatccgcctttgagccatcgcttaggagttagttcctctatgttctcggcccaccttttcgactacagtagcaaacccttgtactaccaccccgataaaaaccacatcatcatcgtcaccacgacctggaaacgacacacgttcccccccaatcttgggcatgtgtatatatataaagaatgggagggagaggacgtggggctcgagaagaaataaacgccaagctcgattcgaaccaaaaaaccacatgtgtattgtgctttttttgtttttacggtggggaaaaggagggggccgtcattaacgaaaaccgtgtatggggtccggacacgaacagtacacagcttatggggaaaaaagctcacagagagaaaaaaacaccaagctcaggcacgcgtacatcattatcatcatcggatatctcaccacgagtcatagtagtaccaaggagtgtgtaacaccattttttcttttttctttgtaacgggataagggacagcaatcatcacgcacaacacccttcactctctttttagtcatccatatcatcgctgtaacacagcatgtcctcgtaatcgggcgtctggcagcgcattaccaccgagtcgtcttcttgcggtaccggtggtggcggcggcggcggcggctgctgctgggttaccgtcgtactgtgattaccgttggcggactgcaccgggatgataggctgcttgtggggaacctggggtggactgccgccgtgagaaggcgacggcgtcatcaagttaagctcaccacggtgactccggacaccggcgaggggccccgggggactgggagggaccgcggtcgtcttgtagacgacggtgtccccgtgtcgatccgtggctcgtaccagatcttgactgctagcgtcgtcactgtcttcgtcctcttccagctcgccctcagagtagtgctgctgcggttgcgacggtggctgggcgggaggagcggcggcgatcattggagagggatgtcgatgactcccttctctgtcctttttatcgtaggctgtcagcgttgctgggtccgtcctgctttccatatttgcgcattgctcatcggtgggatgaatttggtctcctccccgctgttgtccgccggcagtggcgtggttgctggcggttgtcgttgtcgtaccggcaaagacggtgagatccaatagtgactgctcgtcgaagggacagtacgctatcatgaaacgatagggtgccaacgcgcgttggatgcgcagttcgcacatctcgttctgacactcgtggcactgcagggcgcctaggatcaggtccgagacagcgccgcagcggtaggtacccatggcgttgttagtatcgaactggtcaaaaaattggggcgtaccggtgacttgcaacgcgcgacggcgtagcgagacggccacgcgcgagaaagagcacacgtaggccatggcgcggtgcatgggttgcgagaaggtctcgggcggacgcttctgcagatcgcagacgtcgtcgcgtagccaggcgctcatttgacctggctttttgactagccgtttgagcgtgctgcaatggtcgccccagccgtcctggtggtccaggatgcagcccaggtccaggttgttgagtttgttgaagagcagctgacgcatgccgcccaccgtctccagatagggatcgtgcgggttgacgggtagcccgtgcaggtggtggtacttcatgtagctgagcgtttcgtcgatgatggccagcaacgtgtgcaagttgggagcgttgtacacggcgaagatcttttccaccaccagcttgcgcagcaacggttcctccagccaatcgaactgttgacggatgtgcaacaggtagtcggtgtgcatgagctcgtcgtgtgacagcaggatgcgaccgcgcggctgatgatcttgcgggaaggcggtggggaccttgagatcggcggggtagggtgccagacgtagactctcggccgtgtagcgctgaaggtcgtagacgggcgaggtagaactcggtgaggtacccgacgaggcggcgccgcgctgcagacgcgctcttttcttcttttcgatcaaacggctgagttgctgtagttcgtcctcgtccatggcgtccagttcgtcgtcaataagcgccagcatctgttgttgttgcggtccggtggacgatccgtgatgattattggctgaagaggggtgagaagaaccgaaagtcgtaggacaactgggaactcggcgacgaagatgcgtcgaatcgccgccgtgatggtgcggttcgccgtcatcgttgtcgtaagacttaccgtagtgggggttaaggggcaccgaggcggacgcggccacgcgtcgcttgaaagaggaggacgccctatgtccgccacggaagcccgcggtgcccatgatgatgtgtccgccggtgcccccgagtgcgtggcgggaggagggtggaaggggaggaggatagtggtccggatcgccttcggtatcatcgtctttgctgtagcggggtcgtcgtgcggggacgcagggtcggtgatgatgcgaggcggcgccgacggtatcttccgcgagatggtattcgctggcggctgctccgttccgtgtcgacggcgaggttggacttcgctcgcgtcggaacttccgtggcacgggttcgtaatctagacagaagcgccgtgcgcgacgggcgcggcgttcgcgctcgctcagggaagataacgacggagcgtcgtgacggccgcgtgagtgcagctccatggccgccgtcgctaggaaggtcacgttcgggcacgctgatgtatatatagatgagaccgctgccggggggcgggtcaccggcgccgtggaaagtgaggctcagacggcggtcgccggcggcatgggcgcgtcgggcggtctgattttgatggaaatgtggacgtttttggcgttggagtgacactttttggtgaaacagcggctccagaggctggcccagagcgcgtagctgtgctcggtgcgcaggtcgatgaacacctgcacggtctcttgcgggttgcggtgcgtgtagttgagacagcgaaaatcccgcgtgcgcgcgccgtcgcgccgcttgacggccacgcagcaggcgccgtggggctgaaagaggaggacgtggggtgcggtaaactgctcgctgacgtgcggctcgtagtgttgcgtgaggtgctcgagcagcggcggccacacgcgggtgacgacgagccgctgcaagtccgtgtcggaaatcgcagcggcagtggcgccgtcgccaccgtacaggtgataggcgagcacctcggtgagaccgcggcgtcgataacgcgtcacgttaagcgagcgcgtctcgataaagttggcttcggtcgaggggcagattttgtcgcgcacgctgagaatgacgcgtggcggcggcgacaggggcaacgcgggcaggtcgtgcggcgggtggtggtgaagcaggttacgcagatccagttgggcgcgcacaaagcctagcgggtgttcgcggtaggcgtcgggcacgatgaacagcggcaacagacggcgatgcatgaaatagccgtcgtcttggtccattttatacatgtagggcagacgtacagagcgtccatggtggtagatgcctgtgtctaggctgctctcgggatgcgagatggggtccagcagcgtgtgcagttcggcgtcgagacagacggcgtgattgagcacctgcgccacggcgcgtaaaacgctggggtgtacggctacggtgcaggcggggaacggcgtgatgatgcgcagccccagtttgcccttgcagcggcagtaagggggtgacgtgtcaacggaggacgttggtttttgaaaaacgccgttatcagggacgttatttttgtcctctttcccgtcttcgtcttcctctgtgtcgcgctcgtcccggtaatcgagatagtcgtcgtcatcgaaaggcgcgccggccgcgtccacgggcacgctgttgggtgggcacgcgcttttgaagaaatagaccgggtgccggtcggggtgcgtgtagccaaagaggctcgcccatacggtcatccagacgcgtcgtagtccgcgacataattcaaagacggtgtgtcgcgccagaccggagacgccgtcgcgcagtcgtaaatcaaagtcggccacaaaattgaagacgggcagacgttcgttgaagacttcgtgtcgcgtgtagtagaactgtgtctcggggctggtgctggccacgtcgtcgtcgtgtagccacacggtctcggtcagggcctcgtccgagaaacggctgtcgggtacgtgacggagcaggtcgcgcggaaagaggctgcgatgccaggtttcggaggccacggcgcagaagacgtgctggtcattgggcaggtgtacgcggtagacgggcagcggtcgctccagcagcggtgccagcgcgggctcgggtagcaggtagcgacgttgcgagtaacgcgttagcgtgccggtggtgtaggtctgggctgtgcgcagcgaggcgcatagacgtaacaagccggacagggagcgttccagtggcgagaagacagactcggaaagcgtgttgatgcgttcgagctggcgcgccagctgcgtggaggtgccaaagaagcccgccaggtgcgtgccgtcgatgcggccgccgtagccggccagccccaggccgtgcgggctggtcgccgagtggggggattcgtcgagacccagtaggtgcgtctccacgtagtcgtgcagaaagttgtcgagcgagaagtatttttgcatgacgtccagcagctcggtggaaagccggcggcccagaaaacccggttcgcgcgtgcactgcgcttcgggcgccgcgtcagcgtcgtaagccaccacgcgccggtactcgagcaaccgcgcgcgtgccagcgccgtgcggtaggccaggtagacgtagtgcacgcagaccgtgtcgggcagacgcgcacgttcgcggaacgcgttgatctgcgtgtccacctgctctagctcggtgtagtcgcggcggttgcgcgccacggcgtacgccacgaaagcggacacgcgctgacggaagggcgagcccagtagcagacgcgcgaactcgcccatggaggcgtgcgtggggatgatggtgcccaggtcgcgcgtgcagaagctgcgcacgtactcctccacggtggagatggtgctgtactggccctcgaataggtagtaggccatggtcagcagcacctggccctcggtgtgcccgaagacgctgatgaaccacgagggcgaggtggggcagaggaagacctggttgagatgacgtagcacggccgcgtggtgaaagtacaccaggtgcttgaattcgcgcacctcgccgccgtgttcgggcgagagcacgggcgtgcggaagagatgccggtagagcggttgcgtctcggcctcgtccagactggcgatgagcgccgagagggggatgggctggcgcgcggccaggtagcgcgagagctgcagcgtttcgttgttcacggcgaagacgggcgccacccgccgcgagtccgagcacttttgcgtttgtaggcagaaataaacacgtcgcgagacctggtgtttgaccagcagggggaagacgcagtggtccgtcggtgtctgcgagagtacgttggcgactatatgagcagaatcatactctgttgcgaacagaacgagcgtcatcgtcgcgccggcacgatgcagctggcccagcgcctgtgcgagctgctgatgtgccgtcgcaaagccgcgcctgtggccgattacgtgctgctgcagcctagcgaggacgtggagctgcgcgagctgcaggcgtttctggacgagaactttaagcagctggagatcaccccggccgacctgcgaaccttttctcgcgacacggacgtggtgaaccacctgctgaagctgctgccgctctataggcaatgccagagcaagtgcgcgttcctcaagggctatctctcggagggctgtttgcctcacacgcggccggcggccgaggtggagtgcaagaaatcgcagcgtatcctagaggccctggacattctcatcctcaaactggtggtgggcgagtttgccatgtccgaggccgacagcctggagatgttgctggacaagttctccacggatcaggcctcgctggtggaggtgcagcgcgttatgggcctggtggacatggactgcgagaaaagcgcgtacatgctcgaggccggcgcggctgcgacggttgcaccaccgacgccaccggcggtcgttcagggggaaagcggcgtccgcgaggacggggaaacggtcgccgccgtgtcggcctttgcctgtccctcggtttcggactcgctgatccccgaggaaacgggggtcacgcgtcctatgatgagtttggctcacattaacaccgtctcctgtcccaccgttatgaggttcgaccagcggctgctggaagagggcgacgaggaggatgaagtgaccgtgatgtcgccgtcacccgagcccgtgcaacagcagccgccggtcgagcccgtgcagcagcagccccagggacgcgggtctcaccgtcggcgttacaaggagtcggcgccgcaggagacgctgcctacgaatcacgaacgcgagattttggatctcatgcgacacagccccgacgtgcctcgggaggcggtgatgtcaccgaccatggtcaccatacctcctccccagataccctttgtgggttccgcgcgtgaactcaggggcgtgaagaaaaagaaacccacggcggcggccttgctgtcctccgcgtgaacagcctggcacgttttggaaaacgtacgtgatcacggacacgacgagcacggggtttctcatagacgtactttattaggtcagggatgacggggaggtttcgggccgacgtcaaaaataacgtcactcgtgttgacagggctttctgcgtcggagctcttttcatcttcttctgtctcgtcgacgtcatcgtctaccggcgagggtgtccgttgcagcaacgcgtgctcgggcgtgtgggtgaaaccgatgtcgggggtgggcggcacgatcatctgtcctagggggtgactgcccaccggcagataggtaaagcggtgggtggtaaaaaccgctttggctacggtggtgtgtggggagatgcagacggtggtgtgcgaagtgttgaccaccgtcacgccggccgcggtacccgggagccagatggtgggtcgaatgatgagatccgattgactaaactggcgcacgcccactatgagggcgcagataccgggcgcgtgcacgtaggccgcgtcaaaatagacggtttgcgtgtgacccggaccgatcaccagcgtctgacgggtacgtaatgaaaagaaacggtgttcgttgggcggcggcaagttcatgagctgccagggttctggcacaaaacaggggaaaacgccgatatcgccttcgatggtgcccggaaagatggactgaaaagtgtcgttgaggttgacgacatccaactgcgggacttgcagcccggattccagcagctcgggcatgcaaacgaattgcgcgtccaggcatttgtaaaaggtaatgccaaaaaaaccttcggggatatagaggctgactcccagcgaggtgggcactttgcgctcgcgtgatagccaaatgatgtgtttattgtaaaaagccagctgcgtgtggcattgtttaacgatgaaactggaaggcatccacttgtagggaactttgagcggcgacggtaatggcgacgacgcttcatcctctcccggatgctgctctttgtcgtatttctcctcggtcgattggggcagcgtaaatgtggtttgaaaatcgctatcgctagcgaaacgcacgcagtaacgcatgttgacggatttctcggctaggatgatggagcctgatgacggtgcggactcttccttcattattaacgtaggggtctcccagaatcgctgaaaacgggagcgcggcagccgcgacagtaccagttgagagtcgattctgtcggtcaacattgtaagcatcgtggcggtggtgtgatggagtggaaaacagtgatactaggtgtttttgttttatcggtggcagcggggagttctggtaacagctcatccacgtcaacctccgcaactacgttaaaatcgtccagttctagcgtgtcaacaagcaaattgacgacaactgcgacaactacgacaactacgatgagtacgacctcatcgacaactaccactaaaccaagttccactactcacgaccctaatgtgatgaaacgacatactcacgatgatttttacaaggcacattgtacatcgcatatgtatgagctttcactgtccagcttcgcggcttggtggactatgcttaacgctctcattctcatgggagctttttgtatcgtattacgacattgctgtttccagaactttactgcaaccaccaccaaaggctattaagggtggacagatttacagctcgacggtgttccggcggggtaaggtttccataagtgggtgactggagactaaagttacggatctcatctagaaatagcagcgagtctagatagtcccacaggggatctataaacgttctctgaaatcccgttgatggtgacgtaggtgtagtttcggaagccgttttgttttccacgaacatggtttcgttgtaatataaggagctcatatcaagagtaccgtaaatagtgtacggtgtttcattacggattagtacatgcgtgtttttcataaattctgatacggcggttcggttgcggcttgattcacaaaaagggttttgccggtaacgtagagtggtatacacccacgtcgctaggtcccttaattgcgtggtcataatggacttcataaagctactatcaggacgataagcaattgtagacgtggaaacccgccttgcggtggtagtaacactataagttgcgttagtagtgacgttcagagcggttgacgttgtatagggagaatatggcgtagtagtactttgagatttcttactctttttttctgattgttctttgactggagcttgtttacgcttgagttttcgcatagtgtttttcaacttagtaccgttaatatacttagggacgcgaaataaatttcggctcatggcgttaaccaggtagaaactgtgcgtacagttgcgttgcgcgtaacgtagaagcaaggcggttaggcctaaaaagtagatcgtttgactatccacgtttactttcttggaacctacatataacttcgtgttccaacgtggcacattgaaaaacatggggttgaacgtggtgaaattgccgcagccttgttcgccagtatcattacgtttggaaacgtttagcatttcggaaagacaagtcatggaaggcaccgtaccgcaagatgggggtctgaatgttattgttttagccgtatgattgtattctgagaaaacgtacttagccggttttcgaagctgagtgctataaaaatcgaaccaaagataggtaacactgttattttgaatgggtcccgctaaaatgtaataccgtggaaactcggtcatgttcatagtcagatttttaatgtgttgtctggtcatattaaagtattttgtatagatatccttttctagttgtttcaaaatctctaatttgaacttgtctagtctttgcttgcctatcgtagacagtactttacctgaccagtaacgtcctacggataatcgtaccgcagccctacagtttatgaaagagaatagcaggaaagttagcgacataaggaagaataaattaaaaacacctctcatctctccttttctccccatgacagaggaggagaccccgcaccgtccgtctgccttgtggtttggcttgcctgcgtgtactcactgctgattctggtcgttttgctgctcatctaccgctgttgcatcggcttccaagacgacctagtttcccgcaccttggctgtgtaccgagcttgtatccagggaccgatatgtaaccagacccacaacagtacctcgtaaataaagacgcacacacctcacgcatatagtaccatcacaccgtgtggcgtgtactttattacaacgagcaagagtgccccctaactattggggcccgtaccgttttagaaggttttgtgtgaatgtctttaacttctctgtcccttttctcataaactgtcaggtcctacagtcagcatgtcttgagcatgcggtagagcagatagatgccgatgatggccgatagcgcgtagacggacatcatgaggagacgactgtcggtggcgtccacgacaacgtcagttacttctaggaccgtaccgtttttcaaaagcatgaggtagtgagttcgcggagatgagaccaccacttcgttgtagggatccagggcgaaaaggacgtcgtccgagtcgtgcatgtacatgatgttgatgacgccttgcgtgtcgtcgtattctagcagggcgctttggcaaaaggcgcagttttctagggaaatgttgagcgccgctgtgatgctgtgtgtggtatgcatgttgcgcgtcagttcgcatttagtttgactgtccgtctgggtgatgatgaggctttggcctacgacggtggtggagacagggtaggagatacctttgatcaggtactggtttgttacgacataactgacgtgttcggagacggtcagcgcggagaaggattcgcctagtggcagacaaaacaggtcggggaaggtttccaacgtgcttggttgcatggtagataggatggagagggcggcgggaacggtagtggggacggtggcatcggggaagagacgtgtgaggcgttcgagcgagtgatcgcgtcgcccgctactggaacagggtgtgtacaggtcgctgaggtattcgtggtgcggatgagctagcaactgcgtaaagtgtgatagctcggccaatgaacagaggcccgtttctacgatgaagatttcgcgtctctccgtcgtatgtaccagcatggagtggacgaggctgcccatgaggtagagttcttgacgcgcgaaggctgaaagaaaagaggccaggtgcgttttgtgtagttttagggcaaagtcggcgatctgtcgtagtgcccactgggggatgagatgttgctgattctgtttagagagtatgtagaccaggcgtacgaggctggtgatgtcggtgatctgattcggtgtccaaagggctcgtttggccaggtccacggccgtgggatacagtagcaacgtggtgcgtggtggtgtttgtgagaggcaggtgatcataaattcttgtatttgtaagagtgcggcctggcggtctagggcccgtgggacggagacttgggcgccggcctcttcttgtcgggctgctgcgaacagtgctaatgcgtaggcgaaggccatttctaccgtgcggcggtccagcatctgacatcgaccgctcttgagtacatccacggcgtaacggtgaaagctgttacgtagtagtgcgctgaggtctaggtagttgaagtcaagtgcggcgtcaagaaagtccgggtctttgagataagagtgacggttcagttgatctttcttaactagcaccaggagctcgtgtttttcagtttgtcgtagtataaagttgtcgcgttgatagggcgctttgaaaagtacgcgtggaagatggccgaagataagcagcatgggtgtgtcgtcgtctatggacaccgtaactacgaagaagtcctcggtcagtgtgattttaacgtaacgtagttcgtcgatgaggtaaaagccttggtgcaaacaaggtgtgacggtgctgaatagtagatcgtgtccatcaaagaggatacaggtctggttaaagtgtggtcggtgtagtcctgaggtggtatgtgattctgtccagccgtgtggagtggtttgcggtggcatccaaacgtgaggtattgacaggtcaatgggcggtggcacagtggtgggctgttcacctaggctgtcttgtgcctttagctgctgcgaaaaagatcggtagctggccaggtctttggataccagcgcgtaagtgttaagtctctgttggtatctttccagggtttcggtcagatctacctggttcagaaactgctccgccagaggacccgcaaaaagacatcgaggcatatggaatacatagtattgattatagctttggaaaaagttgaaactgatggcgttttccctgacgaccgtgctgttacggaggctgctgttgtaggtgcactgggtggtgttttcacgcaggaagcggatgggtctcccgtaggtgttgagtagtaggtgaaacgcgtgagggtccagcgcttcggatgcggcgtctgcgccatatcgttgcgaaggtaggtgactgaggaggtagacggcgaagacggtgaggtaggaggggaggccgggccgcatagcgcggccgcgccgctgggttcagcggcgtgatccaggtggtggttggcgttacacccgagagaaggagaaaaaggatcccaggaaggagcacccgggtgcggcgctacgggttacaaaagtcgcgtctccgtctatttaatacgatgtcattggccgctgcgaagggagaagaggggacacgcgaataagccatgccgtccgggcgtggggacgacgctgatttgacggggaacgctctgcggagattgcctcacgtgcgtaagcgaatcggtaagcgcaagcacctggacatctaccgtcgtctgctgcgggtctttccctcgtttgtggcgctcaaccgcctgttgggaggccttttcccacccgagttgcaaaagtaccgtcgccgtcttttcatcgaagtacgattaagtcggcggattcccgactgcgtgttggtgtttttaccgccggactctgggtcgcgcggcatcgtgtattgctacgtgattgagttcaaaaccacgtactcagacgccgacgatcagtccgtgcggtggcacgccacccacagcctgcagtacgccgagggcctgcgccagctcaagggcgcactggtggactttgattttctgcgtctgccacgcggtggcggtcaagtctggagcgtggtgcccagtctggttttttttcagcaaaaggccgatcgcccatccttttatcgggctttccgctcaggccgttttaacctgtgtaccgattctgtcctggactatctagggaggcgtcaggatgagtctgttgcacaccttttggcggctacccgtcgccgtcttcttcgagccgcacgaggaaaacgtgctgcgctgccccgagcgcgtgcttcggcggttgctggaggacgcggcggtggcaacgcgcggcgggggctggcgcgaggacgtgctcatggaccgggtgcgcaaacggtatctgcgtcaggagctcagggatctgggtcacagggtgcagacttactgcgaggatctcgaagggcgcgtgtccgaggcggaggcgttgttgaaccagcagtgcgagctcgacgaaggaccgtcgccgcggacgctgctacaaccaccgtgtcgtccgcgttcgtcgtccccagggaccggcgtggcaggagcttccgccgtcccacacggtctttatagtcggcacgatgccatcacgggacccgccgccgccccgtctgacgcggcgaccgcgtcagcggccgccggtgcttcttctacctggctggcgcagtgcgccgagcggccgttgcccgggaacgtacctagctactttggaatcacgcagaacgatccctttatccgctttcacaccgattttcgtggcgaggtggttaacaccatgttcgaaaacgcctccacttggactttctcctttggtatctggtactatcggctcaagcgggggttgtacacgcaaccacggtggaaacgagtgtaccatctggcgcagatggacaacttttccatttcgcaggagctgctgcttggcgtggtcaacgctttggaaaacgtgacggtgtatccgacgtacgactgcgtactctccgatttggaagccgccgcctgtctgctggccgcctacggacacgcgctttgggagggccgcgatccgccggactccgtgacggcggtgttgagtgagctacctcagctgttgccgcgtctggccgacgacgtgagtcgtgagattgccgcttgggaaggccccgtcgccgtgggtaacaactattacgcgtatcgcgactcgcccgatctacgctactacatgcccctaagcggtggtcgccactatcacccgggcacttttgatcgtcacgtgctggtgcggcttttccacaaacgcggcgtcctccagcatttgccgggctacgggacgataacggaggagctggtgcaagagcgtctgtcgggccaggtgcgcgacgacgtgctttctctctggagtcgacgtctgctggtcggcaagctgggtcgcgacgtgcccgtctttgtgcacgaacagcaatatctgcgttcgggcctgacctgcctggctggcctgctgttgttgtggaaggtgaccaacgcggatagcgtcttcgctccgcgcacgggcaaatttacgttggccgacctgctgggttcggatgccgtagccagcggcgggttgcccggggggcgcgcgggcggcgaagaggagggctacgggggacggcacgggcgggtacgtaactttgagtttctggtgcagtactacatcgggccgtggtacgcgcgcgaccccgcggtcacgctgtcgcagctctttcccggcctggctctgttggccgtgaccgaaagcgtgcgcagcggctgggatccctcacgtcgcgaggacagcgccggaggtggcgacggcggcggcgccgtgctcatgcagctcagcaagagcaaccccgtggccgactacatgttcgcgcagagctccaaacagtacggcgatttacgtcgcttggaggtacacgacgctctgctctttcactacgaacacgggctagggcggctgttgtcagtgaccctgccgcgtcatcgtgtgtccactctgggctcgtccctctttaacgtcaacgatatttacgaactgttgtactttttagtgttgggttttcttccgagcgtggcggtgttgtaatttccaccacgtgtcgctcgctgcataaagggcgagcgtccccggagagggtatattcgtttggcgagagcgggcagcggtggtgggtaccatgcacggccccaaggccacactgcaggatatcgttctgcacctggaaccccagaacgagatccccgtggatctgctgggccatggccagctgtctgactccgaagaagagaacgacgagatcgacggcgtgaaccaccagcatctccctgctcgaagagccgagcctcaacggcacaccatgctgtgcatgggatgtaaatgcgaagccagaatcgagctggtggttgagagctcggccgacgacctgagagccttccagcagctgtttctgaacaccctgagcttcgtgggcccttggtgcgccagccagcagatggccagatttgaggaccctacacggcggccttacaagctgcctgacctgtgcaccgaactgaacaccagcctgcaggatattgagatcacctgcgtgtactgtaaaaccgtgctggagctgacagaggtttttgaattcgccttcaaggacctgttcgtggtgtacagagatagcatccctcacgccgcttgtcacaagggcattgacttctacagcagaatacgggaactgcggcactactctgattctgtgtacggcgacaccctagaaaagctgaccaacacaggcctgtacaacctcctgatcagatgcctgcgttgccagaagccactgaacccagccgagaagctgagacacctaaacgagaagcggcgcttccacaacatcgccggccactacagaggacagggccactcttgctgcaacagagctagacaggagaggctgcagagacacggcgacacacctactctgcatgagtacatgctggacctgcagcctgaaaccaccgatctgtacggatacggccagctgaacgacagcagcgaggaggaagacgaaatcgatggccctgccggccaggccgagcctgatagagctcactacaacatcgtcaccttcgggtgtaagtgcgactctacactccggctgtgtgtgcagagcacacacgtggacatctgcaccctggaggatcttctgatgggcaccctgggaatcgtgggccctatctgcagccagaagcctatgttccaggaccctcaggagagaccccggaagctgcctcagctgtgcaccgaactgcagacgacaatccacgacattatcctggagtgcgtgtactgcaagcaacaactgctgcggagagaggtgtatgacttcgctttcagagatctgtgcatcgtgtacagagatggcaacccctacgctgtgtgcgacaagggcctcaagttctacagcaagatctctgaataccggcactactgctacagcctgtacggcacaaccctggagcagcagtataacaaacccctgtgtgacctcctgatcagatgcattaactgccaaaaaccactgtgcccagaagaaaagcagcgacacctggacaagaagcagcggtttcacaacatcagaggcagatggaccgggcggggaatgtcttgttgcagaagctctagaactccaaagctgagatctacagccgctgcactgtactggtataagaccggcatgtccaacatcagcgaggtttacggcgatacccctgagtggattcagagacagaccgtgctgcagcactccttcaacgactgtacatttgaactgagccaaatggtgcagtgggcttacgacaatgacattgtggatgatagcgagatcgcctacaagtacgcccagcttgccgacaccaactccaacgcttctgccttcctgaaaagcaacagccaggccaagatcgtgaaggattgcgccacaatgtgcagacactacaagagagctgagaagaagcagatgtccatgtcgcaatggatcaagtaccggtgcgatagagtggacgacggcggagattggaaacagatcgtgatgttcctgcggtaccagggcgtggacttcatgagcttcctgtctgctctgaaaaaatttctgcagggcatccctaagaaaaattgcatcttgctgtatggcgccgctaacaccggcgccctgtttggcatgagcctgatgaagttcctgcagggcagcgttatctgcttcgtgaactccaaaagccacttcgccttccagcccctggctgacgccaagatcggcatgctggacgacgccacagtgccgtgctggaactatatcgacgacaacttgcggaacgccctggacggcaatctggtcagcatggacgtgaagcaccggcctctggtgcagctgaaatgtcctccactgctgatcacgtccgccatcaacgccggcaccgatagcagatggccttacctgcacaatagactggttgtcttcaccttccctaatgaattccctttcgatgagaacggcaaccccgtgtacgagctgaatgataagaactggcccaagctgagaagcagcgtggccgctctgtactggtaccggaccggcattagcaacatcagcgaggtgatgggcgacacacccgaatggattcagagactgaccatcatccagcacggcatcgatgacagcaatttcgacctatctgagatggtccagtgggcctttgacaacgagctgacagacgagagtgatatggcctttgagtacgccctgctggccgattctaacagcaatgccgccgccttcctcaagagcaattgccaagccaagtacctgaaggactgcgccaccatgtgcaagcactataggagagcccagaagcggcagatgaacatgagccagtggatcagattcagatgtagcaagatcgatgagggcggcgactggagacctatcgtgcagttcctgcggtaccagcaaatcgagttcatcaccttcttgggcgccctgaagagcttcctgaagggcacccctaagaaaaactgcctggtgttctgcggccccgccaatacaggagcctacttcggcatgagctttatccactttatccagggagccgtgatctccttcgtgaacagcaccagccacgcctttctggaacctctgaccgacaccaaggtggccatgctggacgacgctaccacaacctgctggacctacttcgatacatacatgagaaacgccctggacggaaatcctatcagcatcgaccggaagcacaagccactgatccagctgaaatgcccccccatcttgctgaccaccgccatccaccccgctaaggacaatcggtggccttacctggaaagcagaatcaccgtgttcgagttccctaacgctttccccttcgacaagaacggcaaccctgtctacgagatcaacgacaagaattggatggaaactttgtgccagagactgaatgtgtgtcaggacaagatcctgacccactacgagaacgacgccaccgatttgagggatcacatcgactactggaagcagatggcactcgcctgtgccatctactacaaggctcgcgagatgggcttcaagcacatcaaccatcaggtggtgcccacgctggccgttagcaagaacaaggccctgcaggccgctgaactgcagctgacactggagaccatctacaacagccaatacagcaacgagaagtggaccctgcaagatgtgagcctggaagtgtacctgacagctcctaccggctgcatcgccaagcacggctataccgtggaagtgcagttcgacggcgatatctgtaacaccatgcactacacaaacagaaagtacatctacatctgcgaggaaacctccgtgaccgtggtggaaggccaggtggactactacggcctggcctacgtgcacgagggcattcagacatacttcgtgcagttcaaggacgacgccgagaagtactccaaaaacaaagtgtgggaagtgcatgctggaggacaggctatcctgtgtcctacaagcgtgttcatgcagacacctaaagagacactgagcgagcggctgagcgccttgcaggacaagatcatcgaccactacgaaaacgacgccaaagacatcgatagccagatccagtactggcagcttatcgcctgggccaacgcgattttcttcgccgcgcgggaacacggcatccaaaccctgaaccaccaggtggtgcctgcttataatatctctaagtccaaggcccacaaggctgccgagctgcagatggccctgcagggcctggcacaatccgcctacaagaccgaggactggacactccaagacacatgtgaagaactgtggaacacagagcctacacattgttttgccaaaggaggccagaccgtgcaggtgtatttcgacggcaacaaggacaactccatgaattacgtggcccgggattctgtgtactacatgaccgacgccggcacctgggacaagaccgccacctgcgtgtcccacagaggactggcctacgtaaaggaaggttacaataccttttacatcgagttcaaaagcgaatgtgagaaatacggcaacaccggaacgtgggaggcgcactttggcaacaacgtgatcgactgctgagcggcggcgctgtacgccagcggggagaaaagtggcagataaatcacgtcaggttcacacgtcgttagccagcgtcggcatatgaagggcgcgggcggccagtacggcctctgggctgagacaggacgaggcagggtgagaaagaggaggatgggggggaccggggtggtggtgctgctgctgttgtgggtgtggacggtgcggatgccgggacagcgtgccggcgaacgttctgtaatcttccataataaaggtaaaaatgcccgtctcgtgtcgactccgctggatctcgaaggcgtcgggggtaatgcgcatcttgccggtgccgatgagataaaagtaccacattttttgacagatgatgcgaatcaagggttcgtacgcttcggcaccccagtggcgcgtgaagaaggccgccagacgaaacaagcggtgtccgtagagcgtgcctagggagaagaggatgttgccgttgcgcgccaggtcttcggggaaaacgaccggcaggccggtgtggcgctgcacaaagcgcgtcagcagtccgccgctcaagcgcgggtgacacaggcgctggctgagacgggcggcgcgcgtttcatcgaacacggccgcctcaaagtccagccccgggaaggcctgacgcagttcgcggtacagatgaggccagtagggttgcggcgtcttgcgactaagcacggcgtggtccgagacgcccaggttgttcatggtttcgcgcagtagcagcgtttcgagaccgcggtgaaagaggaggacgcagatgaggcgtacgattttgagttcttccaaacgcagcgagctcagcggctgtccgcgcgacatcttctcgctaatctgtaatattagatgattggcgcaagtaaaggagaatttgcccgtgcggacccgcgggacggcggggttctcttcgtcgcgggccatcatcgttcgctcggtgagcgggtagcgacggtgacgacaatgacgatggacgagcagcagtcgcaggctgtggcgccggtctacgtgggcggctttctcgcccgctacgaccagtctccggacgaggccgaattgctgttgccgcgggacgtagtggagcactggttgcacgcgcagggccagggacagccttcgttgtcggtcgcgctcccgctcaatatcaaccacgacgacacggccgttgtaggacacgttgcggcgatgcagagcgtccgcgacggtcttttttgcctgggctgcgtcacttcgcccaggtttctggagattgtacgccgcgcttcggaaaagtccgagctggtttcgcgcgggcccgtcagtccgctgcagccggacaaggtggtggagtttctcagcggcagttacgccggcctctcgctctccagccggcgctgcgacgacgtggaggccgcgacgtcgctttcgggctcggaaaccacgccgttcaaacacgtggctttgtgcagcgtgggtcggcgtcgcggtacgttggccgtgtacgggcgcgatcccgagtgggtcactcagcggtttccagacctcacggcggccgaccgcgacgggctacgtgcacagtggcagcgctgcggcagcactgctgtcgacgcgtcgggcgatccctttcgctcagacagctacggcctgttgggcaacagcgtggacgcgctctacatccgtgagcgactgcccaagctgcgctacgacaagcaactagtcggcgtgacggagcgcgagtcgtacgtcaaggcgagcgtttcgcctgaggcggcgtgcgatattaaagcggcgcccgccgagcgttcgggcgacagccgcagtcgggccgccacgccggcggctggggcgcgcgttccctcttcatccccgtcacctccagtcgaaccgccatctcctgttcagtcgcctgcgcttccagtgtcgccgtccgttctccccgcggaatcaccgccgtcgctttctccctcggagtcggcagaggcggcgtccatgtcgcaccctctgagtgctgcggttaccgccgctacggctcctccaggtgctaccgtggcaggtgcgtcgccggctgtgccgtctctagcgtggcctcacgacggagtttatttacccaaagacgcttttttctcgctacttggggccagtcgctcggcagcgcccgtcatgtatcccggcgccgtagcggcccctccttctgcttcgccagcaccgctgcctttgccgtcttatcccgcgtcctacggcgcccccgtcgtgggttacgaccagttggcggcacgtcactttgcggactacgtggatccccattatcccgggtggggtcggcgttacgagcccacgccgcctttgcattcgtcttatcccgtgccgccgccaccatcaccggcctattaccgtcggcgcgactctccgggcggtatggatgaaccaccgtccggatgggagcgttacgacggtggtcaccgtggtcagtcgcagaagcagcaccgtcacgggggcagcggcggacacaacaaacgccgtaaggaagccgcggcggcgtcgtcgtcgtcctcggacgaagacttgagtttccccggcgaggccgagcacggccgggcgcgaaagcgtctaaaaagtcacgtcaatagcgacggtggaagtggcgggcacgcgggttccaatcagcagcagcaacaacgttacgatgaactgcgggatgccattcacgagctgaaacgcgatctgtttgccgcgcggcagagttctacgttactttcggcggctctccccgctgcggcctcttcctccccaactactactaccgtgtgtactcccaccggcgagctgacgagcggcggaggagaaacaccgacggcacttctatccggaggtgccaaggtagctgagcgcgctcaggccggcgtggtgaacgccagttgccgcctcgctaccgcgtcgggttctgaggcggcaacggccgggccctcgatggcaggttcttcttcctgcccggctagtgtcgtgttagccgccgctgctgctcaagccgccgcagcttcccagagcccgcccaaagacatggtagatctgaatcggcggatttttgtggctgcgctcaataagctcgagtaagagagacgctatatttagggtttccctctcttttttttttctacaccgtgataccctaataaagcacactgcggttattatcaacgtctctgtgtttttattatttagaaataaatacagggaatgggaaaaacacgcgggggaaaaacaaagaagtctctctctagatgcggggtcgactgcgtggggtgctggaagtggaagcggtgctgatgggtgagggtcgtggcgcgggcacggaccgcaacgtgctgctgatgtctgccgcggtacgcacgtcgccgtccatgtcgctgcgcagataagaggtaggtcgtaatgcggcgtgctgcacgctcaccgttaatggtaccaagtcgtcaaggctcgcaaagacgtgccacgaggggatgacgagcgtgagagccccgttgttaccgcttcgacgtctttgtccggtcaggatcagtgcccgggacagtccggcttgggtgtccgagtcctcgtcgccgctggcctcctcgaagccggcaaacatggcttcggacaggggggtcggcgtcggtgtggaggagaggtcatcttcgtcgtcctcttcctcttcttcctcctcttcctcggtgggtggtaatccgggggactgcgggagaaactcggagacggcgccgcgcacgacgtcgctccgtggaaagagaccggcgcgcagctgcacctggggacgcttgattttgtccggtttaccgggtgtgagagtccaaaacccacggcggaaaaagtggatgcggcctagcggctgtcggtgttccaaatgaacggcctggtcgccggtcagcgtgacgcggagggtgattcgcacacgatcgggtagcgggccggcttctatggagacgcccgggatgttttccgggaaaaagatggtgtcgtgagtctgattggtttcgaaagcattctggatctgcacgatgtactcgggatgtatgcgcgtcagcgtaaaacttttgggaatcaacagctggaagccgttgtccggcaagcgtcgtaggtgcgggtacggattgtgtcgcgccaccacctcggcgcgatgcgtgtaaaccgaaaagtgcagaaacacgctggtcggcgggtgcggtgagtcgtgatgcagaaacagcatgatccattggcctcgttcgtccgtctccgttttgtggatgtacgtgttagggtccgaacaggccagctgctccagggcgtctaccagcgtcagcgggatggcgccggcgcgaaaggcgaactggctgacaaagatctgccctgcctccaaactgctgtcggttctgcggcgccagttcggcgtcacggtcagtcgcacggcccagtggtgagccgtgcggcggatgatggcgcgcgcctccattcgcggccgattttcttcgccgccgcgccgctggctctgaaagaggtgcagtccgctaacgggcacgcggtctagcggcagcgcaaaggccagcaccgagaccgtgttgttttctgagcctggcgtcaggcgtcgtgggccaaagttgttgaggtccaccagcagtcggtcccgttcgcccaccacgcagcggcccttgatgtttaggtcggtcaggtctacggtgtcgtgcggagatttgttctcctgaaaacagcagagaaccgagggtcggctcacctctatgttggtacgcaggtccaggagtcgcagacgaccggcttccagcgagccgccttccacgttggtgatgagccgaagcacctggcagtgcaggcgaccaaagctgccgctggcggcttcggcctcgctgatcgcggccgcttccgacgagggtccctcaccgggcgaggacgatgcctgagacattgcgaaggcgggatggggggagggtcaggggatgcgcaaaggtgaacgggtcttcgtgggaggtcgggaagggttccggcaactgtcgcaaatatagcagcggcgacaggtgtggcgcccaaaagtcgcgtgtctgagtggacgtgggtttttatagagtcgtcttaagcgcgtgcgcggcgggtggctcaacctcgatgctttttgggcgtcgaggcgatgcatggcccgggcagggcttcttgccggtggcggcgacgtttgggttgcgcagcgggctgccatacgccttccaattcggcgaagatgcggtagatgtcgttggcgtcccagaagaactcctggtacttcagattctgaccctgaaccgtagccaccatgggcaccaggttgcgggccaggatgccggcctgccagggcggccaggtgaacacggccggattgtggatttcgttgtcggaatcctcgtcggtgtcctcttcgggcgcgacggtggactcggccttaaggcggccgcgtgtcataacgcccgccgtgcacgccgtcgccgaggatgctgatttgcgtttgcggcccgcggaagtggaggcgcccgccatggcgccgccgccggtaacgcggggcgtcttgcgctcggtggttacgagttcttcgtcggagtccgatccgctggtccagacgtcgtcgtcgccctgggcggcaccctcgtcgtgccggtcccaggtgtgtcggtactcaagcttgccctggatgcgatactggctggtgaaggtggggtgctcgctgtactgaggcccgcgctgcagcagcaagtcgatatcgaaaaagaagagcgcagccacgggatcgtactgacgcagttccacggtctcgcgtatcgcttgtacctccaggaagatctgctgcccgttcatcaacaggttacctgagatgctcaggcccgggatgctcttgggacacagcagcccaaaatgctcgtgtgaggtaaaagccacatccagcatgatgtgcgagatcttgcccggtttgattatcatatttttgggacacaacaccgtaaagccgttgcgctcgtgggggcgcatgaagggttgcgggttgcgggtcatcgtcaggtcctcttccacgtcagagcccagcgtgacgtgcataaagagcttgccggagggcacgtcctcgcagaaggactccaggtacaccttgacgtactggtcacctatcacctgcatcttggttgcgcgcgtgttctccatggagcaaaccagctcgtgcgcgcacaccacgtgccgcagtgccacgtccttggtgggaaacacgaacgctgacgtgtagtagacgtcgggctctttccactggttctgctgacgcgtccaggccagtcccgagaccgtgagacgcgcctgccacatctgcttgcccgacgcgtgaatcacagcgtcagctacgggcaggtgtcggtgtttgcgctcggccgccgacgggtagtggtgcacgttgatgctggggatgttcagcatcttgagcggcagcgcgtacacatagatcgacatgggctcctggctggggcagatgcttcggcccgtggggttgtgcacgttgaccgacacgttctccacctcgctgcccgtaaagtacgtgtgctgcacctgcagctgattgtcgccgcggtggcatggcgtcgagtcgggcgtgtactgcgacaccaggatcagcgagggctggctcacgcgtacgtggatacccgtctgcaggagtcgcgtctcgtgcggcagcaccggcgtgtcgccgcgactaaacacggctttcagcacgtgccccgaaatgggacccagtacggatatcatttcgggacaacggcgaccgcgcgactccatgctgcctgcgcgtacgggtgtaggcgactgagcggcgcgccctttgcggccgccgccttacataggcaggcgaccaaacgcggaacccgaaataaaaacgttctacacagagacaaccgcggattattgagtgtctttttttattacaaaaaaaaagaggcaaagccccaccgtcaccacaccccatcacacaccaccaccgattttttttgttttaaccccgtatcgcgcggacgcctagtgtccgtttcccatcaccagggtcctctgtttagagatcgccgcagaccatggctaaagtgacaggactcgttttctctgtcgtattttccgtgagcttacagtcttgcggttccgtctccggggacgccagtcgcatgggcagcaggtcctccagcgcgatggaagcgcccagcaccgagagctgctgttgcgacggcgaatgggacgtggaccgcgagtgtagcgtggatttgacttggtgcgtcattgctgacaggcaaccgcgattcagcgtatgctttgacgagataaaatagaggcgtcccaggagcgcgtcccgtgggaacgtggcgccgttctcgtcgctcaccagtacggttaattccaaccaggagcgcggtagccagaccgtaacgggcattttgagtccctgacggttgtgtggtacaaaaacacccagataaggcccgtaaaagcggcggtagatacgtaacgtgtgcgagtttttcagcgtcaattcgtaagggacgcgcacctccagtccctcgtccgccgcaccggagcgtggcggtacaaagtaaggcagtggcgcgtccgaaaagaagggtcgtcgcaccgtttcgcgtcgcagccgcaggcgaaacgccactgggtcggctggcgcctcggtgcggtcgcaggtcacgttgaaacgtaacatgccgtcttggtatagcgtgagtgacgacagcgtcaggtccggcggtgattcgttcgggtctagctccaatcgtccaaagacggagggtcccaatgtcttggcagtggtttccgagaggcgcgccgagatacggctggtgagtccacgcggccccgagatgccgccttccactcgatgccagcacagcgcgtgtcgtacgcgcaccgtcagcgtgggcgtcagatccgcgtccgttgattccgcggtatcagcgacggaagccgcgttctccgttacgttgtttatatccagcgtcggctcgaacgtgagttctggcagatgcagcgcaagacagtcgtgtaacgccgtgtgatgcgcggctttacgtcgtagcggtagccgtttcagcagcggcgtgatgatacggagcgcgaagagattgagtgataagcgcacgatggccatgcgcgtcagttgctggtcgattactgagcgcaggatatggcagcctgggcgtgcgggaaagagagagaaggccgggcgtacgtcagaatcctcgttagataccacgcatagaatgccgcgttcacgatcgtcgttgcggtcatcctcgtcctcttcttttttcttctctttttccttttttttctcgggctcatgggaagccgccgtttcttcttcttgcgacgtcgcggagtcggtttgagactcgccgttcgcttcccccaattgcagcggcgtagagagcagaatctggaagggatcccgcaattcttcgggtcggaggtcgaggtgcaactggatcagatggtaggtgccgcggtgcacccgaggctgacggatgtcgtgtttatccgtcagtgtgaggatggtctgcggcgagccgctgtgcttgtccagctcgtccggcgttttcaggaggaggctgtcgtcgtcggtactggcgacgcccatcatggtcgtggtggtagtggtggcgaggaaagtgagcggcggcgctgacagagctcggcgttggcggcggcatttgccgctgtgtcggctgctattgctgccaacgccaccgccgccgcctcgtctggctcgtggccggcgggcccgattccgaaggttggggtcgacgcgtggcatgcttggtgtctgcgggcgcgagagggctggctcagcctttaaatatgcaggtcgcggatttgttatcgggtgaaacgtcacacaccgtgaagacgacctgttcgcggatgaggtcatccagctgtcgcagcatgacgaaaagcgccgacagccgcgcgatctcgtcgtcgggcgacacgtgctgcggccgcgcgggcgtgcgcggctcgccgacgctgcgctcgcggtccagccgcatcagcagctcctggcacttgacgagcagcatggagctgtcctctagcgccaacttgcgcacgtaggtcatggtcagctccgaggctaggttggccaccatggacatggagaggcaggcggtcttcatgtcgatcagcaggtgctggtcgatgaccggatcggggatggtgaaggtggcgtcgcgaaaagtaatggtctgcagctgctgcacggcagcctttacctcctcgtacgaacggtcgagcgagaagaggcccatgatgagtagtcgctggttgatttccagcgccagtggcatgggtacgatccagggcagcaccagctcccactggcccagcgtcagcaggttctcgcgcgccagcggtccgtggaagagcggcggcagcacgcatagcgcgtcgcccttctcccaagtcacgggtcccgtgttgaggacggtgtagagcagtccgtgcgtcggtacgtgtaggaggatctggttgccttctacgcgccgcatcaacgtcagcgtcatattgcgcagcaggccgcgcagtcgtacgtagccgcgggtgtgatctacgaactggtgtaggcccagctggtagtgtttgatgagatgtagacgctgcggaatgggcacaacggccgctactagcttggtcagtttgcctacgtcggcgatgctgagcttgtggtcgaaagtgcagaagatgttggcctccatggccgccatagcggcggtgaaatcctggccgcgacggaggagaagcagagacgaacaacgtctgcaccgggcgcggcgtcagagcgagcgtggcgcgtccgggcccgcgtttgcgtctaggtgattcgccgttaacctgcggtcgtcgccgtcctcctcaccggacggcctcacgagttaaataacatggattgctgtagcgggatgatttcgcctacgacgtagttaccaaagtgcgtttcggacgtggcaaaagccccggcgccacccttgagtttggtctccatcagcgccagcgtggtggtgctgaggatcggtagcgcttcctgcgtcagacggcacgggttttcgatgagttgttccgtgccttcgacgcagacgtactgcgtgtccgtgtcgccgcggatgcagtccttggcgcgtagcaggtactcgtcgatggttttgaagagcgttttgttggccgcgataatctcttccgtgttaaagtactgcgcgcaggggctgtagaatttggagttgtagcctagacgttcgcgatgtcgggtgttgtagagtacgtcgctcagacagccggcttgcgaggcccaggggttgtgtgtggccgcgaaagtctgtgcgtccgcttcgcgatggtcgtagatggccttggtggcggcctccgtgtcgtacggatcgacggccagcatgcaggaggcacgcccgcgcgggttgttggggatcttaaagtaattaacgtccatcgtcaccggcgtaaggattagttcgcacgcggccttttgtccgtgcaccgtggcggcggcattgcgctcggacatgctgccgaacgtcagcatggagatggtctccgtgtctaacagttgcggccgttctacgccggccgcgtgccggatccagcggtccacctcgtcgtgccggtacacgttcatagggaagacgcgaaagaggtcctgcacgcggacgcccatgtcggttcgcacgcggtttacgtaggctacgcaggtatttgacgtgtaacccagacccatgtctacggtgttaatgttctgcgtgacgtggtacgtggtgctgatgtcgcgttcctccttggtcacgatagggttgttgatgataactgacgtgcacgatttgccgctgtagagcagcatgtccacctcgaaggtgtcggtgcgtacggccgtgagtgcgaatcccgggtggatgtgcgccttggtctgcagcaccagtgaaactggtgagattttgtataacatggcggccagcgtcatgactgagtgcaacacgttgggacaggtggccgagtaacgcgaaaagggcgagcgcagccagttgtggtactcgtgtgcgaaggctgtgggtagcgggaaaccaccgtcgtgacggtgatagtgcgggaactcggtcacgtagcgtttaatgtcgtcgctcaacgccgcgcagatggtggggtttgagtagaaacggtggaaaggtacgggtaggctgtactcgatcaacgtcttaggcgccgtcacgacgcagcagccgttgtaaagcacgtgctgacgtgagataaagtccggcaggccctgacgttgcgcgtggtccagaggcgcgcgcacttcgagcaccttgacgtgctcgcccacgaattgcacggccaaaaacagttcacgacaggcctgcagcagcggcgtgtgcgcgtcggtggcgacgtcctccaccagctcggtcagcatctcgcctacggcttgacgttgcgccgctatcgagtcttcgggggtgacgccgcttgtgctctctttcgacgtcgtacctgacgtggagaccgcggtggcggccggcatcaggagaaacgccggtcggtaaaagaggtctactagcagcgtcttgaggttgagtcccaggccgcaggcccggttgttggtcatggcgggcatgaggcagagataaaagaccttttgtaacgtccattcgtcgtcggtggcacggtaatcgtccacaaacagcggctcgtcggcatccatggcgcccaaacgcggtacgtcagaaacgccgtggtgtcgcgcctcgatgttggccgggttcaacggttgccggtcggccactacctgtacgccttccatgttacgcggcaggtgcgtaacgaaggggggccacagccggtggtcgtgcagcgcgttcacgtaagccgatagcggttcctcagccagttgaccgttgttaagtcccggcagcgctgagatgcgcgttaccagacgcagcacggcgaccagattgcggtagtgaaagagcaactgcggtggtagggcgccatcagccaggtgttcggcgatcaacgtcaccagcgcgtagctgtgcgcaaaaaccagcagctgacgtgtgtgaaacatgttgacgatacaacgtgctacgaaagtgcggattagcaaaaaagcgtcgacgttgccgtgtaccagcacgtcgaccaggtagcagagctcggggtaattggggcttgtcacggtggttttgaaaagtcgcaacgtctcttcgtagtcgggtggtggccgcagtcgcatgtgttccatgatctcccaggtgcgcagttcgtggaaggggcccggtgccagtccatctggcaaattaccgatgacgatacgcggtgtacacagcgccaccgtttcgctgttttcctggcagtgcgtaaagtcgaagaaggggtgcagctcggtgtagagcgtgatgttgcccaccttgtagaagtcggtgaccacaaagtcctgcttcatttcgttcaccgtgcgcgggacctcgcgtcgtacgcggtaaaaatgcggtatgcggcgcgccgcaccgcccatgggttcctgctgaaaacgacactcgagcagtcgttgcatggcgggttccgagggcggtccgcgttccgtgaaggtctgtagacagggcgcgggctcgtgcagcaccgggtggcacagcgtcttgagcgcgtccacaaagtctatcttttgtacggcacggtcccggtttagcaggtaggccgtggtgggcaacgcgttgcgaacggtgtcgttaagcttaactttgctttccaccgtggtgtaaccgcgatcctcgggcagatacagccctacggggaagaaaaacgtcaggtccacgttacgttctagcggatctttggtatcggtgtttttgtagacgcgccgcaagttttccataatcaccgttttttcgcccagtcggatcacgtccatgctcagcggcgttaagctgtgcgccccggcctgcgaaagcgagtcgttgggcaaatgcggttggcccgaagtcagatgagccttgtacgagttgaaatcggccaggatcgagtgataggatatggcagtgacggcattttcgggactgagtacaaaattgccgtaggtggccggcgccgagaccgtttctttggtgatgtggcttgagagcagcgacatgatgatctgcataacgttggccgtgcttaccatcacgccgctgatcttggcccccgagctcgtggtgtacgtcgtggggttgtctaggatgctatcggtggccgcttcggctagacgcgtgaggaacttgagcacatagtcgcgatcgcgcgtgcgattcagcaaaaagagcgtggccagcattttggccttgaagctctgcaagatgttgcttcgctggatgcggttcagcgcctgtcgcgccagcgtggcgttctctaccagcgtctgcactacaaagtacggcggcgccttgcgtagcagtgtctgtaaaaagctgtgaatcaagccgcgttccatggcgtcggccgtgtttttgagcgcgcgcagcaccgtgtgcatagcttccacgttgaggatcttgtccaggatggtgccttcgaacgtctcgcgcagatacgtgaggcaggctgcgctgagctcaaaggggatggtgatgggggatttttcactgtatttggtgaccataatggtggtctgacgactggtgggcaaaccggcgccgctggccacacgcggcacctgcacgtggaacagcatttttcccgtagtcagtttattgaggtcgtggaacttgatggcgtgcgccgccgcggccaagccgctggtcaaaaaataaacccattccaggcgattgcagaaggtgccgaagatggcttcgaagtgaatattgtaacgctcggggtcgtcgccgtagtagatgcgtaaggcctcgaacatctcctcgccggcgctggtcttgacgtgcgtcagaaagtcagtgggaatgcctactttaggcaggagctcgagcgccgaccagttctccatcgcggcggcggcgtgagcgcgaggcgtcggagctcggggaaagcagcgcgacccggagaatggccggcgctgcgccgcgccgcctcggctgtgacgctctaatagtcgtcggcggctccgctacgccgcgccgggttttacacgtccccgtgcacgttcgcgcctgcaacctcacccaagagctatcgacgggcgaggacgcccgcttctgtcgtccgcgacccgttaacgtcgaacgggtgcgcgctgtttttgcggctctctaccgtgcctgtccgatacacgtgaggaccgagcccgagcgtgtcaagctggtactgggtcgtctgttactgggacccgtggccgtaccctgtttttgcgacggtgaagtggagggccacggtgaacatctggtacctacgacgcagttttgtcgcgggccgctgctctacgtgcaccgacgttgttgttgcggatccgtgaccgccgggcgcgcgctgtcctaccacgttctcgaaaaccacgtggccacgcatgtgctacgcggattgctctcgctgacggaatggaatcgagaattgccgagcctcttttgcgactgtcctggcggcggtggcgcctcgggaaccgaggaacgctacgccatggcctgcctgccgcgcgacctcagcctgcacctggacgactatccttacctgatggtggaaatcggacgcgtactcagtgtcagcgaggtagacgactacgtaaccgccgtctccggctacctgggcgaggccgcggcgccgcgcattcaggttcactacaagctgctctttggactcaacgtgcgtccgcaagcgccgtgcgcgttggacgctacacgcgacttttttctgctggagctgcaaaagctttggctgggcgttgaatatcaccacgaagtgacgtcggagtttttcggtcgcgtgctggcccagctgcatcgcgaccgcgcccgcgtcatgatggcgcttcgcttgcccgagcagacggtgtgccacctgagcaccttcgttctcagtcgcttcaagcgacaggtactgtacttcaagttacaggtgagctacggcaagtgccggactggccacgctgacagaagtgggggaggggggaacggtggaaatcagggacaccacaacctactgtgttatcgacgccttagcgtcacatttgccgacacagacacggtgtggagaaaccttttctacgtttattacgaactagctcgggatctggggtcccatgggacagagaaccgacccgtaaaccgcggttacggtgtttcttgcgctccgaggacgtcgcggctatcaccgtcagaatcgacggtggtttcggcgaacggacacgcgctgtcttccaccgcgctcccgacgacgagcgcgggtcacaagctgtcactgccgcgcgacccggccgccgatcgcgttcgacgttacgtgtgcattatctcgcgtctcatgtacgctcggtacggggagagatggcgtaaacaccgtcaacggcggtcggagacgggagaagaggaggaggaagagacgctggaatcgggggagactgacgccacgccgccatttgactttacggggcagcagctgcgccgggcctatcaggaacaccgacgtcgtaaacatctagccgtgcagcgttacgcgccgtgccgtcgtaagctcatcggcgggatggagtttgccgaggtgacgggcgtgagtctggaccgcatcgccgtcaacgctttcaacaccaaccgcgttatcaatatgaaggccgcgctctcgtccatcgccgcgtcgggtctcggcgtgcgcgcgccgcggcttcccaagaacatgacccacagttttgtgatgtacaagcacacctttaaggagcccgcttgcaccgtcagcacctttgtttccaacgacgccgtctacatcaactcgctcaacgtcaatattcgcggttcctaccccgagtttctgtactcgctgggcgtgtaccggctgcacgttaatatcgatcacttttttctgccggccgtggtgtgcaatagcaactcctcgctggacgtgcatggactggaggaccaggcggtgatccgctcggagcgcagcaaggtgtactggaccaccaactttccgtgcatgatctcgcatactaacaacgtcaacgtgggctggttcaaagcggctacggccattgtgccgcgcgtctcgggcgctgacctggaagccattctgctcaaagaactctcgtgcatcaagaacatgcgcgacgtgtgcatcgattacggtctgcaccgcgttttcacgcaactagagctgcgcaattcgtaccagatccccttcctggccaagcagttagtgctgtttctgcgtgcttgcctgctcaagctgcacggtcgagagaagcggctgcagttggaccgcctagtatttgaggcggcacagcggggtctctttgactacagcaagaacctcacggcgcacaccaagatcaagcacacttgtgcgctcatcggcagtcgtctagccaacaacgtgcccaagatcctggcccggaacaaaaaagtcaaattggatcacctgggccggaacgccaacgtgctgacggtgtgtcggcacgtggaagcccacaagatccctcgcacgcgcctcaaagtgttagtcgaggtgctgggcgcgttgcagagtatcagcggtacgccgcacacgcgcgaagtgatccaccagacgttgtttcgattgtgctcggcggccgcagccacatcgggcctgtgttcatcccctcccccattgtgtgtgtcctcatcttcctccgtcccctctgtcccaacctccgtcagcgttgacggcagttctgaacccacgtcgccgcgagcgcggtttgcatcacgatgatggaagccgcggccgctgccgccgcggcgtttcgtccggaggagcgtccgacgccgggttggcacgacgcggcgttgttaatggacgacggtacggtgcgcgagcacgcgtttcgcaacggaccgctgtcgcaactgattcgccgtgtgttaccgccgccgcccgacgccgaagacgacgtggtttttgcatccgagctgtgtttttattgcagcggtcgttttaaccgcaggtcgtccgtcttctccatctattggcagaagcatagcgatctggtgtacgcgcttacgggcattacccattgcgccaagttggtggtggaatgcggtcagttggggagtagtaggctacggtggcgcgacggtgatgcgagtggtgaggagcgccggggagacgacgacagcagggacgagctgtacgacgtgccgggcatttatatgattcgcgtcaacgacggcggcagcaccggtcccagacacgttatttggccgggtaccagcgtgctttgggcgccggacgttgtgatcactacggtgcagcgacgaatctcggcggcgcgcgccctggtgaacacgttccgccaatatttttttttgctggaacggcgctcgcacgaggagctggttctttgtccgcccgagatggaggagcgtctagcgccgttgttgcagagtgccacgcgcggtgattcggacatgtttgacggtgtggtggccagcgcttatcaccgtttgcgaatgagtaatattccgcgttcatccgcccgtctgctggagcactgcgtggggctggcgggtgctaagaagctgctcttgctcgacgtgccgcgtctggagaactattttctttgtcaggtctgtctttacgagctggacgaggacgagatgggcgaggagatgctgggcatgttggccggaaagcccgaggatgccgccgtctcgggcgcaagcggcggttttctgctacatcgcaagacgatgaagctggccgcctgtctgtgtttgttgctcaattcgctgcatttgcaccaggaggcgctggaggccttggatcctccgccgccgcgcgtcgaggagaacgaccttgtcaacgtggtgctgcgccgttattatcgcagtcacggcggcgtgcaggcgcggacgctggcggcggcccgggctttgttagccgactacgctgaaacgttttcgcctttggggagttttacgcgcctgggttacgatcgtctcgtttctgccgatgccggcgtcagtcgccggcacctggtggctctgctgcgtgcctagctgaccctgaaacggatggcgtgtatatcgtcacacaggtaggtggccatgatgacggcgatgataagatcgtccgagatacgattctggcgcttggccgagtagcgtgccgtcgtgccttcggccagcgtgacgcggtgcaggttctgaatctgctccagaagatactcgatggggtcgtggctcagcttgatggtgtaggagacgagctcttgcgaggctttgatgtagcccgagttgaaacgcgagatgaactgttccacggccagcgccttgtcgcggcccatgaggtagaagggctgttcgatgtggttctggtcgggcgtgtggtagaagagcacgcggatgagcgtgctgctctgcacgctctgtcggatgaggcaggcgatgcgcacggccgccgcctggttggtgttgccctccacggcgatacgcagttcgtccaggtaagggtgcaggctcagcaccgagatgatcatgtgcgccgcgcactcggcgatggctacctcagaactctcggagaggtcgcgcaaaaagaaatgctctaggccgtaaatgagaaactggtgtcggtaggcgcctacggccgccacgcccgtgcccgaggccttgcggttggtggtgaaggccgggtccagatacacgtaaagcgtcttgccgaaataatcgtaggcgttggtgttgagcgtgctgtaacgcaaaatatcgaactcttcgcggctctggtccgtgatgagcacggtgttctgcgagattttattggtaccgccgatgatctcgtccatgaaagcgcccggcataaacatgttggccgtcttgcgcacctgcgagttgaggctgatgaaggtgggcttgtgcagtcggtagcaaggacacgccgtggcgtcgcccttctccgtgaagctgtgcaggtgctcttcgcacacgtaagagaccacgttgagcatgtcaaagggcgcattgttgaggcgcgtcaagaaacacgtggcgtcactggtagtgttggtggacgatatgaagatgatcttggtggtattctgggccaggaaccccagaatggtgttgaaggcctctttcttgatgaagtgcgcctcgtccaccagcagcaagtggaagttttgtcctcggatgctctgtgtagagaggagacagaaaagggactcttataattacgcacgctcggctggaagcctacagagtcggggtggggccggacaggtgagccaggtgagccgccaggtgaggcgggatcaccgtgtgccaaccgggctgcgacctgaaaaccggaaccaatccgccgacaccggcgccgcgtgacgcgcgcccataaaaacgaaagtgtcgtcgtcgcgacccgccacagccgccatgaactcgttgctggcggaactcaaccgactgggggtcgcgcacgccactacggaggatgtttttatctttgtcgaccgcctctttcaacacttttccttccttttccaggccgaggagtcaggcccgcgccgcttggaactggtcgcgtccgtgttcgagcacctgacggtggagtgcgtcaacgacatcctggacgcctgcagccatccggacgtgaacgtcgcggagacaagcaacacctgtcgtccctgcccttctcctgccccctccgcccccaaaactgtcagcgacgctcagacgtcatgtgcgacgcctcgggcgcctgtgacatgaggcacgtccagaacgcgtttaccgaggagatccagttacattcgctctacgcgtgcacgcgctgctttcgcacgcacctgtgtgatctgggcagcggctgcgcgctcgtctccacgctcgagggctccgtctgcgtcaagacgggcctggtatacgaggctctctatccggtggcgcgtagccacctgttggaacccatggaggaggcctcactggacgacgtcaacatcatcagcgccgtgctcagcggcgtgtacagctacctcatgacgcacgcaggccgttacgccgacgtgatccaagaggtggtcgagcgcgaccgcctcaaaaagcaggtggaggacagtatttacttcacctttaataaggttttccgttctatgcataacgtcaaccgtatttcggtgcccgtcatcagccaactttttattcagcttatcatcggtatctactcaaagcagaccaagtacgacgcgtgtgtcatcaaggttagtcgtaagaagcgcgaggacgcgcttctgaaacagatgcgttccgaatatggaaacgcacctgtattcggatctggcgtttgaagcgcggttcgctgacgatgagcaattgcctctacatctggtgctcgaccaggaggtgctgagtaacgaggaggccgagacgctgcgctacgtctactatcgtaatgtagacagcgctggccgatccgcgggccgcgctccgggcggagatgaggacgacgcaccggcctccgacgacgccgaggacgccgtgggcggcgatcgcgcttttgatcgcgagcggcggacttggcagcgggcctgttttcgtgtattaccgcgcccactggagttgcttgattacctacgtcaaagcggtctcactgtgacgttagagaaagagcagcgcgtgcgcatgttctatgccgtcttcactacgttgggtctgcgctgccccgataatcggctctcaggcgcgcagacgctacacctgagactggtctggcccgacggcagctatcgtgactgggagtttttagcgcgtgacctgttacgagaagaaatggaagcgaataagcgcgaccggcagcaccagttggccacgaccacgaatcaccgtcggcggggcggactgcgtaataacttagacaatgggtcggatcgccgtttgcccgaagcggctgtggcttctctggagacggccgtcagtactccattttttgaaattccgaacggagcaggaacctcctccgcgaacggcggcggcagattcagtaacctggagcagcgggtagcgcgtttgttgcgcggcgacgaggaattcatctatcacgcgggtccattggagccgccttccaagatacgcggtcatgagttggtgcagctgcgcctggacgtaaatccagacctcatgtacgccaccgatccgcacgaccgcgacgaggtcgcgcgtacggacgagtggaagggtgccggtgtctcgcgtctccgcgaggtctgggatgtgcagcatcgcgtgcgcctccgtgtgctgtggtacgtcaattccttttggcgcagtcgcgagctgagctacgatgaccacgaagtcgaactataccgggcgttggacgcttatcgggcgcgcatcgccgtcgagtacgtgctgattcgcgccgtgcgcgacgagatctacgctgtactacgacgggacagcggcgcgttgccacagcgtttcgcctgctacgtgccacggaacatgtcctggcgcgttgtttgggaactttgccgtcatgccttggcgctctggatggatcgggcggacgtgcgtagctgtattattaaggcgctaacgcctcgtctgagccggggtgccgccgctgccgctcagcgagctcgtcgccagcgcgagcgctcggcgcccaaaccgcaggagctgcttttcggaccgcggaacgagagcggtccgcccgccgaacggacttggtacgctgacgtggtgcgctgcgttcgcgcgcaagtggatttgggcgtggaagtgcgcgcggcgcgttgtcctcgcaccgggctttggatcgtccgtgatcgtcgcggacgcttgcgacgttggctctcgcaggccgaggtgtgcgtgctctacgtcacgccagacttggacttttactgggtgctgccgggcggctttgccgtctcttcgcgcgtcactcttcatggcttggcgcagcgggctttgcgagaccgattccagaactttgaagcagttcttgcaagaggaatgcatgtggaagctggtcggcaagagccggaaacaccgcgagtatcgggccgtcgcttgccgttcgacgatctttagtccggaggacgacggctcgtgtatcttgtgccaattgctgttgctctaccgcgacggcgaatggatcctctgtctttgctgcaacggccgttatcaaggccactatggcgtgggccacgtacatcggcgtcgtcgacgcatctgtcatttacctaccttgtaccaactgagcttcggaggtcctttgggtccagccagcattgatttcttgccaagctttagccaggtgaccagcagtatgacgtgcgatggtattacgcccgacgtgatttacgaggtctgcatgttggtgccccaggatgaagccaagcgcatcctggtcaagggtcacggtgccatggacctgacctgtcagaaggcagtgacgctaggcggcgccggcgcctggttgctgccgcgtcccgaaggctacacgcttttcttttacattctgtgctacgacctgtttacctcatgcggcaatcggtgcgatatcccttccatgacgcggctcatggcggcggccacggcctgcgggcaggcgggttgcagcttttgcacggatcacgagggacatgtagatcccactggcaattacgtgggttgcacccccgatatgggccgctgtctttgttacgtgccctgtgggcccatgacgcagtcgctcatccacaacgatgaacccgcgacttttttctgtgagagcgatgacgccaagtacctatgcgccgtaggttctaagaccgcggcgcaggtcacactgggagacggcctggattatcacatcggtgtcaaggattctgagggccgatggctgcccgtcaagaccgatgtgtgggacctggtcaaggtagaggaacctgtgtcacgtatgatagtgtgttcctgtccggtgcttaagaacctagtgcactaacggggtctgacagttcacggggagaagaaacaagaaataacaaaaaaaaaaagaggacatggactcgccacggtttgtggcaaggcgtatgttatcatcatggagctactcacgttggtgttgtagcaactggcaaaaagcgccgtgctcttggcgccgcggtggtcgatgctgatcacgttgtccttgttctcgaccacgtagtcgcgcgcgaaggtgtggcggcagcggaactcgacctctttgagcacaaactgcgacacgtgcttttggtgcgccacgtagccgatgctgatgccgatcatgtgcttaagcagaaacgagataatggggatgatgaaccaagtcttgccgtgacgtcgcggcaccaggaacacggtggctttctgcttaaagatgtcgatggaggtctgcgagaggaagtcgatctggaaggcgtggatgaggtactgcagcacgcgattggccagcacggggatcttggtcacggctataaaaaagatgacgtgtatcaataaattcttttgaaacggttcgagtcggatggcttttgcgtcgccctcgacggcggtactgaagccgccgtcgagccactttttaaagtcggtcatgaagttgttgatctgctgaaactgcggatcgcggtagagctcggtcaacgcgtccagcttctggtaggaggcgcgctgctcctcggagcacgggcgaaacgtcagttcatcgagcgcgctcttgaggcgctcgtgaaacagcagctcgcgctggctttcctcgggcgagttgtagtcgcggtggcggccgcagaaggccatgagcggcaggaaggcctcgttgcacgagtgggccagcccgagttcggggtgcatcatctggtagcgcttgcggcacagcgccgccacattggtgaaggccgtggagatgcaggaggtggggtggctcttgcgcttctgcagctccgcgtagcgctcctggatcttggcggccgaatctccgcgcaacatgatggcggcggcggtggtgcgagcggaggttaggcggcagcggcgagaggagaggaaaaagatggcgtccgcgaggacgacggaggatccacccgaaaaccacgttgtcgcggacgtggcttgtgggacgggcgccgtcactcgttcgtcttcgtcgtccttagtggtgtcgtcctcctcggcgtcaggctcagacgaatcttcatccgcctctcctctcagtttccccgtctcctccccctcaactgccgtcaggtctccggggtccgccggggtttcaacgtccctgtgctcggtggaacggatggtcgagctgtcggcgcagtctccggccgccgatttctcggtctccgaggcttggcgcttcgaggaggccgtaaatatggcgctggtggcctgcgaggccgtgtcaccttacgatcgctttcgcctaattgaaacgcccgacgagaatttcttgttggtcactaacgtaattccgcgcgagtcggccgaggtgccggtgttggatagcagtagcagcggtggcgatagcgggccggaggacaaaaagaaaaacgtcgggaataaaaccgcgggggaaaagaacggcggtgggtctcgggccaaacgccgtcgtagacgacgcgctccgaaaaacgacgccgccacgccgtcttttctacgtcgacacgacgtgctggagcgtttcgcggccgcggctgagcctttgccgtcgctttgtgtgcgtgattatgtgttacgcaatgctgaccgtgttacctacgacggcgaattaatctacggcagttacctgttgtatcgcaaggctcacgtggagctgtcactctccagcaacaaggtgcaacacgtggaagccgtgctgcgacaggtgtacacgccgggcttgttagatcatcacaacgtgtgcgacgtggaggccctgctgtggctgctgtactgtggaccgcgaagcttttgcgcgcgtgacacctgtttcggtcgcgaaaagaacggctgtcctttccccgcgttgttgcccaaactcttttacgaacccgtgcgggactatatgacctacatgaatctggctgagctgtacgtctttgtttggtatcgcggctacgaattccctgcgccgacgccgcaggcgacgacggcgggtggtggtggtagtggtggcggcggcggggccggcgcttgtgcggtcgagacgagcgcgtcagcaggccgggtcgatgacgccggcgacgaggtgcatttgcctttaaagcccgtctcgctggaccgtctcagagaggtattgcaggcggtgcgcggccgcttctcggggcgcgaggtgcccgcctggccggcctcgtcgcgcacctgtttgttgtgcgcgctctacagtcagaaccgtctctgtttagatctcgcgcgtgacgaggcgcggaccgtgagttatagccccatcgttatccaagactgcgccgcggctgtcaccgacgtcactttgagccacatcttgcccggccagagcaccgtctcgcttttccccgtctaccacgtcggcaagttgctggacgctctctcgctgaacgacgcgggtctcatcacgttgaatctatgacgtcggtcaacaaacagctcttaaaggacgtgatgcgcgtcgaccttgagcgacagcagcatcagtttctgcggcgtacctacggaccgcagcaccggctcaccacgcagcaggctttgacggtgatgcgtgtggccgctcgggaacagacccgatacagtcagcgaacgacgcagtgcgtggccgcacacctgttggagcaacgggcggccgtgcagcaagagttgcaacgcgcccgacagctgcaatccggtaacgtggacgacgcgctggactctttaaccgagctgaaggacacggtagacgacgtgagagccaccttggtggactcggtttcggcgacgtgcgatttggacctggaggtcgacgacgccgtctaacaggtatagcaatccccgtcacgcctctgttcatattttattaaaaaaaaacacaacataacgacagtgtcggtgtggtagctagtgcagccctaggaacagggaagactgtcgccactatgtcctccgcacttcggtctcgggctcgctcggcctcgctcggaacgacgactcagggctgggatccgccgccattgcgtcgtcccagcagggcgcgccggcgccagtggatgcgcgaagctgcgcaggccgccgctcaagccgcggtgcaggccgcgcaggccgccgccgctcaggtcgcccaggctcacgttgatgaaaacgaggtcgtggatctgatggccgacgaggccggcggcggcgtcaccactttgaccaccctgagttccgtcagcacaaccaccgtgcttggacacgcgactttttccgcatgcgttcgaagtgacgtgatgcgtgacggagaaaaagaggacgcggcttcggacaaggagaacctgcgtcggcccgtagtgccgtccacgtcgtctcgcggcagcgccgccagcggcgacggttaccacggcttgcgctgccgcgaaacttcggccatgtggtcgttcgagtacgatcgcgacggcgacgtgaccagcgtacgccgcgctctcttcaccggcggcagcgacccctcggacagcgtgagcggcgtccgcggtggacgcaaacgcccgttgcgtccgccgttggtgtcgctggcccgcaccccgctgtgccgacgtcgtgtgggcggtgtggacgcggtgctcgaagaaaacgacgtggagctgcgcgcggaaagtcaggacagcgccgtggcatcgggcccgggccgcattccgcagccgctcagcggtagttccggggaggaatccgccacggcggtggaggccgactccacgtcacacgacgacgtgcattgcacctgttccaacgaccagatcatcaccacgtccatccgcggccttacgtgcgacccgcgtatgttcttgcgccttacgcatcccgagctctgcgagctctctatctcctacctgctggtctacgtgcccaaagaggacgatttttgccacaagatttgttatgccgtggacatgagcgacgagagctaccgcctgggccagggctccttcggcgaggtctggccgctcgatcgctatcgcgtggtcaaggtggcgcgtaagcacagcgagacggtgctcacggtctggatgtcgggcctgatccgcacgcgcgccgctggcgagcaacagcagccgccgtcgctggtgggcacgggcgtgcaccgcggtctgctcacggccacgggctgctgtctgctgcacaacgtcacggtacatcgacgtttccacacagacatgtttcatcacgaccagtggaagctggcgtgcatcgacagctaccgacgtgccttttgcacgttggccgacgctatcaaatttctcaatcaccagtgtcgtgtatgccactttgacattacacccatgaacgtgctcatcgacgtgaacccgcacaaccccagcgagatcgtgcgcgccgcgctgtgcgattacagcctcagcgagccctatccggattacaacgagcgctgtgtggccgtctttcaggagacgggtacggcgcgccgcatccccaactgctcgcaccgtctgcgcgaatgttaccaccctgctttccgacccatgccgctgcagaagctgctcatctgcgacccgcacgcgcgtttccccgtagccggcctacggcgttattgcatgtcggagctgtcggcgctgggtaacgtgctgggcttttgcctcatgcggctgttggaccggcgcggtctggacgaggtgcgcatgggcacggaggcgttgctctttaagcacgccggcgcggcctgccgcgcgttggagaacggtaagctcacgcactgctccgacgcctgtctgctcattctggcggcgcaaatgagctacggcgcctgtctcctgggcgagcatggcgccgcgctggtgtcgcacacgctgcgctttgtggaggccaagatgtcctcgtgtcgcgtacgcgcctttcgccgcttctaccacgaatgctcgcagaccatgctgcacgaatacgtcagaaagaacgtggagcgtctgttggccacgagcgacgggctgtatttatataacgcctttcggcgcaccaccagcataatctgcgaggaggaccttgacggtgactgccgccaactgttccccgagtaaccgggacgcggaacgtgacggttgctgaggggaaaggcaacagagaaggtacaaacccaccggcggggaaaataccgaggcgccgccatcatcatgtggggcgtctcgagtttggactacgacgacgatgaggagctcacccggctgctggcggtttgggacgatgagcccctcagtctctttctcatgaacacctttttgctgcaccaggagggcttccgtaatctgccctttacggtgctgcgtttgtcttacgcctaccgcatcttcgccaagatgctgcgggcccacggtacgccagtagccgaggactttatgacgcgcgtggccgcgctggctcgcgacgagggtctgcgcgacattttgggtcagcggcacgccgccgaagcctcgcgcgccgagatcgccgaggccctggagcgcgtggccgagcggtgcgacgaccggcacggcggctcggacgactacgtgtggcttagccggttgctggatttggcgcccaactatcggcaggtcgagctcttccagttgctggaaaaggaatcgcgcggacagtcgcgcaactcggtgtggcatctgttgcgtatggacacggtctcggccaccaagttctacgaggccttcgtcagcggctgtctgcccggcgccgcggcggcggacggttcgggtggcggcggctcgcactacacgggctcgcgcgccggcgtctcgccgggcatccagttcggtatcaaacacgagggcttagtcaaaacgctggtggaatgttacgtgatgcacggacgcgagccggtgcgcgacggcctcggtctgctcatcgaccccacgtcggggctgctgggcgcttccatggacctgtgcttcggcgtgctcaagcagggcagcggtcgcaccttgctggtggaaccgtgcgcgcgcgtctacgagatcaagtgccgctacaaatatttgcgcaaaaaggaggacccctttgtgcagaacgtgctgcggaggcacgacgcggcggccgtggcctcgctgttgcagtcacacccggtgccgggcgtggagtttcgcggtgaacgcgagaccccgtcggcacgcgagtttctgctttcgcacgacgcggcgctcttcagggccacgctcaagcgcgcgcgcccgctcaagccgcccgaaccgctgcgcgagtacctggccgatctgctgtatctcaataaggccgagtgttcggaagtgattgtgtttgacgccaagcacctgaatgacgacaacagcgacggggacgccacgaccactattaacgcgagtctcgacctagccgcgggcgacgccgctggcggcggcgctgatcaccacctgcggggcagcccgggcgattcgccgccgccgatacctttcgaggacgaaaacacgcccgagctgctgggccggctcaacgtgtacgaggtagcgcgcttttcactgccggcttttgtcaatccgcgtcaccagtattactttcagatgctcattcagcagtacgtgctcagccaatactatataaagaagcatccggacccggagcggatcgatttccgtgacctgcctaccgtctacctggtctcggccatcttccgcgagcgcgaggaaagcgaactgggctgcgagttgctggccggcggtcgcgttttccactgcgaccacattccgctcctgctcatcgtcacgcccgtggtctttgaccctcagtttacgcgccatgccgtctctaccgtgctagaccgttggagtcgcgacctgtcccgcaagacgaacctaccgatatgggtgccgaactctgcaaacgaatatgttgtgagttcggtaccacgcccggtgagcccctgaaagatgctctgggtcgccaggtgtctctacgctcctacgacaacatccctccgacttcctcctcggacgaaggggaggacgatgacgacggggaggatgacgataacgaggagcggcaacagaagctgcggctctgcggtagtggctgcgggggaaacgacagtagtagcggcagccaccgcgaggccgcccacgacagctccaagaaaaacgcggtgcgctcgacgtttcgcgaggacaaggctccgaaaccgagcaagcggtcaaaaaagaaaaagaaaccctcaaaacatcaccaccatcagcaaagctccattatgcaggagacggacgacctagacgaagaggacacctcaatttacctgtccccgcccccggtcccccccgtccaggtggtggctaagcgactgccgcggcccgacacacccaggactccgcgccaaaagaagatttcacaacgtccacccacccccgggacaaaaaagcccgccgcctccttgcccttttaactcataaactttcaggtctcgcgtacgattcgcgagtcgggaatgggacacccgtgggtgtttctccgtgtgtatattatttttttttgtgtgtgtgtgtgtttgcgcccccgtgtgtctaatgtgctgtttgaaacacgtaaagtagctggtggaagaacagataaacctttaataaaaaaaaagtatgtgctcccgacccacggtctgcgtgtctcttttttatgtccatgtctccaagtctggtgcgggtggcggcggggtcaagcgtcctcgaagtcttcatcatcgtcgtcgtcctcttgttcgcggaggcgacggctttccaagctgtcgtggtgactgagcgcagcgacttcttcgccggaggctgtggccagcgcctggtacttgacactgccgctaccgcgtccgcgaaagtagcggacggcgcgacacgtcgtaaacatggcccatatgaaaaagagcatgccgaacgaccagctgatgccggtgcggtattcgttgctgaggaaggtatcgtactgcacgatggggtagatgaggccgcagagtccaaagaaggcgcccaggtggtagccgaattgcaccttgacgtattgaaaaaagacggcctcgatcagtaaaaagtagatgatggagatgatagcgtagaccacgaagacggctaacaccatgtggcctgtacgcacgaaaaagttgtttccgaagccgtagcacagggccatggctaccacggtggtgttgaaaccaagcgctacctccaccaggttgacgatgagcgtgcggaactgcaccgtacctttgagcttggggtgcagacgcgagaagaaaaagagtgagcgtttgtagctgcggtactgcgtgaccatgctcacgttgaaaatggtcaggcagaaaaagtgcacggcggccatgaaggcgatcatgctgggcagccgaaatgacatggtcagtgtgaatagttggaacgtgtccatgctgagaatgaagaggaaggctgtgaggctgtcgcccatgtacgaaatgtcgcgtgtcgactggtttaggctcatgcctttgtccttgcgcatgctgatcttgatccagcataccaggtagtagatggtcacggctaaaaagacgagctgcatgaacacggcgtagcacaccaactgcaccgagtctaagaaaagcataggcgtgtgcaggtgcattacgttgtaggccgacatgttgagcctttcaaagtccacgacgtgatagtagacgcaggggtagcccaggtgcggaaaattgctcagcactagatgcacgctgacgttgacaaaagtgagcaccatgaaaacgatagaagcgctccatgtccgtgtattcactttatccacgtgcgagggggccatggcgatagcggcggcccgctcgctcgggaggcgatgggggcgcgccgatgacgacaggctcgcgggtcgttaaatactacgatgggagccgccgcggctcacgacgcggtttgagcacgtccgggcgatcggtgaaaaaagaccccgcgggccttcgcgactctcttctgtccgaggatgaccgctcagccgccgctgcaccaccgccaccacccgtacaccctgttcgggaccagctgtcatctcagctggtacggccttctggaggcctcggtgcctatcgtacaatgtctgtttttggatctgggtggcggccgtgccgagccgcggcttcacacgttcgtggtgcgcggtgaccgtctgccgccggctgaggtgcgtgctgtgcatcgtgccagctacgccgcgctggcctcggccgtgactacggacgccgacgagcgccggcgcggcctagagcagcgtagcgccgtgttggcgcgcgtgttgctagaaggcagcgcgttaatccgcgtgttggcgcgcaccttcacgccggtgcagattcagacggacgctagcggcgtggagattttggaggccgcaccggcactgggcgtggaaaccgcagcgctatcgaacgcgcttagtcttttccacgtagccaagctagtggtcatcggctcgtatcccgaagtgcacgagccgcgtgtggtcacgcatgccgcggaacgcgtctccgaagagtatggcacccacgcgcacaaaaaattgcgtcgcggttactacgcctacgatttggccatgtcgtttcgcgtcggcactcacaagtatgtgctggagcgcgacgacgaggccgtcctggcacgcctctttgaggtgcgcgaggtgtgttttttgcgcacctgtctgcgtctggtcacgcctgtcggtttcgtggccgtggcagtgaccgacgagcagtgttgtttattgctgcagtcggcctggactcacctttacgacgtgcttttccgtggtttcgctgggcagccgccgttacgcgactacctggggccggacctttttgagacgggcggcgcccgttctttcttttttcccggtttcccacccgtgcccgtctacgcggtccacggtctgcacacgttaatgcgcgagacggcgttggacgcggcggctgaggtgctctcgtggtgcggcctgcccgacatcgtgggctcggccggcaagctggaggtggaaccctgcgcgctctcgctcggcgtgcccgaggatgagtggcaggtcttcggcaccgaggccggcggcggcgccgtgcgtctcaatgccacggcttttcgcgagcgaccggccggcggcgatcgtcgctggctgttgccgccgctgccgcgtgacgacggcgacggtgaaaacaacgtcgtggaagtcagcagcagcaccggcggtgcgcacccgccgagcgacgacgctactttcaccgtgcacgttcgcgacgccacgctacatcgagtgctcatcgtggatttggtcgagcgcgtgctggccaagtgtgtacgcgcgcgcgacttcaatccctacgtgcgttatagtcatcgactccacacttatgcggtttgtgaaaagtttattgaaaatctgcgttttcgctcgcgacgcgccttctggcagatccagagtctgctgggctacatctccgagcacgttacgtcagcctgcgcttcggccggccttttgtgggttctgtcgcgtggacaccgcgagttttatgtctacgacggctattcgggtcacggacccgtctcggccgaagtgtgcgtgcggactgtggtcgactgttattggcgcaaactttttggcggcgacgatccgggtcccacctgtcgtgttcaagagagcgcgcccggcgtgctgttggtttggggcgacgagcggttggtgggtcccttcaacttcttctacggcaacggcggcgccggtggtagtccgctccacggggtggtgggtggtttcgcggcgggacattgtggcggcgcttgttgcgcgggctgcgtcgtcactcaccgccattctagcggtggcggcggcggtggtggtggcgtgggcgacacggaccacgcgagtggcggcggtctagatgccgctgccgggagtggtcataacggcggtagtgatcgggtttctccctccacgccgcccgcggcgttaggtggctgttgctgcgcggccggtggcgactggctctcggccgtgggtcatgttctgggccggctgccggcgctgttacgggagcgcgtgagcgtgtccgagctggaagccgtgtaccgcgagatcctctttcgcttcgtggctcgccgcaacgacgtggacttttggttactgcgcttccagcccggtgaaaacgaagtaaggccgcacgccggggtgattgactgcgcgcccttccacggcgtgtgggccgagcagggccagatcatcgtacagtcacgcgatacggcgttagcggccgatatcggctacggcgtctatgtggacaaggcctttgccatgctcacggcttgcgtggaggtctgggcgcgagagttattgtcgtcctccaccgcttccaccaccgcttgttcttcttcttccgttctctcttccgccttgccgtccgtcacttcgtcctcttcgggcacggcgacggtgtctcctccgtcttgttcttcttcgtcggcgacttggctcgaggagcgcgacgagtgggtgcgttcgctggcggttgacgcgcaacacgctgctaagcgggtggcttccgagggcctgcggtttttccggctcaacgcttaacgagtcacgtaggggaactacgtgggtaagtgacgtggatactagtaaaaaaagtgcgtcaaagttctcagcgtgtgacgtggatactagtaaaagggacgtcaaagctcactacgtgttgcgtgttttttttttttctatgatatgcgtgtctagttcgcttctcactcttcctctccccgttcccagcgcggtggcagcttggggggtgagggcaaattggggtagttggcgttgagcacgtctagcaggcccaggcccacgggccaaccgtccacggtcttacgctcggtcagcttgaggctaaacgagtgtgcctcgtcttgaccggtaaggcggaaaaagaagcgtgctaccagctgcaggcaggtatgccgcgtctgctggaagagcacgaaggtagcgggcacgtactgcacaatgtgcggttctttttcctcaaagagtaggtagagcgcgctgcagatcagccgccgggcgctgtggtgcagcagccggccgaagctttcgcgcacgttcactgcgtccaggtactggagcaggtcgtgcaggcacttgcgcgttaagttgcaattttccacgcatgaaataacggtacagagcgcgaagtgcagcaggttgtcggccttgacgatgccgcagcggtgtttgagccgcagatccgagagcctcacctgcgtgacgacgtcttcggtctcgagcaaaaacacggcggagtagcccagaaaggccgaggtgcacagcaactcgctgcggtactcggccatggaaaccagcagcccgtgctccgtgtgcagccacagcttgtcgccgcgcaccgtaaagtcgagcacttgcggctccatgatcatcacattctgtctagtgaaatccgtatggacctccagcacgccgcggatcatcagggcctccatttcgaaatcggccgacacgctctgggccgcgccgctcctcgtctgccgtgatcaagcggcgcggcgcggacctttcaagcgttcctgggccgccgctcgaggcagttcccctttctggcactccgcccgccgcttcgcggctcatttggcgccggcgcgccttctcgcggctgcaaatcagctccacgtatcggcaaaacttgctgtcgtcgtaggcggcggccacgatctcgccgaaggagagctgcaggtaggcttcgggtacggggtccagcgtgcctagcgccaggatgtgacacagatagggcagggtcacgcgctctaccgtgtaattggagtagacgatggcctcttcggccccttgatgcgtgaccagacgccgtaggcgaaaggtgcggaaatactcgttttcccacaactgcgtgaggaagcgttctagcgactcggtgccaggcacgaactgcgagaagaagctgttggccaccaggcggttgtcttccaccgccagcggacggaagggcgccgcgtcgcgcgccttgcgcacggcctccaacacgggcaggtggtagagttcggcgtcgcgcgcgcccaggctcatggagtcctcgcgccgcgaggcgtagcgcgtgagcaggtcgcgcagctcgcgcacgcgattctcccaggtctggttgagcgtgcgcaggtcctggatctcgtccacctgcgactggatctgctcctccaggcacttgatgacctgcttcttaaacaggtcgcggatgtcccgctcgggcgccgccgggccgggtggcggcggcagcagcccgacgtggcccgcgggtcctcccaccacggcgccgccgggtcccaccacgccgggtccacccggaccacgcgcgggtagtagacggttttggtccaccagcgagggggtcaggtcctgcagaaaggactcgacgctgtcctcgatgccgatgcgcgatttgctgtccgagacgttaagcaaaaacttcataatggactttttggcgtcgctgccccggtcgtgctgctccatcatctccaccagcttcttgcagttgagctcgtggcggctggcggtcaccactttcacaggaaaggtattgagcagctggcagatcttttggtggcggcagagcccgtcgtagcgcagaatctcctcgtgcaggtgtgccaccggcgtggtgaacagcagcttgtcgcgctcataagccagcggttcggccgccacgtacaagcggatgtgcttgccgcgcagctgcgcctccagccgctccgagcgcaccttcttgaagacgcgtacctcgggcgcgttggctacgcgcacggcgcccaggcgctcggccacctgcagcagcagcgccaggttagcctgcagcaggtcctgcgccagcgggtgtgtctcggtggcccgctgcacggccgcgcgtacaaattgcgcccgctcggccgcctcgctcggcttggttttcacgtccagcagcggtaccagtcccaccgttacgcaccaatccacgtagagaccatagtcgtcgttatcggcgtactgatataaaatgtcgcggagcgcgcccagcacgcccgtttgcacgctctggcgcaacgaggcgctccacaccaacagatactgctccaggtcctcttcgtccagcgcgcggtagggaaacagcgccgcgtgtaacttccactcctcggccacgcgccgcaccgtgatggtgtcaaagagcgtcttgcacactccgtagagcagctgcttgcgcagcacgcacgggtcgcgcagcacctggtgcatgctctggccgcgacacgtccccagaaagccgtgcagcaaccgcaggaagctcatcgtctggcccgtggggaaaatgtcgatgacggcctcgtcatccacgccgcggcccacgcccaagtacgacgacgccttgatcctcaacctctcgtcggctgccaagatcgaacggatcgtcgacaaggtcaagtctctctcgcgcgagcgctttgcgcccgaggatttttcgttccagtggtttcgctccatcagtcgcgttgaacgaacgacagataacaacccctctgccgcaactaccgccgcggcaacgacgaccgttcactcctccgcctcctcttctgccgccgctgccgcttcgtccgaggccggcggcacgcgcgtaccctgcgtcgaccgttggcccttctttcccttccgcgcgctgctcgtcaccggcacggcgggcgccggcaagacttccagcatccaggtgctggcggccaatctagattgcgtgatcaccggtaccacggtgatcgccgcgcagaacctcagcgcgatcctcaaccgcactcgctcggcgcaggtcaagaccatctaccgcgtcttcggtttcgtcagcaagcacgtgccgctggctgatagcgccgttagccacgagacgctggaacgctaccgcgtgtgcgagccgcacgaggagaccaccatccagcgcctgcagatcaacgatctgctcgcctactggccggtcatcgccgacatcgtggacaaatgcttaaatatgtgggagcgcaaggccgcttcggcctccgccgcggccgcggccgccgcctgcgaggacctctcggagctgtgcgagagcaatatcatcgtcatcgacgagtgcggccttatgctgcgctacatgctgcaggtggtggtgtttttttactacttttacaacgccctgggcgacacgcgactttaccgcgaacgccgcgtgccctgcatcatctgcgtcggttcgcccacgcagaccgaggcgctggagagccgctacgaccactacacgcaaaacaagagcgtgcgcaagggcgttgacgtgctctcggcgctgattcagaacgaggtgctcatcaactactgcgacatcgccgacaactgggtcatgtttattcacaacaagcgttgcaccgacctggactttggcgacctgctcaagtacatggagttcggtatcccgctcaaggaggagcacgtggcctacgtggaccgcttcgtgcggccgcccagctccatccgcaacccctcgtacgccgccgagatgacgcggctttttctctcgcacgtcgaggtgcaggcttacttcaagcggctgcacgagcagatccgcctgagcgagcgccaccgtctcttcgatctgcccgtctactgcgtggtcaacaaccgcgcgtaccaggagctctgcgagctggccgacccgctgggcgactcgccgcagcccgtcgagctctggttccgccagaacttggcgcgcatcattaactactcgcagtttgtcgaccacaacctctccagcgagatcaccaaggaggcgctgcgccccgcggccgacgtcgttgccaccaacaactcctccgtccaggctcacggagggggaggatctgtcatcgggagcaccggcggcaacgacgagacggcgtttttccaggacgatgataccaccaccgcgcccgatagccgtgagacgctgctcaccttgcgcattacctacatcaagggcagttcggtgggagtcaactctaaggtgcgggcctgtgttatcggataccagggcacggtcgaacgtttcgtggacatcttgcaaaaggacacgtttatcgaacgcacgccctgcgagcaggcggcctacgcctactcgttagtttcgggcctgctcttctcggccatgtactacttctacgtgtcgccctacacgaccgaggagatgttgcgtgagctggcgcgcgttgagctgcccgacgtgagttcgctctgcgccgctgccgccgccacggccgccgctcccgcttggagcgggggagagaatccgataaataatcacgtcgacgcggattcttctcagggcggccagagcgtgccggtatctcaacggatggaacatggccaagaggaaacccacgacatcccctgcctgtccagccaccatgacgactcggacgccatcacggacgccgaactcatggatcacaccagtctgtacgcggatcccttttttctcaaatacgtcaagccacctagcctggcgctgctttctttcgaggagacggtgcacatgtacactaccttccgcgacatttttctcaagcgctaccagctcatgcagcgtctcacgggcggtcgcttcgccacgttgccgctcgttacctacaatcgccgtaacgtggtgttcaaggccaactgtcagatcagctcgcaaaccggctccttcgtgggcatgctttcgcatgtgtcgccggcgcagacgtacacgctcgagggctacaccagcgacaacgtgctcagtctgcccagtgaccgccaccgcatccaccccgaggtggtgcagcgcggtctttcgcggctggtactacgcgatgcgctcgggttcctctttgtgctcgacgttaacgtctcgcgcttcgtcgagtcggcgcagggcaagagtctgcacgtgtgcaccaccgtggactacggcctcacttcgcgcacggccatgaccatcgccaagagtcagggcctgtcgctcgagaaggtggccgtggactttggggaccatcccaagaacctcaagatgagccacatctacgtggccatgtcgcgagtcacggaccccgagcacctcatgatgaacgttaacccgttgcgactgccctatgagaagaacaccgctatcaccccctatatctgtcgcgcgctcaaagacaaacgcaccacgcttattttttgacacaacaccgtgtaagcaaaacgtgactttattgagcagggtaaaaaccacgtacaagaaccacgttgtctatccccccaaaaaaaaacacaccgtcagggaacacatcgcctatagatagcggcactttacataaaaccaccgtacctgcatcacggtggctcgatacactggaaattcaataaaaaccaccgtgtccacgttacggtacttgccgggtcagcgtccttctcttgagatttctgttcgcaaacttatccgtttccccggtccgcggtgtctcctcgcgaggctgacagtctacgagtggtatctacaagagaaagaaacccgggtgggagcgacgccgtcgctgggtatcaaccccgcggctgaccgtcgtccggtaaaggaacaacccgtcgtcgcaagccgggttcgaccaagagaaaaaaacccgggtgcggggggagacgggtcgtcctttggtcgttcgcggacggcgtacatgccgcgtgggtcagtcgacggcgtcgctccgtgcggtcggtcatcattctgcttcacatatatgggttgtttgtgttttttttataatgaatacgcactcatcctatccgtgactgcgcgtgtggcagagaggatgccttataacatgtattttgaaaaattgccaacagctataatttctctcatgtagcagaatagagaccttttgtcgtctttttgtttgtcattacttgttttccagggaattagagagagggaaccgcgcctccggcggcggtgcccgcggaccccggccccttctcgcgtgcgcggtgtgactggttgagcgaatgagcagctaggcttggtggtgctccgcgtgcgggggagaagacgattaacaacaaaaaataagtggaagtggccggtgggtctttgtccgcgtgcgcgcccatccgtcgccgggaccgagcagaaagtgatgtggtggtacattgattttttccttgacaggaaagaaaaaaaagagttttgttttcctatgtgagaggagaaaggtatgtgaggagatgttcgatgatcgtatgttacagttatgctgtaaggaagcttttatcgtgcgtcctgtttttcatttgatgtatatgacacaattgaaacctatcgataggcgtatatcgaggattcatcaattcttagaatcgtcgtctttttggctaattggactttgcccatgttggttgtcattcgtggcctgaggtcatcgtcgtccacgacgacgtgtctatagcgtgcggtgtgatcattgtgtcgagccagagaaagcgcgcctcgcacgacgtttgcggatcggctcgcgggtgtgtggaattcctaagaacataatcagctggtcgtctttctttgatgtgttgttgtcgtcgaggtcttgcttcgttttcttttttctttttagtcgatggaacttttcttcggtacgggttcttgttatggaagcttgtgttttcgaacatgaattcgaaaaaataaaaaggcctatcttcgtttcaaaaaaaggacagatatcaatcttcttaacttatatcatggtaaattcagaatcctatggtgtcttattatctctaaagtagtcaacattatggtctaacttgtatttccctgacgagatatatatgatccttataacctggctactatcatgaacaacaatatccttacttacagtcatcttcgtgagttaatgaagtataatatcggtcatctatcaacttatctgctatgtaacgtacccttttaggtattttgcgtttcttaacgagtgtacccgcctgtgtgaggcgaaactctgagaagtctaccgagtcgagttacaagtcactaaaacacttacacgagttatctatactaaaatcactatctatgttgtttgcttacctaattattatcctacatgacgaagctacctcccaacgtaaggtagggggagaggagacagaacaataaaaagtaactaatgtttcttagaacttacccgctaaggacttaccaaactatattcaccaaaaaacaacagctacgtgtttcatttgttttaatctaccgaagtaaaaaaaaaaaagatgattagctatccagaacctacttacttcttaatgttttaactaaggatgcctatgggattggaaaaaaaatcacagcaacttgctactaatcagttgacagcgaagagactcataacaaagatttctgggtaatacggttataataatgcttatggactaaaggatacttggaaaaaaagaacgggctatgactatagagattcgtcgagatattaaacttcaaataggcggctatcattcatggttgtggtgactatatcgtggagaaaaaatgtgatcgttagttagctaggtgagacttacagctatccatccgtctagtttttcgttgtaatgatgatagtacgtctatggtggtgatcgattttggttaacaatttgttcgtttaaaggcttaatgtacttatgctacatgatgtattattctttgattcatcgttcctcctaagggggtgtatgtatgtatgtactagtcgtatagtgttcctaacatcatgattattcagactatggcttcatctatcgtgtctaaagttcacttattctactattactatatatatgcactactatgtaactaggatatggtcctataaggtgtcttctatcacggtggcttgtttatcgcttggcggttacgagcaagagttcatcacggaccagccgtgaggcagggcacacgcgggtcggcggcgatgatgtcccccgcgaaggggacaacaaaaacaagacaagaggccgccggccgcggccacggacgcgtagcggttacacaatgtttggttgagcgttttgtttcatcgtcgtggtggtggttttgttgttctctgtatatatcgtgtggtggctttatcgtcatcattattatcatcattcttgtttccatcatcacgatgagttttctccgttttcctctcctccagtggtagtcgtgtatcatcatcaatcatcgtagtgacgtcgttgctgctgctgctcttgccttcatggcggtatttctcttcctcccccctaaccccatattaactcgtgagtgtgatggttagagtggctgcttgtttttttttcttttctctttggaacaacaaaagaggataaagatggtcggtgaatgtattattattattatcatcattatgatacggtcgcggtcttcttctccgatgacgaaacctgcgcacatcgaagaaaagacgagcgcgcgaaccgatagccgtccgtctgggacgaaggagaagatgatggggagaggaggagagccccagaagccagagcgagaagggagacgacagacatacgtcgtcaccgtcctctggaggaggcacggcggcgctgtttgttgtttggatgcttgattatatcctgttctatggggtagattattatcaataggcttggttttcaaaggtcagcctgtgtattgtcgtgtcttttttttcgttctcatgatcgcggagaccacacagacgtgcgcgtctcccaatggctaggcgttctttttaggtagtaattttttgatcttttttttttcttaacaagtctggcttgatttcttttatctatgatcgattcttctttttctcgggggttgcatcttccgtgaaagtaaagtgacactactctaaatggtaaccatattatctgttgattaggagaaaaaataattttttcgcacgaaatcgatcctaagtgaggtgatttacttgctatcacacgaaatgattatcttttgctgctaacgtactgaattttttaacagaattgcttctccgtaactatttccgcagattcagacagattgtcaaaaaaaaatacggcacagaaatagtgggtctgtggcttttggttcgtgtacattcgcgtttgcgtgtcgagatttctacggtatgtttattcttcctgcgatgatgtagggtccttggtgtaagtaggatttcgagtatctctcttagagcgaacaaaataatcaaaaaacaacagctaggaaatcgagggttactctacgataaagtgtctctacaaagtgaagaatgttacgttgtggtggaataataagactcgcgtgatcgatgagtgatcgagagcggctcgaaccttctttaagagctttgtttagtgcaactttaaattacaaggagtagaaagctgaaatgaatctatgaaggtgctattctttgaatatcttactttgtacgcttcacattcgttatttggatagagagttgtctagagaaaatctgtgattctctatgagtgttatttttattatccttttggggactacgatttttcttcttgttctacataccactactactcgtaatcacatacatggacgaaaaaaaaattcgtcaggcagtagataccagattctccgacgttacggcgtctttttttcttttgagagagtatctgctgagattgtccgtggtgtatctagtcgctatttttgttgttactagtagttttgcacacagtttattcagtataatttttcttcttgccatgatcaattgagcccaccaccttttttttttagagaggaggaatttcgtcttgatctccagccggagacaacggcggcggtggtggtggcgggagagatttcaaggcaatgaaaaaaaaaatttcgttttgccatcaagtggtgacgataacccgtcagattgataattggttcctacagaaactattctaaccgcggaagaaagaaattgaaaaaaaaaattgacaaaaacatcataacataaaggaccacctacctgggacgcgcagttgggcggcggactggggcggcatgctgcggcgatgctgtcggtgatggtctcttcctctctggtcctgatcgtcttttttctaggcgcttccgaggaggcgaagccggcgacgacgacgacgacgataaagaatacaaagccgcggtgtcgtccggaggattacgcgaccagattgcaagatctccgcgtcacctttcatcgagtaaaacctacgttggtaggtcacgtaggtacggtttattgcgacggtctttcttttccgcgtgtcgggtgacgtagttttcctcttgtagcaacgtgaggacgactactccgtgtggctcgacggtacggtggtcaaaggctgttggggatgcagcgtcatggactggttgttgaggcggtatctggagatcgtgttccccgcaggcgaccacgtctatcctggacttaagacggaattgcatagtatgcgctcgacgctagaatccatctacaaagacatgcggcaatgcgtaagtgtctctgtggcggcgctgtccgcgcagaggtaacaacgtgttcatagcacggtgttttacttttgtcgggctcccagcctctgttaggttgcggagataagtccgtgattagtcggctgtctcaggaggcggaaaggaaatcggataacggcacgcggaaaggtctcagcgagttggacacgttgtttagccgtctcgaagagtatctgcactcgagaaagtagcgttgcgatttgcagtccgctccggtgtcgttcacccagttactttaataaacgtactgtttaaccacgttgcgtcgtgacgttgtttgtgggtgttgctaggcgggctggaaagatgatgtataaatagagtctgcgacggggttcggcgctctgccggctgcggcggcactcgctccacggcctccgacgagcgttgcgctcgcgctttgcgccgccgcgtcatggatctgcctactaccgtcgtgcgaaaatactggacttttacgaatcctaaccgcatcctgcatcagagcgtcaatcagactttcgacgtgcgccagttcgtctttgacaacgcccgtctggtcaactgcgtggacggcgatggcaaggtgctgcaccttaacaagggctggctctgcgctaccattatgcagcacggcgaggcttcggccggcgccaagacgcagcagggcttcatgtccattgacattacgggcgacggggaacttcaggagcacctctttgtacgcggcggtatcgtctttaacaaatccgtctcctcggtggtgggctccagcggacccaatgagagcgcgctgctcactatgatttccgagaacggtaatttgcaagtgacttacgtgcggcattacctgaaaaaccacggcgaatcctccagcggaggcggtggttgcggtgccgcgtctaccgcctccgccgtctgcgtgtcctcgctgggtggcagcggcgggactcgcgacggcccttctgcggaggaacagcaacggcgaaggcaggaacagcgtcacgaagaacggcgcaaaaaatcgtcctcgtctgccggtggtggtggaggcggcggcactggtggtggcggtggcggcggcgggagcggcggtcagcactcctcggactccgccaacggactgctgcgggatccccggttgatgaaccggcagaaggagcggcggccgcctccctcctccgagaacgacggtgagtcccggccctcctcgcgtcacggtgctttccgagtggactcgtgagccccccgtagcgcacgagcgagcaggcgagcggtgttggtgcgctggtggttgtgtggatgataaccatgtgctttttcgtgcgctatgtgtcgtcccgtctgtaggctctcctcccctccgggaggcgaagagacaaaagaccaccgcacagcacgaaggccatggcggcggcggcaagaacgagacggagcagcagtccggtggtgctggcggtggtggtggcggcggcagcggccgcatgtcgctgccgctggacacgtctgaagcggtggcctttctcaattactcgtcctcatcctccgcggtctcttcttcctccaacaaccaccaccaccatcatcaccaccataacgccgtgacggacgtggccgccggcaccgacggtgcgttacttctacccattgagcgcggagcggtggtttcgtcgccgtcgtcgacgtcgccgtcgtcacttctttcgctccctcgacccagcagcgcccacagcgcgggcgagacggtgcaggagtccgaggcggcggcgacggcggcggctgcggggttaatgatgatgaggaggatgaggagggctccggctgaggcggcggaggcaccaccgcagtcggaggaggagaatgattccaccactccagtctctaactgccgtgttcctccgaattcgcaggaatccgcggcgcctcagcctcctcgcagtccgcgttttgatgacattatacagtcattgaccaaaatgctcaatgattgtaaggagaaaagattgtgcgatctccccctggtttccagcagactcttgccagagacgtcgggcgggactgtcgtcgtcaaccacagcagcgtcgcgaggaccgccgcagctgtctccacagccggcgttggccccccagcagccgcatgtccgccactcgtcaccaccggtgttgtaccctcaggttccgtcgccggtgtcgcgcccgttgccgccgcagtcgaaacaccagctgctcctccccggcccgtgtgtgaaatcaagccctacgtggtaaaccccgttgtcgccaccgccgcggctgccagtaactcttcctcgtcttcttcggctccactgccgccgccgccaccaccgccgggcggacgtcggggtcgggcccggaacaatacccgaggaggcggcggtggtggcggtggtagaaacagccggcggcaggccgcatcgtcgtcgtcctcctcctctcggagatcgcgacggagaaacaaccgccacgaggacgaggacaacgatcctctgctccggttgtcgcaagtcgccggcagcggccgccggcgagggccctcgttcctcgaggacggactcgaaattatcgatcccagcgaggaggctgcgatcgccgccgcctcgatcgcggcgtttttcgacgattaaaaaaccgagccgagaccggaaaaattatgaaacaggacgcgcttggacatttgggtttccacccctttcggtgtgtgtctatatatattgtggtcactgattttttttttacaataaagagatagacatcacagttcaccatcttgtctccccggtgtgtctattatcatcaatcacccacagagtcgccagtccatggtctctcggtaatgcgtgtccagatacgcgttggccagtataaagtggtcgttgcccacgaaggcgcgggtggtgttgcgcggcgacgggtggcaggacttaagtaccaagtgccgccgtcggtcgatcaggtactcgcaggtgtgcgcgtcggcgccccacagcatgaacaccagatgctcccggcgctctgacagcctccggatcacatggttactcagcgtctgccagcctaagtgacggtgagatccaggctgtccgtgcaccacggtgaacacggtgttgagcagcagcacgccgcgtcgcgcccaggcgtccaggcaacccgaggccggacgctgaaacccgtccaccgtacgcgccagttcgcgaaacacgttgttgagggagggcggcggcggtcggcccgccagcgtgccgaaggccaggccgctggcgctgccgtcgcagtacgggtcctggcccacgatcaccacgcgcacctgctcgggcggacacagatagctccagcggtgtacgtgctcgggtgccgggtacaccatctcgagttgccgcgcgccttccaccgccgccaccgtgtcgcgcagcagcaccgtgtcgtggtcgggcaagctgaggaagcggatccagtcggcgctcagacaaaacacgcgagcctgctcgtcgggggttaacagagagcctttattatcagcaatgttagcgagcatccactgcttgagggccatagcgcgagtgagccggcaggttgacgcgcgtctgcttcagctcgggcggcagtccggcgtagtatttatctaggtggcgtagcagcggcgggtccagctggtgacgcaggcagaattccttcaccgcgttgtacaggccgtaaaagagcgtgatgccctcgggcgcggcagcggtgctcacgggcagacgcacggcgcggttggtacgcgtggcttcgttgcgtatggccaccaccacgttaaagagagacggtggcaccagctcgaagcctaacacgtgttccgtgaagatgctgcgcccgtatgacagtcgcgtgaggtcgtagccgcggcacaggtcgtccacgcacgtgtacacggccggcgagccatcgccgcactcgctgtagccgcgcattaccgtcatccagcgcggcgctgtgtccgagcttaacagcgtcagcagggcccgcaattgatccggattgttgtacagcagggccagagtgtccaggaaagcatcgtccaacagcacggagttggcggcctccggcgtaacgggacggtaacggataagttgcgatagcgggccatcgcgcccggtaacattcaccaacgggcgcagccaactttcatacttgtcaccctgaaacacctcacccaacaggcatcggcgcgttagttcggggcactccgcggggactttctcggcggcggtaggagcgacgctgacggcggctgaggaaacaatggacagcagaaggcaacaccacagcagtatcaccggtccaggtgagaaagagaagccgcaatccgggcggcggcacatcaagtctgcggcacgatgagagtgtgacggtaaggagccagttggcgccgaaagttggcactcaggtcttcgatccctaaaacgttatatattgcatccagcaggtgagccaggctaaacggattcacgtaccaggtttggttacccgcgacgatgacggccagaccgtgggcgctacagttggagaggttcctgggtacgaaggtaactgagtcgatgtcgcgccacggggggaatgagacagacgactggcgcacgctgtaatcacaactgtgattgacgtattgtagcgtgtaatttaggttgcactcagcctcgaagtagagggggaaccacagttcgtcgtactcgtcgtcgtcctccagttctggctcttcttcatccaccgcaatgtctacgctgctttgagattcctcttcgtacaggatgattgacaggttatggctacaaaggtcctgggcgggaggacgcgtgggagcgcgggtggtggtaatgttttccagatcaaaagttggagtgtagtcggatgttacatccccgttgttggaggtggtagaagttgcggccggtgtcgcggtggtaagtatggatacagaaggggagggggaagtagcgttcgtaccgatggttgtggtattattattccctgtgtttcttgttccagaaaccgttgacgttgagatgggaatcgacgtggtgctggacgtcagattgctgaccgaggaaaccgtggtgggagtggtgacggtgttactcgtggttgaagtgacgttaggggaggtagtagtggtaccggtggtggcgacggtagtgtttgtcgtggcggcggcagcggtggtaccggtaacggtggtcgcgttggtttccaccgcttcacacagtaagcaaaagcacagagccaggaaaagcaaccagccccgccatcgccgccgccgcttcatgaggtgggcaggcgaaagctggtgaattcgttgtacagcggcaagtggggcgccgcgatcgaagggtacgtcaacaagctgacgttgatattaaatacgtctggctgcttttctacgatggaagcgcacagggttacggcgtcaaacaggtctttcttggtggcgcccgagacccacatctggtatacacccgtctcgtggtacgaagtagagcgcggcaccaccggacggatgcagtccagaacgcggttgggatcttggtgaaagaatttgaacgtggctacggcctgtggcgtgtgcggcatcgtctgcgtgatgagctgctggcccgctaacacggtgacgttgtgcaacttgagcagggcactcttgagggcctggaaagcgttgccgcacgaggcgctgatctgcagctgcacggccgtggagtcgtgcagccgcatgagacgtgacacctcttcgaagacgtacttatacttactggcaaagagtggcgcgtatcgacagtcggccggcaaaatgtaggtggcgttgccgccgttggtagccacggcgggcgcagcggccgcggaggccggcgtaaacagcgtcagcggccggtggtggctggtaaggtcgatcatgggcggcgtggtgaccgtggcggtggcgggcatgacggggtttgcggcgacgggcactccggccacagcggcggccgcggcggccacggcggcgctggccgagcccacacccgccggcagtcctccgctacccatgacgccgccgggcagagcgtcgcccagacagacttccacagtggcgggcgcgctctcggcggtcagtacggtttgccgatcgacctcgcgacgaaagctggtgaggaactcactatgatccatggccgcagggcccgagatcccgggattctgcgggtgctgaccgagtgcgggccgagttatatggaagacgattagcttggagcggagttttgcgtccctagctgacctgcggatcagcgacgtaccatagggatagactgtgagcggcggccgcaacggcggggtcggccgccgttcgtcgtcacggggcggcgcgagggaggaggaggtggtgggtacgatcttgacgtggttgacgtcctgcccgtccgggggaatacgcaaaaaaacccgtcgcggcgctaccacgatggtgcgatgggtctttctcttgttggccggggccagggacttgcagatgcgtgtggagccgtagacgatctggacgtggtcctgggagaacatgaccatcgccgccaacgctcagcggggggacgtattgggaacacagaggatgagggaaaactccgtagaagtcagcgaaataaagacaacacagcagccactcctctcgtctcgggccctaccactgcttgaagtagggcaccgggtgtttcttttcctcaacgggctcctccagtctcttataggaccagtcccgccggcgcgccagcatgtaggtcacgtacaaaagaataatcaccatgaacaccaggaaagccagcacgccgtaggccagcagccggtcctcgaacagcgggtcgctcttgataaacacgtaggtggtggtaaaacttcggcccgcgatctgaacgtggagacgcacgacagtatacgtgccgttgaggtagaagacaaactcgcgtaaccgttgtccgttatacgtcacgttactaatattccacggcggaatgagctggttgccctgatgcagatgcacggtgctgttggggtgatagaggctgctaccgttgagcaagcagtgttcgtgttcctgaagcagcacgcggacccgcatcgtggtagcgttcaagcgagtcccgtacacggcgtagatgggataggtgaaaaggtcccaagtggcgttgtgatggcggccccagctgaagaaagagcacgtgtactcagtggtctcctgcggcctgagtcccgagataagcagctcttgagcagtagcgttgtaggagagatgtagttttcctgtggaaaaaattaatgagttgtttattttgttagcaggttggcgagggaggaaggagaacaaaacagaaaggtacgtgttacttacctttatcgttggagggaaaagcgctaagataccccacctgagtgaagggacccttgcagtctgtccgtgcataacaagtaactgataaaatgtctggatttttggtattattcaacaggattactttgcaggtggcgtttagagacacttggtcgtagctgtagctggcttcgcaattcacagtatacaggtgcccctctttctgcgtcgtggctatcacggaggtggaggcggacgaggtagaggtttgtaccgtggtggtgacagcagaagtgacgttgttagaggtacttattgacgtagtagacgtgacggtggtattactaggggaagtgacggcgcttgtggtgctacttttcactctcgggtgcatgtcgcccaagagcgcaactacgagcgcgatcgccagcacggaacacatgttgccgtgtgacgagacggcgtgtggacgagctatatgtggcaggaggtcgcgtcacctcttgtgacgcctaaacgtccagctccagataaaagaggcgttaataatgaagaccacaaaaaccacttgcgtcagtatgacaatcataaaggctcggtgattgctacgcctaaagtacgcgggattatccaccagttcatcctgctgaacaaagtggatgattgacgtgctggtgttaccggccgtcgtattgatcatggattttactaagaaagttttggcaccaaaagtcccgttagagccccagcaggtaacgctgccgttcacataggctcccggtgcccctgtcagcatgcgtttcagttcatgagtataatttttcctatcgttatacatatcatcactgtagttgactttgctggtgagaaactgtgtgttctgtggaatactgatcatcatccccgaggccaaaaagggcgaatcgcaagctgtagtgttacagaaaatagtcaggttagtgtcattatgctcatacatataagccacgctaacctggggctcataccacccaatcgcaaccgccagcacgtcccatctcccgacatttatcaccgccaccactaacaacgtcacccccgcacggtacatagttaccctctcgacgtcgccggctgtcaatgacgtgcctgcgtcagtggctatgatttatagcttttggacacaaccgcaacggatctgtcgtaatctaccttccacagggccgccgcgacgatgctgaacgacaggatcagacagacggcgtataggagtcctaggtcggcgtcgacgcggcaagtgcggatgtctcgcagggtgggtagatgggcgatgcacaactctttctccccccgcacgtacatcccatctcgtatcagcagccgtagcgtagcattaatggtcagcggggtaaccaaagaaatcacatagggatgtgtacaggaagtacagtgacgggtatccgtgagatgtaagtcatcacccttctcactgttatcatgaaagaccaggactcgggtaagacgacccgatgaatactggatctcccaccacagtctttggtccaacaccgagagggcacaagagattctaagtctccctgggttgggggagcagatgtaagccccgtgtgtgcccctcgccatcagaaccatacacatgagggggaaaaggacaagtatccgggaccacccgcacccccacatcacgagaccagagacggagatgtataaaaaaaagctacttttattaaacagcattctcaccacacgttaatactgtcacggggaatcactatgtacaagagtccatgtctctttccagtttttcacttactgagacttgttcctcaggtcctggatggctgcctcgatggctaggctcagggtgtccaggtcttcgggaggggtctcggtgggctgctcaaactgccccacgtcgtaggccttcgcggccgtctcgtagataggcagcatgaacccaccctggttggtggagaagatgcgcaccatgacctgtttgggaaacttttgcatcaggggcaggcacaggttgagagcgcccaacaggtccacgggggtggcagcgtggatgatcatgttgcggtaatcggaagaacgggggcataattggtgggtgtgcaattctttgaggctccacgcggccttgacgccttcgttacaagcatcggccgtgcgctgcgccacttcgggtgggtgtgtcacaggcatggtgtgctccatgagaaagggagtggagagggccaggttgcacatggtgcccaggcgacaccgcaccgcatccacctcactcttcacctcatgattgcgggtgtagatgatctggatgcccttgttgttcacctgcatggttttgcaagctttgatggcctcatctaacacctggtgcatactgggaatcatgaagggcaggttcttgtattcaagagagcgattggtgttgcggaacatgcggctcacctcgtcaatcttgacgcgaccccgccgagtctgcacgttgggtgtgcagaagggggtgttcttatctttcatgatattgcgcaccttctcgttgtccaactcggagatgcgtttgctcttcttcttgcggggtccggtgctcgccccgccgctgctctgatggccgcagctcagcagagaggaggaggccgcgccaccaaaaccgccgcgcccatggtggctcgaggtcacggatgctcctccgccactgctgcatttcatctcctcggactcactctccgagtccgaagccgaactgcaggaggaggaagacgaagaggaactatcttcatcgggccggcccaagggatcgggaagaggagggtggttcatctgggagagcgggtgcgtgggagaggtcactcgcggcgtgccgctgccggtggaaggggaagacgcggtagcaccgcgggtttcgacttcttcaccctgttcttcctcgctatcagagatcacgatacagccggcggtatcgataatcttgttgcggtactggatggtaaagtcgggctcgggcttgatgtcttcctgtttgatgaggaggggcagcatgataggcgcgggaggcacgggcggtttaataatcaccttgaaaggacgcgtggttttgcgcggtttcttacgcgggctgagctcgggagtagcggatgccccggggagaggagtgttagtaaccgcgacgctggtgggggtcggcttgttaagaggggcgctgctaacgctgcaagagtgggttgtcagcgtgtggccggtgctactggaatcgataccggcatgattgacagcctgggcgaggatgtcacctgatggtgataagaagacacgggagacttagtacggtttcacaggcgtaacacgtttattgagtaggattacagagtataacatagagtataatatagagtatacaatagtgacgtgggatccataacagtaactgatatatatacaatagtttactggtcagccttgcttctagtcaccatagggtgggtgctcttgcctccagaggtggtgggttcctcagcaccatcctcttcttcctctgaggcaacttcccctatctcagacactggctcagacttgacagacacagtgtcctcccgctcctcctgagcaccctccccctgttcctcatcactctgctcactttcttcctgatcactgttctcagccacaatcactgaggacagagggatagtggcgggtacaggggactctgggggtgacaccagagaatcagaggagctagcaccagcggtggccaaagtgtaggctgcaatagcatcttcctcatctgactcctcagcgatggcccgtaggtcatccacactaggagagcagactctcagaggatcggcccccagaatgtactgggcaaagaccttcatgcagatctcctcaatgcggcgcttcatgacattgataacctcaggcttggttatcagaggccgcttggccagcatcacactagtctcctctaagatatagcagcacagcacccgacaaaactcacttaagagagagatggacccgtacatggtcatcatgcaagcgtcactggtgaccttgtactcattacacatggtttccacacatgtagtgaggatatccataaatatgtgatcaatgtgcgtgagcaccttgtctctctcctcatccaaaatcttaaagattttctgggcataagctataatctcatcaggggagcactgaggcaagttctgcaatgccgccatggcctgactgcagccattggtggtcttagggaaggctgagttcttggtaaagaactctatattcctgtagcacatataaatcatttttctcttaagttcatccttcttagcacgggccttagccttcagtgcaccccctaacttgttagcggcgcccttggtcacatcatgcagctccttaatacaagccatccacatctcccgcttatcctcgggtacaatgtagttctcatacatgctctgcatagttagcccaatacacttcatctcctcgaaaggctcatgaaccttatctaagatatctaaggcattctgcaaacatccccccatcatattaaaggcgccagtgaatttctcttccgtctgggtatattttttcagcatgtgctccttgattctatgccgcaccatgtccactcgaaccttaatctgtttgactgtagaggaggataacaacacatataagtatccgtcctcctgactcatttatcgctatctcgatgccccgctcacatgcaagagttaatcttcactctatctgacatacacaagtaaatccacgtcccatgcaggttagtatatatcacatacatgtcaacagacttaccgagttctgccaggacatctttttcggggttctcgttgcaatcctcggtcacttgttcaaaggttttgagagattcttcggccaattctgggaacagcgggtctcccaggctcagctgactgttaacctccttccttaacatagtctgcaggaacgtcgtggccttggtcacgggtgtctcgggcctaaacacatgataaacaaagtcataagcacatgggtcacatacaggaaatatgtatataacattaaagatataactttttattaaaaaaaggggaacacaagtcccgacacgtaccgtggcaccttggaggaagggccctcgtcaggattatcagggtccatctttctcttggcagaggactccatcgtgtcaaggacggtgactgcagaaaagacccatggaaaggaacagtctgttagtctgtcagctattatgtctggtggcgcgcgcggcagcaacgagtactgctcagactacactgccctccaccgttaacagcaccgcaacaggagttacctctgactctcaacagaacacaactcagctgcctgcttcttctgctgctgctgccttaaatcttccatctgcgtcagcggtgcaagcccattccccgagctcattttcagacacataccctaccgccacggccttgtgcggcacactggtggtggtgggcatcgtgctgtgcctaagtctggcctccactgttaggagcaaggagctgccgagcgaccatgagccgctggaggcatgggagcagggctcggatgtagaagctccaccgctaccggagaagagcccatgtccggaacacgtacccgagattcgcgtggagatcccacgctatgtttaataaaaactgcgggcactggggacggtggtgttgtatatgtgaatttgtaaataataaatgagaccccatcctgtaaaaatacagagtccgtgtcagtctctgaaggacagagtattggcatatagccaataaagatagttgtggcaaagagccatgttatggattagtaatggaaagtatcgtcaccaataggggagtggtcaataatggtcaataacccacacctataggctaagctataccatcacctatagcataaggaagcgggggtgtatagaccccaagccaaaaacagtatagcatgcataagaagccaagggggtgggcctatagagtctataggcggtacttacgtcactcttggcacggggaatccgcgttccaatgcaccgttcccggccgcggaggctggatcggtcccggtgtcttctatggaggtcaaaacagcgtggatggcgtctccaggcgatctgacggttcactaaacgagctctgcttatatagacctcccatcgtacacgcctaccgcccatttgcgtcaatggggcggagttattacgacattttggaaagtcccgttgaatttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccgtaaggtcatgtactgggcataatgccaggcgggccatttaccgtcattgacgtcaatagggggcgtacttggcatatgatacacttgatgtactgccaagtgggcagtttaccgtaaatactcctcccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaccatggcggtcatattggacatgagccaatataaatgtacatattatgatatggatacaacgtatgcaatggccattagccaatattgatttatgctatataaccaatgactaatatggctaatggccaatattgattcaatgtatatatcgatatggattggccatgtgccaacttgatgtcgcctctatcggcgatatggcctcatatcgtctgtcacctatatcgaaactgcgatatttgcgacacacagaatcgcccaggtcgccaaagtcgtctatcgccatcccccgtaaacgatataagcgctatcgccagatatcgcgtatgcccaaaaatcacttttggaaaaatggcgatatcagttacacagagactcacatcggcgacattttcaatatgccatattttcaaatatcgatttttccaatatcgccatctctatcggcgataaacaccactatcgcgcgacatgaatttagtcggcgacagaaatctcaaaacgcgtatttcggacaaacacacattttattatattatttcccgtgacgcaggctagttggcaaagagccgcacgctgaactcgaggctccgggcgtgcggcgccagcgaaccggcggcgttgaacgtggtccttttgttggtgccgccgcgacggttctgacgtctaaagtcgctgatgagcaacgacacctcggtcacgttgattctgcaagcacaggttccgaacgtcatttcacaccccatgcggttacctacccgttacccgttcgcccttaccttcccgttgtcatacacctttagcgcgtaccctcacctcttgagcacgtcaaagttgtccaagccgtggctcgcatcgtagtggtagttcaacgtgaggtccacgagctgttccacatacttgtaacgggtttggtcgggcagcgcgcgagagcacgcgtcccagtaatgcggtactcggtaataatcgtttttttctgcggtctcccgctggcactgacccagcaccacggcgcacagacaaacagacagccacacccgatacagccgcatgttgcagactgagaaagagagctttattatgagacatcatacacatagtataggcgaggtaatggggcggggaaagagttggaaccgaaagacaaaaaaaaaagcctagtcgtactcgggatctctgagcgagacggattgcgtagcaactttcattagtttgggaatctgccagctggtgctgttggaaggttcttccatttccgaggcggtcagttcatcgtacaccgagacgtagtacctgatggggtcctcctcattgtccgagaggtgagattcgatggtcaaaggcgagcctctcccataattgggattcacgaacgacgtgtccaagttgccatcctttctgaaatagatgacgttctcaggatcatgtttcatgcgctcgcgggccgcggacgcctcctcctcctcgtcccagtcccgagtttccaaccgctgataagggctcgaggaacaaaatccggcggggatctgagaacctcgtcgggaaccgctgccaaacgggctgctgccgccactatcgtccgtgtcgtccaacaggttgacggcctcttcgtcggcgaaacgaaagcggcccgggtgcttgcaacacgaggagtaaactaccgcgatgagtaccgctatgaagctgaaaatggaggtgcctgtcacaatgtagaagaggatagccagcactttcatgatttcgtcattgcgcgcgtcgtgaacggaagattcgcgggcggtggtcatgttggtttcggttgtaggttcgctactcgtagtgctctcgacggtatttctgctgctggtgctagtagggacgtttgtgctgctggtcatatttgtagcgtcgctgaagtccatgtgaagcagcaacccgaacgcgaccaggaccaggaatgttgcgcgaaggagaccccgcggggccggcattcttgagacgtggcgacgtggatttcttgttatgtccgcgaacgacgtgtgacgaggacgtggtttccgcaagcctctaccgacgccgcgacaccaggtaggttatgaaaacgcgagcccatatcgccgccatcattgtaatcagcaatgtgttgaggtactgcacgatgaatctgtctagtgacaccagccaaccctctgcttttgcgggcaagcgcgctttcggtgacagggtgtatcgtacgtagccgcgggtcaggcgcgcgttgtagcggtacacgcagaaatctatccacaggccaacgcccggctgtagcttcggatggtggataatagcgcggtgacgtacgccgcggggctttagaatctccacctgtaaggccatctcctccaggtagtgggtctgactgcgacgcagcgtccagttcatgtaaaagtcggtctcgccgtgtccggccacgaagaggctgcttactaaatcgggcgccagagctaggtcaggcgtatcaaattccactgccaggcgacctgattctaacggttccacgatccgggagagcgtttctagatatagagcaaagcgtaccacgtctacctgcggtgtaaaaaactgttgtgggcgttcaccgtcgttgaccacgtaggccacgtagaggccaacattttccaccacgggttctagctgcaggcggcacgtaaagcttagaaacgacggctgtacggtttggttcccgtgaagctgaagcgtcacttccttgccggggctcaccgtgctgtaacgtcgcaccgagtcggtcatctgctccagatcggtagaccagaaaggcgtgcaatgcatactgtcccagtcgcgacacacagcccagcctagctcggtgaagggtcgacgcacacccgaaaaagtgtgcttgaagaccagggggtcgcctcggtagctcagtagccgaacatgcacatagtcgcggctagcgttgacagacggcccgtggagggccagtaggacgagcgtgaacagcaagcgcaacatgctgcgcgggttaggaaatgcggcgtgccggccaccgcccgactcataaacgctaccagcatgacgtctcagatcacacaagtgacgaggagcgtaccgcaaatcactagggaaaaggccagcagagcccgatagtcttgctcttcgcgaacgatctcgtccggttcctcgcagtcttcgtggtccacagaagatgaggagcaggattcttcgttaatctctgccaggatactagtgctataccacaccagagcgctcagcgtgcccagggctaccgcacggtaaaatagggacatgatcaccagcgcagtctaaagtagtggtaattaagtttcttggcgtatttccagagaaaggctttgtaggccgtagggactggccaggcaccgaactcaatattggtagacactacgtcgtaaatgcgttgttcctcatctaagattaaccgaaaaaatagccggttgatgtgacggcgcacggcttgcgcgttaggattgagacacttggtgcccttgtcctttaaaatagccagcacttcctgacgattgcagctttcgctcgctgcgattggcttaagcagttgagttccgactggcagggtattcaacagaatttggttgttgcaacggcagcgcctgtcgtaatcttctagttctaaaacatggacggctaggggacatatggtaagtaacatatatgcgattaatgacaggtatcgtaccgataacagattgatatgcgagtttgaaaccggatggtgcaaccatgttagtaccatattaaacacatactgtaatattttgttttaacgaacttgtctgtttgaaaacatacattaaatattatcctctaacacctatcaaggttatatttattcgtctcggttctggtgatttcgttatgttaacattataccacctattatcgttgcgtttgtctaaccattttgagaagaggtgatcgggcgataaacatactccatgtccaggcggcttccttccgtctggatacaataaatgttcatttttatcgcatccgggccctctgggatcgcgatgaagccaataattacccaatataattttattaggcggccattttctatgaagacatctgcagcgtaattctgttccattcacctcataatgatacacatatgctaaaaaaataatcaacgcaccaaaaattaatcgcattataattttattatctacgtcactaccagtaattcgtaatatccggtattcccggaaaatcactcaaaactgcgtccatgacacatcaattcccgataagtacccccctttgaaatcggatccccccacataccaatcaatcacacaacacacaggtttaaaaatcgatcacacgtcaattaggtttcaaaatcgatactgtttattatcaggaatctagactaattctacaatgacagctctgaatttctctcttgtctttcttgtcaggttctcatcatcagtcatcacttccacccatcgaggagtcatcgtcgctccaaaatcctttggggtcgctagttggaaaagtctctgacacgatccaggcaccccgcacccagtccgactgatctagcttgcggagcatctcaacaggcatgagctgcagggccacggctgtcacggcactgtatcgatgtaacactagggactttctttgcgatgtagccatcaacacggcgtatgccccatagttcgcgtgatacgacgcatgatgggttaaacgttcccatccggcagtgccgtctcgggtccgtgcacacaacagctgcacagcgttatgatgcttaaaattaaccataacgctgggactactgatgaaggagtagtaatgagccaggacgccgtacatcgaaggcaacaagaaagagtgacagcacgatagcaccgggctcttatgtaggcgacagcttatttttcctgacgtcggcaaaaagtacctaaattccccacagatattcagacacggttccgtaaagtgcttctttttttagtgcaggaattggaaaaaataataaaaaatatgaacagctcatctgtaattatctgtgtgacttcatcgtaccgtgatgtaaaaacaacaacaggaagcttacagggtgcggtagaaaaatttgccgattgtgcaacactgttggcatctctcactccgataggcggctataagatagagaattaaaagtatgatacccacaagaaagatgaagagggacaaccaggctagagtatgacgaccgcttttcctttgtttgacggttacatgtgcggtatgattttgctgtcgttgcttgtgatgttggacacctggagtggaaaacgacgtatgattcttagatgcgcatatggtgttattagtggaagtgcagttacgaaccgtgatctgagtgtcgttacattgagtacaattagtacagttgtaaagccctgtgagataagtaccgtttgggcacagtgtacacgttatgccactattctctgtacacacttttgtaactttttgtcctgatccgcatggcgggcaacattgattacccagcttcacctcatcgggcttacacattttacttcccccaagctgtagtaaaaacataccgaagcagatgagcatcaccagaggcttcatgcctcctaccggaagaataaaaataactcatagggccgaacggtgtcatcctctccgcggtttgtaatacgagattgcaaacgtaaataaatgacataacttcactaacacgcatactacaaagtccacctacgacgctgaaagttcttccaggacagaacaggatagtcagccatcttcacagtctacctcttaggccgtatccaggagcataggtaatcagtttccagccacagtacagcgagcccaggaaaccgcacacggtccctgccgggaacacgtaccaccacatcgattcgtcgtgccgtagaaccgtagagttttccgaacttttatacacgccggtggcgttagggccgtgtgtgctgctgtgattggaggttttgtgagctaggtaacagctgtgatttcacctgtcgccaacactgacagcgattacccaggtggagcacaatcacatagctgatggacgttggttgatccgttgattcccatggacattttaacggcgacagtacagctcccgttaaacattagaataatagacgttagtggataacagcatgttattcgcccaagtgtgatcgtggttatacactttcttgttttttgctcatatgctgtaaggtgttcgaggatcgtggggagtatatgtgttgaatcggaatcatgtttactgaccgcgccatacttcgtatacgaacctaaccggcgtaaagtgttttccgatatataaactggcgcctattgtggctgtagcgcccataggtatggcgtatacccacggtgatgttgtgttattcgttttttgtgataaaacgtagcttatgtttaacgtgtgttccgtcacgttatgtgtgtcgttaaaagacggcgcctgtacagtatggctttgagttgtatcttgaattgttattgcatctggaggtgttgtgtacagagtggttgttgcgtgttgaggtgttgttacgttttgaggcacagttgcggtgtacacgggctccaaggtgtagttacggagtctttctatgcaggtagtgttgagatatttttgaatgctggttatgttcgattctgtgaggttaaagtgtgtactatttatgacggtgtaatttagacggtcttgccatcccgaggatattagtgttaggtaattcgtgttgttcacgtttgcttgatatgtataggtaggtgtactgtttgtgaggtcgcaagtgtgattttcttgcagagattttatccatcttgtgtgaaaatattgagatacgcgatgaatgttttcgctatctatattataaagcgtttcagtgtcacctaggggttgtttgttgtaacttttattttggaccctgggtgtgaaccatgattccaatgtttgtatagtaaggtgtcctactaataaagacgaactgattcctaccgtaatgttataccgcacacccagggtgccgtttacaaacacggaaatgtttccgttacaaaccacgttggcagatgaattagattccaggtggtaacgataggataatgaccgttcgctcccaacggatgacacaaagtatccgaataaccaacacgcccattcaatccgcatattttaatcacactattcacacctcacacactgcattttttaacatcttatttttttattttatgcgtgttctcacctcttcatctttttaacaccggggtaactatcgtaagtcggtaggcgtcgatagccctcaccacctcgtcgtccccttcccggcgtggggcaccagcgtccacagcactgcaggtaacacaggtagcataggaaacatacggtgaaaatactccaaaatcccaaaaatgccgcgattccccgagtggcccagggagacatcccggtgtctatgtcggccggcggtgctggcgtcaccggtaaaaatttcggcgggtgtggctgcgaacggtagcagtcgccggggagccggtaacgctgtatcactgtccaacagcggtcgggttcctcgtccggacatgcgggtttccagcaatcctcggcgtcggcgcgtccgatatagaagtagttgcgctgaaaaccgcggtacatcccgcagtcgtgattccgtagacgccagggcgtcggcgaccagatctggtctcccagcgagtagcgacctaacgccggcgtgcagcaaggttcgtcgggccggctgagcgtctccagttgcgtgagaattacgaagcgttgcatgatgaggccgtggctgtagttgcgcagcacgcattcgtacatgccggccgtgtccgtcgatacgttgaaagtcagcgagaatatttggccgagatgcaattgcgagaaattccaagtggcgtacggcaggcggtactggagtccgttcatcagccgatggcctttgacggcgtccaggatgagctcgtcgctgccgtcgtgggaacgacagaaacgtgcgcgaatggagaccatgggccaggagtgtgtcatgaccgtgcaggggatggtaacttgctctccctcggcgaccaacaccggcgccggcgacgtggtctcataattctcggcccacatcttttcggcaatgtcagcggtggcgaaggggaacgaagaggaagaatattcgaggagtcgcgggcagctcaacagcacccagaacagccacggcagagttcggagcgactcccggcggcacatgatgattctttccttccctttttcgcagagacgctgcgcgcctgctcctgctccgtgtgtcggccgctcaaacgtcgggccggcgtggtggtgaccaccgtgcgacgcagcttctcgcccgggatgcccgcgactgagcgtccggtttttttgcaggtcttttttgctgcctcctcctcgccgtcgccgtcgcggccgacgtggtggaccagcaccgcgcaggaactctcgcgtcgccggcggtacgcgacctgtctcattgctacctcggatgtttaagaaggaacgttcatctgcgtcacagggtctgatgaagctgccaagagtcgtggctgtggcgcagcgcgttctgtacggcgcgtttcaccgctttctgcatggccgctaccacgtcgggcgggagcggctccggcggaagctcgatgagcagttgctgcgagtctcggcgctcggcgtccgccgtttcgtcggacgtggcgtagaaaaccgaggtggtcgcccagtcgtccacgctgtcgacggcctctgtcagtgccgggttgtcaaaaccgccatcggacgcgggtgataaaagaacgtacgatgacacgctgttagtacgattctcgtcgtcgctctgggaacgacgtgatggacgacggtagatgacctcgtcttgccacgcgtcgaagcgatcgcagcagcgctggatccaagcgcagcgaagcagcttacggaacacgtcgttgttccaaaagtagagcataaagagaaagaaaagtagcgtaacgatgaagccgaaaacgacgagggtcggcagggcactaccgccgctgccgttttttgtgtcgtgcgggtgcacggtggtagtggcgttagtctgagctggggtcatgacaagtctgaagagatgagagcgtgggtgctcatcagggacagttgaggtctctccctaccgaagccttagcctctacggtgttttatgatcaacgtgtctacgaacgtcattgtgaaagtgacgtctcaggctttccgaaaccgcgtcagattcaacgtgggtttcggtttagcctgcgtcaccgaggcggaggtggaaatgagccgtcctgtgggggagtgtacgaccctgtagtgcccatgggtaacgtcgcgtcggaagaagtgaatgcggcattggtgtacgcgtgggttgttttgctctctgactcggaggagttgccgcagcagctgcagattttacgtactagccaaaagcagcaaaagcagcaggtaaataagagaaggagtccagataatgtccagccgctagcggcaaacagcgcaagttgcgcgactgtccaattactaccaccaaaactctcaacacattgaatcgacgctgaggttggtgttgcagtgctgttgctactagtggatgaagacgaagtagattgactggaattagagctggtacctgtagtggtttcacttgccgatgcggcaagtgcaaataaaactaatatccacagcatgttcgttactatataattgatatacgaacccgtttgtcgtaacaatcagcgttatatacgctgtatcggcatcgttttaccggaaagtttatcgtaatgtaacccgcgttgtgtacattcgtactgaaagggaacccccggtgatgtgcacattatactctttcattctggggtttcccaatgacgtaaaaatttccactatacaataaaattacggaatcatgtgaaaagtgtgctttttattaacagagcagagggtttacagtagatatatgtttgccagggccactgttttctaacaccgatcaccgccaccattaccacccgttgaactccacacccgggagccgcctgatcgccagggactcctcaccgtccatcgtccgaacaagctcccgccaccgatgctgccaccatcaccgagagaaagaaccgcttgctgcagatacgcttgggctcgcctccgtgcggacgccgtttcgtgcagacgctgagtagatcgagcagagaatgtcaaaacgacattaccgcgatccgctcccctcttttttctttttctcattcacgtgtattcttgatgataatgtaccatggctacggtggtgaactgcgtcgcggatcccgtcacgggtttcaacagatcgacgtcggtcagcggcgccgtcaccgccatgtccggcggaggcacgctgtttctctggttagcgacgtggaccgacgacgaagacgatgaacccgcgcggcggtctgttatccgcgacgacgcgtagctgcactgggaagacacttcctcccaacggaccaagatctcatcgggccgttcggagaaacggtatcgtctgtccgactcccgccgtacggcgccgaggcccagcgacgacaggtccgcgaaccggcgctcgtattccccgtacagctcgcaacagcggatcagccagcggtagctcaaaaacatgcgcaccagtttgaaggtgtcgtgccaatggtaagctagatagcagagaatggccacgatcagcacgagcatcacgccgatgatgggtaacccgacgttcagcggcagatcgtccatggtgaccgtcctctgtccggatctacgtcccagtctctctcttttgtacagcactcgcgcgggaacggccccctcaaccctcttacgtagcgggagatacggcgttctcccgcgggccacttacttgcacggtcgcttgaacggcggcttggactgccacatgcaccgcatccatccatttcggcagcagcgcgttcgacgatgtcgtacgagtcgcggatgatgttaccccgccagcacctccgccggcaaccgcgtcgtcgttgctatcgtcgccggtttcgggcgatgacagcgccggcggcgcgggtctcgtctcgtccaccatttccaccgtgtcgaagcgacagccgctgccgtagtacatagctccgttcaacggccggcgggccgggtcgccgagttccgggtcgggcacatccatggctcgccgtctccttctttgccgctcgtggtgccgacggcacttctcgggataatgacagccgcaaaatagatcgtggagcatgtctcgccaactgtcctggtgataatatcttaagtacgcgatgagcgcgccgatggccataatcataagcgtaagcaaaacggcacagataacgtgaaacaccgcggtcatccaagtcgggcggcgtcggggacgcggtgggtcggtttctcttacgccggcgtcactcagccaccacacccgtagccgacattcccagaatcggtgaatgcgactcaaggcctttcgacgccgccatttatttccaacgtccaagtcccacgtcatttctggcatctccacgcccttgactgacatactctctttctctctcttagctgcggtgaaaaagagggaaggcgtgtgctgctatacaactgtacaacggacgcgctcgctctttcggtctcaggtcatctgcatcgactcggcgtccttcatgacgctctgcaccgccttttccaacagttcctcgatgtccgaccatcgaggaggcggggctaactcggaaaccgacacgataggcagcgtggtcggctccgttggtgtgcggggtcggggacagggacacgagagtcccaccttcgagagattctccagcccgacggtgcgcggcagtctcggattccgcggcggcttttgcggcgtcggcgttttcgggaagggcctgggcgtcaccggcggtgtccagccgaccggcttgggtttcgtgggcggcggtgttttcttggtgagcggcgtgctcaggttcttacgcggcgcgggtatcggcgtcgggggcctgtgcgacgacagccgcgtggtgggggcccggaccggcggcgtaggcggccgcttcttgcgcccgggcggcggaggtggcttccaggatggtggcggctgatgcagcaccgtgtcgacgctggtcgaggacgacaaagagctcgacgaggaacaatgcgacggagatcggccgatgctggttggcgttcccggagtggatacgtcggggatctcgaaccgcgccggaggaaactcgggtttatctatcggcagaccatcctctcctatgtagagcgacgtacaccgcggcacctgcggcgtcggcgggtgggtggccacccgcatgagccccagttccagatccagcggctcgacgacgtcttctttcggattgcgatagcagcacgcgcaggcaccacgcttatcagaagcagcacccgggagccggcctcgcgacgaagtctcgtcggatcgcttgcggcctcggcgctgggtaaataaggaaatggccaggaccagggaagccagtccggtaccgccgaggagcccgacgccgagccacagccacaccatgatcttctctcctgcttggaatctcaaactccgtgtcgggaagggccggtgtacggacatttatgccttggatttctggaaacgtcattttttggcaaggaatgtgtttattgtccaaacactgaggaaggagatgtgggccaagtcggaaaattccttatcacaccgggggcgggttacgttccggtctgatgctgctgctgttgttgtagagccgcggccacggccgtctgcacggcagcttgtaccgcctcggccacgccgggtggcatctgcggcatggcggggggaggcgcgtcgggcggaccgccgggcatcgccgtcggctgtgacggtggttgtgaactcaccgtcggctcgcacggaggtttgttcttcggtctacccctcggtttgtctttcgccctacctttcttcggtttgggttccgatgtcggtgttggcggctgcggtgggatgacgggctggtgggactcctccgacggcggggggacgaacaccgtcggcgccgaaaccgggggactctcgactatctcgcagatcaccctgtcaggatcgtcgccgtgcccgggacgccgtcgatgaccgtattggaccatgtcgtaaatcatcgtctccttgtaacacgctgaacagcagcggctgcaggggcccgagatgcatttacagctgcacttacagctgcagctgcagtagcgcacccatcggcaagttaaaatgtcgattatggaatctttgaaaaattcccggtagcggatgaggtacgcgcagaggaaaatcatgaaaacggaacagacgaccacagccgcgatgccaggtccagaaaaaatattcgctgatgaacccgccaaacaccaaattcccaaggccgcgcatatcatccagatcacaatgatcgcggggacgccccattggcattggcacgaaggatcttgcacatcgcaacccatcgctactgcgttctcccacaaacgccatcgcactatttatccctacagcggctgccgagtcacgtccgccggcgcccatcggccgcggcgatctcctagtaacactcgtccgacacttccaccatctccagctcggccggcggttcggcatcctccaccagcggcgtcgtctcatctttgccgcagcagcggacgcacaccttctccaggcagaacgccaccagctgccgccgaacgtaccacaggtacacgtgcagacctgcgaacaggactacggaggtcatgaccaccacgacgcacacgggaatccaaggatcgagattgtcgctggaactcatggctatcgccaccgacgtgcccgcgtctgtctcaccgccgctcgcccgatgtcgcacggcttgttatacgctagcccgtcgccgcctcggggcacggtgccctcctacccacgtaacttcctccgtgacttaaagtcgcgtgtggtagatctcctgctccgtggacgaaccgttcggcaggatagcggttaaggattcggtgctaaggccgtgtcgccaacgtcgaatgctacgttgcaatagcttcgacggacggccatcctccctctcatcgcaataataaaacaccagcagcgcacacgacgcgatcacggtgacccccatgactagacccacgcagatagccagccccgctagcgtatccagcgccatcccgttcgctcccgtcgtcgtctcctgaacaaagcaactccgcagtccccgttttcaaccgttttcaaccgtttttgtttccttctccgcgactagatgttaacgcccgcggtctttccggccgtgctctacctcctggcgcttgtcgtctgggttgagatgttctgcctcgtcgccgtagccgtcgtcgagcgcgagatcgcctgggcgctgctgctgcggatgctggtcgttggcttgatggtggaagtcggcgccgccgccgcttggaccttcgtgcgttgcctcgcctaccagcgctcctttcccgtgcttacagccttcccctgaaacccgcgtcaatcgactgtcccgaaaacgccggcgttaacacaggaaaaaaaaaccacgcaggaaccgcgtaggaaccacgcggaacatgggacactatctggaaatcctgttgaacgtcatcgtcttcactctgctgctcggcgtcatggtcagcatcgccgcctggtacttcacgtgaaccaccgtcgtcccggtttaaaaaccatcatcgacggccgttataaagccacccggacacgtgccgcggcacttgcctacggcgctgctccagggaaactcctcttccttctgctcttcctccttcaccgcagggaccgtctccctcgaccagggacccgccgaagcaaccgccggaacaacctggaggaggcgcggcatgacggcacccaagtgtgttacgaccactacttatctggtcaagaccaaggaacagccctggtggcccgacaacgccatcaggagatggtggatcagcgttgccatcgtcatcttcatcggagtctgtctggtggccctgatgtactttacgcagcagcaggcacgcaacgggagcggcagcggctagataagtctctggcggctacagctccaagcgccgtagccggcccgcctgccgatcgcgacgtcgtggaccatcgaacagagactcacgcgtacgagaccccgaggtacgccacgcggtgcctaacgcggtataccacacccgtacggtctgcagtgcggcgtacaacgtgtggaaaacgcgttgcgtcgcagagtcagccacgtccccgtcttgtcgctccccaatcggctcccgcacaccccccgcggcacccagagggcgggtgagccaagtattcttaaggccgttctctgttccatatcccataaattgtttattccggagctcgttggcgcggaaatagccggataaggggagcaacaaccgtcggcgaaagccgtcccgctcattcagtccgggtttcgcgtctagtcggaggtgtgaccgttggccaacggaacggcgtttcacgatcaaaatcgtatcgggtagtgtaggagacgtcggcggtgcagaatgcgactcgcggcgtagctcgccgtcgctatgcggctcgtcgccgtgtggcgcggcctggccggctgtctgcgcccagatctgttggccttttgggtcctctggctgctgctgcgtgtgtgctttggcagacgcggtggcaatttgcggtctgcggtaagtgaggatatcgccgagcaagcgcatttgcggcacgtgggcggcacgcgtgttattgttcgttcgttgccagatagcaagtgctgtcgacagcagacgttgtgggcggttggtgtatttttgcgggttgcggtgaaagtcggcagccggcgtcttgtgaagtatcttaaccatctgtgttgctttttgcagcgtccagaaaagcgacgcgactttggggatggcctcgtgctcaccttcgcggagagcgccgccggacctgctcgtcagcagcgagctacgcagacggaatatctggaggagagttacgtgtgtcacaggggagcgcgggtctccggcggtaacgacggcggtatcgtcgacacgtgtgcggcctgctgtgctctgcggaaaagcgccggtctcggagaccgtggacgaaaaagagaacgcagcagctaccgctggcggcggcgtcgttaatgctgccgttgatgatcgacgttgtgagtactcggaaacagcggtgaggcagaagctcggttctccagggaacgaccgtcgatgcgtggtaggcgcagcaggtgaggttggggcggacaacgtgttgcggatcgtggcgagaacgtcgtcctccccttcttcaccgccccacccaccctcggttggtgtttctttttccttgtgttctgcagatagttccacggacagcgacggcaagtccataagcaccggtgtgcaagtggtggagcacgacgaagatatcaccgcgccgcagagtttgtggtgcacggcgttcaaggaagccctgtgggatgtggccctgctggaagtgccgcgttgggtgtggcagggctggaagaggtggcgcaacagcgagtccgggcgtcgatggagtgctgggtctgcgtcggcttccagcttgtctgacttggcgggcgaggccgtgggagaattggtgggctcgctcgtcgcgtacgtcatcctcgaacggctgtggttggcggcccgaggctgggtgtgcgaaacgggtgtgcaagccgaggaggccatggcgcgacggcgacagcgcatgctgtggcggatgttctctcgtggaggcgacggcgaatgcagcacacggtgtgcgatggagatggcgtgcgaggaagaaagcgccgtgttgtgagccgacggcgcgggacgcgggccggcgcagcgcgtgggccacgtgtggtggcaggcggcgtcgtccgcttgcggccgtcgccgcgccgcacagacgcaaacacatgtcgccgtcaagagaaacagtctgagcatagccgtctgcagcggtccgcgtgtagaagcggggggagaacgacgttaataaagaatagcggcggtgccgatagggcgaccgctgaagcgagctgcgtgtgcgtgcctgttttgttccccgtcgccgccgaaaagctacgcgcggcccccgtccctagccttgagcgcgccacagcacgccgcaaactcggcgtcgcgcttacgtcccgcaaacccccctcagcctcgtcccgccaaccaataccgtggcacgcagtgccatagcgccgcgcgtcaaggcgcttacacccccctcagcccggtcccgcaccggcgtcggtctgggtgtggcgggggtgcggctgggtgggtgtgtgccgggtgcggctgggtgtggcgggtgtgtcgcgggtgtgtcggctggctgtgtggcgggcgcgtgccgggtgtgtcgcgggcgtgtgccgggtgtgtcgcgggtgtgtcaggggtgtgtcggcggggtgtgcgcgcggccagatggaagcagtgtgccccggggcccgcgatccccccccccgccccggcgcgggcgcttcttctgcgtgtgtcctcgacgcgggtctgtgcgcctgcctgccggtcccggcagactgggctgcggcttcctcgttttttttttccgcctgtggccgtccccggggacttcctcttttccgcgtccgatcttcgcgtccccagggagtcgcgccgccgtcccctcgggaccgcttcctcttttccccggggactcaaagacacgcaagacagacgcgcgactgaaagagacgcaagacacacgcgcgtctgggtttcgccgtgcgcgccgcacggcgcttttattcgccgtcgccgtcccccgccaccgccaacttcccaaattcccacatttcacccccccgatgaaaacacccccccgcccctcggggacccagcacacggcccggaatggaggtcaggcgtccacctaggtgtgcgcgcgctcggcggcccgttgttggtggcttgtcgcgcatcttctttcggttttttcacggccttccagactgcgcggcggcaaggcggcgccagcaagcgccgtgcacgtcgctgcctataaaagccaggtgcgtgtcgcccgcggcacacgggcgacggaggcgtccgcgtgtgtaaacggcgtggtcgctgacgcgggtttgcttcctatatatacagagtggacgtcggaggcgtccggcggccatggcccagcgcaacggcatgtcgccgcgccccccgcccctcggtcgcggccgcggagccggagggccttcgggggttggttcctctcgttcttcttctttggaagcgacgtcaacagcggggactagtacgagtactgcgggtacggcgacgccggcccacgccgtccaccgggtagaaccccgcgggccgccgggcgcccctccgggtagcggcaacaacagcaccttttggcacggcccggagcgcttgctgctgtctcagattccggtggagcgccaggcgctgacggagctggaataccaggccatgggcgccgtgtggcgcgccgcgtttttggccaacagcacgggccgcgccatgcgcaagtggtcgcagcgcgacgcgggcacgctgctgccgctcggacggccgtacggattctacgcgcgagtgacgccgcgcagccagatgaacggcgtgggcgccacggacctgcgtcagctgtcgccgcgggacgcgtggatcgtgctggtggcgaccgtggtgcacgaggtggaccccgcggccgacccgacggtgggcgacaaggccggccatcccgagggtctgtgcgcgcaggacggactgtacctggcgctgggcgccgggttccgcgtgttcgtgtacgacctggcgaacaacacgctgatcctggcggcgcgcgacgcggacgagtggtttcggcacggcgcgggcgaggtggtgcgcctgtaccgctgcaaccggctgggcgtgggcaccccgcgcgcgacgctgctgcctcagccggcgcttcgccagacgttgctgcgcgccgaggaggcgacggcgctcggacgggagctgcgccggcggtgggccggcacgacggtggcgctgcagacgccgggcaggcgactgcagccgatggtgctgctgggcgcgtggcaggagctggcgcagtacgagccgttcgcgtcggcgccgcaccccgcgtcgctgctgacggccgtgcgtcggcacctgaaccagcgtctgtgctgcggctggctggcgctgggcgcggtgctgccgtcgcggtggctgcgctgcgcggcagggccggcgacagggacgacggcggggacgacgacgacgatgacggcggggacgacggcgatggcgacggggacgacgttgctgccgggggcgagcggcacggagacggaggccgccggcggggacgcgccgtgcgcgatggcgggagccgtggggtctgctgtgactttacctccgcagccgtacggcgccgccggcgggagcgcgatttgcgtgccaaacgcggacgcgcacgcggtggtcggaacggatgcggcagcggcagcagcggcggcgccgacggtgatggtgggtccgacggcgatggcgggtccggcggcgtcggggaccgtgccgcgcgccatgctggtggtggtgctggacgagctgggcgccgtgttcgggtactgcccgctggacgggcacgtgtacccgctggcggcggagctgtcgcactttctgcgcgcgggcgtgttgggcgcgctggcgctggggcgcgagtcggcgcccgccgccgaggccgcgcggcggctgctgcccgagctggaccgcgagcagtgggagcggccgcgctgggacgcgctgcacctgcacccgcgcgccgcgctgtgggcgcgcgagccgcacgggcagtgggagttcatgtttcgcgaacaacgcggtgaccccataaatgatcccgtcgcatttcgtctttcggacgctcgaactctcggtctcgacctcaccaccgtcatgacagagcgtcaaagtcaattgcccgaaaagtatatcggtttctatcagattaggaaacctccttggctcatggaacaacctccacccccatctcgccaaaccaaaccggacgctgcaactctgcccccaccgctcagtgctcaggcaagcgtcagccacgcactccgatacgatgacgagttgtggcgcccgctcagtacagttcacgaccacaaagcctggttggatctcgacgaatcacactgggtcctcggagacagccgacccgacgatataaggcaacgcagactgctgaaggccactcaacgacgaggcgccgaaatcgacagacccatgcctgtcgtgcccgaagaatgttacgaccaacggttcactaccgaaggccaccaggtcatcccgttgtgcgcgtccgaacccgaggatgacgacgaagatcctacctacgacgaattgccgtcgcgcccaccccagaaacataagccgccagacaaacctccgcgcttatgcaaaactggccccggcccacctccgctgccgccaaagcaacggcacggttccaccgacggaaaagtttctgcgccccgacagtcggagcatcataaaagacagacccgaccgccaaggccgccaccgcccaaattcggggatagaaccgcggcccatctctcgcaaaatatgcgagacatgtacctcgatatgtgtacatcttcggcccacaggccacggccgccagcacctccgcggccgaaaaaatgtcaaacacacgcccctcaccacgttcatcattgaaagtctctccagtccatatgttgtcaggacgtgctgtcgttctccgcttgctgcgaagcccgttcttccgagtcgtgtcgctgcgtccagcgtcgcgcccaagatgggaatttgggtctcttcacgcgtagcctcctccaccacggctgctgatcgccgtcactaaggaccgacacggaggatgacgaggagcttctccccgactccgcggtccgcgaccggctacgtagcgcgtgtccctgccagtctccgcagttacaccacacgtcgtgagcagcgtgcacctgctgccgccactgggcctcggcgtgctcgggccacccgccggagcccggtctgagctccgacgcaggatgcgcgtactcaacgtgcgccttccagtccatacagcaacaccataggtcgtgcgagtcgtcggctacccgccgccaggccagttcccgcatgggaaggctggacacgccgaccgagaggtcaccgagcccggacgccatctcttcttcctctccgtcgctgtcattaagcagccaggtcacctcctccgctccgcggtccgccggtctcgacggaccgcgccgccgtcggcaacacggaaaacagtacgccagcccgagccgctaaggccgcatgcccctgccgcccaactgaacacgcatatcccgctcaactgcgttttgccacccctgccagtgctcccgctcgagcaccaccccgcatctcccaacctttttccaataaacgaaaccgacatgacacacgtaatgggtactcgtggctagatttattgaaataaaccgcgatcccgggcgtctcagcacacgaaaaaccgcatccacatcatagacaagttacagtccacagtcacatacacgataaacaataccaacagggtaatgtttatggagtaaaacactattgtccaggccacatgcgtgtatgacttccgcaccatcccgtactgcatgttccacatgtacgcgctagacgtgtaatccactcgcagttcggggacgcaacgcagccagatcacatccccttgcagtaccagacgcagggctagcgtctcgaagatcggcatcacatctaagttccgcacgttccactttaacgactccccgggaacgaactccacgtcgtcggcgtgtacgtacaggttctctcccacgccgccataatcggccttcggatcgaagacgaaccgactcatgttgcccacgatgctcccccgagcaaacaacttgccgttgtcaatgtagcaccggttgtcctcgatttgaaaccagggatgcttggccgtggacttccagggccggagcgcgtcttccccggctttagtgattccatcgggcaggcggatcaagggacccatggaggtccaaagacccacccaggctttccagagattgttcatggtgaaacagcgtgtggactgtacgctctttcccaatttatatcccagagtagtgacgtgagcccagccacctcccagattcctgacgttttggttgtctttcctgccaattcctcccgtaaacttatgattatcctagcccattcccgataaaaatacacggagacagtagatagagttacgaataaaccggtttatttattcaagtgtctcaggagattattgaacgagcgtggataccacgccgtcgtcagttcatggtggcattgagcagccatagcaccagagtcccggcgcccggtatcagacacgctgacctaccgggcgccttcgagtccgtaccccgcggcctgggtgttagagtccgtaccttgcagcccaggtaggtttcaggtaccagctggttcgtacctgttaaataaatcgcagacgggcgctcacccctacggtcaggagcacaagaacaaccagagagaacagatatacgagcagggttctgaacagcagaccccaattgtcgtctctcatgcttcgctgaaggtaccagttgatggtctgagagctatagtccatcctcacctgaggaacacacgcggcatatttcttggggtctccccacctcgtagacaacgtgatgtccaccatatccacggtgtgcgtcaccgggtgcccaccgatgttccactcgaaataggctccgcgctcatcatggtggtactgctcaccggacacctgcagtctgtccatgtaagattgagagacgatacccacgttcacaaagtgtttctcggtgaagttgcccgacatcctccccttgaagtacagcatgcccatatggaaccagcattggttctcctccactcgaaagtgggccgatctgatctccgataccaccacatccaggggccggggcaccgagtccgcgagtctcaggaacaagacggccaggatcgcgagcaccaacaccggcttcatggctccgaaggtccgctgctcggctccgctcaccgctccggtctggctgcagcagtgcttcgctgagaagcagcgtgtggactgaacggtgtttttgaatatatagcgtttcttggtgacgttgtttcccctacgtagtaggcaactacgtgccaaaagaggcgttacggtactttccgtactgggatttccaaaccgggactttccacacggcggtttcaacaccgggacttttcacacggtgatttcggcaccgggactttccgcacggcggtttcgccaccgctgacgttctcatcgccgcccacgtcaacggtggcgacaccgtactttcccatgcggtttataaacgtcaagagtcacgtcagtcgcccacccccattacacggcgatatcccgatagggcatgaggggacccgggtgtcgcgacatgtcgacgacaggtgcggattagtggtcgtgtcgcgacatggacgtgcagggggatgtctgtcgcgatagagttgatgtgacagcccgctacacctctctgtcgcgacatgcatacacaacgggccggcttgtcggcgattgtcgcgacatatcgttatcagttagcgaccggagttgtctatcgcgacatatcgtcgactatcgcgacagaaaaaataccgttcgtagagaatgccgtgttgaaggaacgcgcttttattgagacgataaaacagcatcaggagccacaacgtcgaatcccacgtccagtcgattcgtatgttatgctgcacagcaatgctagaataacaaccagcagggtaatcccgcaacataaatacaaagtcacagcgaagaatccgtgtcgttctatcaagcgaaacgcgttccaaacggccccgtcacagacgcagttattcataagcgttaacaaccggtggctaggatgaatatccaaatcacagggcagtagccgacggactcgttgacaggtcagcctaccctcaaggttcctatcgttcggacgggatttgtgcgttttaggcctctttttcgccgcctgcaagcattggtgcgcaaagtcctcacccagctgtttccagctatcatctgcatctgtgcagtcccctgtatcgttgtaacaaacgggtctgtgcgacttcgttctcggaacacaagcttgttgtcgcggagacagagagagaagggttttcgggtcacgcgaagaccgctcaccgggggtcggcaacgcacacatcaacagaaaaccgagacgaatcaagagatccatagtgaaggagtgatatcgacgtgcttacgaaacggcgattatatatgttctcaacaataccgccctacgttgtatgatgtaacgtgtgacgtgagtctgatccaacactgaacgctttcgtcgtgtttttcatgcagcttttacagaccatgacaagcctgacgagagcgttcatcggggcatgaagtacgcattacacaaactccatatatttgttacgatagaatacggaacggaggaggctttcgccacacctatcctgaaagcgttgcattctttatgataggtgtgacgatgtctttaccattcccacggctgctttgcgtgatgatgacattcatcatgtatttccattcacacataccttttgtgcatacggtttatatatgaccatccacgcttataacgaacctaacagtttattagcccttgacaggataggtcaaaagattatatgtaggttttccggtaaaccgaattgtgatatttctctgcaggaaatagaacagcctggtacctataaaacggacaatgcagtactgtagcagcgtaaccaagtaggtccacatgaacacgtacaaaattatggtaagccatcgtttttcataccacagcctgtagctgtcgtacatgaatgaggacggtcgaggaacccagggtagttgtaattgggggcgacattcgtactgtccagaagacaattgcacgggtttcagtgagatgagtactttagcgatgtcggcgggggcgctacgtttcaccgtgacggtgagaacttgaccgtcgttttgtatttcatgaggcacgttatacaagccactggtatcatgaaggatgacctctgatgcgatgtgaggattaaattgtccctcaaaccgccaaacgctggtcatgtttccaccgtcaattacgcagctgacggtgtgagataccacgatgttggacttaggtttgggggctaattgcctttttacaaattcccttctgtattgcaggtcctgctgccactgcttttccgtgcggaaagtcgccatgtcttccacacgtgtggcgacgatagacgccaccaaggtagctaccagaagcagctggatccgcatggcattaccgtatgtcaattagaaagttgagcggacacggttatcgttcctggcggatataagtatataaacgcgagttagcctttcccgtccgttttgtacacccgttccccacacaaatgacgaatacgacctttttttttataaaaataaaccacgtgtattatataaaaacatttacatagaaaagagacacactctagattaattaagggccggccgcatcagcttgatatcgaattcctgcagatctgctagataacttcgtataatgtatgctatacgaagttatggatcaacataaggacttttcacacttttggggtacacaggcgtgccaccgcagataataagcgctggatacacggtacacagtcctggccagcacgtatcccaacagcagcaccatcgccatctgtatggcgatcacgaccccgagctctaagtgtctgtattcatagtgtagtcgtcgcaggttatccactgaattcccgtagctgaaataacgtatatggtaccgaggctggcaccacatgggtttgcatttggagcacggcaccaaatgcagagtgagatggtccaagtccgtgggcacccactggcgcaaacggaatacggcttcggtggtctccacgaggcactccggggcttgcagacggccccactttcgtccgtgacggcccgaccagccgacccgagccactatccctttctcgggatagaacgtaccctgtacacgccatacagcgtccaacacgccgtctttgacgacgcagctggcctgatagctggacacgttgttaagcggcggaaagcgaaactgacgtgccggcggagccacatagttcggttcaccgtgttgtcgcggttcgtcctccctatagtaatagtagtcgtcgtcctcataggggttgccggcgtgagccagcgttacccaacagcagcccaggccgacgaggaggcgcagccaccgcctcatggcggcttcgccagtcaatcgtctttagcctcttcttcccgtgaggtccttccggtggcgcggtgccgacctcggacccagggacgtatccacctcaggtacacacagtaggctacctggacaccgaagctgaacaaggctacatgtttcacaaactgcaccagtaccacatagaggaatgtcaggtagcgtctctccgcaaacagccgttccaagtctgagggcgttacccgcagcggcaaccagggcagcctggacgccggccggcaatggagcacgctccggttacaggcactgcaggggtaaacggttaacatcacgtaagagagtcgagcgtccacctgtgggagctcagtttcgtaacgtagagccccgtcattttccagctggggtgcgccgaccttgaaatgggtcgcgctccgttcgttaccccaggtgccgtaggctctcggggccgtatcggagaagttgccgtgcacaagccaggcggccacgagtaccccgtgctggacgtaacattcggacacggaactggagacacggtagccggacacgtccccaaacccgcgagggtactggggcagacggacggacttgctatttgacaacggacagatacgagacgacgaggacgcagacgactcgtcgctggaccacgacaaccggagcgactccttggagcggctcgagagtacacttactgcgatcagacaccagtgccagaagaaggaacaggtggacggggaccacaggatcatagccgccggcaccgcggccggccgcaggaagccgcccggcgcgtcgtctgtgtgcgggagccgaaacaccgtgcctctttatatcgtcccgacgtgacgcgagtattacgtgtcaggggaaaaccccgtcatgacgaacgtgattcgtaagtgacgcggggtgctgacggggttcggctcgagaagtgacggagcgcctcacgtcagtatgatgtccgatccgcgtcagccccgacgtggttatggtcaccgaaacccacgtttatatggacgttgagaacagcgcctgaccacatgattcatcataccatttctcggaatcgggcccatgccgggaaagcacattccttttcagtaaacaacaatgacatcataacaaatcattttattcgcgaggtggataataaccgcatatcaggaggagggatcgggtgatgacgcaggccccgcaaaacagtccgaaataaatttttagtatcgccccgtagtcgcctagataccagaggtacgtcaagttcatcaaaacgcccatcggcgtcccggaatcgtataccgggcacacgaagcgttcataacaatcccgggaggcgagtgttagggtagcagaatagtttcggggtcggtttccttccggcgacgacagctccgtgggcagcagaatgtagagcgcctcggtagccgtcgcggtgccttccacgaggatgggctgccggtgcctttcgtgattttctccgtcgtgtagccaagccgaggcccgcaaagtcttaggcgaggggaattgtccatagactttcaccgcacccttcagtacatggttctgaataacacagccgcacgtgaagtaggtcggttctctcgtctcctccgtggctgccgccaccactcccagccaccacaacaggcaggtcgcgagagggttccggaggcttccccggcgtagcatggtttcgggttaaagcaaaaagtctggtgagtcgtttccgagcgactcgagatgcactccgcttcagtctatatatcaccactggtccgaaaacatccagggaaaatgtcggtgcagccaacctttcacatacagcccccaaaacacttgaatcactgccaccatcatcagcgtatactgcgccgacttaatcgtgagcgcgtagtacgccattagacggcgatcttcgaacaatagtcgttcgatgtcctctaacgagctccacagaggaacccaaggcacgaggcaccggggttcgcactctacataataagtttggcattggtggcagggggaaaagtagaacaacacgagttttgtgcgttggggaacacgatagtcccggagccagtagcgttttgcgacgaggctttcggagacgtcctccaccggcgtcggcactcgatccgcgtagccctccagcgtctggtagtacacccggggtgtcggcgtgggcacggacaggttcccgcgcagggtccacagagcctccagtcgaccgcccgatcggagcacgcagcgcgcctcggaatactctactcggtactccgaaacatcgggcagaggcggtaacggctccgtctccaccaagggcggaggttcatcgaaaagagtcaaggataattcaggcatactacctgcgaccggggcccagagggctaggataagcattacaagattcattctgtcttacaagggaaggctgttcccctgtctagactcaaaagctgtaaggctgtcttatagcatgtagtcttgcacgtcacggggaacagggtggtgatctagtgacgtcgggagaacacggtgttttagggtgcgggggacaaaggacagtacgacagattaggtgatagaaacgtttttttttatttatgaaaaagccagtgtgccgtgcggcctagggccccggcgtagtttggataccagatgggggccgtcaggggtactaccacgagcagaaacataataacttggtccatgtatagcagcatagcggtgcgtagcaggtcgccgtccgtgtagcaatttgacggtgagcgataaagcaccgttaatgtgtcgcggataagcacgatcttgaggccgtagatgaagctcacagtcagtgctaaaatgatgcgttggtatggttcccaggactgcacggcgatgaagagccagagtatgggaagcatgaagcttagcaaacagaggatggctaaccgtcgttgcatgttccaggccatgagccaggctaggcccgtacaccagacgcagagcatggatgacaggacataggcctggattaccacggtgcgatcgaaacacagcccgatggtggacacggatatcgtagtgagggtggtatataccatgaccagcatcagggtcccgggtaggcgccgacgttccagccagtacgcgtggcaacgcagagcgcagggtagcagtgtgctccagaagggcagtgtatcgcgcaggtagggggtcgtcacgcgccacggtatgagcatgaaaaggatggtagtggctatggtggcgctggtctggaacacgacggtgccgtagagacgtaccatccagagaaagtgttgaacgctccgcagggtgtcttcatctttggtgattacggtgactcgacggatcggcggtggtgacggcggcgacacgggtgggggtttctctttcttatggccgagtggctcgccttggtgaaactggatctgtaccatgacgggtgctcgacgaacagtcgtcggggctttaggtacccggcaagttttatagagaaagggggacgatgggtggtggctacgagccaccgccaccttcgcaatacgaggatctgaaggcggcaaagacggtcgtccagggcaggtgccagaggttgggactgagcacgatcagcgtgattttaaacatggtcaccagtcctacgtagatcagcagcgagccacgtaacgtctgagcagccggcagttcgtcgcggatgtaacgcgtgccgtagaaagtcacggtcatcataaggaagacgatggcgccgtagccgtagagtagaatacgctgatgatggaacacggtctggtcgccgataacccagagcgtgatgaaaaaaacgctggtgagcacccgtgtgcatatgagctcccaacgcttagcgcgaaagctgtccccaaccatgacagcgccggtgcaagctatccacagcgtgaggaccagtgtgtagtcgatgaggatggcgggcaggtcggagcaccaggtgtagaaaaccgtggtaacggagaggaggcctacgtagcccatggtcaataccacgtcgtcggggtgcctttcgccctgtatcaagaccaaacaccagagaagggagggggcaaaaaccagcagcagaggggaagattcatgttgacatatgttgtgggaatcggggatgcccagccaaatcattccgcagaaagccgtactgatggcgatgtgaaagaccactagggcgtagacccggacgaggacagcaaaacggcgcagccacataaggccgtggtgcagctgcaggagagaagcccattgcggcgaatgtagcgacggcagcggcgggtccatgaggcgggtgatgcacccgagtgaacgggtgagcgtctcggtggagtcttcttataaaccagcggagctcaggcagtcttgctctggagcgtcgcagtggtggtgttgaggatgacgctgagcgtgccgttgtcaatccggtaatgatgataggtgccaagcttggccaggtagctgaacatttggtcccagcgtgccgaccacaccacgggcgtgagcatcaggagtgtggtgtgataaatgagtgtttcggtggcgtaaagtatcagcgagctgcggatgatgtggctcacgggcattttggtggcgatgtagcgcacgtcttggaaaagaacggccaggatgcagcccacgaacacggtatagagacacagcagagtcttatgcaaccaggtgtaagtagaagccaggacgctgaccatcaccgtcaaaagtgtggaggtaaaaagcgcgtcacgccacacggagctgagacggtgctcccaagccacgccgttgcaggccacgaacaacgtccacgttaagatgaggctggaaacgccaatgggcgctgtggcgcacaggttgagcccggcggtggtgaacgacagaagcgccacatacagcgcaaacaccaggccgttgctggggtgtctatgatcggtaagctccagcgcgcccagaaccaacaccggtgtgcagctaagcaataacggcgaaggatcgtcgcggcactcgtagcccagcgaggggtaacccagccaaaccagcgcgctaatgagcacgctaaaagcggtttccagcgtcagcaatccgtagacacgcatgacgatcgcggtccgccgtagccaacacaccgcatcttcggaagctgtggacgctgtttccgaataccgggaggagatcgtgcttccctcttccaaggatcggaaagtagcgtccgtcgtttccgcagacgcggcttccctggtacgctccgtttccgacgacgcggtttcccgctgcgtggaaactgtctccatgtcgggaccgcagcgcccggcggcgtatccgcaaggtctcgaagctacagcttgtcagaggaaaagtaggtttgcaaaaaggtgcgcagggtcatgattctcagcaccatcagcagagtgaaaaccaggctgagaaacaccttgacggccgccaaaagcgcgcgttccagcggcgtctcgtagcgtacagccagggccgcttcgtggaaatgcgagacggctagacaggtaatgagcacgctgaaggacaagacgatcttaaagcaccaggaccaaccacgcctcaagatgaccaccacgattgccgtgaaggtcaacgtgatcaaagcatggatgaccacgatctgacggcggacggtacgttcgggagccaacaacgctacgccggtgcagctgagaaaggccagtaaggtgaacaacgcggccgagatgaccaacgtaccgtccaggcagagacatatcacgatcaacggcggcacgtgaagcagcgtgtaaaagagcagaacgccgatattgctgggatgcgatgtttcgtaacagtgaatgaagatcaccgacgtgacgggtatgataaagacgaggctgggcgaggactccgtgagacacagacgggaatggtgaaaccacgtcgcgggcgccgcgtagcagaaggcgctcaacaacgcggtcaagccggccagctgccaacccacggcgccataggtgtgcagcgccacgcggcaacagtcgacccaagccagactgcgggtcgccagccgggtctcttggatcccggggggcacgtagatgaccgtgccatcggtgggtacctgaaaccctttttctcttctcatggtgcgctgcgttctctggaaacggctgctctgtccgaaaaccagttccgaacgaaaatctagggcgagagggtggacaacggcgtcgacgacgaagcatgggacaggtcgttcggcgttaacgtcatcgcgtcggacgacggtagttctaagagacgtagatcgctcagcaggtcctgacagttgcggattcgcaagatcagaaaaaaaagggaaatgaacgtaataaagagctgtagcgacgtatgcgctacatcgcgtggcataagaacgtgacggacgaaaaggacctgctgcgaaaagtggccggcaaagataaggcccaccgtgctgtagaagcccaaaagcagccgcaggggccaagtccagggccgcgtaaagacgatgagaacgttaaccagaaagaccacgacccagacgccgttgatgagggtaaattgatcggacagggtgcagttgtcgcgacagatgaagactacttccgcgcagagcaaggtgatgaccaatgtgagcacaaacgacgtcaacacctcgcggggctcctggcaggcacacgtgacacctagcgccgggatgtgcgccaggaggccggcgagtaatagcaccagctgtcggaacggacgacggcagcgcgggtgccggtttcgctgagcgagaaccggtcgctcataacggaaatacacgaagagcgcggaggccacaggcaccaggaggagcacctcgggcgcccagacgacgtgacaaggaaagcctggacgcgacttaagagtcgctgtagggaagaccagagagaagctacccaagacggccaccgccgcggagatttggaagaggagcaagccggcgattcggacgacaacctcgaagcgatgcacccagcccagcacggccaccacggccgcttcatcatagtcgtcgttgttgccgctgtcgaacagccgccgaaacacgatctgtcgctgggtcgcggtgggaaagcgcagacccatgacagccggaggctatatgaccgcgcgtctaaggcgcgagatccgtggggggacttttagatgtttgggcggcccgcggttctaacaggcttgattggtggagacggccggcgcggcgggtgggggaaacgacgagtttttccgttacgccatggttcgcgtgaggtttctctgtacctcccgcaaaaggtcacagcccgaaatggaggccgcgttggtggccccggtggcgcgtgacgataaccaggtcatccaagtgatgagtttgtctaatgagtcctcggtggtgaagaggataagaatgagcaggtacaagtacaccaggttctcatagagacacaaggtgagcaggtcggcctcggaccacgcgatctcaaacaggcgtgtggtgtcaaagaccgtaacgaccagcatgaagctgagcgccatggcgtaatagcccaaaaaaagtttgtgccccaacggtacgggctgcaggtaaagtgcgatcaagaacgcgataacgccgatcacaaacagcgtgacgatgacctgccatcgacggcgattatggccggctagacccgtgacgcagctgcagaggctaaaaagcacgcaagccaagaggcccgagaaggtcaccagcgtagaggaggagcaggcgctggccacgatcaccgaaagcgtcgtgagcacgctataaatggtgagcaggcccgggctcggcggcgacgtaaacgatccttcatcgcgtttgccatgcagcagggccaaacagatggtgggcaccatcaaactcaagggcggcataaagccggtgcaacagagaaagacggtgcctttaagatgcggaaaagccagcaccaggcccagacagagcaagaaggtgcaggtgccctgcacggccacggtgctgtagacccgcatacaaagtaaaaagcgacgtacgtcgttcgtcgagacggaggaaatcataatgactccgcgcgagggtcgcgggggtgggggcgcccaggccgtcccggtggcctctgagttcggagacatgacggcggtggctatcaaaaggcgcgtatgagaaaccgtttatagagtgtaatagaatcaccgtcattcccacacggcgttcccccataaagtcacgtcacactcgagtaagcgtgaaaaagctttattattgaataaaaaacacgagtacaacaccgagttgcggtgtcctgtctactgggtgggggaggtttatcgtctgtctctagagggaaggtggggaacgtctaagcgagcgggagcgtgtcatctcccccatctttttacaacaagctgaggagactcacgccgtcgatgcgtccgccgtgtttctcggcgtactgctgcacccagacgtggccgctaaagatggcgacgctcatgtttaggagactcatgacgatggtgtacaacacgacgctgacacagacgctgtttttagacagcgttccacgctggtagatgagatccagggtctcgtaaataagcacggccgaagcggcggtcaccaccaggacgtagagtccgctgtagatcttgctgacccacagcacgggcgaaaagtaaagcaataggtaaaagacgatgacggaccagccgtagccaatcccgatgactttccagcgcgtgggattgttgccggccaggtaggtgagaccgctgcagagaacgaaaaagaccatcaccagggcaaacgacagaccgatgacgcgcctttctccgcaaaagcccgtgcacacggtgatgccggtgttgatcagcaggcatgccaccgtgagatgagcaaaattggtggtgtgtgggcgaaactcggcgaaaccgcgtagcatggccagcgtggacacgggcacgatggaggacagggctggcactatgccgttggcgcactgtccctgcacatcggggaaggcgagccaagccagcaggcagaccgtgagggtacaagccagctgccacacgagcccgtgatagacctccatgagcagcttgaagcgtttcaaccactggaagagctgctgttcggccaccagcgcgtggctgcgatggagcggcacgatggtgaccgtcggcgactcatggtgttcggaaaccgaggcggtgtcgcccatgctgccgcttacgaccgctgtcggtctaaggtaggcgtcgatgaaacagtccgtcttatcagcacccggttaccgcggatttgattgacgtcacgagtgtggtcaaaccgtggcggcaccctgtatccgacccgtcgtcatgggctccacaaccagagcctcagaagatggtacatgccgatgaataaagccacattttcgacatagaggcgtagcgagggctgaaaactctccgggaaagaactctgacaggtgatcagggacagatcgtgaattagcatcagcgtcaccgtcaacagcgtcgtcgcgtgtaaaccgagaaagaacggggtcgcggcccgcagcagccaaagtcccagcgccgtagcgcagagcagagacaggaccgacggtagccacagccgccggagagacgcgccaggatcgcaacccaaaagcgaggcccccaggcagccgagatctaccgccagggcgagaagagccgcgccgagaaaggcctgcggcgacggctggcacatcagcaaggtcagaaaggctagcgcgtgcggcaggcagtaagccaacaggagtgggagtttgcggggacaacggtcgatagacggaccgcgtagcagcaggaacaggcagccgacgggcacgacgaggctgagatgagaaagcggcggtgggtcgtcgtcccgtccccgctcgcatagctcggccaccggtggcggcatgagccaccagctgagcacgctgagggcgacggtggcggtaagctggaaggcgacgaggacggaggcgcgcagccataccgccagcctctctaagtaggggactacctcctcgacggtccattctagcgggacgacatgaagcatggcgacaagcgcggctgctgtgaaaatgagcgcggttttataggcattaggacttcccgatcgtactggcggctgtcaaagtcccgttgtccaaagacgcgccgtccgaaagactaatccaacggggacccgagagcatgagcaacaacgtgagaaagatggccatgctgtccaggtagagacagacggcatgacggatgcactggttaggtgggcagaaaaagatgaccatgagactgtcgtaggccagaatacccaaaaagaagctgatggagaaggcgcacaacgtcaccactatcttctgcagccagtcggcgtcgcttagcagagcgagcgtgaggaacgaaagcagcatcaccacgtagacgcagctgatgcatttccaacgacgtcggtcacggccacctagaaacgccagccccgtaaaggagataaacaacgccagggtcatcacgtaggaacctactagtacgcggctttcagagcacatttggaagatggccgccgtcaggctgttggccaacagatagatgaaaagcaccgtggcgttactagggtgctcgttgcccaaagtgtacgtgatgaacatgcagacgatgggcacgagcacggtgagaaagaagctgtagttctcgacgcaaaagttgcggttttgtgggaaccccaaccaaaaaacgcttcccaagccgaagctgaaagccaactgaaagatgaagatggcgtacacacgcagccatacggtgaactttttgaaccactcgagagcctccatgcgggagagcagcagcgcgttagcctcctgcgcctgcatggtggcgacggtctcggcacaaagccgctgcggcgcacctacccttctcttatacacaagcgagcgagtggggcacggtgacgtggtcacgccgcggacacgtcgattaggagacgaactggggcgacgccgctgctgtggcagcgaccgtcgtagcgaccgtcgtctgagcagtgtgggcgctgccgggctcggagggcatgaagtagagcacggagacaaagaggtacatgaggtccatgtacaagcagagcgcgcccgggatataactctcatactcgatgtcgtgcaggatgtcctgcgtatcgcacaccaccgaggtcacgatgacggccaaaccggctatcatcaccaggatctcacttaccgcctcgggaaaaagagaaaatacggcgaacagtaagagaatcagcgtggatgcgcccgtcaatagggaacgctgtaattccacgtcgcgggcaaacagatacgtagcgagcgtaaggaaacaaaatagcgttactgtggccaccatggcataaatgactgaacgatgactaaaatggaagcctgacgccgtgacagccacgctggtaagcaacgtgtacgtcagtaagatccatacgtttttgggaaagttgggctcggcccaacgcaacagacctaggcacacgatggagatcattaagcaagacagcgtcagacgcacgctggaaaagagctgctccagccggtgcggcaacaccagccagcaaaaggcgcagacgctcataaggatgaggcattgcacccagataaggatgtagatgcgcagcaggaagaccgaccgggctatctggacctgaccgcggagcgacatggcggcaacgccggcggttatcgccgagattcgtctaaatacacgaagcgaactagaaaacgcacacacgttatttgcaaaaagaaagcagctgccggcttattattttattaaaaatttatctgtgcagaatcataagtttatgatgaataaaaacggggaaagggaatctgcttttagggacccgggtctggtccgtcgtctcccatctggtcgggttcggggatggggacctgtttcagcgtgtgtccgcgggcgtgcatggcttttgctcgccggccgcgctgtaaccaggcctctttctctgtggtcggcgagtcttccgacgggtagggagtctgggagtccatcgcttcaggcccaccgctcgttccctcgaccgtcgtgtcgtcctcgttttcgctattacacggggtttctggagtatcgcctatacggttggcgattctccgggggtggccgctctcgtcctcgtcgctgctatcgccgcccggtaattcgacgccgcattcgttgtacggagcgcggcacatgggcggcggaaagaacttgggcatgcgaaagcagcgttgtccatccacggtctgcgtggtttcatcattatcctcccataatcccccctgtagcgccggcagcgtttcgacgctgtgagaggggaaggcccagttctggttgtcttgcagcgcgcccgtgggcagtaggtccgtgcggccccaggcgctgctgttgttgggtaccttgtcagtgccgcgagtaggtcgcagaaaccagtccagagcgctctctagctgcgagcgtgtgatggtgcccagtgcgccgtgccagcgcagcacgtctcttttcagcgtgtggtgacagacgggcagctcctccaaccgacactcgccgcgcaatccgcggtcgaagcggcagagaccacgcaatttaagcagaccgcacttgagaaacatgtgaaaattatcggcaatgcgatacaggtctgagtcctcgatcttgtgtaggtagaccacgccaaacttgtcgagcagcaccaggccgctgggcacaaaaggcccgtaggccaggtaatagcccacgaggccgacgacgtaccactcgcagcacaagcgttgacgaataaagttcagaagatcgcgaaagtccgcggccggcatgtggtcaaaaggccggcaggcgcgcaggccctcgatggagcccagcatgagcaacggctccacctcggtgcgacccggcgtgcggatgaccaggttgagaccgctcatttcgcgggccgtcttggccacggccgcagcgtcagtggggtcggtgcagaggaatttttgcacatgatagcgcggttcggtggtggcgaacggcgtttgtgggtgccgatacacatattcgcaccagagtaggccgttcttggaaaaggctttgatatcactggccacctcgtagagcccgtcggtctcccagtcgtagacgtagacggtgccgtaatgacttagcatgagcacgcagggcagttcctgcgcctgcttggtgtttcgtgttagatcgctgtcgggtggacgcacggctagtacaccgacggcttccagggtgtcatcgcagcagagatagtcggcggccagagaacgtgcgtaaatctgcgggatggcggcctgttcgcgcatcactaggaaccagttggcggggttgcgcagtgctacggtggttccttggtggcgctgcacgtaggttctcagcgccggaggatcgtactggcgcagatagaggccttgcagcatcgataacgtcttttgaaagacggtgtttctaaattggaaaacgccgtagtcgcagcggatagcatcttcgcagcgctcgtcgcgctgtcggagataggtgccccaggcttcggcggcggctttggtgagtagggacatgccggcggagccgtctcgacagcgagtcggataaagcgcgctgcgcgaaagcttaatataggagcagcgtcagacgaatcgcggctggtggcccggggggtgggacgcgccgcctacacaaaatgctcccgaaaatcgaaactcttgacccactccggagacaaatccgtattcagattgatgcgtcgcgcttccacttcggcttccgaaacctcggcctccgtccggtaggcgttaacaatacgctgacccaggtgccaacgctctttctctgccaaacgccgttgctcaaaccactcgtctacgtccttgaggtcaaagacagtgtcctcctcaaggtcaaagcctaggtcttcccactcgtcgtcatcgctctcgtggccggcggccatacgcgcggcaaccgcgtcttcccctcctcttctttcaacgttgggtaccacgttgttttcttcgggttccatgggttctgcgccactatcgtcatcgtcctctccctgctcctcatcgtccgccaaggcgtcgtggatcacctccaggttctgattgtcgggtacgacgtggttatcttcgtcgtcgtcgcgtggcatgggcggcggccgacggcggacgaccggcatggcgcggccgtcgtttccttcgtcttcctcttcaccgtctcccaaggaacgcggtcgacgacgttccgcgaagtcgccgcggaccacgcgcgcctgccaaatggtaaacgcgtcccaaccgtcccagttattgagcatttcggcgcgaaaacggtcgcctcgacagagccagcgaaactgccgcgcgtagtcgcggtctacgccgctgtcgaacatggtaaagtgcagacgcgccgcctcgcccatgtgtacgcagcctccattgcgttccagcctggccgcgcgccgcagaccgtgttcgtagcggcgacgcacgtacaccttcatgaggccggcgcgaaaaagttcctctaggctgtcggccagacggtagatttcaccggctagacgctgcaggggcggcgagcggtccagatgcgacttgacaatcaccacgtaaaaacgacagaaacggtcgaagatgatgaggaaggacgtgtcaaagaaaccaccggcgcggtaggagcccacggcgcctagcaggtaccagcggcaacgcagttgcagcgtgacgtacatttcgcactcggccaagcgggcggctggcgctacctcgaagggccagcaatccgtcaagcagccgaaactggtcaggagtttcaacgttttggcatggcgcccaggtgtgtgaaagttcacgtcgcgtccgtggtgttcgccaacgcaggcggccaacgcgtcggcgtcatgagcgtgacgcagcagcatcgctaccacgtcgtgcggtacccgcgtagcaaacggcgtctgtggctgacggtatacggcttcggtgtacatcataccgtaacgtgccagctcgtccagatgacgcgcgcacagcagcagaatctcttgcgagggttcgtagatgtagaggcgcgtaccgccccccatgcagagcaccagctccgtctcttcgtagtgatcttccaccatgatcacgcacttgcctagcacgataaggcgttcggggcaacaaatcacgtcgtccagcagttggtcgcgcagctccggcatggtgctgccaggccgcacctgcaggaaccagttgtgcggaatgccgagcgacaacacctggtcgacgtggttacggacccagtcgcgaagcacgtcggcgctgtactggcactcaaagatgccctgaaagtcgctcatgacccgcagaaaagtttcgtagcgcgtgtggcaatagaggaattcatcgtttcgcgtgaacgtgggagctccgtcttcccaacgtgtacgccacatgtcaaaagaggccgccagctagacaccccagaaaagaagcagagaaagagagttctttgtgcgacacgttttattccgcgtcctccgctcgacgctcaaatctggatgtactcgcgcacacccgtcaggctctttaagggaaaagggtccgagtacgtcactaaccgcgactgatgcaccagggcggtaatcacccgctctgcgccctcgcgcgtcgacgaacgcgtcgtcaccaggcagtgcagccgcgggcccgtatcgtcctgatgaccagcggcctcgcgctcggctgcttccacaccgacaatgtcgggatccaacacgtagctctgcgagttggtgtcgtagcggtgtaacaccaacgtgttggggtccagacgctcccacgcgccctcgtgcgggtcaaaacgctccgttaaacagagccagtcatactgctgctgcagaatacgccgctcgcgctcgcgtcgctcatcgggcaacgcagcgtcttcgttgaagagaatgtcccgcttgtggtctacggcacgctcgtggtggtgcgggcacagatgacggtgttccatacgcgtctgacgctgacgctcgcgttcgaagcgccggtgtcgaaagaccattttcagcaaccccatgcggaaaaactccgtgatggtgttggcaacgcgccgcacatagtggttggggtcgtccatctggatggcgtacacggcaccgaaccagtccagcagtaccagcacttcggccacaaagttgcgtcccggtcgcggacgtcccgtcacgcctagcacataccacggcgtggccagattagcacggacagcccaccaccaacgacggctctccacctcggtgagcgcacagaagggccaaatgcggtgtaactgctgcaccgttttcatcagccgcataatcaccgtaccgtaacccggtgtatgcaacttcacgtcgcaacccaggattcgttcggccgtggcgtacgagccctcgggcgtggtgtcattgagaaacaaaacatgcatggtacgcgcgcccttagggtatcgtcgcggaacaggtaccgtcattctccgcagagtggtgtgaatcacgtcgcgatacgcaatctccgaacgcgacacaccgtaacgtgccagttcatccaagttgtgcgatactaacaccatgtacttttcacgagtgtcgtaggcgtagacgcgagaaaagcgacccataaaaaccacgtacggggtagccaccatgccatcatggtgatcgcgacgtggctcgggcaacaaaataacagcgtatcccaacggcgtcagcggctcgcggcaacagatgagctttgacgccgcctgtctggcggcggtaatgatcccgtcctccgtacgtaacatcacatgccagcccttggggggacccaaggacagacaacgtccctcgttacgatgaacgtaacgcgtgatttccattggctccaggcaaaagaacagttccttaaaatcccgcaacacttgtcggtataacgccatgggatcctcggccgccacaggcagcgcggggagctccggcggcacaactgcagcgccgtcagggccagaacccgcagccggatccatcattacgcgacactctcagccggacaaccggcgtcactgacagaagccgagccaaatacagagaaagcaacgctacaccgtcaccccgctcccaagcgccgcggaaagtgctccgatttttcaccgtcgttcgcgacgttgatttgcctcggtctgagaaccgacctagcgttcggaccggtgcgcagaaacagccggcggtccgagccactgagcggttcacagccccggccgccgatagttaccggagagacgttcgagctgcaggtacatcggcgctccccgcttcgccaccccgcgcccgccccagtttatactctccgacgccccgtccaacgcgcctgtggagggccaatcggaccgcgggagctctccaagtggatgacaggcacagccgggtgcccgaccgtgaagagccctcatccacctgaacagaccgctaaccgaaggaccccgagtcgcgtccgtcggtcccgacgtccgtcgccatctggctccctgctgttggctacctctcggatttcaaaaaagagcacgtgccgatgacggtgcacaggaaagagccaaagtgtcacggcgtctttttttatttgtattcctttcctgttttgtactcgtaaactgttgacgttgtttttacatccaaaagggcaagtaagaaacaggatgaggcatggtaggtttgggcgtggggcggccctccagcacggcggcccgggccgcccggcgggtgagcacccggcgttgcgccgtatctatcttgtgtttcttctgtgtctttttcctatcttgttccgcgacggcctctttcatcacgttcagcatgcgttcctcgacgccctccagggatcctggggaggagggagtcctagtgaggcttccaatgttgttttgtggattttcggtttcctcttcttggtcgtcatcgtcggacgtgtcgtcttcctcttgatcctcttcttcgtccgagtagtagacgcatagtccctggttcatcaggctgggattcatcaggttctgacggggaatccgctgttgtagacgtttaaccgcccgttccaggcgagagctcatgccgcaccagacgctgtaacgccgcacgggcccgtagcgggctgtttgttcgcgtacatgatcgttgagctcttgccaatattgtttggcacactccagatcggaggtttgtggatagtcgggtcggatccgcggatcccaactgacatcggcggtgccagagacttcgtccagactgttacgcatagagcaccagtcgggtcggacgataaacctgtccttgcggattaaccatttataacgtagttcgtgatggcgtgtagaggcccgtacacgctccacggtcccaaagcggtcccagaagggaaagttttcgtgggggcagcgacccggcacttccaaacgttcggcgtcgtccacggcgtagtggaaacgccggccggcctggtaaattttgagcagacccacggttaacaacatatccacgctgtcagccaaccgccagatctcgcgccgagatacgtcaaaatagaaaaattcgcaggctcggtcgaccaggatcacgaaatcggcgtgaaaaacgccggagggtagcgactcgcccaccacacccattatcatggtttcacagcataagcggtccacaaagaacttcaacaggtcgttgaattgctccgtctccatacagatgaagggccagacgcctttgaggttctcggcctggccgcagagcagcaacggacgcgtcatctcgcctggagtgcgcagaggcacgcattcgccgcgataacgacaggtcacacgctgcagttcgctgatgctgttgtcgtgcaggcgaaggtcgcagataatatgatccggttgcgtggttagcagcggcgtgcgcatttgctcgccgtagatggcctcgcagtgcaatagcccgtgtcgtgcaaaatcgtccagactgtgcgccaggtagtaaagcaccccgcgatcgcggtctagacaccacacggtttcgtaacgtcctagcagaagcaccagacgggcctggctaggtggctcaatttcctctacatacacgaaaaagtcgtcatcgtccgagtcctcgtcctcagaagaggaccgcggcccgtgtactctgggcaacacggtggtagagaactgcaggacgcccagagactcgagcgactcttcgcagcagatgagctgaccccagggcgtttctggcccgtcggtgacagccgcgctgccaaagatgtcctcaaactctacaaaatctagacgccatccgggtggcgctgaaacgggaaggctaatgttcatatcagcatagctacgaactaagtggcggatgtcctgccgcaagtcttggcagagaatgagctttcgtaaacccttgagggtcctccgaacaacggccccagacgcgtagcgataggactggcgcatggtgccgcggcgtggagcggcacttggcagcctattttatggagtttcttcagtgacgtggcttgttcacgtcgttcgtgggctgcggttggcagctccggtctgtaaaccacccgaaaagactgacatcgacgtcaaagacccacgtaatttggaacatgtgcgaccgcaaagtgcgtcagaataacacgtggctttaggacataaaaagtaccgtgaggtccagacgtggtttttgtgattgacacttacaccaggtaagccaagggacggtgaaactgtatgtgaggaacctgggtgcttagacgactaacgtgtaatgctttttacaggactgttcgacaggtgatagtacctgtaaggtgatgaccacctctacaaataatcaaaccttaacacaggtgagcaacatgacaaaccacaccttaaacagcaccgaaatttatcagttgttcgagtacactcggctcggagtatggttgatgtgcatcgtgggcacgtttctgaacgtgctggtgattaccaccatcctgtactaccgtcgtaagaaaaaatctccgagcgatacttacatctgcaacctggctgtagccgatctgttgattgtcgtcggcctgccgttttttctagaatatgccaagcatcaccccaaactcagccgagaggtggtttgttcgggactcaatgcttgtttctacatctgtctttttgccggcgtttgttttctcatcaacctgtcgatggatcgctactgcgtcatcgtctggggtgtagaattgaaccgcgtccgaaataacaagcgggctacctgttgggtggtgattttttggatactagccgtgcttatggggatgccacattacctgatgtacagccataccaacaacgagtgtgttggtgaattcgctaacgagacttcgggttggttccccgtgtttttgaataccaaagttaacatttgcggctacctggcgcccattgcgctgatggcgtacacgtacaaccgtatggtgcggtttatcattaactacgttggtaaatggcacatgcagacgctccacgttcttttggttgtggttgtgtcttttgccagtttttggtttcctttcaacctggcgctatttttagaatccatccgtcttctggcgggagtgtacaatgacacacttcaaaacgttattatcttctgtctatacgtcggtcagtttttggcctacgttcgcgcttgtctgaatcctgggatctacatcctagtaggcactcaaatgaggaaggacatgtggacaaccctaagggtattcgcctgttgctgcgtgaagcaggagataccttaccaggacattgatattgagctacaaaaggacatacaaagaagggccaaaaacaccaaacgtacccattatgacagaaaacatgcacctatggagtccggggaggaggaatttctgttgtaattcgatcctctctcacgcgtccgccgcacatctatttttgctaattgcacgtttcttcgtggtcacgtcggctcgaagaggttggtgtgaaaacgtcatctcgccgacgtggtgaaccgctcatatagaccaaaccggacgctgcctcagtctctcggtgcgtggaccagacggcgtccatgcaccgagggcagaactggtgctaccatgacgccgacgacgacgaccgcggaactcacgacggagtttgactacgatgaagccgcgactccttgtgttttcaccgacgtgcttaatcagtcaaagccggtcacgttgtttctgtacggcgttgtctttatcttcggttccatcggcaactttttggtgatcttcaccatcacctggcgacgtcggattcaatgctccggcgatgtttactttatcaacctcgcggccgccgatttgcttttcgtttgtacactacctctgtggatgcaatacctcctagatcacaactccctagccagcgtgccgtgtacgttactcactgcctgtttctacgtggctatgtttgccagtttgtgttttattacggagattgcactcgatcgctactacgctattgtttacatgagatatcggcctgtaaaacaggcctgccttttcagtattttttggtggatctttgccgtgatcatcgccattccacactttatggtggtgaccaaaaaaaacaatcaatgtatgaccgactacgactacttagaggtcagttacccgatcatcctcaacgtagaactcatgctcggtgctttcgtgatcccgctcagtgtcatcagctactgctactaccgcatttccagaatcgttgcggtgtctcagtcacgccacaaaggtcgcattgtacgggtacttatagcggtcgtgcttgtctttatcatcttttggctgccgtaccacctgacgctgtttgtggacacgttgaaactgctcaaatggatctccagcagctgcgagttcgaaaaatcactcaagcgcgcgctcatcttgaccgagtcactcgccttttgtcactgttgtctcaatccgctgctgtacgtcttcgtgggcaccaagtttcggcaagaactgcactgtctgctggccgagtttcgccagcgactcttttcccgcgatgtatcctggtaccacagcatgagcttttcgcgtcggagctcgccgagccgaagagagacgtcttccgacacgctgtccgacgaggtgtgtcgcgtctcacaaattataccgtaataaaaaagcgctacctcggccttttcatacaaaccccgtgtccgccccttttttccccgtgcccgatatacacgatattaaacccacgaccatttccgttcgattagcgaaccggaaaagtttatggggaaaaagacgtaggaaaggatcatgtagaaaaaacatgcggtgtttccgatggtggctctacagtgggtggtggtggctcacgtttggatgtgctcggaccgtgacggtgggtttcgtcgcgcccacggtccgggcacaatcaaccgtggtccgctctgagccggctccgccgtcggaaacccgacgagacaacaatgacacgtcttacttcagcggcacctctttccattcttccgtgtcccctgccacctcagtggaccgtcaatttcgacggaccacgtacgaccgttgggacggtcgacgttggctgcgcacccgctacgggaacgccagcgcctgcgtgacgggcacccaatggagcaccaacttttttttctctcagtgtgagcactaccctagtttcgtgaaactcaacggggtgcagcgctggacacctgttcggagacctatgggcgaggttgcctactacgggggttgttgtatggtgggcgggggtaatcgtgcgtatgtgatactcgtgagcggttacgggaccgccagctacggcaacgctttacgcgtggattttgggcgcggcaactgcacggcgccgaaacgcacctaccctcggcgcctggaactgcacgatggccgcacagaccctagccgttgcgatccctaccaagtgtatttctacggtctgcagtgtcctgagcaactggttatcaccgcccacggcggcgtgggtatgcgccgctgtcctaccggctctcgtcccaccccgtcccggccccaccggcatgacttggagaacgagctacatggtctgtgtgtggatcttctggtgtgcgtccttttattagctctgctgctgttggagctcgttcccatggaagccgtgcgtcacccgctgcttttctggcgacgcgtggcgttatcgtcgtccacttccaaggtggaccgcgccgtcaagctgtgtcttcggcgcatgctgggtctgccgccgccaccgtcagtcgcaccacctggggaaaagaaggagctaccggctcaggcggccttgtcgccgccactgaccacctggtcactaccgccgtttccgtccacgcggatacctgacagtccgccgccaccgtaccagcttcgtcacgccacgtcactagtgacggtacccacgttgctgttatatacgtcatccgacatcggtgacacagcttcagaaacaacgtgtgtggcgcacgctacttatggggaacccccggagcccgctcgatcgacggctacggttcaggaatgtaccgttcttaccgctccaaattgcggcatcgtcaacaacgacggcgcggtctctgaaggccaagaccatggagatgcggttcaccatagcctggatgtggtttcccagtgtgctgctgatactggggttgttgacgcctccgagtaacggctgcactgttgatgtcggacgaaacatgtccattcgagaacagtgccgccttcgaaacggtgcgacgttctccaagggagacatcgaaggtaacttcagtgggcccgtcgtcgtggagttggactacgaagacatcgatattactggcgaacggcagcgacttcggttccacctcagcggactcgggtgtcctacaagggagaaaataagaaaagataatgaaagcgacgtcaacggtggaattcgctgggctctatatatacaaaccggcgacgccaagtacggtattcgtaatcagcatttgagtatacggttaatgtatcctggggaaaaaaatacacaacagctgttgggttctgatttcagttgcgaacgtcaccggagaccgtccacgccgttgggaaagaacgccgaagtgcctcccgcgacccgcacgtcttctacatacagcgtcctcagcgcttttgtagtgtggatcggatccggcctcaatatcatctggtggaccggcatcgtgcttctggcggtggacgctctcggacttggcgagcgttggctgaggttagcactgtcccaccgggacaaacatcacgcatcgcgaaccgcggcgctccagtgtcaacgcgacatgttacttcggcaacgtcgacgggctcggcggctgcatgccgtttctgaaggcaaactgcaggaagagaagaaacgacagtctgctctggtctggaacgttgaggcgcgaccctttccgtccacacatcagctgattgtgctgccccctcctgtagcgtcagctcctcctgcagttccctcgcagccccccgagtattcgtctgtgtttccgcctgtataaaaataaagagacgggaggctgatcgcggccttcagcgtctcatttgtctttactctcgagtgcggtcggtgtctcgtcggtgagacgaggccgccgcccgacaagttcgatctcatgtcgctcttggagcgcgaagagagttggcgtcgcgtagtcgactactcgcacaacctgtggtgtacgtgcggtaactggcagagccacgttgagattcaggacgaagagcccaactgcgagcagccggagcccgcacactggctggaatacgtggcggtccagtggcaggcccgggttcgcgattctcacgatcgctggtgtctctgcaacgcctggcgtgatcacgctttgcgcggccgttggggtacggcgtattcctcgggttcctcggcctcttcctccggtttcgtcgcggagagcaagttcacctggtggaaacgactgcgccacagtacccggcgctggttgtttcgccgccggcgagctcgatacactccatctaactgtggggaaagtagcactagcagcggccagagtagcggtgacgagagtaactgcagtctacgcacccacggcgtgtacacacggggtgaacaacactaatcgataagtcgcgtgtaggcgactggctacatcaaccggatatctgcggggatttaaaaagacgacccgttgtcatccggcttagagcaaaccgtccttttatcatcttccgtcgccatggctatgtacacatccgaatccgaacgcgactggcgtcgtgtaatccacgactcgcacggcctgtggtgcgactgcggcgactggcgagagcacctctattgtgtgtacgacagccattttcagcgacgacccacgacccgagccgaacggagggccgccaattggcggcgacagatgcggcggttacaccgtctgtggtgtttttgtcaggactggaagtgtcacgcgttatacgccgagtgggacggcaaagaatccgacgacgagtcgtcggcgtcttcctcgggcgaagcgccagagcaacaggtccccgcttggaagaccgtgcgggccttctcgcgggcctaccaccaccgcattaaccggggtctgcggggcacgcccccaccgcgcaacttgccgggatacgagcacgcctccgagggctggcggttttgcagtcgacgggaacggcgagaggacgatcttcgcacgcgggctgagccggaccgcgtggtgttccagttagggggagtacctcctcgtcgtcaccgagaaacttacgtgtaagaacacggcgtgacaataaacaacatagcgtaaatccccgtgtgatgtgtgtgattgacgttcgggaaacatgtccccatcatcagcgtcacaactgacgtgggttggtcactgacgtgcaggatgttgcgcgagtcagagaatcgcataagaacggggtggtgagcgggttcccacaggagtctctggcgcaaaagcaccatgagcctcaggttccccgagagggcgggttacgagaaactgggataccgcccgcatgccaaacgcgtgcgggtgcatgacccgttgggattgacgcggtttatcatgaggcaactcatgatgtacccgctggtgttgccgttcacctttccgttttacgtgccgcggtcctagcacgtcagtggtgatgctgataattgcaacatggcccatgacgaacccgcttgggacgaacgtcaataccacgtcaaaccaccgtgacttggctgaacgttgaaacataaagccaaagcgccgtcggcacttggcttcagagcagcgcctcggggcgatgcgacggcgatgaacttagagcaactcatcaacgtccttggtctgctcgtctggattgccgctcgtgctgtcagccgcgttggtccgcatggctccggactcgtttatcgtgagcttcatgatttctacgggtatctgcagctggaccttctgggaccagtggtggcggggaatcgctcagtccggacctggaaagagcaggcggaccgagccagagggaccttcgttcggcgttcaggccttaatactagctacatcttacctgtcggcggcctgtctgggggctccggtaccttacccgtcggcctgtatcgtcccgaagaagaggtgttcctcctcttgaaccgctgccatgggccactgtcaacgccgaaaaatgcttgtctggctgaggtcggtgtcgctaatgccacttttttgtctcgcttcaatgtcggtgattttcacggagcgtcatgggaaaacggtaccgctcccgatggagagcccggggtatgctgaaattcctcttaagattccgtaaacgacgttgtccagtcgttgtgccgcgattcgtacggttcatcgtctacgtcgttttgttcaccgtcgctgtgcaacgcgtgaaacaagagcgtgatgcgcaccttcggcggtatgaagaacggttacggaaaaaccgcgcacggcgtcggcagtcttttccgtgacttggggcgatgggtccgagctgcggtatgggtcacggcggcgtgtgttttattgacgaagatgccgatgtgtgactaaaaacgtcccagccctagagcgatgtgtttcaataaaaattatgtcgtatcatagtatgcgtgtcctggtttttcatttttggatgtatttgtgacataaaaggcgatagaatgtggggacgaaacatatccagatacacagttttgttattcgaacaaaacccgtgtgatgcagaaaacagtactgcaggatgaaagtcccatgggggggggggggcagacagtagtcgtttttgccgctgggcgtacgctatgcttgtatttatgactataatatgtgcactcgtgtgtcgatgttcctattgggaagggtgtcaatgtaggaggtataaagaatggtgggatgcggagaggcatcgctagacacaggttgatcgctgtgctagccccacctgatcagcgtcatgggtaaagcggtgattaagcgtgaaaacaccgtaagggggggggggcagacaggaagcttggtggcagtggccgttagatgcattacgtgtctgtattggtacatttgcaaaccgtcgggtgtggcggtatagtttagcgatgattatattatgtatgtgccgtatagaatggcctaaaacattgtaacacgaaacgttacaatgatgggaaagatgccgataaaaaacacataaaaggcatatacacgaattactagttacacgtttgtctatgtgcgagtttaaggacgcttgtataatgcgtatgacggcaaacggccgcggaaacgatggggggggggggtagtaactgtattaattatacgtcttgcagtacacggtattgtgtgctggtgcgcgtattacgacacgaacggcatagcgctataaccgggtgtatggtatttatatgtgcgtctagcatccttgcgagattctgaaagtcttcttgtaagcgtaattaaaacggtgtatgttctgcgtaaagtgcattcaaacaacgtaacagtatgggatgaatgccaataaataacatataaaagcgagaagtatacatataagggttgctagacacaggtttgtttctgtgctagcccaatggcacttgtacaatccatgcaagcaaaaaaaggatgcgaaaccaacatcgtggggtggggggggggtaaaagcaatgttaatcattggtctcgcggtgcaagttgctgcgttttacgtgtattgttacacgggttgcgtatcggtataatcggatgtgtgttactcattcgtggcgttgttatagtattgtgaaaaagaattctcgtaagcatgttgacaactgcaaaataaaaccattttattgagcattgtaatggtagtgtgtcgctacattagaaaacgtgacgcgtcgcatgtcgcggcacaatctggcagcggggtcggggtagggtacggtgggaggcatgtacacagatggaacaaaagcagaagtaacgtgagacggagcatatagtccagtatccagcggttcctgagtagcaccacccatcaactgaatgccctcatgagtaaaagtctgcgggcggcagcccttggggaccgttggcatgggacgatcgatctccaaaccacagcgtaacacggttttcttccaacgtcgttgatacacgtcgtttttacggttactccccagaacccagaaagtctcgtccaagtcgtaccaggagtcttccccagggagacgtggcggtttccaatcctcatcgtcccgtcgcaaagcacgtcccaaactggcttggggagtcaacggtggttctgtgggtcgggtgtagcgcgagtgttttccgttcatgagcgattcgtcctccttgcctttaggctttttggcctttttgtgtatcatctggccgccggcctccataaccaccgtggccaagtccagtcccagagcttgagcgtcggcgcggcgtcgggcgtcttgcaggtagtcttccacatttgcacagatggccgggtgtttggtggctagggtgaggacctcagcctcgccgcggcccggacgtagcaaaaaagctaactgcccgtgcggctcgcgcgcccacagcgcggcgcgcgggtgcaggtgcagcgcgtcccagcgcggccgctcccactgctcgcggtccagctcgggcagcagccgccgcgcggcctcggcggcgggcgccgactcgcgccccagcgccagcgcgcccaacacgcccgcgcgcagaaagtgcgacagctccgccgccagcgggtacacgtgcccgtccagcgggcagtacccgaacacggcgcccagctcgtccagcaccaccaccagcatggcgcgcggcacggtccccgacgccgccggacccgccatcgccgtcggacccaccatcaccgtcggcgccgccgctgctgccgctgccgcatccgttccgaccaccgcgtgcgcgtccgcgtttggcacgcaaatcgcgctcccgccggcggcgccgtacggctgcggaggtaaagtcacagcagaccccacggctcccgccatcgcgcacggcgcgtccccgccggcggcctccgtctccgtgccgctcgcccccggcagcaacgtcgtccccgtcgccatcgccgtcgtccccgccgtcatcgtcgtcgtcgtccccgccgtcgtccctgtcgccggccctgccgcgcagcgcagccaccgcgacggcagcaccgcgcccagcgccagccagccgcagcacagacgctggttcaggtgccgacgcacggccgtcagcagcgacgcggggtgcggcgccgacgcgaacggctcgtactgcgccagctcctgccacgcgcccagcagcaccatcggctgcagtcgcctgcccggcgtctgcagcgccaccgtcgtgccggcccaccgccggcgcagctcccgtccgagcgccgtcgcctcctcggcgcgcagcaacgtctggcgaagcgccggctgaggcagcagcgtcgcgcgcggggtgcccacgcccagccggttgcagcggtacaggcgcaccacctcgcccgcgccgtgccgaaaccactcgtccgcgtcgcgcgccgccaggatcagcgtgttgttcgccaggtcgtacacgaacacgcggaacccggcgcccagcgccaggtacagtccgtcctgcgcgcacagaccctcgggatggccggccttgtcgcccaccgtcgggtcggccgcggggtccacctcgtgcaccacggtcgccaccagcacgatccacgcgtcccgcggcgacagctgacgcaggtccgtggcgcccacgccgttcatctggctgcgcggcgtcactcgcgcgtagaatccgtacggccgtccgagcggcagcagcgtgcccgcgtcgcgctgcgaccacttgcgcatggcgcggcccgtgctgttggccaaaaacgcggcgcgccacacggcgcccatggcctggtattccagctccgtcagcgcctggcgctccaccggaatctgagacagcagcaagcgctccgggccgtgccaaaaggtgctgttgttgccgctacccggaggggcgcccggcggcccgcggggttctacccggtggacggcgtgggccggcgtcgccgtacccgcagtactcgtactagtccccgctgttgacgtcgcttccaaagaagaagaacgagaggaaccaacccccgaaggccctccggctccgcggccgcgaccgaggggcggggggcgcggcgacatgccgttgcgctgggccatggccgccggacgcctccgacgtccactctgtatatataggaagcaaacccgcgtcagcgaccacgccgtttacacacgcggacgcctccgtcgcccgtgtgccgcgggcgacacgcacctggcttttataggcagcgacgtgcacggcgcttgctggcgccgccttgccgccgcgcagtctggaaggccgtgaaaaaaccgaaagaagatgcgcgacaagccaccaacaacgggccgccgagcgcgcgcacacctaggtggacgcctgacctccattccgggccgtgtgctgggtccccgaggggcgggggggtgttttcatcgggggggtgaaatgtgggaatttgggaagttggcggtggcgggggacggcgacggcgaataaaagcgccgtgcggcgcgcacggcgaaacccagacgcgcgtgtgtcttgcgtctctttcagtcgcgcgtctgtcttgcgtgtctttgagtccccggggaaaagaggaagcggtcccgaggggacggcggcgcgactccctggggacgcgaagatcggacgcggaaaagaggaagtccccggggacggccacaggcggaaaaaaaaaacgaggaagccgcagcccagtctgccgggaccggcaggcaggcgcacagacccgcgtcgaggacacacgcagaagaagcgcccgcgccggggcggggggggggatcgcgggccccggggcacactgcttccatctggccgcgcgcacaccccgccgacacacccctgacacacccgcgacacacccggcacacgcccgcgacacacccggcacgcgcccgccacacagccagccgacacacccgcgacacacccgccacacccagccgcacccggcacacacccacccagccgcacccccgccacacccagaccgacgccggtgcgggaccggg

SEQ ID NO: 18 - protein, 5 aa, Alphapapillomavirus 9
LYCYE

SEQ ID NO: 19 - protein, 5 aa, Alphapapillomavirus 7
LLCHE

SEQ ID NO: 20 - protein, 5 aa, synthetic construct
LYGYG

SEQ ID NO: 21 - protein, 5 aa, synthetic construct
LLGHG

SEQ ID NO: 22 - protein, 6 aa, Alphapapillomavirus sp.
RRETQV

SEQ ID NO: 23 - protein, 266 aa, Alphapapillomavirus 7
MHGPKATLQDIVLHLEPQNEIPVDLLGHGQLSDSEEENDEIDGVNHQHLPARRAEPQRHTMLCMGCKCEARIELVVESSADDLRAFQQLFLNTLSFVGPWCASQQMARFEDPTRRPYKLPDLCTELNTSLQDIEITCVYCKTVLELTEVFEFAFKDLFVVYRDSIPHAACHKGIDFYSRIRELRHYSDSVYGDTLEKLTNTGLYNLLIRCLRCQKPLNPAEKLRHLNEKRRFHNIAGHYRGQGHSCCNRARQERLQRYPYDVPDYA

SEQ ID NO: 24 - protein, 243 aa, Alphapapillomavirus 9
MHGDTPTLHEYMLDLQPETTDLYGYGQLNDSSEEEDEIDGPAGQAEPDRAHYNIVTFGCKCDSTLRLCVQSTHVDICTLEDLLMGTLGIVGPICSQKPMFQDPQERPRKLPQLCTELQTTIHDIILECVYCKQQLLRREVYDFAFRDLCIVYRDGNPYAVCDKGLKFYSKISEYRHYCYSLYGTTLEQQYNKPLCDLLIRCINCQKPLCPEEKQRHLDKKQRFHNIRGRWTGRGMSCCRSSRT